SEQUENCE LISTING (694 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68 - protein, 193 aa, synthetic construct
MAAEPVEDNCINFVAMKFIDNTLYFIAEDDENLESDYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 69 - protein, 208 aa, synthetic construct
MAAEPVEDNCINFVAMKFIDNTLYFIAEDDENLESDSGGPGPAGMKGLPGSYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 70 - protein, 196 aa, synthetic construct
MAAEPVEDNCINFVAMKFIDNTLYFIAEDDENLESDPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 71 - protein, 196 aa, synthetic construct
MAAEPVEDNCINFVAMKFIDNTLYFIAEDDENLESDPLGMFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 72 - protein, 199 aa, synthetic construct
MAAEPVEDNCINFVAMKFIDNTLYFIAEDDENLESDPLGLAGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 73 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPGPAGMKGLPGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 74 - protein, 276 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEPLGLAGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 75 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPGPAGMKGLPGSNIVPVLLGPKLNHVAVELGKNVRLDCSALLNEEDVIYWMFGEEDGSDPNIHEEKEMRIMTPEGKWHASKVLRIENIGESNLNVLYDCTVASTGGTDTKSFILVRKADHHHHHH

SEQ ID NO: 76 - protein, 280 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNESGGPLGLAGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 77 - protein, 275 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEGGSGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 78 - protein, 284 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEGGGGSGGGGSGGGGSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADHHHHHH

SEQ ID NO: 79 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 80 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 81 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 82 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 83 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 84 - protein, 157 aa, synthetic construct
YFGKLESKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 85 - protein, 157 aa, synthetic construct
YFGKLKSKLSVIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 86 - protein, 157 aa, synthetic construct
YFGKLESKLSVIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 87 - protein, 52 aa, synthetic construct
TTNPGVSAWQVNTAYTAGQLVTYNGKTYKCLQPHTSLAGWEPSNVPALWQLQ

SEQ ID NO: 88 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 89 - protein, 36 aa, synthetic construct
MAAEPVEDNCINFVAMKFIDNTLYFIAEDDENLESD

SEQ ID NO: 90 - protein, 36 aa, synthetic construct
MAAEPVEDNSINFVAMKFIDNTLYFIAEDDENLESD

SEQ ID NO: 91 - protein, 36 aa, synthetic construct
MAAEPVEDNAINFVAMKFIDNTLYFIAEDDENLESD

SEQ ID NO: 92 - protein, 36 aa, synthetic construct
MAAEPVEDNCINFVAMKFIDNTLYFIAEDDENLPLG

SEQ ID NO: 93 - protein, 108 aa, synthetic construct
SNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKAD

SEQ ID NO: 94 - protein, 108 aa, synthetic construct
SNIVPVLLGPKLNHVAVELGKNVRLDCSALLNEEDVIYWMFGEEDGSDPNIHEEKEMRIMTPEGKWHASKVLRIENIGESNLNVLYDCTVASTGGTDTKSFILVRKAD

SEQ ID NO: 95 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 96 - protein, 36 aa, synthetic construct
MAAEPVEDNSINFVAMKFIDNTLYFIAEDDENLPLG

SEQ ID NO: 97 - protein, 36 aa, synthetic construct
MAAEPVEDNAINFVAMKFIDNTLYFIAEDDENLPLG

SEQ ID NO: 98 - protein, 108 aa, synthetic construct
SNIVPVLLGPKLNHVAVELGKNVRLNSSALLNEEDVIYWMFGEENGSDPNIHEEKEMRIMTPEGKWHASKVLRIENIGESNLNVLYNSTVASTGGTDTKSFILVRKAD

SEQ ID NO: 99 - protein, 108 aa, synthetic construct
SNIVPVLLGPKLNHVAVELGKNVRLDASALLNEEDVIYWMFGEEDGSDPNIHEEKEMRIMTPEGKWHASKVLRIENIGESNLNVLYDATVASTGGTDTKSFILVRKAD

SEQ ID NO: 100 - protein, 14 aa, synthetic construct
SGGPGPAGMKGLPG

SEQ ID NO: 101 - protein, 15 aa, synthetic construct
SGGPGPAGMKGLPGS

SEQ ID NO: 102 - protein, 16 aa, synthetic construct
EAGRSANHEPLGLVAT

SEQ ID NO: 103 - protein, 10 aa, synthetic construct
PQASTGRSGG

SEQ ID NO: 104 - protein, 10 aa, synthetic construct
PQGSTGRAAG

SEQ ID NO: 105 - protein, 10 aa, synthetic construct
PPASSGRAGG

SEQ ID NO: 106 - protein, 16 aa, synthetic construct
RSGVPLSLYSGSGGGK

SEQ ID NO: 107 - protein, 14 aa, synthetic construct
RSGMPYDLYHPSGK

SEQ ID NO: 108 - protein, 14 aa, synthetic construct
RGPDSGGFMLTSGK

SEQ ID NO: 109 - protein, 15 aa, synthetic construct
RGSGHEQLTVSGGSK

SEQ ID NO: 110 - protein, 14 aa, synthetic construct
RSGRAAAVKSPSGK

SEQ ID NO: 111 - protein, 20 aa, synthetic construct
RGSGISSGLLSGRSDNHSGK

SEQ ID NO: 112 - protein, 14 aa, synthetic construct
RGDLLAVVAASGGK

SEQ ID NO: 113 - protein, 15 aa, synthetic construct
RGGISSGLLSGRSGK

SEQ ID NO: 114 - protein, 15 aa, synthetic construct
SGGGKKLADEPEGGS

SEQ ID NO: 115 - protein, 13 aa, synthetic construct
GGGKFLADEPEGG

SEQ ID NO: 116 - protein, 8 aa, synthetic construct
ARLQSAAP

SEQ ID NO: 117 - protein, 14 aa, synthetic construct
ARLQSAAPAGLKGA

SEQ ID NO: 118 - protein, 17 aa, synthetic construct
GSGGPGPAGMHGLPGGS

SEQ ID NO: 119 - protein, 16 aa, synthetic construct
GGGSHTGRSAYFGGGS

SEQ ID NO: 120 - protein, 15 aa, synthetic construct
SGGPGPAGLKGAPGS

SEQ ID NO: 121 - protein, 8 aa, synthetic construct
VPLSLYSG

SEQ ID NO: 122 - protein, 11 aa, synthetic construct
SGLLSGRSDNH

SEQ ID NO: 123 - protein, 11 aa, synthetic construct
SGRSDNIGGGS

SEQ ID NO: 124 - protein, 15 aa, synthetic construct
LQESLRSKESGRSDI

SEQ ID NO: 125 - protein, 13 aa, synthetic construct
ISSGLLSGRSDNH

SEQ ID NO: 126 - protein, 8 aa, synthetic construct
LSGRSDDH

SEQ ID NO: 127 - protein, 13 aa, synthetic construct
ISSGLLSGRSDQH

SEQ ID NO: 128 - protein, 13 aa, synthetic construct
ISSGLLSGRSDNI

SEQ ID NO: 129 - protein, 6 aa, synthetic construct
PLGLAG

SEQ ID NO: 130 - protein, 8 aa, synthetic construct
GPAGMKGL

SEQ ID NO: 131 - protein, 8 aa, synthetic construct
LSGRSDQH

SEQ ID NO: 132 - protein, 8 aa, synthetic construct
LSGRSDNI

SEQ ID NO: 133 - protein, 13 aa, synthetic construct
ISSGLLSGRSDNH

SEQ ID NO: 134 - protein, 7 aa, synthetic construct
GPLGVRG

SEQ ID NO: 135 - protein, 7 aa, synthetic construct
GPLGLAR

SEQ ID NO: 136 - protein, 8 aa, synthetic construct
GPAALVGA

SEQ ID NO: 137 - protein, 8 aa, synthetic construct
GPAALIGG

SEQ ID NO: 138 - protein, 8 aa, synthetic construct
GPLNLVGR

SEQ ID NO: 139 - protein, 8 aa, synthetic construct
GPAGLVAD

SEQ ID NO: 140 - protein, 8 aa, synthetic construct
GPANLVAP

SEQ ID NO: 141 - protein, 8 aa, synthetic construct
VPLSLYSG

SEQ ID NO: 142 - protein, 10 aa, synthetic construct
SGESPAYYTA

SEQ ID NO: 143 - protein, 7 aa, synthetic construct
GGPRGLG

SEQ ID NO: 144 - protein, 6 aa, synthetic construct
HSSKLQ

SEQ ID NO: 145 - protein, 7 aa, synthetic construct
HSSKLQL

SEQ ID NO: 146 - protein, 8 aa, synthetic construct
KRALGLPG

SEQ ID NO: 147 - protein, 5 aa, synthetic construct
LEATA

SEQ ID NO: 148 - protein, 10 aa, synthetic construct
GGAANLVRGG

SEQ ID NO: 149 - protein, 10 aa, synthetic construct
SGRIGFLRTA

SEQ ID NO: 150 - protein, 5 aa, synthetic construct
PLGLA

SEQ ID NO: 151 - protein, 8 aa, synthetic construct
ESPAYYTA

SEQ ID NO: 152 - protein, 6 aa, synthetic construct
RLQLKL

SEQ ID NO: 153 - protein, 7 aa, synthetic construct
RLQLKAC

SEQ ID NO: 154 - protein, 5 aa, synthetic construct
SGRSA

SEQ ID NO: 155 - skipped

SEQ ID NO: 156 - protein, 5 aa, synthetic construct
GGGRR

SEQ ID NO: 157 - protein, 4 aa, synthetic construct
GFLG

SEQ ID NO: 158 - protein, 9 aa, synthetic construct
HSSKLQEDA

SEQ ID NO: 159 - protein, 10 aa, synthetic construct
LVLASSSFGY

SEQ ID NO: 160 - protein, 10 aa, synthetic construct
GVSQNYPIVG

SEQ ID NO: 161 - protein, 10 aa, synthetic construct
GVVQASCRLA

SEQ ID NO: 162 - protein, 6 aa, synthetic construct
DPRSFL

SEQ ID NO: 163 - protein, 6 aa, synthetic construct
PPRSFL

SEQ ID NO: 164 - protein, 4 aa, synthetic construct
DEVD

SEQ ID NO: 165 - protein, 5 aa, synthetic construct
DEVDP

SEQ ID NO: 166 - protein, 8 aa, synthetic construct
KGSGDVEG

SEQ ID NO: 167 - protein, 6 aa, synthetic construct
GWEHDG

SEQ ID NO: 168 - protein, 7 aa, synthetic construct
EDDDDKA

SEQ ID NO: 169 - protein, 9 aa, synthetic construct
KQEQNPGST

SEQ ID NO: 170 - protein, 6 aa, synthetic construct
GKAFRR

SEQ ID NO: 171 - protein, 4 aa, synthetic construct
DAFK

SEQ ID NO: 172 - protein, 4 aa, synthetic construct
DVLK

SEQ ID NO: 173 - protein, 4 aa, synthetic construct
DAFK

SEQ ID NO: 174 - protein, 7 aa, synthetic construct
ALLLALL

SEQ ID NO: 175 - protein, 10 aa, synthetic construct
SGAKPRALTA

SEQ ID NO: 176 - protein, 10 aa, synthetic construct
SGLRLAAITA

SEQ ID NO: 177 - protein, 10 aa, synthetic construct
SGESLAYYTA

SEQ ID NO: 178 - protein, 10 aa, synthetic construct
SGESPAYYTA

SEQ ID NO: 179 - protein, 10 aa, synthetic construct
SGESLRYYTA

SEQ ID NO: 180 - protein, 10 aa, synthetic construct
SGRSLSRLTA

SEQ ID NO: 181 - protein, 10 aa, synthetic construct
SGRSLRRLTA

SEQ ID NO: 182 - protein, 10 aa, synthetic construct
SGAVSWLLTA

SEQ ID NO: 183 - protein, 10 aa, synthetic construct
SGAPSWLLTA

SEQ ID NO: 184 - protein, 10 aa, synthetic construct
SGAVRWLLTA

SEQ ID NO: 185 - protein, 10 aa, synthetic construct
SGANISDLTA

SEQ ID NO: 186 - protein, 10 aa, synthetic construct
SGNRYSSLTA

SEQ ID NO: 187 - protein, 10 aa, synthetic construct
SGHMHKALTA

SEQ ID NO: 188-189 - skipped (2 entries)

SEQ ID NO: 190 - protein, 12 aa, synthetic construct
GEEGEEPLGLAG

SEQ ID NO: 191 - protein, 7 aa, synthetic construct
GPLGLAG

SEQ ID NO: 192 - protein, 16 aa, synthetic construct
EAGRSANHTPAGLTGP

SEQ ID NO: 193 - protein, 17 aa, synthetic construct
GEAGRSANHTPAGLTGP

SEQ ID NO: 194-231 - skipped (38 entries)

SEQ ID NO: 232 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 234 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 448 aa, synthetic construct
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 240-241 - skipped (2 entries)

SEQ ID NO: 242 - protein, 118 aa, synthetic construct
QGQLQESGPSLVKPSQTLSLTCTVSGDSITRGYWNWIRKHPGKGLEYIGYISYTGSTYSNLSLKSRVTISRDTSKNQYYLKLSSVTAADTAVYYCATSTGWLDPVDYWGQGTLVTVSS

SEQ ID NO: 243 - protein, 107 aa, synthetic construct
DIVMTQSPDSLAVSLGERATINCKASQNVDTSVAWFQQKPGQPPKALIYSASFRYSGVPDRFSGSGSGTDFTLTISSLQAEDVAVYFCQQYYGYPFTFGQGTKLEIK

SEQ ID NO: 244 - protein, 123 aa, synthetic construct
EVQLVQSGGGLVKPGGSLRLSCAASGFTFSSYTMNWVRQAPGKGLEWVSSISSGSDYLYYADSVKGRFTISRDNAKNSLYLQMNSLRAEDTAVYYCARNELRWYPQAGAFDRWGQGTMVTVSS

SEQ ID NO: 245 - protein, 109 aa, synthetic construct
QSVVTQPPSMSAAPGQRVTISCSGSSSYIESSYVGWYQQLPGTAPRLLIYDDDMRPSGIPDRFSGSKSGTSATLAITGLQTGDEADYYCEIWRSGLGGVFGGGTKLTVL

SEQ ID NO: 246-247 - skipped (2 entries)

SEQ ID NO: 248 - protein, 453 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 216 aa, synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 250 - protein, 5 aa, synthetic construct
SYTMN

SEQ ID NO: 251 - protein, 17 aa, synthetic construct
SISSGSDYLYYADSVKG

SEQ ID NO: 252 - protein, 14 aa, synthetic construct
NELRWYPQAGAFDR

SEQ ID NO: 253 - protein, 13 aa, synthetic construct
SGSSSYIESSYVG

SEQ ID NO: 254 - protein, 7 aa, synthetic construct
DDDMRPS

SEQ ID NO: 255 - protein, 10 aa, synthetic construct
EIWRSGLGGV

SEQ ID NO: 256 - protein, 360 aa, synthetic construct
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSEPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 759 aa, synthetic construct
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 258 - protein, 942 aa, synthetic construct
QMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSEVQLQQSGPELVKPGPSMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSQMQLVQSGAEVKKPGSSVKVSCKASGGTFSSYAISWVRQAPGQGLEWMGRIIPILGIANYAQKFQGRVTITADKSTSTAYMELSSLRSEDTAVYYCAKPRDGYNLVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRQLVLTQPPSVSGAPGQRVTISCTGSSSNIGAGYDVHWYQQLPGAAPKLLIYGDINRPSGVPDRFSGSKSGISASLAITGLQAEDEADYYCQSYDSSLSGGVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - skipped

SEQ ID NO: 260 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 261 - protein, 48 aa, synthetic construct
QETNPTENLYFQQKNMQCQRRFYEALHDPNLNEEQRNARIRSIRDDDC

SEQ ID NO: 262 - protein, 61 aa, synthetic construct
QTADNQKNMQCQRRFYEALHDPNLNEEQRNARIRSIRDDCSQSANLLAEAQQLNDAQAPQA

SEQ ID NO: 263 - protein, 38 aa, synthetic construct
QETKNMQCQRRFYEALHDPNLNEEQRNARIRSIRDDDC

SEQ ID NO: 264 - protein, 38 aa, synthetic construct
QETFNKQCQRRFYEALHDPNLNEEQRNARIRSIRDDDC

SEQ ID NO: 265 - protein, 38 aa, synthetic construct
QETFNMQCQRRFYEALHDPNLNKEQRNARIRSIRDDDC

SEQ ID NO: 266 - protein, 37 aa, synthetic construct
QETFNMQCQRRFYEALHDPNLNEEQRNARIRSIKDDC

SEQ ID NO: 267 - protein, 35 aa, synthetic construct
QETMQCQRRFYEALHDPNLNEEQRNARIRSIKDDC

SEQ ID NO: 268 - protein, 34 aa, synthetic construct
QETQCQRRFYEALHDPNLNEEQRNARIRSIKDDC

SEQ ID NO: 269 - protein, 33 aa, synthetic construct
QETCQRRFYEALHDPNLNEEQRNARIRSIKDDC

SEQ ID NO: 270 - protein, 20 aa, synthetic construct
QETRGNCAYHKGQLVWCTYH

SEQ ID NO: 271 - protein, 20 aa, synthetic construct
QETRGNCAYHKGQIIWCTYH

SEQ ID NO: 272 - protein, 6 aa, synthetic construct
  VARIANT 6: note = A or P
LLQGPX

SEQ ID NO: 273 - protein, 8 aa, synthetic construct
GGLLQGPP

SEQ ID NO: 274-329 - skipped (56 entries)

SEQ ID NO: 330 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 331 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 332 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 333 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 335 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 339 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 341 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342-345 - skipped (4 entries)

SEQ ID NO: 346 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 349 - protein, 111 aa, synthetic construct [sequence omitted]

SEQ ID NO: 350 - protein, 113 aa, synthetic construct [sequence omitted]

SEQ ID NO: 351 - protein, 107 aa, synthetic construct [sequence omitted]

SEQ ID NO: 352 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 354 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 355 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 357 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 443 aa, synthetic construct
QVQLVESGGGLVKPGGSLRLSCAASGFTFSNYGMSWIRQAPGKGLEWSTISGGGSNIYYADSVKGRFTISRDNAKNSLYLQMNSLRAEDTAVYYCVSYYYGIDFWGQGTSVTVSSASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 361 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 365 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 369 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 370 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 373 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 374 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 375 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 376 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 377 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 378 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380 - protein, 5 aa, synthetic construct
NYYMY

SEQ ID NO: 381 - protein, 17 aa, synthetic construct
GINPSNGGTNFNEKFKN

SEQ ID NO: 382 - protein, 11 aa, synthetic construct
RDYRFDMGFDY

SEQ ID NO: 383 - protein, 15 aa, synthetic construct
RASKGVSTSGYSYLH

SEQ ID NO: 384 - protein, 7 aa, synthetic construct
LASYLES

SEQ ID NO: 385 - protein, 9 aa, synthetic construct
QHSRDLPLT

SEQ ID NO: 386 - protein, 5 aa, synthetic construct
NSGMH

SEQ ID NO: 387 - protein, 17 aa, synthetic construct
VIWYDGSKRYYADSVKG

SEQ ID NO: 388 - protein, 4 aa, synthetic construct
NDDY

SEQ ID NO: 389 - protein, 11 aa, synthetic construct
RASQSVSSYLA

SEQ ID NO: 390 - protein, 7 aa, synthetic construct
DASNRAT

SEQ ID NO: 391 - protein, 9 aa, synthetic construct
QQSSNWPRT

SEQ ID NO: 392 - protein, 9 aa, synthetic construct
FTFSNYGMS

SEQ ID NO: 393 - protein, 10 aa, synthetic construct
TISGGGSNIY

SEQ ID NO: 394 - protein, 9 aa, synthetic construct
VSYYYGIDF

SEQ ID NO: 395 - protein, 11 aa, synthetic construct
KASQDVTTAVA

SEQ ID NO: 396 - protein, 7 aa, synthetic construct
WASTRHT

SEQ ID NO: 397 - protein, 9 aa, synthetic construct
QQHYTIPWT

SEQ ID NO: 398 - protein, 8 aa, synthetic construct
GLTFSSSG

SEQ ID NO: 399 - protein, 8 aa, synthetic construct
IWYDGSKR

SEQ ID NO: 400 - protein, 6 aa, synthetic construct
ATNNDY

SEQ ID NO: 401 - protein, 11 aa, synthetic construct
RASQSVSSYLA

SEQ ID NO: 402 - protein, 7 aa, synthetic construct
TASNRAT

SEQ ID NO: 403 - protein, 9 aa, synthetic construct
QQYSNWPRT

SEQ ID NO: 404 - protein, 447 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 405 - protein, 218 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 406-423 - skipped (18 entries)

SEQ ID NO: 424 - protein, 20 aa, synthetic construct
  VARIANT 1..20: note = This sequence may encompass 1-10 GS repeating units
GSGSGSGSGSGSGSGSGSGS

SEQ ID NO: 425 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 426 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 427 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 428 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 429 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 430 - protein, 15 aa, synthetic construct
GGGGSGGGGSGGGGS

SEQ ID NO: 431 - protein, 20 aa, synthetic construct
GGGGSGGGGSGGGGSGGGGS

SEQ ID NO: 432 - protein, 14 aa, synthetic construct
GGGGSGGGGSGGGG

SEQ ID NO: 433-500 - skipped (68 entries)

SEQ ID NO: 501 - protein, 8 aa, synthetic construct
KRALGLPG

SEQ ID NO: 502 - skipped

SEQ ID NO: 503 - protein, 5 aa, synthetic construct
LEATA

SEQ ID NO: 504 - protein, 10 aa, synthetic construct
GGAANLVRGG

SEQ ID NO: 505 - protein, 10 aa, synthetic construct
SGRIGFLRTA

SEQ ID NO: 506 - protein, 6 aa, synthetic construct
PLGLAG

SEQ ID NO: 507 - protein, 6 aa, synthetic construct
  VARIANT 6: note = Any amino acid
PLGLAX

SEQ ID NO: 508 - protein, 8 aa, synthetic construct
ESPAYYTA

SEQ ID NO: 509 - protein, 6 aa, synthetic construct
RLQLKL

SEQ ID NO: 510 - protein, 7 aa, synthetic construct
RLQLKAC

SEQ ID NO: 511 - protein, 5 aa, synthetic construct
SGRSA

SEQ ID NO: 512 - protein, 4 aa, synthetic construct
DAFK

SEQ ID NO: 513 - protein, 5 aa, synthetic construct
GGGRR

SEQ ID NO: 514 - protein, 4 aa, synthetic construct
GFLG

SEQ ID NO: 515 - protein, 4 aa, synthetic construct
ALAL

SEQ ID NO: 516-517 - skipped (2 entries)

SEQ ID NO: 518 - protein, 5 aa, synthetic construct
GLLYA

SEQ ID NO: 519 - protein, 8 aa, synthetic construct
GGPRGLPG

SEQ ID NO: 520 - protein, 6 aa, synthetic construct
HSSKLQ

SEQ ID NO: 521 - protein, 7 aa, synthetic construct
HSSKLQL

SEQ ID NO: 522 - protein, 9 aa, synthetic construct
HSSKLQEDA

SEQ ID NO: 523 - protein, 6 aa, synthetic construct
DPRSFL

SEQ ID NO: 524 - protein, 6 aa, synthetic construct
PPRSFL

SEQ ID NO: 525 - protein, 4 aa, synthetic construct
DEVD

SEQ ID NO: 526 - protein, 5 aa, synthetic construct
DEVDP

SEQ ID NO: 527 - protein, 8 aa, synthetic construct
KGSGDVEG

SEQ ID NO: 528 - protein, 6 aa, synthetic construct
GWEHDG

SEQ ID NO: 529 - protein, 9 aa, synthetic construct
KQEQNPGST

SEQ ID NO: 530 - protein, 6 aa, synthetic construct
GKAFRR

SEQ ID NO: 531 - protein, 4 aa, synthetic construct
DAFK

SEQ ID NO: 532 - protein, 4 aa, synthetic construct
DVLK

SEQ ID NO: 533 - protein, 4 aa, synthetic construct
DAFK

SEQ ID NO: 534 - protein, 7 aa, synthetic construct
ALLLALL

SEQ ID NO: 535-578 - skipped (44 entries)

SEQ ID NO: 579 - protein, 196 aa, synthetic construct
MAAEPVEDNAINFVAMKFIDNTLYFIAEDDENLESDPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 580 - protein, 196 aa, synthetic construct
MAAEPVEDNSINFVAMKFIDNTLYFIAEDDENLESDPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 581 - protein, 202 aa, synthetic construct
MAAEPVEDNSINFVAMKFIDNTLYFIAEDDENLESDSGRVANLVGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 582 - protein, 203 aa, synthetic construct
MAAEPVEDNSINFVAMKFIDNTLYFIAEDDENLESDRGRRVANLVGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 583 - protein, 199 aa, synthetic construct
MAAEPVEDNSINFVAMKFIDNTLYFIAEDDENLESDSGRVANYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 584 - protein, 200 aa, synthetic construct
MAAEPVEDNSINFVAMKFIDNTLYFIAEDDENLESDRGRRVANYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 585 - protein, 199 aa, synthetic construct
MAAEPVEDNSINFVAMKFIDNTLYFIAEDDENLESDSGRPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 586 - protein, 200 aa, synthetic construct
MAAEPVEDNSINFVAMKFIDNTLYFIAEDDENLESDRGRRPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 587 - protein, 205 aa, synthetic construct
MAAEPVEDNSINFVAMKFIDNTLYFIAEDDENLESDSGRVANLVGGSGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 588 - protein, 206 aa, synthetic construct
MAAEPVEDNSINFVAMKFIDNTLYFIAEDDENLESDRGRRVANLVGGSGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 589 - protein, 203 aa, synthetic construct
MAAEPVEDNSINFVAMKFIDNTLYFIAEDDENLESDRGRRVANPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 590 - protein, 206 aa, synthetic construct
MAAEPVEDNSINFVAMKFIDNTLYFIAEDDENLESDRGRRPLGLAGGSGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 591 - protein, 208 aa, synthetic construct
MAAEPVEDNSINFVAMKFIDNTLYFIAEDDENLESDRGRRVANPLGLAGSGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 592 - protein, 199 aa, synthetic construct
MAAEPVEDNSINFVAMKFIDNTLYFIAEDDENLESDSGRPLGYFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 593 - protein, 199 aa, synthetic construct
MAAEPVEDNSINFVAMKFIDNTLYFIAEDDENLESDSGRPLGYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 594 - protein, 199 aa, synthetic construct
MAAEPVEDNSINFVAMKFIDNTLYFIAEDDENLESDSGRPLGYFGKLESKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 595 - protein, 199 aa, synthetic construct
MAAEPVEDNSINFVAMKFIDNTLYFIAEDDENLESDSGRPLGYFGKLKSKLSVIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 596 - protein, 199 aa, synthetic construct
MAAEPVEDNSINFVAMKFIDNTLYFIAEDDENLESDSGRPLGYFGKLESKLSVIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 597 - protein, 193 aa, synthetic construct
MAAEPVEDNSINFVAMKFIDNTLYFIAEDDENLESDYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 598 - protein, 217 aa, synthetic construct
  VARIANT 215..217: note = This region may encompass either of the following sequences: GGH or GGK-24x polyethylene glycol azide
MAAEPVEDNSINFVAMKFIDNTLYFIAEDDENLESDRGRRPLGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNEDGGGGSGGGGSLPETXXX

SEQ ID NO: 599-600 - skipped (2 entries)

SEQ ID NO: 601 - protein, 17 aa, synthetic construct
SSRGPASNRRLPLGLAG

SEQ ID NO: 602 - protein, 17 aa, synthetic construct
SSRAVFRKNLGPLGLAG

SEQ ID NO: 603 - protein, 15 aa, synthetic construct
SGRVLRKAGPQPLVD

SEQ ID NO: 604 - protein, 16 aa, synthetic construct
SGRVLTLRKAPWGLLE

SEQ ID NO: 605 - protein, 12 aa, synthetic construct
SGRVLGPAGLVG

SEQ ID NO: 606 - protein, 12 aa, synthetic construct
SGRVLGPANLVG

SEQ ID NO: 607 - protein, 11 aa, synthetic construct
SGRVLPAGLVG

SEQ ID NO: 608 - protein, 12 aa, synthetic construct
SGRVLGPLNLSG

SEQ ID NO: 609 - protein, 12 aa, synthetic construct
RQRRSAPLGLAG

SEQ ID NO: 610 - protein, 11 aa, synthetic construct
RQRRSPLGLAG

SEQ ID NO: 611 - protein, 17 aa, synthetic construct
SGRVLTLRKAGPAGLVG

SEQ ID NO: 612 - protein, 17 aa, synthetic construct
SGRVLTLRKAGPANLVG

SEQ ID NO: 613 - protein, 15 aa, synthetic construct
SGRVLRKAGPAGLVG

SEQ ID NO: 614 - protein, 15 aa, synthetic construct
SGRVLRKAGPANLVG

SEQ ID NO: 615 - protein, 11 aa, synthetic construct
SGRVLPANLVG

SEQ ID NO: 616 - protein, 9 aa, synthetic construct
SGRVAGLVG

SEQ ID NO: 617 - protein, 9 aa, synthetic construct
SGRVANLVG

SEQ ID NO: 618 - protein, 13 aa, synthetic construct
PASNRRLPLGLAG

SEQ ID NO: 619 - protein, 13 aa, synthetic construct
SSRVFRKPANLAG

SEQ ID NO: 620 - protein, 16 aa, synthetic construct
SGRVLTLRKAALPLAM

SEQ ID NO: 621 - protein, 13 aa, synthetic construct
SSRGRRGPYMLQG

SEQ ID NO: 622 - protein, 10 aa, synthetic construct
SSRGPYMLQG

SEQ ID NO: 623 - protein, 11 aa, synthetic construct
SGRVLPLGMRA

SEQ ID NO: 624 - protein, 11 aa, synthetic construct
SGRVLPYAMTA

SEQ ID NO: 625 - protein, 12 aa, synthetic construct
KWGKSAPLGLAG

SEQ ID NO: 626 - protein, 12 aa, synthetic construct
RYGKSAPLGLAG

SEQ ID NO: 627 - protein, 13 aa, synthetic construct
RQRRSAAPLGLAG

SEQ ID NO: 628 - protein, 9 aa, synthetic construct
RQRRSVVGG

SEQ ID NO: 629 - protein, 8 aa, synthetic construct
SPLGLAGS

SEQ ID NO: 630 - protein, 10 aa, synthetic construct
RGRKVANLVG

SEQ ID NO: 631 - protein, 10 aa, synthetic construct
RQRKVANLVG

SEQ ID NO: 632 - protein, 10 aa, synthetic construct
RGRRVANLVG

SEQ ID NO: 633 - protein, 11 aa, synthetic construct
RGRKSPANLVG

SEQ ID NO: 634 - protein, 10 aa, synthetic construct
RGRKPYMLQG

SEQ ID NO: 635 - protein, 11 aa, synthetic construct
RGRKSPYMLQG

SEQ ID NO: 636 - protein, 10 aa, synthetic construct
RGRKPQPLVD

SEQ ID NO: 637 - protein, 11 aa, synthetic construct
RGRKSPQPLVD

SEQ ID NO: 638 - protein, 10 aa, synthetic construct
RGRKSQPLVD

SEQ ID NO: 639 - protein, 11 aa, synthetic construct
SGRVAPYMLQG

SEQ ID NO: 640 - protein, 9 aa, synthetic construct
SGRVYMLQG

SEQ ID NO: 641 - protein, 11 aa, synthetic construct
SGRVAPQPLVD

SEQ ID NO: 642 - protein, 9 aa, synthetic construct
SGRVQPLVD

SEQ ID NO: 643 - protein, 6 aa, synthetic construct
RGRRGP

SEQ ID NO: 644 - protein, 6 aa, synthetic construct
PLGLAG

SEQ ID NO: 645 - skipped

SEQ ID NO: 646 - protein, 9 aa, synthetic construct
SGRVANLVG

SEQ ID NO: 647 - protein, 12 aa, synthetic construct
SGRVANLVGGSG

SEQ ID NO: 648 - protein, 10 aa, synthetic construct
RGRRVANLVG

SEQ ID NO: 649 - protein, 13 aa, synthetic construct
RGRRVANLVGGSG

SEQ ID NO: 650 - protein, 6 aa, synthetic construct
SGRVAN

SEQ ID NO: 651 - protein, 7 aa, synthetic construct
RGRRVAN

SEQ ID NO: 652 - protein, 6 aa, synthetic construct
SGRPLG

SEQ ID NO: 653 - protein, 7 aa, synthetic construct
RGRRPLG

SEQ ID NO: 654 - protein, 9 aa, synthetic construct
SGRPLGLAG

SEQ ID NO: 655 - protein, 10 aa, synthetic construct
RGRRPLGLAG

SEQ ID NO: 656 - protein, 13 aa, synthetic construct
RGRRVANPLGLAG

SEQ ID NO: 657 - protein, 15 aa, synthetic construct
RGRRVANPLGLAGSG

SEQ ID NO: 658 - protein, 13 aa, synthetic construct
RGRRPLGLAGGSG

SEQ ID NO: 659 - protein, 10 aa, synthetic construct
RGRRVANPLG

SEQ ID NO: 660 - protein, 13 aa, synthetic construct
RGRRVANPLGGSG

SEQ ID NO: 661 - protein, 13 aa, synthetic construct
RGRRSGRVANPLG

SEQ ID NO: 662 - protein, 19 aa, synthetic construct
RGRRSGRAANLVRPLGLAG

SEQ ID NO: 663 - protein, 6 aa, synthetic construct
AANLVR

SEQ ID NO: 664 - protein, 16 aa, synthetic construct
RGRRSGRAANLVRPLG

SEQ ID NO: 665 - protein, 13 aa, synthetic construct
RGRRAANLVRPLG

SEQ ID NO: 666 - protein, 16 aa, synthetic construct
RGRRAANLVRPLGLAG

SEQ ID NO: 667 - protein, 10 aa, synthetic construct
RGRRAANLVR

SEQ ID NO: 668 - protein, 13 aa, synthetic construct
RGRRSGRAANLVR

SEQ ID NO: 669 - protein, 6 aa, synthetic construct
HSSKLQ

SEQ ID NO: 670 - protein, 10 aa, synthetic construct
RGRRHSSKLQ

SEQ ID NO: 671 - protein, 13 aa, synthetic construct
RGRRSGRHSSKLQ

SEQ ID NO: 672 - protein, 13 aa, synthetic construct
RGRRHSSKLQPLG

SEQ ID NO: 673 - protein, 16 aa, synthetic construct
RGRRSGRHSSKLQPLG

SEQ ID NO: 674 - protein, 13 aa, synthetic construct
RGRRHSSKLQVAN

SEQ ID NO: 675 - protein, 16 aa, synthetic construct
RGRRSGRHSSKLQVAN

SEQ ID NO: 676 - protein, 12 aa, synthetic construct
SGRVANPLGLAG

SEQ ID NO: 677 - protein, 14 aa, synthetic construct
SGRVANPLGLAGSG

SEQ ID NO: 678 - protein, 12 aa, synthetic construct
SGRPLGLAGGSG

SEQ ID NO: 679 - protein, 9 aa, synthetic construct
SGRVANPLG

SEQ ID NO: 680 - protein, 12 aa, synthetic construct
SGRVANPLGGSG

SEQ ID NO: 681 - protein, 12 aa, synthetic construct
SGRAANLVRPLG

SEQ ID NO: 682 - protein, 15 aa, synthetic construct
SGRAANLVRPLGLAG

SEQ ID NO: 683 - protein, 9 aa, synthetic construct
SGRAANLVR

SEQ ID NO: 684 - protein, 9 aa, synthetic construct
SGRHSSKLQ

SEQ ID NO: 685 - protein, 12 aa, synthetic construct
SGRHSSKLQPLG

SEQ ID NO: 686 - protein, 12 aa, synthetic construct
SGRHSSKLQVAN

SEQ ID NO: 687 - protein, 6 aa, synthetic construct
HHHHHH

SEQ ID NO: 688 - protein, 8 aa, synthetic construct
WSHPQFEK

SEQ ID NO: 689 - protein, 9 aa, synthetic construct
AWAHPQPGG

SEQ ID NO: 690 - protein, 5 aa, synthetic construct
GGSGG

SEQ ID NO: 691 - protein, 5 aa, synthetic construct
GGGGS

SEQ ID NO: 692 - protein, 17 aa, synthetic construct
  SITE 1: note = Acetylated residue
  SITE 8: note = 3-((3-((2,5-dioxopyrrolidin-1-yl)oxy)-3-oxopropyl)d isulfaneyl)propanoate modified residue
  SITE 17: note = Amidated residue
  SITE 4..14: note = Intrachain disulfide bond
RGNCAYHKGQLVWCTYH

SEQ ID NO: 693 - protein, 17 aa, synthetic construct
  SITE 1: note = Acetylated residue
  SITE 8: note = 4-oxo-4-((3-oxo-3-(phenylthio)propyl)thio)butanoate modified residue
  SITE 17: note = Amidated residue
  SITE 4..14: note = Intrachain disulfide bond
RGNCAYHKGQLVWCTYH

SEQ ID NO: 694 - protein, 34 aa, synthetic construct
  SITE 1: note = Acetylated residue
  SITE 31: note = 3-(((3-oxo-3-(phenylthio)propyl)thio)carbonyl)benzo ate modified residue
  SITE 34: note = Amidated residue
  SITE 5..34: note = Intrachain disulfide bond
FNMQCQRRFYEALHDPNLNEEQRNARIRSIKDDC

(19) United States
(12) Patent Application Publication
(10) Pub. No.: US 2025/0197467 A1
(43) Pub. Date: Jun. 19, 2025

(54) ACTIVATABLE IL-18 IMMUNOCYTOKINES AND USES THEREOF

(71) Applicant: Bright Peak Therapeutics AG, Allschwil (CH)

(72) Inventors: Vijaya Raghavan PATTABIRAMAN, Volketswil (CH); Bertolt KREFT, Kleinmachnow (DE); Arnaud GOEPFERT, Sierentz (FR); Tiziano ONGARO, Basel (CH); Jean-Philippe CARRALOT, Saint-Louis (FR); Kea MARTIN, Istein (DE)

(21) Appl. No.: 18/813,104

(22) Filed: Aug. 23, 2024

Related U.S. Application Data
(60) Provisional application No. 63/534,273, filed on Aug. 23, 2023.

(51) Int. Cl. C07K 14/54; A61K 47/68; A61P 35/00; C07K 16/28

(52) U.S. Cl. CPC C07K 14/54; A61K 47/6873; A61P 35/00; C07K 16/2818; C07K 2317/92; C07K 2319/30; C07K 2319/50

(57) ABSTRACT

The present disclosure relates to activatable IL-18 immunocytokine compositions comprising antibodies or antigen binding fragments specific for immune cell antigens and activatable IL-18 polypeptides. Also provided herein are methods of treatment with and methods of manufacture of activatable immunocytokine compositions. Activatable IL-18 polypeptides usable in immunocytokines are also encompassed by the present disclosure.

### CROSS REFERENCE

This application claims the benefit of U.S. Provisional Application No. 63/534,273 filed Aug. 23, 2023, which application is incorporated herein by reference in its entirety.

### SEQUENCE LISTING

The instant application contains a Sequence Listing which has been submitted electronically in XML format and is hereby incorporated by reference in its entirety. Said XML copy, created on Aug. 21, 2024, is named 94917-0156_737201US_SL.xml and is 490,306 bytes in size.

### BRIEF SUMMARY

Described herein are activatable immunocytokine compositions which contain an antibody or antigen binding fragment thereof, an activatable interleukin 18 (Act-IL-18) polypeptide and a linker. The antibody or antigen binding fragment thereof can be specific for (e.g., binds specifically to) an immune cell associated antigen. In some embodiments, the Act-IL-18 polypeptide comprises an artificial polypeptide attached to the base sequence of a mature IL-18 which, when attached, lowers the IL-18-related activity of the molecule. In some embodiments, the artificial polypeptide is cleaved by a protease, such as a protease upregulated in a tumor microenvironment, thereby removing all or a portion of the artificial polypeptide and enhancing the IL-18 related activity (e.g., IL-18 receptor signaling) of the molecule. In some embodiments, an activatable immunocytokine may exhibit fewer side effects or off-target effects compared to a non-activatable immunocytokine owing to the lessened activity of the IL-18 portion of the molecule before cleavage activation, thus providing advantages in some contexts over other immunocytokines.

An exemplary, non-limiting mechanism of action of an activatable immunocytokine provided herein is shown in FIG. 1. In the exemplary embodiment, the immunocytokine composition comprises an anti-PD-1 antibody as the antibody or antigen binding fragment of the activatable immunocytokine. In this embodiment, the anti-PD-1 antibody portion of the immunocytokine selectively binds to PD-1 present on the surface of an activated T cell (e.g., a CD8⁺ T cell). This binding prevents the checkpoint interaction of PD-1 and PD-L1/2, thus preventing attenuation of activity of the T cell. Concomitantly, after activation of the IL-18 portion of the immunocytokine by tumor microencironment proteases, the IL-18 concentration is effectively in a high local concentration near the T-cell due to the linkage, further activates the T cell through IL-18R signaling. The activatable immunocytokine shown in this figure is depicted with two activatable IL-18 polypeptides attached to the antibody (DAR2), however activatable immunocytokines having only a single activatable IL-18 polypeptide attached (DAR1) are also expressly contemplated as within the scope of the instant disclosure. While the exemplary embodiment shows the mechanism of action of an anti-PD-1 antibody, antibodies or antigen binding fragments specific for other immune antigens provided herein can function according to a similar mechanism. Further descriptions of activatable IL-18 polypeptides by themselves are described elsewhere in the instant disclosure.

In one aspect, provided herein, is an activatable immunocytokine composition, comprising: an Act-IL-18 polypeptide; an antibody or an antigen binding fragment thereof; and a linker, wherein the linker comprises: a first point of attachment to the antibody or antigen binding fragment thereof; and a second point of attachment to the Act-IL-18 polypeptide. The antibody or antigen binding fragment thereof can be specific for an immune cell associated antigen.

The Act-IL-18 polypeptide can comprise an artificial polypeptide attached to an IL-18 polypeptide. The artificial polypeptide can comprise a protease cleavage site. Cleavage at the protease cleavage site can convert the Act-IL-18 into an active form of the IL-18 polypeptide. In some embodiments, when the artificial polypeptide is intact, the Act-IL-18 is inactive.

The Act-IL-18 polypeptide can be activated in response to certain conditions or stimuli found in a target area of a subject after administration. In some embodiments, the artificial polypeptide is cleaved at the protease cleavage site under one or more conditions associated with a desired target area (e.g., a tumor microenvironment), thus releasing an active IL-18 polypeptide. In some embodiments, the protease cleavage site is preferentially cleaved at or near a target tissue of a subject. In some embodiments, the protease cleavage site is preferentially cleaved in or near a tumor microenvironment. In some embodiments, cleavage at the protease cleavage site enhances an IL-18 related activity of the activatable immunocytokine. In some embodiments, cleavage at the protease cleavage site converts the Act-IL-18 polypeptide into an activated form of the IL-18 polypeptide with an enhanced IL-18 related activity.

In some embodiments, the specific cleavage site is specifically cleaved by a protease. In some embodiments, the protease is found at higher concentrations and/or demonstrates higher proteolytic activity within the tumor microenvironment relative to non-tumor tissue. In some embodiments, the protease is selected from: kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, and a tumor cell surface protease.

In some embodiments, the Act-IL-18 polypeptide comprises a protease recognition sequence comprising the protease cleavage site. In some embodiments, the protease recognition sequence has a sequence at least 80%, at least 85%, at least 90%, at least 95%, at least 96%, at least 97%, at least 98%, at least 99%, or 100% identical to a peptide sequence set forth in Table 2A or 2B. In some embodiments, the protease recognition sequence comprises the amino acid sequence set forth in any one of SEQ ID NOs: 646, 647, 648, 649, 650, 651, 652, 653, 657, 658, or 659.

In some embodiments, the cleavage at the protease cleavage site removes the entire artificial polypeptide from the IL-18 polypeptide. In some embodiments, the cleavage results in a portion of the artificial moiety remaining attached to the IL-18 polypeptide. In some embodiments, cleavage of the artificial polypeptide at the protease cleavage site leaves no amino acid residues of the artificial polypeptide attached to the IL-18 polypeptide. In some embodiments, cleavage of the artificial polypeptide at the protease cleavage site leaves 1, 2, 3, 4, 5, 6, 7, 8, 9, or 10 amino acid residues of the artificial polypeptide attached to the IL-18 polypeptide.

In some embodiments, the artificial polypeptide comprises multiple protease cleavage sites. In some embodiments, the artificial polypeptide comprises 2, 3, or 4 protease cleavage sites.

In some embodiments, the IL-18 polypeptide comprises substitutions of one or more amino acids at or near the N- or C-terminus of the IL-18 polypeptide which form part of a recognition sequence for the protease cleavage site. In some embodiments, the substitutions of the one or more amino acids at or near the N- or C-terminus of the IL-18 polypeptide comprises amino acid substitutions of residues 1, 2, and/or 3 of the IL-18 polypeptide, wherein residue position numbering is based on SEQ ID NO: 1 as a reference sequence

In some embodiments, cleavage of the artificial polypeptide at the protease cleavage site leaves no amino acid residues of the artificial polypeptide attached to the IL-18 polypeptide. In some embodiments, cleavage of the artificial polypeptide at the protease cleavage site leaves at least 1 amino acid residue of the artificial polypeptide attached to the IL-18 polypeptide. In some embodiments, cleavage of the artificial polypeptide at the protease cleavage site leaves 1, 2, 3, 4, 5, 6, 7, 8, 9, or 10 amino acid residues of the artificial polypeptide attached to the IL-18 polypeptide. In some embodiments, the artificial polypeptide comprises multiple protease cleavage sites. In some embodiments, the artificial polypeptide comprises 2, 3, or 4 protease cleavage sites. In some embodiments, the artificial polypeptide is 2 to 35 amino acid residues in length.

In some embodiments, the artificial polypeptide is attached to the N-terminus or the C-terminus of the IL-18 polypeptide.

In some embodiments, the artificial polypeptide comprises a blocking moiety. In some embodiments, the blocking moiety is positioned such that cleavage at the protease cleavage site releases the blocking moiety from the Act-IL-18 polypeptide, thereby allowing the IL-18 polypeptide to interact with the receptor (or interact with the receptor to a higher degree). In some embodiments, the artificial polypeptide comprises a linking peptide between the protease cleavage site and the blocking moiety. In some embodiments, the Act-IL-18 polypeptide comprises a linking peptide between the IL-18 polypeptide and the protease cleavage site.

In some embodiments, the artificial polypeptide is attached to the N-terminus of the IL-18 polypeptide. In some embodiments, the blocking moiety comprises an IL-18 propeptide (e.g., the N-terminal portion of immature IL-18 which is endogenously cleaved by caspases to produce mature IL-18) or a portion thereof, or a variant thereof. In some embodiments, the artificial polypeptide is attached to the N-terminus of the IL-18 polypeptide, and the blocking moiety comprises the IL-18 propeptide or a portion thereof, or a variant thereof. In some embodiments, the IL-18 propeptide is linked to the N-terminus of the IL-18 polypeptide. In some embodiments, the IL-18 propeptide is a human IL-18 propeptide or a variant thereof. In some embodiments, the IL-18 propeptide is linked to the protease cleavage site which is cleaved by a protease other than a caspase (e.g., a protease which is upregulated in a tumor or tumor microenvironment). In some embodiments, the IL-18 propeptide comprises an amino acid sequence having at least 80%, 85%, 90%, 95%, 96%, 97%, 98%, 99%, or 100% sequence identity to the sequence set forth in SEQ ID NO: 89. In some embodiments, the IL-18 propeptide comprises a substitution of the cysteine residue in SEQ ID NO: 89. In some embodiments, the IL-18 propeptide comprises the sequence set forth in SEQ ID NO: 90, 91, 96, or 97.

In some embodiments, the Act-IL-18 polypeptide has a structure BM-CS-IL-18, wherein IL-18 is the IL-18 polypeptide, CS is a moiety comprising the specific cleavage site, and BM is the blocking moiety, and wherein the orientations is shown in an N-terminal to C-terminal order. In some embodiments, the artificial polypeptide is connected to the N-terminus of the IL-18 polypeptide.

In some embodiments, the artificial polypeptide is attached to the C-terminus of the IL-18 polypeptide. In some embodiments, the blocking moiety comprises a domain of an IL-18 receptor subunit or a portion thereof, or a derivative thereof. In some embodiments, the artificial polypeptide is attached to the C-terminus of the IL-18 polypeptide, and the blocking moiety comprises the domain of an IL-18 receptor subunit or a portion thereof, or a derivative thereof. In some embodiments, the domain of the IL-18 receptor subunit comprises the D3 domain of the IL-18 receptor alpha subunit (see, e.g., Tsutsumi et al., Nature Communications 5:5340|DOI: 10.1038/ncomms6340, published 15 Dec. 2014, for a description of IL-18 receptor domain architecture), or a variant thereof. In some embodiments, the blocking moiety comprises an amino acid sequence having at least 80%, 85%, 90%, 95%, 96%, 97%, 98%, 99%, or 100% sequence identity to the sequence set forth in SEQ ID NO: 93. In some embodiments, the artificial polypeptide is attached to the C-terminus of the IL-18 polypeptide, and the blocking moiety comprises an amino acid sequence having at least 80%, 85%, 90%, 95%, 96%, 97%, 98%, 99%, or 100% sequence identity to the sequence set forth in SEQ ID NO: 93.

Use of an activatable immunocytokine comprising an Act-IL-18 polypeptide might allow modulation of an immune response preferentially in a target area of the subject, such as a cancer or tumor microenvironment. In some instances, the Act-IL-18 polypeptide exhibits fewer side effects associated with systemic administration and distribution, compared to a corresponding active IL-18 polypeptide.

FIG. 2 depicts an exemplary mechanism of action of an activatable IL-18 polypeptide as provided herein, wherein the IL-18 polypeptide comprises an artificial polypeptide which renders the Act-IL-18 inactive and upon cleavage of the artificial polypeptide, the active form of the IL-18 polypeptide results. FIG. 3A illustrates a similar concept with artificial polypeptide attached to the N-terminus of the IL-18 polypeptide. The artificial polypeptide depicted comprises a linking peptide B attaching the protease cleavage site having a specific cleavage site to the N-terminus of the IL-18 polypeptide. The artificial polypeptide further comprises a linking peptide A linking the protease cleavage site to a blocking moiety (labelled “mask” in the figure). Upon cleavage of the specific cleavage site, the mask and linking peptide are released from the IL-18 polypeptide, but linking peptide B remains. This results in an active form of the IL-18 polypeptide being formed. FIG. 3B is analogous to FIG. 3A, but the artificial polypeptide is linked to the C-terminus of the IL-18 polypeptide.

In some embodiments, the active form of the IL-18 polypeptide displays reduced binding to IL-18 binding protein (IL-18BP), compared to WT IL-18. In some embodiments, the active form of the IL-18 polypeptide displays enhanced binding to IL-18R or ability to activate IL-18R, compared to WT IL-18. In some embodiments, the active form of the IL-18 polypeptide displays a binding to IL-18R or ability to activate IL-18R which is reduced by at most 100-fold relative to WT IL-18.

In some embodiments, the IL-18 polypeptide comprises one or more modifications to the sequence set forth in SEQ ID NO: 1. In some embodiments, the IL-18 polypeptide comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1. In some embodiments, the IL-18 polypeptide comprises at least one substitution at residue Y1, F2, E6, C38, M51, K53, D54, S55, T63, C76, E85, M86, T95, D98, or C127, or any combination thereof. In some embodiments, the IL-18 polypeptide comprises a Y01G, F02A, E06K, V11I, C38S, C38A, M51G, K53A, D54A, S55A, T63A, C76S, C76A, E85C, M86C, T95C, D98C, C127S, or C127A amino acid substitution, or any combination thereof. In some embodiments, the IL-18 polypeptide comprises E06K and K53A amino acid substitutions. In some embodiments, the IL-18 polypeptide comprises a T63A amino acid substitution. In some embodiments, the IL-18 polypeptide comprises a V11I amino acid substitution. In some embodiments, the IL-18 polypeptide comprises substitutions at 1, 2, 3, or 4 residues selected from C38, C76, C98, and C127. In some embodiments, the IL-18 polypeptide comprises an amino acid sequence set forth in any one of SEQ ID Nos: 2-67. In some embodiments, the IL-18 polypeptide comprises the amino acid sequence set forth in SEQ ID NO: 30. In some embodiments, the IL-18 polypeptide comprises an amino acid sequence as set forth in any one of SEQ ID NOs: 79-83. In some embodiments, the IL-18 polypeptide comprises the amino acid sequence set forth in SEQ ID NO: 30. In some embodiments, the IL-18 polypeptide comprises an amino acid sequence as set forth in any one of SEQ ID NOs: 84-86.

In some embodiments, the Act IL-18 polypeptide exhibits a half-maximal effective concentration (EC₅₀) for IL-18 receptor signaling activity which is at least 1,000-fold higher, 2,000-fold higher, 5,000-fold higher, 10,000-fold higher, 15,000-fold-higher, or 20,000-fold higher than the activated form of the IL-18 polypeptide. In some embodiments, the Act IL-18 polypeptide exhibits a half-maximal effective concentration (EC₅₀) for IL-18 receptor signaling activity which is from about 10-fold higher to about 100-fold higher than the activated form of the IL-18 polypeptide. In some embodiments, the activated form of the IL-18 polypeptide exhibits a half-maximal effective concentration (EC₅₀) for IL-18 receptor signaling activity which is within about 10-fold of the IL-18 polypeptide. In some embodiments, the Act-IL-18 polypeptide exhibits a half-maximal effective concentration (EC₅₀) for IL-18 receptor signaling activity which is at least 10-fold greater than WT IL-18.

In some embodiments, the linker comprises a polymer. In some embodiments, the polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises poly(ethylene glycol). In some embodiments, the polymer has an average molecular weight of about 0.1 kDa to 2 kDa. In some embodiments, the polymer has an average molecular weight of at least about 0.1 kDa, at least about 0.5 kDa, at least about 1 kDa, at least about 5 kDa, at least about 10 kDa, at least about 20 kDa, at least about 30 kDa, at least 40 kDa, at least 50 kDa, at least 60 kDa, at least 70 kDa, at least 80 kDa, at least 90 kDa, at least 100 kDa, at least 120 kDa, at least 130 kDa, at least 140 kDa, at least 150 kDa, or more.

In some embodiments, the second point of attachment is at a residue which is not the N-terminus or the C-terminus of the IL-18 polypeptide. In some embodiments, the second point of attachment is to a residue in the region of residues 2-156 of the IL-18 polypeptide, wherein residue position numbering is based on SEQ ID NO: 1. In some embodiments, the second point of attachment is to a residue in the region of residues 38-144 of the IL-18 polypeptide. In some embodiments, the second point of attachment is at residue 38, 68, 69, 70, 76, 78, 85, 86, 95, 98, 121, 127, or 144 of the IL-18 polypeptide. In some embodiments, the second point of attachment is at residue 68, 69, 70, 85, 86, 95, or 98 of the IL-18 polypeptide. In some embodiments, the second point of attachment is at residue 68 of the IL-18 polypeptide. In some embodiments, the IL-18 polypeptide comprises an amino acid substitution at the second point of attachment. In some embodiments, the amino acid substitution at the second point of attachment is for an unnatural amino acid. In some embodiments, the amino acid substitution at the second point of attachment is for a natural amino acid. In some embodiments, the amino acid substitution at the second point of attachment is E69C, K70C, E85C, M86C, T95C, or D98C.

In some embodiments, the first point of attachment is at a position of a K246 amino acid residue, a K248 amino acid residue, a K288 amino acid residue, a K290 amino acid residue, or a K317 amino acid residue of the Fc region (EU numbering). In some embodiments, wherein the first point of attachment is at the K248 amino acid residue of the Fc region (EU numbering).

In some embodiments, the antibody or antigen binding fragment thereof is a monoclonal antibody, a humanized antibody, a grafted antibody, a chimeric antibody, a human antibody, a deimmunized antibody, or a bispecific antibody. In some embodiments, the antibody or antigen binding fragment thereof an antigen binding fragment, wherein the antigen binding fragment comprises a Fab, a Fab′, a F(ab′)2, a bispecific F(ab′)2, a trispecific F(ab′)2, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (a domain antibody), a single chain binding polypeptide, a scFv-Fc, a Fab-Fc, a bispecific T cell engager (BiTE), a tetravalent tandem diabody (TandAb), a Dual-Affinity Re-targeting Antibody (DART), a bispecific antibody (bscAb), a single domain antibody (sdAb), a fusion protein, or a bispecific disulfide-stabilized Fv antibody fragment (dsFv-dsFv′). In some embodiments, the antibody or antigen binding fragment thereof comprises an IgG, an IgM, an IgE, an IgA, an IgD, or is derived therefrom. In some embodiments, the antibody or antigen binding fragment thereof comprises the IgG, and wherein the IgG is an IgG1, an IgG4, or is derived therefrom. In some embodiments, the antibody or antigen binding fragment thereof binds specifically to an immune associated antigen. In some embodiments, the immune cell associated antigen is 4-IBB, B7-H3, B7-H4, BTLA, CD3, CCR8, CD8A, CD8B, CD16A, CD27, CD28, CD33, CD38, CD39, CD40, CD47, CD70, CD80, CD86, CD96, CD163, CLEC-1, CLEVER-1, CTLA-4, D40, GITR, ICOS, ILT2/3/4, LAG-3, MHCI, MHCII, NKG2A, NKG2D, NKp30, NKp44, NKp46, OX40, PD-1, PD-L1, PD-L2, PSGL-1, SIGLEC-9, SIGLEC-15, SIRP-α, TCR, TIGIT, TIM-3, VISTA, or VSIG4.

In some embodiments, the immune cell associated antigen is PD-1. In some embodiments, the antibody or antigen binding fragment thereof comprises tislelizumab, baizean, OKVO411B3N, BGB-A317, hu317-1/lgG4mt2, sintilimab, tyvyt, IBI-308, toripalimab, TeRuiPuLi, terepril, tuoyi, JS-001, TAB-001, tamrelizumab, HR-301210, INCSHR-01210, SHR-1210, temiplimab, cemiplimab-rwlc, 6QVL057INT, H4H7798N, REGN-2810, SAR-439684, lambrolizumab, pembrolizumab, MK-3475, SCH-900475, h409A11, nivolumab, nivolumab BMS, BMS-936558, MDX-1106, ONO-4538, prolgolimab, forteca, BCD-100, penpulimab, AK-105, zimberclimab, AB-122, GLS-010, WBP-3055, balstilimab, 1Q2QT5M7EO, AGEN-2034, AGEN-2034w, genolimzumab, geptanolimab, APL-501, CBT-501, GB-226, dostarlimab, ANB-011, GSK-4057190A, POGVQ9A4S5, TSR-042, WBP-285, serplulimab, HLX-10, CS-1003, retifanlimab, 2Y3T5IF01Z, INCMGA-00012, INCMGA-0012, MGA-012, sasanlimab, LZZOIC2EWP, PF-06801591, RN-888, spartalizumab, PDR-001, QOG25L6Z8Z, relatlimab/nivolumab, BMS-986213, cetrelimab, JNJ-3283, JNJ-63723283, LYK98WP91F, tebotelimab, MGD-013, BCD-217, BAT-1306, HX-008, MEDI-5752, JTX-4014, cadonilimab, AK-104, BI-754091, pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, AMG-404, IBI-318, MGD-019, ONCR-177, LY-3462817, RG-7769, RO-7121661, F-520, XmAb-23104, Pd-1-pik, SG-001, S-95016, Sym-021, LZM-009, budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-pembrolizumab, 89Zr-Df-Pembrolizumab, [89Zr]Df-Pembrolizumab, STI-1110, STI-A1110, CX-188, mPD-1, MCLA-134, 244C8, ENUM 224C8, ENUM C8, 388D4, ENUM 388D4, ENUM D4, MEDI0680, NVP-LZV-184, or AMP-514, or a modified version thereof. In some embodiments, the antibody or antigen binding fragment thereof comprises nivolumab, pembrolizumab, LZM-009, or cemiplimab, or a modified version thereof. In some embodiments, the antibody or antigen binding fragment thereof comprises Tislelizumab, Sintilimab, Toripalimab, Terepril, Camrelizumab, Cemiplimab, Pembrolizumab, Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotclimab, Cadonilimab, Pidilizumab, LZM-009, or Budigalimab, or a modified version thereof. In some embodiments, the antibody or antigen binding fragment thereof comprises LZM-009.

In some embodiments, the immune cell associated antigen is PD-L1. In some embodiments, the antibody is Avelumab (Bavencio, 451238, KXG2PJ551I, MSB-0010682, MSB-0010718C, PF-06834635, CAS 1537032-82-8), Durvalumab (Imfinzi, 28×28×90 KV (UNII code), MEDI-4736, CAS 1428935-60-7), Atezolizumab (Tecentriq, 52CMIOWC3Y, MPDL-3280A, RG-7446, RO-5541267, CAS 1380723-44-3), Sugemalimab (CS-1001, WBP-3155), KN-046 (CAS 2256084-03-2), APL-502 (CBT-502, TQB-2450), Envafolimab (3D-025, ASC-22, KN-035, hu56V1-Fc-m1, CAS 2102192-68-5), Bintrafusp alfa (M-7824, MSB-0011359C, NW9K8CIJN3, CAS 1918149-01-5), STI-1014 (STI-A1014, ZKAB-001), PD-L1 t-haNK, A-167 (HBM-9167, KL-A167), IMC-001 (STI-3031, STI-A-1015, STI-A1015, s), HTI-1088 (SHR-1316), IO-103, CX-072 (CytomX Therapeutics), AUPM-170 (CA-170), GS-4224, ND-021 (NM21-1480, PRO-1480), BNT-311 (DuoBody-PD-L1×4-1BB, GEN-1046), BGB-A333, IBI-322, NM-01, LY-3434172, LDP, CDX-527, IBI-318, 89Zr-DFO-REGN3504, ALPN-202 (CD80 vlgD-Fc), INCB-086550, LY-3415244, SHR-1701, JS-003 (JS003-30, JS003-SD), HLX-20 (PL2 #3), ES-101 (INBRX-105, INBRX-105-1), MSB-2311, PD-1-Fc-OX40L (SL-279252, TAK-252), FS-118, FS118 mAb2, LAG-3/PD-L1 mAb2), FAZ-053 (LAE-005), Lodapolimab (LY-3300054, NR4MAD6PPB, CAS 2118349-31-6), MCLA-145, BMS-189, Cosibelimab (CK-301, TG-1501, CAS 2216751-26-5), IL-15Ralpha-SD/IL-15 (KD-033), WP-1066 (CAS 857064-38-1), BMS-936559 (MDX-1105), BMS-986192, RC-98, CD-200AR-L (CD200AR-L), ATA-3271, IBC-Ab002, BMX-101, AVA-04-VbP, ACE-1708, KY-1043, ACE-05 (YBL-013), ONC-0055 (ONC0055, PRS-344 S-095012), TLJ-1-CK, GR-1405, PDIACR-T, N-809 (N-IL15/PDL1), CB-201, MEDI-1109, AVA-004 (AVA-04), CA-327, ALN-PDL, KY-1003, CD22 (aPD-L1) CAR-T cells (SL-22P), ATA-2271 (M2821XXPDIDNR CAR T cells), or a modified version thereof. In some embodiments, the antibody is Durvalumab, Atezolizumab, or Avelumab, or a modified version thereof. In some embodiments, the antibody or antigen binding fragment thereof comprises Avelumab, Durvalumab, Atczolizumab, Sugemalimab, Sasanlimab, Envafolimab, Lodapolimab, or Cosibelimab, or a modified version thereof. In some embodiments, the antibody or antigen binding fragment thereof comprises Durvalumab.

In some embodiments, the antibody or antigen binding fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence of Table 1.

In some embodiments, the IL-18 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 1. In some embodiments, the IL-18 polypeptide of the immunocytokine provided herein comprises an amino acid sequence of any one of SEQ ID NOS: 2-67. In some embodiments, the IL-18 polypeptide comprises an amino acid sequence of SEQ ID NO: 30. In some embodiments, the IL-18 polypeptide comprises an amino acid sequence of SEQ ID NO: 79-83. In some embodiments, the IL-18 polypeptide comprises an amino acid sequence of SEQ ID NO: 84-86.

In some embodiments, the active form of the activatable immunocytokine composition exhibits a binding affinity (K_{D}) to at least one Fc receptor which is within 10-fold of the binding affinity of the antibody not attached to the IL-18 polypeptide. The active form of an activatable immunocytokine composition can be formed upon activation of the Act-IL-18 polypeptide comprised by the activatable immunocytokine composition, such as by cleavage at the protease cleavage site. In some embodiments, the active form of the activatable immunocytokine composition exhibits binding affinity (K_{D}) to the antigen of the antibody which is within 5-fold of the binding affinity of the antibody not attached to the IL-18 polypeptide. In some embodiments, the active form of the activatable immunocytokine composition exhibits an ability to induce IFNγ production in a cell as measured by half-maximal effective concentration (EC₅₀) which is within about 100-fold of the corresponding IL-18 polypeptide not comprised in the active form of the activatable immunocytokine composition, and wherein the active form of the activatable immunocytokine composition exhibits a lower EC₅₀ than WT IL-18. In some embodiments, the active form of the activatable immunocytokine composition exhibits enhanced anti-tumor growth inhibition compared to the antibody alone. In some embodiments, the active form of the activatable immunocytokine composition exhibits enhanced anti-tumor growth inhibition compared to the antibody and the IL-18 polypeptide administered in combination.

In some embodiments, the activatable immunocytokine composition provided herein, further comprises a second linker, wherein the second linker comprises a third point of attachment to the antibody or antigen binding fragment thereof, and a fourth point of attachment to an additional cytokine. In some embodiments, the additional cytokine is selected from a second IL-18 polypeptide, an IL-7 polypeptide, or an IL-2 polypeptide. In some embodiments, the additional cytokine is a second Act-IL-18 polypeptide. In some embodiments, the second Act-IL-18 polypeptide is substantially identical to the Act-IL-18 polypeptide. In some embodiments, the third point of attachment is to a different heavy chain of the antibody or antigen binding fragment thereof from the second point of attachment.

In one aspect, provided herein, is a pharmaceutical composition comprising: an activatable immunocytokine composition as provided herein, and one or more pharmaceutically acceptable carriers or excipients. In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form. In some embodiments, the one or more pharmaceutically acceptable carriers or excipients comprises one or more of each of: a carbohydrate, an inorganic salt, an antioxidant, a surfactant, a buffer, or any combination thereof. In some embodiments, the pharmaceutical composition comprises one, two, three, four, five, six, seven, eight, nine, ten, or more excipients.

In one aspect, provided herein, is a method of treating cancer in a subject in need thereof, comprising administering to the subject an effective amount of the activatable immunocytokine composition as provided herein, or a pharmaceutical composition as provided herein. In some embodiments, the cancer is a melanoma, a lung cancer, a bladder cancer (BC), a microsatellite instability high (MSI-H)/mismatch repair-deficient (dMMR) solid tumor, a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), a primary mediastinal large B cell lymphoma (PMBCL), or a combination thereof. In some embodiments, the cancer is a solid cancer consisting of bladder and ureteral cancer, bone cancer, brain and spinal cord cancer, breast cancer, cervical cancer, colorectal cancer, esophageal cancer, eye cancer, gallbladder or bile duct cancer, germ cell tumor, head and neck cancer, kidney cancer, liver cancer, lung cancer, metastatic brain tumor, ovarian cancer, pancreatic cancer, pediatric cancer, peripheral nerve sheath tumor, pituitary cancer, prostate cancer, skin cancer, stomach or gastric cancer, soft tissue cancer, testicular cancer, thyroid cancer, uterine cancer. In some embodiments, the cancer is a carcinoma, and wherein the carcinoma comprises a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), or a combination thereof. In some embodiments, the cancer is a leukemia, lymphoma, myeloma, or a combination thereof. In some embodiments, the cancer is leukemia and the leukemia comprises, acute lymphoblastic leukemia, acute myeloid leukemia, chronic lymphocytic leukemia, small lymphocytic lymphoma, or chronic myeloid leukemia. In some embodiments, the cancer is lymphoma and the lymphoma comprises Burkitt lymphoma, chronic lymphocytic leukemia/small lymphocytic lymphoma, cutancous lymphoma, diffuse large B cell lymphoma, follicular lymphoma, Hodgkin lymphoma, peripheral T cell lymphoma, Waldenstrom macroglobulinemia, lymphoplamacytic lymphoma, marginal zone lymphoma, B cell cutaneous lymphoma, extranodal natural killer T cell lymphoma, T cell lymphoblastic lymphoma, peripheral T cell lymphoma, or T cell cutaneous lymphoma.

In some embodiments, the method of making an activatable immunocytokine composition comprises a) covalently attaching a reactive group to a specific residue of the antibody or antigen binding fragment thereof; b) contacting the reactive group with a complementary reactive group attached to the Act-IL-18 polypeptide; and c) forming the composition.

Further provided herein in an aspect is an activatable IL-18 (Act-IL-18) polypeptide having a structure BM-CS-IL-18; BM-LP₁-CS-IL-18; BM-LP₁-CS-LP₂-IL-18; or BM-CS-LP₁-IL-18; wherein IL-18 is an IL-18 polypeptide, CS is a moiety comprising a specific cleavage site (e.g., a protease recognition sequence), LP₁ is a first linking peptide, LP₂ is a second linking peptide, and BM is a blocking moiety, and wherein the orientations are shown in an N-terminal to C-terminal order, preferably wherein the Act-IL-18 polypeptide as a structure BM-CS-IL-18. In some embodiments, the blocking moiety the blocking moiety comprises an IL-18 propeptide or a portion thereof, or a variant thereof, preferably wherein the IL-18 propeptide is a human IL-18 propeptide or a variant thereof. In some embodiments, the IL-18 propeptide comprises an amino acid sequence having at least 80%, 85%, 90%, 95%, 96%, 97%, 98%, 99%, or 100% sequence identity to the sequence set forth in SEQ ID NO: 89. In some embodiments, the IL-18 propeptide comprises a substitution of the cysteine residue in SEQ ID NO: 89. In some embodiments, the IL-18 propeptide comprises the sequence set forth in SEQ ID NO: 89, 91, 96, or 97. In some embodiments, the IL-18 polypeptide comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1. In some embodiments, the IL-18 polypeptide comprises the amino acid sequence of any one of SEQ ID NOs: 1-67 or 79-86, or another IL-18 polypeptide as described herein. In some embodiments, the specific cleavage site is comprised within a protease recognition sequence provided Table 2B. In some embodiments, the specific cleavage site is the protease recognition sequence comprises the amino acid sequence set forth in any one of SEQ ID NOs: 646, 647, 648, 649, 650, 651, 652, 653, 657, 658, or 659.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 illustrates a non-limiting mechanism of action of an activatable anti-PD-1 antibody/IL-18 immunocytokine provided herein, wherein an activated T cell shows enhanced activation through concurrent blockade of PD-1 and stimulation by IL-18.

FIG. 2 shows an illustration of an exemplary mechanism of activation of an Act-IL-18 as provided herein.

FIG. 3A shows an illustration of an exemplary mechanism of activation of an Act-IL-18 polypeptide having a blocking moiety (“mask” in the figure) linked to the N-terminus of the IL-18 polypeptide.

FIG. 3B shows an illustration of an exemplary mechanism of activation of an Act-IL-18 polypeptide having a blocking moiety (“mask” in the figure) linked to the C-terminus of the IL-18 polypeptide.

FIG. 4 illustrates the mechanism of action of IL-18 on IFNγ and IL-18BP production, and IL-18 inhibitory activity by IL-18BP.

FIG. 5 shows the IFNγ induction ability of an IL-18 polypeptide of the disclosure compared to a wild type IL-18 polypeptide.

FIG. 6 shows IL-18BP inhibition of an IL-18 polypeptide of the disclosure compared to a wild type IL-18 polypeptide.

FIG. 7 shows a schematic representation of coupling of a bifunctional probe to an IL-18 polypeptide provided herein.

FIG. 8 shows a conditionally activatable IL-18 with terminal protease recognition sequence linked to a blocking moiety and an orthogonal conjugation handle. Such an activatable IL-18 can be incorporated into an immunocytokine.

FIG. 9 shows a conditionally activatable IL-18 polypeptide with an N-terminal protease. Such an activatable IL-18 can be incorporated into an immunocytokine.

FIG. 10A shows a conditionally activatable IL-18 with a C-terminal protease recognition sequence and blocking moiety and an orthogonal conjugation handle. Such an activatable IL-18 can be incorporated into an immunocytokine.

FIG. 10B shows a conditionally activatable IL-18 with a C-terminal protease recognition sequence and an orthogonal conjugation handle. Such an activatable IL-18 can be incorporated into an immunocytokine.

FIG. 11 shows an illustrative example for a conditionally activatable, conjugatable IL-18 polypeptide with three N-terminal residues substituted with protease recognition sequence and blocking moiety. Upon proteolytic processing in a tumor microenvironment (TME), a functional IL-18 mutein is revealed. Such an activatable IL-18 can be incorporated into an immunocytokine.

FIG. 12 shows an illustrative example for a conditionally activatable, conjugatable IL-18 polypeptide with three C-terminal residues substituted with protease recognition sequence and blocking moiety. Upon proteolytic processing in TME, a functional IL-18 mutein is revealed. Such an activatable IL-18 can be incorporated into an immunocytokine.

FIG. 13 shows an illustrative example for a conditionally activatable IL-18 polypeptide with three N-terminal residues substituted with protease recognition sequence and conjugation handle. Upon proteolytic processing in TME, free functional IL-18 mutein is revealed. Such an activatable IL-18 can be incorporated into an immunocytokine.

FIG. 14A shows SDS-PAGE gels showing activatable IL-18 polypeptides provided herein both before and after MMP treatment.

FIG. 14B shows SDS-PAGE gels showing activatable IL-18 polypeptides provided herein both before and after MMP treatment.

FIG. 14C shows SDS-PAGE gels showing activatable IL-18 polypeptides provided herein after MMP treatment and resin purification. FT indicates flow through and E indicates eluate.

FIG. 14D shows a representative SDS-PAGE characterization of Act-IL-18 variants C316 and C317 described herein digested by MMP2, MMP7, MMP9, matriptase, and uPA for 48h.

FIG. 14E shows SDS-PAGE gels showing activatable IL-18 polypeptides provided herein both before and after MMP9 treatment.

FIG. 14F shows BLI characterization of Act-IL-18 variant C185 before MMP9 treatment (left) and after (right) tested against IL-18 receptor alpha subunit.

FIG. 15A shows dose response curves for IL-18 receptor activation for activatable IL-18 polypeptides provided herein.

FIG. 15B shows dose response curves for IL-18 receptor activation for activatable IL-18 polypeptides provided herein.

FIG. 15C shows plots measuring the ability of the MMP9 cleaved (solid black round symbols and black lines) and uncleaved (open round symbols and dotted black lines) masked IL-18 polypeptides to bind to the human IL-18 Binding Protein (IL-18BP). The figure shows free IL-18BP AlphaLISA signal on the y-axis, and dosage of IL-18 polypeptides on the x-axis.

FIG. 15D shows plots measuring the ability of the MMP9 cleaved (solid black round symbols and black lines) and uncleaved (open round symbols and dotted black lines) masked IL-18 polypeptides to stimulate the secretion of IFNgamma by NK-92 cells. The figure shows mean IFNg signal on the y-axis and dosage of the IL-18 polypeptides on the x-axis.

FIG. 15E shows plots measuring the ability of the MMP9 cleaved (solid black round symbols and black lines) and uncleaved (open round symbols and dotted black lines) masked IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (OD 620 nm) on the y-axis, and dosage of the masked and unmasked IL-18 polypeptides on the x-axis.

FIG. 16A shows plots measuring the ability of the MMP9 cleaved (solid black round symbols and black lines) and uncleaved (open round symbols and dotted black lines) masked IL-18 polypeptides to stimulate the secretion of IFNgamma by NK-92 cells. The figure shows mean IFNg signal on the y-axis and dosage of the IL-18 polypeptides on the x-axis.

FIG. 16B shows plots measuring the ability of increasing concentrations of human recombinant IL-18 binding protein (IL-18BP) to inhibit the secretion of IFNgamma by NK-92 cells stimulated with 1 nM of the MMP9 cleaved (solid black round symbols and black lines) and uncleaved (open round symbols and dotted black lines) masked IL-18 polypeptides. The figure shows mean IFNg signal on the y-axis and dosage of IL-18BP on the x-axis.

FIG. 16C shows plots measuring the ability of the MMP9 cleaved (solid black round symbols and black lines) and uncleaved (open round symbols and dotted black lines) masked IL-18 polypeptides to bind to the human IL-18 Binding Protein (IL-18BP). The figure shows free IL-18BP AlphaLISA signal on the y-axis, and dosage of IL-18 polypeptides on the x-axis.

FIG. 17A illustrates a non-limiting mechanism of action of an activatable anti-PD-L1 antibody/IL-18 immunocytokine provided herein, wherein the immunocytokine disrupts PD-L1/PD-1 interaction and effectuates IL-18R signaling.

FIG. 17B illustrates a non-limiting mechanism of action of an activatable anti-PD-L1 antibody/IL-18 immunocytokine acting on an immune cell expressing both IL-18R and PD-L1.

FIG. 18 illustrates an immunocytokine composition provided herein with two different protein payloads (e.g., IL-18 and IL-2) linked to an antibody (e.g., PD-L1 antibody) acting on an immune cell with simultaneous activation of two different receptors (e.g., IL-18R and IL-2R). An activatable IL-18 polypeptide can be used as one of the protein payloads.

FIG. 19 shows a representative immunocytokine composition with a drug antibody ratio of 1 (DAR1).

FIG. 20 shows an illustration of a site selective introduction of a conjugation handle to an antibody as provided herein.

FIG. 21 shows exemplary reaction schemes which can be used to prepare antibody/IL-18 immunocytokine compositions provided herein with drug antibody ratio of 1 (top) or 2 (bottom).

FIG. 22 shows a reverse phase HPLC chromatogram of a purified activatable IL-18/anti-PD-1 antibody (LZM009) as provided herein with a DAR of 1 attached at residue K248 of the Fc region of the antibody (EU numbering). The composition shown in the figure is Composition L.

FIG. 23 shows a mass spectrometry trace (Q-TOF) of a purified activatable IL-18/anti-PD-1 antibody (LZM009) as provided herein with a DAR of 1 attached at residue K248 of the Fc region of the antibody (EU numbering) . . . . The composition shown in this figure is Composition L.

FIG. 24A shows SDS-PAGE analysis of various activatable immunocytokine compositions as described herein, as well as corresponding control molecules.

FIG. 24B shows SDS-PAGE analysis of various activatable immunocytokine compositions as described herein, as well as corresponding control molecules.

FIG. 25A shows plots measuring the ability of the MMP9 cleaved (solid black round symbols and black lines) masked PD1 IL-18 immunocytokines to bind to the human IL-18 Binding Protein (IL-18BP). The figure shows free IL-18BP AlphaLISA signal on the y-axis, and dosage of IL-18 immunocytokines on the x-axis. The tested non-masked IL-18 polypeptide and PD1-IL18 immunocytokine are native IL-18 wild-type (SEQ ID NO: 1) and reference Composition A. The tested masked PD1 IL-18 immunocytokines are composition J and composition K.

FIG. 25B shows plots measuring the ability of the MMP9 cleaved (solid black round symbols and black lines) and uncleaved (black round open symbols and dotted black lines) masked PD1 IL-18 immunocytokines to bind to the human IL-18 Binding Protein (IL-18BP). The figure shows free IL-18BP AlphaLISA signal on the y-axis, and dosage of IL-18 immunocytokines on the x-axis. The tested masked PD1 IL-18 immunocytokines are compositions E, G, H and I.

FIG. 25C shows plots measuring the ability of the MMP9 cleaved (solid black round symbols and black lines) masked PD1 IL-18 immunocytokines to bind to the human IL-18 Binding Protein (IL-18BP). The figure shows free IL-18BP AlphaLISA signal on the y-axis, and dosage of IL-18 Immunocytokines on the x-axis. The tested masked PD1 IL-18 immunocytokines are compositions L to O.

FIG. 25D shows plots measuring the ability of the MMP9 cleaved (solid black round symbols and black lines) masked PD1 IL-18 immunocytokines or uncleaved (black round open symbols and dotted black lines) non-masked immunocytokines to bind to the human IL-18 Binding Protein (IL-18BP). The figure shows free IL-18BP AlphaLISA signal on the y-axis, and dosage of IL-18 immunocytokines on the x-axis. The tested non-masked PD1-IL18 immunocytokines are composition S, and reference Composition A. The tested masked PD1 IL-18 immunocytokines are compositions Q, R and T.

FIG. 26A shows plots measuring the levels PD-1 surface expression on wild type NK-92 cells (left) and on NK-92 cells transduced with human PD-1 (right).

FIG. 26B shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokine on the x-axis. The masked IL-18 PD-1 immunocytokine tested is Composition E.

FIG. 26C shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Composition F and G.

FIG. 26D shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Composition H and I.

FIG. 26E shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Composition J and K.

FIG. 26F shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Composition L and M.

FIG. 26G shows plots measuring the ability of the cleaved (black round symbols and solid black lines) and uncleaved (black round open symbols and dotted black lines) masked IL-18 PD-1 immunocytokine to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Composition N and O.

FIG. 26H shows plots measuring the ability of the cleaved (black round symbols and solid black lines) and uncleaved (black round open symbols and dotted black lines) masked IL-18 PD-1 immunocytokines to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the masked IL-18 PD-1 immunocytokines on the x-axis. The masked IL-18 PD-1 immunocytokines tested are Compositions Q and R.

FIG. 26I shows plots measuring the ability of the cleaved (black round symbols and solid black lines) and uncleaved (black round open symbols and dotted black lines) masked IL-18 PD-1 immunocytokines to stimulate the secretion of IFNgamma by parental PD-1^{negative} (left panel) and by engineered PD-1^{positive} NK-92 cells (right panel). The figure shows mean IFNg AlphaLISA signal on the y-axis and dosage of the IL-18 PD-1 immunocytokines on the x-axis. The tested unmasked molecule Composition S and the masked molecule Composition T are shown.

FIG. 27A shows plots measuring the ability of increasing concentrations of human recombinant IL-18 binding protein (IL-18BP) to inhibit the secretion of IFNgamma by parental PD-1^{negative} (left panel) and by engineered PD-1^{positive} NK-92 cells (right panel) stimulated with 1 nM of the MMP9 cleaved (solid black round symbols and black lines) masked IL-18 immunocytokines. The graphs depict mean IFNgamma signal on the y-axis and dosage of IL-18BP on the x-axis. The masked IL-18 immunocytokines tested are compositions E, G, H and I.

FIG. 27B shows plots measuring the ability of increasing concentrations of human recombinant IL-18 binding protein (IL-18BP) to inhibit the secretion of IFNgamma by parental PD-1^{negative} (left panel) and by engineered PD-1^{positive} NK-92 cells (right panel) stimulated with 1 nM of the MMP9 cleaved (solid black round symbols and black lines) masked IL-18 immunocytokines. The graphs depict mean IFNgamma signal on the y-axis and dosage of IL-18BP on the x-axis. The masked IL-18 immunocytokines tested are compositions J, K and L.

FIG. 27C shows plots measuring the ability of increasing concentrations of human recombinant IL-18 binding protein (IL-18BP) to inhibit the secretion of IFNgamma by parental PD-1^{negative} (left panel) and by engineered PD-1^{positive} NK-92 cells (right panel) stimulated with 1 nM of the MMP9 cleaved (solid black round symbols and black lines) masked IL-18 immunocytokines. The graphs depict mean IFNgamma signal on the y-axis and dosage of IL-18BP on the x-axis. The masked IL-18 immunocytokines tested are compositions M, N and O.

FIG. 27D shows plots measuring the ability of increasing concentrations of human recombinant IL-18 binding protein (IL-18BP) to inhibit the secretion of IFNgamma by parental PD-1^{negative} (left panel) and by engineered PD-1^{positive} NK-92 cells (right panel) stimulated with 1 nM of the MMP9 cleaved (solid black round symbols and black lines) masked IL-18 immunocytokines and non-masked uncleaved (black round open symbols and dotted black lines) IL-18 immunocytokines. The graphs depict mean IFNgamma signal on the y-axis and dosage of IL-18BP on the x-axis. The masked IL-18 immunocytokines tested are compositions T, Q and R. The non-masked IL-18 immunocytokine tested is compositions S.

FIG. 28A shows tumor volume (mm3) on the y-axis versus time (days) on the x-axis. Human PD1 knock-in (huPD1 ki) C57BL/6 animals bearing sub cutaneous MC38 tumors were treated intravenously with a single dose (SD) of 1 mg/kg on day 0. Tumor size at start of treatment was ˜90 mm³ (n=6; Mean±SEM).

FIG. 28B shows body weight change (%) compared to day 0 body weight on the y-axis versus time (days) on the x-axis. Human PD1 knock-in (huPD1 ki) C57BL/6 animals bearing sub-cutaneous MC38 tumors were treated intravenously with a single dose (SD) of 1 mg/kg on day 0. Tumor size at start of treatment was ˜90 mm³ (n=6; Mean±SEM).

FIG. 28C shows tumor volume (mm3) on the y-axis versus time (days) on the x-axis for individual human PD1 knock-in (huPD1 ki) C57BL/6 animals bearing sub-cutaneous MC38 tumors. Mice were treated intravenously with a single dose (SD) of 1 mg/kg on day 0. Tumor size at start of treatment was ˜90 mm3 (n=6; Mean±SEM).

FIG. 28D shows IFNgamma (pg/mL) released in plasma on the y-axis versus time (hours after dosing) on the x-axis. Human PD1 knock-in (huPD1 ki) C57BL/6 animals bearing sub-cutaneous MC38 tumors were treated intravenously with a single dose (SD) of 1 mg/kg on day 0 and plasma samples were harvested at different time points after dosing (n=3; Mean±SEM). IFNgamma values in plasma were quantified using the Legendplex Mouse Anti-virus Response Panel according to manufacturer's recommendations.

FIG. 28E shows total drug levels (ng/mL) present in plasma on the y-axis versus time (hours after dosing) on the x-axis. Human PD1 knock-in (huPD1 ki) C57BL/6 animals bearing sub-cutaneous MC38 tumors were treated intravenously with a single dose (SD) of 1 mg/kg on day 0 and plasma samples were harvested at different time points after dosing (n=3; Mean±SEM). Drug in plasma was detected using a sandwich ELISA.

### DETAILED DESCRIPTION

Disclosed herein are activatable immunocytokine compositions comprising an antibody or antigen binding fragment specific for immune cell associated antigens linked to an Act-IL-18 polypeptide. In some instances, the activatable immunocytokine compositions provided herein are useful as potent stimulators of one or more immune cell types (e.g., T cells, macrophages, etc.). In some embodiments, the activatable immunocytokine compositions can act by one or more modes of action. In some embodiments, the activatable immunocytokine can minimize off-target effects owing to the target-selective activation of the IL-18 polypeptide.

In some embodiments, the antibody of the activatable immunocytokine composition allows for targeting of the activatable immunocytokine composition to an immune cell. In some embodiments, the activatable immunocytokine composition can inhibit an activity of the immune cell associated antigen (e.g., inhibiting a checkpoint interaction such as a PD-1/PD-L1 interaction) through binding to the immune cell associated antigen. In some embodiments, the activatable immunocytokine compositions induce IFNγ production in immune cells (e.g., T cells or NK cells). The antibody or antigen binding fragment-Act-IL-18 containing activatable immunocytokine compositions of the disclosure can have synergistic efficacy and improved tolerability by a subject. In some embodiments, the antibody or antigen binding fragment-Act-IL-18 containing activatable immunocytokine compositions can significantly reduce the therapeutic dose of the antibody or antigen binding fragment, the IL-18 polypeptide, or both for a subject with a disease, such as a cancer, as compared to a treatment with one or both entities individually or in combination. In some embodiments, the activatable immunocytokine compositions provided herein are associated with fewer side effects than administration of one or both entities individually or in combination, potentially due to the targeting nature of the antibodies for an immune cell.

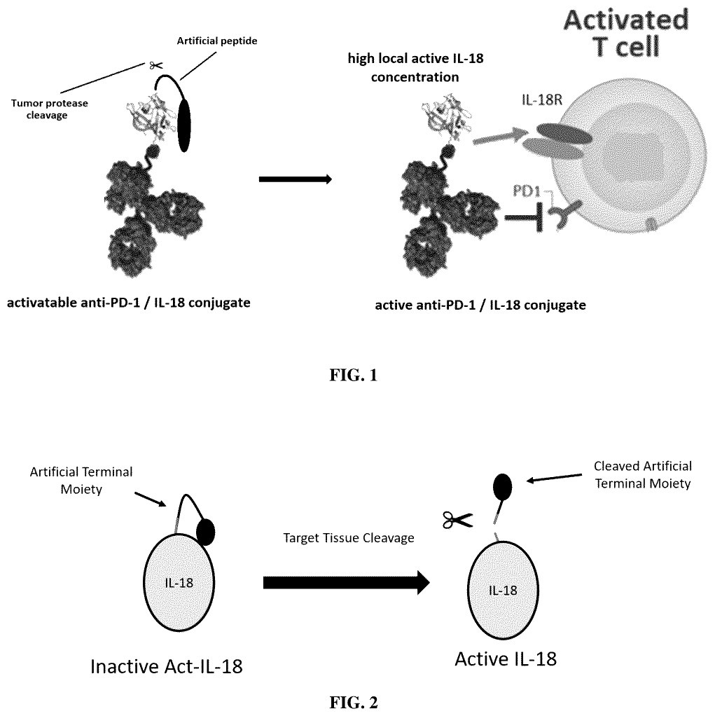

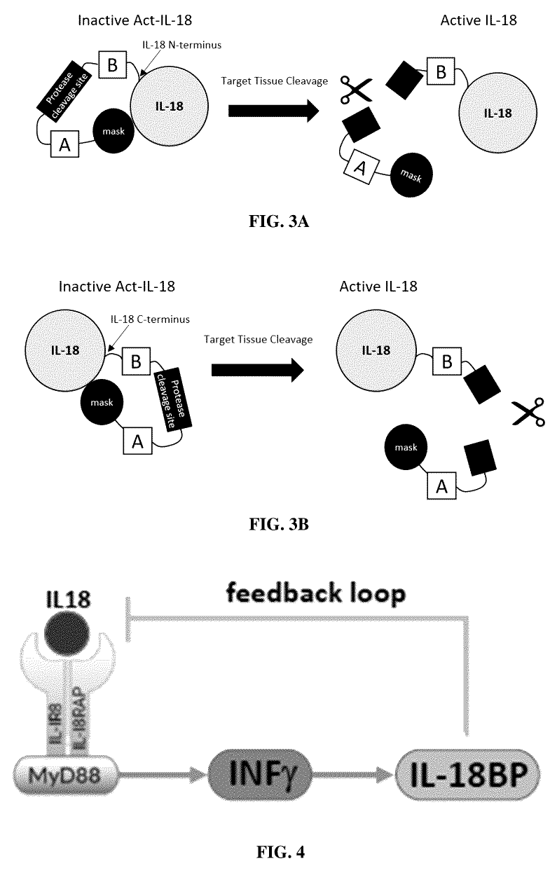

An exemplary, non-limiting mechanism of action of an activatable immunocytokine provided herein is shown in FIG. 1. In the exemplary embodiment, the immunocytokine composition comprises an anti-PD-1 antibody as the antibody or antigen binding fragment of the activatable immunocytokine. In this embodiment, the anti-PD-1 antibody portion of the immunocytokine selectively binds to PD-1 present on the surface of an activated T cell (e.g., a CD8⁺ T cell). This binding prevents the checkpoint interaction of PD-1 and PD-L1/2, thus preventing attenuation of activity of the T cell. Concomitantly, after activation of the IL-18 portion of the immunocytokine by tumor microencironment proteases, the IL-18 concentration is effectively in a high local concentration near the T-cell due to the linkage, further activates the T cell through IL-18R signaling. The activatable immunocytokine shown in this figure is depicted with two activatable IL-18 polypeptides attached to the antibody (DAR2), however activatable immunocytokines having only a single activatable IL-18 polypeptide attached (DAR1) are also expressly contemplated as within the scope of the instant disclosure. While the exemplary embodiment shows the mechanism of action of an anti-PD-1 antibody, antibodies or antigen binding fragments specific for other immune antigens provided herein can function according to a similar mechanism.

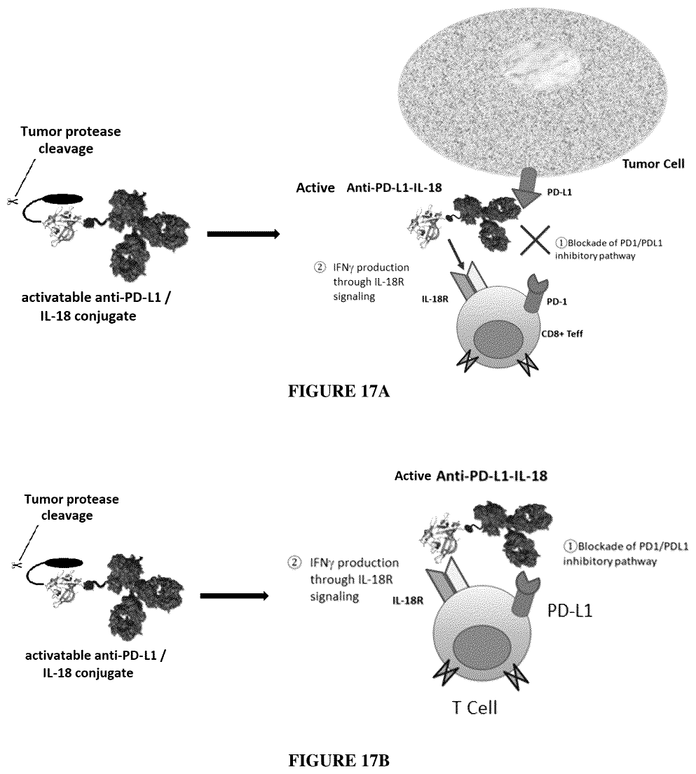

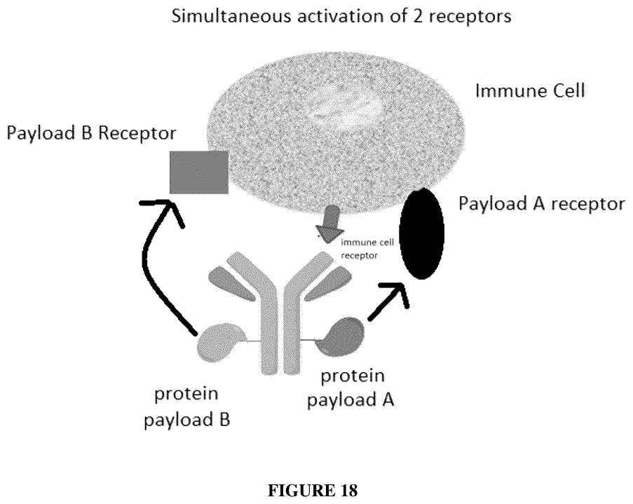

Another exemplary, non-limiting mechanism of action of an anti-PD-L1 antibody/IL-18 activatable immunocytokine is shown in FIG. 17A. In the exemplary embodiment, the anti-PD-L1 antibody portion of the immunocytokine binds to PD-L1 expressed on the surface of a tumor cell. When a T cell comes into contact with the tumor cell, an interaction between PD-1 on the T cell and PD-L1 on the cell is blocked, preventing attenuation of the activity of the T cell. Additionally, the IL-18 portion of the immunocytokine, upon cleavage by tumor proteases to activate the IL-18 polypeptide, signals through IL-18R on the immune cell, thereby inducing production of IFNγ and further activation of the immune cell. Though this exemplary embodiment is demonstrated for PD-L1, other immune antigens provided herein in immunocytokines may display similar mechanisms of action. Analogously, FIG. 17B illustrates a non-limiting mechanism of action of an anti-PD-L1 antibody/IL-18 activatable immunocytokine acting on an immune cell expressing both IL-18R and PD-L1. FIG. 18 illustrates an immunocytokine composition provided herein with two different protein payloads (e.g., IL-18 and IL-2) linked to an antibody (e.g., PD-L1 antibody) acting on an immune cell with simultaneous activation of two different receptors (e.g., IL-18R and IL-2R). One of the protein payloads can be an activatable IL-18 polypeptide as provided herein.

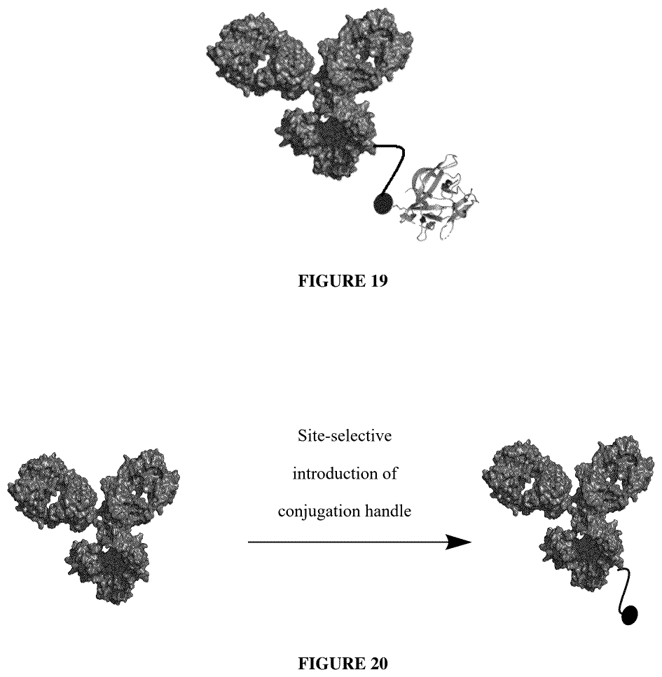

Also disclosed herein are methods of manufacturing the activatable immunocytokines provided herein. In some embodiments, the activatable immunocytokine compositions are prepared using chemical linkers which can attach the two moieties of immunocytokine composition to each other at pre-selected sites of each moiety with high fidelity. In some embodiments, the methods provided herein can be used on a wide variety of antibodies or antigen binding fragments in order to rapidly and easily generate a wide variety of immune antigen specific antibody or antigen binding fragments linked to activatable IL-18 polypeptides as immunocytokines. In some embodiments, the methods can be used on readily commercially available antibodies to allow for rapid linking with Act-IL-18 polypeptides provided herein. One non-limiting illustration of an immunocytokine as provided herein is shown in FIG. 19, which depicts an IL-18 polypeptide linked to an antibody as provided herein with a point of attachment of the linker to the IL-18 polypeptide at a side chain of a residue and to a side chain of a residue in the Fc region.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Immune Antigen Specific Antibodies Linked to Act-IL-18 Polypeptides as Activatable Immunocytokines

Provided herein are antibodies and antigen binding fragments which bind to immune cell associated antigens, linked to Act-IL-18 polypeptides as activatable immunocytokine compositions. The activatable immunocytokine compositions provided herein are effective for simultaneously delivering the IL-18 polypeptide and the antibody or antigen binding fragment to a target cell, such as an immune cell. This simultaneous delivery of both agents to the same cell has numerous benefits, including improved IL-18 polypeptide selectivity, enhanced therapeutic potential of the IL-18 polypeptide, and minimized risk of side effects from administering IL-18 therapies. In some embodiments, the activatable immunocytokine compositions act through multiple modes of action, including without limitation disrupting an activity of the immune cell associated antigen (e.g., immune checkpoint evasion) and/or enhanced activation of immune cells in or around a tumor microenvironment.

The activatable immunocytokine compositions provided herein utilize linkers to attach the antibody or antigen binding fragment to the Act-IL-18 polypeptide. In some embodiments, the linkers are attached to each moiety (i.e., the antibody or antigen binding fragment and the IL-18 polypeptide) at specific residues or a specific subset of residues. In some embodiments, the linkers are attached to each moiety in a site-selective manner, such that a population of the activatable immunocytokine compositions is substantially uniform. This can be accomplished in a variety of ways as provided herein, including by site-selectively adding reagents for a conjugation reaction to a moiety to be conjugated, synthesizing or otherwise preparing a moiety to be conjugated with a desired reagent for a conjugation reaction, or a combination of these two approaches. Using these approaches, the sites of attachment (such as specific amino acid residues) of the linker to each moiety can be selected with precision.

Additionally, these approaches allow a variety of linkers to be employed for the composition which are not limited to amino acid residues as is required for fusion proteins. This combination of linker choice and precision attachment to the moieties allows the linker to also, in some embodiments, perform the function of modulating the activity of one of the moieties, for example if the linker is attached to the Act-IL-18 polypeptide at a position that interacts with a protein which binds to the IL-18 polypeptide (e.g., IL-18 binding protein).

In one aspect, provided herein, is an activatable immunocytokine composition, comprising: an Act-IL-18 polypeptide and an antibody or an antigen binding fragment thereof specific for an immune cell associated antigen. In some embodiments, the activatable immunocytokine composition comprises a linker. In some embodiments, the linker comprises a first point of attachment to the antibody or antigen binding fragment thereof. In some embodiments, the linker comprises a second point of attachment to the IL-18 polypeptide.

The present disclosure describes antibodies or antigen binding fragments linked to Act-IL-18 polypeptides as activatable immunocytokine compositions and their use as therapeutic agents. IL-18 is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a T helper type 1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Rα), which is the ligand binding chain for mature IL-18. However, the binding affinity of IL-18 to IL-18Rα is low. In cells that express the co-receptor, IL-18 receptor beta chain (IL-18Rβ), a high affinity heterodimer complex is formed, which then activates cell signaling.

The activity of IL-18 is balanced by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 4 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit IL-18 activity.

In some embodiments, the IL-18 polypeptides of the activatable immunocytokines provided herein display reduced binding to IL-18BP and retain binding to the IL-18 receptor. The IL-18 polypeptides with this property provided herein are able to retain IL-18 receptor signaling activity (including inducing production of IFNγ) even in the presence of IL-18BP. This allows the immunocytokines provided herein to retain IL-18 signaling activity well beyond a short period of time after administration, or upon repeat administrations. In some embodiments, the modified IL-18 polypeptides with this property comprise a modification (e.g., substitution, polymer attachment, or deletion) at one or more amino acid residues which convey this property to the IL-18 polypeptide. Examples of IL-18 polypeptides with this property are provided herein, as well as those otherwise known, such as those described in Patent Cooperation Treaty Publication Nos. WO2019051015, WO2022094473, WO2022172944, WO2022172944, WO2022172944, WO2022172944, and WO2022172944. In addition to IL-18 polypeptides provided herein, these otherwise known IL-18 polypeptides or their analogs may similarly be modified with points of attachment to the linker as provided herein, or, in some instances, combined with artificial peptides as described herein.

### Immune Cell Specific Antibodies

In some embodiments, an antibody or an antigen binding fragment of an activatable immunocytokine of the disclosure specifically binds to an immune cell associated antigen. An immune cell associated antigen provided herein is an antigen which is associated with expression on immune cells or associated with activity of immune cells (e.g., an antigen associated with immune cell activation or deactivation), or both. In some embodiments, the immune cell associated antigen is expressed at a level of at least 25% greater, at least 30% greater, at least 40% greater, at least 50% greater, at least 60% greater, at least 70% greater, at least 80% greater, at least 90% greater, or at least 100% greater in the immune cell than another cell. In some embodiments, the immune cell associated antigen is expressed at a level of at least 2-fold greater, at least 4-fold greater, at least 6-fold greater, at least 8-fold greater, or at least 10-fold greater in the immune cell than another cell. Non-limiting examples of immune cell associated antigens include 4-IBB, CD3, CCR8, CD8A, CD8B, CD16A, CD28, CD80, CD86, CD96, CD226, CTLA-4, D40, GITR, ICOS, LAG-3, MHCI, MHCII, NKG2A, NKG2D, NKp30, NKp44, NKp46, OX40, PD-1, PD-L1, PD-L2, SIRPA, TCR, TIGIT, TIM-3, and VISTA.

An antibody selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. In some embodiments, an antibody or an antigen binding fragment of the disclosure can inhibit the action/activity of the substance to which it binds. In some embodiments, an antibody or antigen binding fragment of the disclosure can agonize the action/activity of the substance to which it binds (e.g., an immune cell agonist antibody or antigen binding fragment such as one specific for CD16A, NKG2D, NKp30, or other targets).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a C_{H}3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a C_{H}3 domain can be from an immunoglobulin selected from the group consisting of an IgG, an IgA, an IgD, an IgE, and an IgM, or is derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some instances, an antibody or antigen binding fragment comprises an IgG2 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“K” or “K”) or lambda (“2”), based on the amino acid sequences of their constant domains.

A “variable region” of an antibody refers to the variable region of the antibody light chain or the variable region of the antibody heavy chain, cither alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (Kabat et al., Sequences of Proteins of Immunological Interest, 5th Ed., 1991, National Institutes of Health, Bethesda Md., pages 647-669; hereafter “Kabat”); and (2) an approach based on crystallographic studies of antigen-antibody complexes (Al-Iazikani et al. (1997) J. Molec. Biol. 273:927-948). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat et al., Id. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196:901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Id. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, grafted antibodies, multispecific antibodies, heteroconjugate antibodies, humanized antibodies, human antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies. In certain embodiments of the methods and conjugates provided herein, the antibody requires an Fc region to enable attachment of a linker between the antibody and the protein (e.g., attachment of the linker using an affinity peptide, such as in AJICAP™ technology).

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the co-expression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)2, a bispecific F(ab′)2, a trispecific F(ab′)2, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides). In certain embodiments of the invention, a full length antibody (e.g., an antigen binding fragment and an Fc region) is preferred.

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 424), (GGS)ₙ (SEQ ID NO: 425), (GGGS)ₙ (SEQ ID NO: 426), (GGSG)ₙ (SEQ ID NO: 427), or (GGSGG), (SEQ ID NO: 428), (GGGGS)ₙ (SEQ ID NO: 429), wherein n is 1, 2, 3, 4, 5, 6, 7, 8, 9, or 10. For example, a linking peptide can be (GGGGS)₃ (SEQ ID NO: 430) or (GGGGS)₄ (SEQ ID NO: 431). In some embodiments, a linking peptide bridges approximately 3.5 nm between the carboxy terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quatravalent antibody or antigen binding fragment.

An antibody or antigen binding fragment described herein can be isolated, purified, recombinant, or synthetic.

It is contemplated that generic or biosimilar versions of the named antibodies herein which share the same amino acid sequence as the indicated antibodies are also encompassed when the name of the antibody is used.

The antibodies described herein may be made by any suitable method. Antibodies can often be produced in large quantities, particularly when utilizing high level expression vectors.

In one embodiment, an antibody or an antigen binding fragment of the disclosure comprises a fusion protein or a peptide immunotherapeutic agent.

### Immune Cell Associated Antigen Specific Antibodies

In some embodiments, the antibody or antigen binding fragment thereof is specific for an immune cell associated antigen. In some embodiments, the immune cell associated antigen is associated with an immune cell subtype (e.g., lymphocyte, neutrophil, macrophage, etc.). In some embodiments, the immune cell associated antigen is associated with a T cell, a monocyte, and/or a natural killer (NK) cell. In embodiments, the immune cell antigen is associated with a T cell. In some embodiments, the immune cell antigen is associated with an effector T cell, a cytotoxic T cell, a helper T cell, a regulatory T cell, and/or a memory T cell.

In some embodiments, the immune cell associated antigen is an immune checkpoint molecule. In some embodiments, the immune cell associated antigen is a costimulatory antigen. In some embodiments, the immune cell associated antigen is an macrophage cell surface antigen. In some embodiments, the immune cell associated antigen is an NK cell surface antigen. In some embodiments, the immune cell associated antigen is a T cell surface antigen (e.g., CD8A, CD8B).

In some embodiments, the immune cell associated antigen is 4-IBB, B7-H3, B7-H4, BTLA, CD3, CCR8, CD8A, CD8B, CD16A, CD27, CD28, CD33, CD38, CD39, CD40, CD47, CD70, CD80, CD86, CD96, CD163, CLEC-1, CLEVER-1, CTLA-4, D40, GITR, ICOS, ILT2/3/4, LAG-3, MHCI, MHCII, NKG2A, NKG2D, NKp30, NKp44, NKp46, OX40, PD-1, PD-L1, PD-L2, PSGL-1, SIGLEC-9, SIGLEC-15, SIRP-α, TCR, TIGIT, TIM-3, VISTA, or VSIG4 . . . . In some embodiments, the immune cell associated antigen is PD-1. In some embodiments, the immune cell associated antigen is CCR8, CD8A, CD8B, CD16A, CD96, CD226, CTLA-4, ICOS, LAG-3, NKG2A, NKG2D, NKp30, NKp44, NKp46, PD-1, PD-L1, TIGIT, or TIM-3.

In some embodiments, the immune cell associated antigen is 4-1BB. In some embodiments, the immune cell associated antigen is B7-H3. In some embodiments, the immune cell associated antigen is B7-H4. In some embodiments, the immune cell associated antigen is BTLA. In some embodiments, the immune cell associated antigen is CD3. In some embodiments, the immune cell associated antigen is CCR8. In some embodiments, the immune cell associated antigen is CD8A. In some embodiments, the immune cell associated antigen is CD8B. In some embodiments, the immune cell associated antigen is CD16A. In some embodiments, the immune cell associated antigen is CD27. In some embodiments, the immune cell associated antigen is CD33. In some embodiments, the immune cell associated antigen is CD38. In some embodiments, the immune cell associated antigen is CD39. In some embodiments, the immune cell associated antigen is CD40. In some embodiments, the immune cell associated antigen is CD47. In some embodiments, the immune cell associated antigen is CD80. In some embodiments, the immune cell associated antigen is CD86. In some embodiments, the immune cell associated antigen is CD96. In some embodiments, the immune cell associated antigen is CD163. In some embodiments, the immune cell associated antigen is CLEC-1. In some embodiments, the immune cell associated antigen is CLEVER-1. In some embodiments, the immune cell associated antigen is CTLA4. In some embodiments, the immune cell associated antigen is D40. In some embodiments, the immune cell associated antigen is GITR. In some embodiments, the immune cell associated antigen is ICOS. In some embodiments, the immune cell associated antigen is ILT2/3/4. In some embodiments, the immune cell associated antigen is LAG-3. In some embodiments, the immune cell associated antigen is MHCI. In some embodiments, the immune cell associated antigen is MHCII. In some embodiments, the immune cell associated antigen is NKG2A. In some embodiments, the immune cell associated antigen is NKp30. In some embodiments, the immune cell associated antigen is NKp44. In some embodiments, the immune cell associated antigen is NKp46. In some embodiments, the immune cell associated antigen is OX40. In some embodiments, the immune cell associated antigen is PD-1. In some embodiments, the immune cell associated antigen is PD-L1. In some embodiments, the immune cell associated antigen is PD-L2. In some embodiments, the immune cell associated antigen is PSGL-1. In some embodiments, the immune cell associated antigen is SIGLEC-9. In some embodiments, the immune cell associated antigen is SIGLEC-15. In some embodiments, the immune cell associated antigen is SIRP-α. In some embodiments, the immune cell associated antigen is TCR. In some embodiments, the immune cell associated antigen is TIGIT. In some embodiments, the immune cell associated antigen is TIM-3. In some embodiments, the immune cell associated antigen is VISTA. In some embodiments, the immune cell associated antigen is VSIG4.

In some embodiments, the antibody or antigen binding fragment thereof is an anti-PD-1 antibody or antigen binding fragment. Programmed cell death protein 1 (also known as PD-1 and CD279), is a cell surface receptor that plays an role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is MQIPQAPWPVVWAVLQLGWRPGWFLDSPDRPWNPPTFSPALLVVTEGDNATFTCSFSN TSESFVLNWYRMSPSNQTDKLAAFPEDRSQPGQDCRFRVTQLPNGRDFHMSVVRARRN DSGTYLCGAISLAPKAQIKESLRAELRVTERRAEVPTAHPSPSPRPAGQFQTLVVGVVGG LLGSLVLLVWVLAVICSRAARGTIGARRTGQPLKEDPSAVPVFSVDYGELDFQWREKTP EPPVPCVPEQTEYATIVFPSGMGTSSPARRGSADGPRSAQPLRPEDGHCSWPL (SEQ ID NO: 331).

In one embodiment, an anti-PD-1 antibody or an anti-PD-1 antigen binding fragment of the disclosure comprises a combination of a heavy chain variable region (VH) and a light chain variable region (VL) described herein. In another embodiment, an anti-PD-1 antibody or an anti-PD-1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-1 antibody or an anti-PD-1 antigen binding fragment of the disclosure comprises a modified Tislelizumab, Baizcan, OKVO411B3N, BGB-A317, hu317-1/lgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYOR, 6QVL057INT, H4H7798N, REGN-2810, SAR-439684, Avelumab, BAVENCIOR, 451238, KXG2PJ551I, MSB-0010682, MSB-0010718C, PF-06834635, Durvalumab, IMFINZI®, 28X28X9OKV, MEDI-4736, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberclimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, POGVQ9A4S5, TSR-042, WBP-285, Serplulimab, HLX-10, CS-1003, Retifanlimab, 2Y3T5IF01Z, INCMGA-00012, INCMGA-0012, MGA-012, Sasanlimab, LZZOIC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotclimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDI0680, or AMP-514.

In some embodiments, an anti-PD-1 antibody or an anti-PD-1 antigen binding fragment of the disclosure comprises a Tislelizumab, Sintilimab, Toripalimab, Terepril, Camrelizumab, Cemiplimab, Pembrolizumab Nivolumab, Prolgolimab, Penpulimab, Zimberclimab, Balstilimab, Genolimzumab, Geptanolimab, Dostarlimab, Serplulimab, Retifanlimab, Sasanlimab, Spartalizumab, Cetrelimab, Tebotclimab, Cadonilimab, A Pidilizumab, LZM-009, or Budigalimab. In one embodiment, an anti-PD-1 antibody or an anti-PD-1 antigen binding fragment of the disclosure comprises a modified Tislelizumab, Sintilimab, Toripalimab, Terepril, Camrelizumab, Cemiplimab, Pembrolizumab Nivolumab, Prolgolimab, Penpulimab, Zimberclimab, Balstilimab, Genolimzumab, Geptanolimab, Dostarlimab, Serplulimab, Retifanlimab, Sasanlimab, Spartalizumab, Cetrelimab, Tebotclimab, Cadonilimab, Pidilizumab, LZM-009, or Budigalimab.

In some embodiments, the anti-PD-1 polypeptide is Nivolumab, Pembrolizumab, LZM-009, Dostarlimab, Sintilimab, Spartalizumab, Tislelizumab, or Cemiplimab. In some embodiment, the anti-PD-1 polypeptide is Dostarlimab, Sintilimab, Spartalizumab, or Tislelizumab. In some embodiments, the anti-PD-1 polypeptide is Nivolumab, Pembrolizumab, LZM-009, or Cemiplimab.

In some embodiments, the anti-PD-1 polypeptide is LZM-009. The VH and VL of LZM-009 are provided in Table 1 below as SEQ ID NOs: 376 and 377, respectively. The full length heavy chain of LZM-009 has the sequence QVQLVQSGAEVKKPGASVKVSCKASGYTFTSYYMYWVRQAPGQGLEWMGGVNPSNG GTNFNEKFKSRVTITADKSTSTAYMELSSLRSEDTA VYYCARRDYRYDMGFDYWGQGT TVTVSSASTKGPSVFPLAPCSRSTSESTAALGCLVKDYFPEPVTVSWNSGALTSGVHTFP AVLQSSGLYSLSSVVTVPSSSLGTKTYTCNVDHKPSNTKVDKRVESKYGPPCPPCPAPEF LGGPSVFLFPPKPKDTLMISRTPEVTCVVVDVSQEDPEVQFNWYVDGVEVHNAKTKPRE EQFNSTYRVVSVLTVLHQDWLNGKEYKCKVSNKGLPSSIEKTISKAKGQPREPQVYTLP PSQEEMTKNQVSLTCLVKGFYPSDIA VEWESNGQPENNYKTTPPVLDSDGSFFLYSRLT VDKSRWQEGNVFSCSVMHEALHNHYTQKSLSLSLGK (SEQ ID NO: 404). The full length light chain of LZM-009 has the sequence EIVLTQSPATLSLSPGERATISCRASKGVSTSGYSYLHWYQQKPGQAPRLLIYLASYLESG VPARFSGSGSGTDFTLTISSLEPEDFATYYCQHSRELPLTFGTGTKVEIKRTVAAPSVFIFP PSDEQLKSGTASVVCLLNNFYPREAKVQWKVDNALQSGNSQESVTEQDSKDSTYSLSST LTLSKADYEKHKVYACEVTHQGLSSPVTKSFNRGEC (SEQ ID NO: 405).

In some embodiments, the anti-PD-1 antibody is Pembrolizumab. In some embodiments, the anti-PD-1 antibody is modified Pembrolizumab.

In some embodiments, the anti-PD-1 antibody is a biosimilar of Tislelizumab, Sintilimab, Toripalimab, Terepril, Camrelizumab, Cemiplimab, Pembrolizumab Nivolumab, Prolgolimab, Penpulimab, Zimberclimab, Balstilimab, Genolimzumab, Geptanolimab, Dostarlimab, Serplulimab, Retifanlimab, Sasanlimab, Spartalizumab, Cetrelimab, Tebotclimab, Cadonilimab, A Pidilizumab, LZM-009, or Budigalimab. In some embodiments, the anti-PD-1 antibody is a biosimilar of any one of the antibodies provided herein.

TABLE 1 provides the sequences of exemplary anti-PD-1 antibodies and anti-PD-1 antigen binding fragments that can be modified to prepare anti-PD-1 immunoconjugates. TABLE 1 also shows provides combinations of CDRs that can be utilized in a modified anti-PD-1 immunoconjugate. Reference to an anti-PD-1 antibody herein may alternatively refer to an anti-PD-1 antigen binding fragment.

In some instances, the SEQ ID NOs listed in Table 1 contain full-length heavy or light chains of the indicated antibodies with the VH or VL respectively indicated in bold. Where there is a reference herein to a VH or VL of a SEQ ID NO in Table 1 which contains a full-length heavy or light chain, it is intended to reference the bolded portion of the sequence. For example, reference to “a VH having an amino acid sequence shown in SEQ ID NO: 332” refers to the bolded portion of SEQ ID NO: 332 in Table 1.

An anti-PD-1 antibody or an anti-PD-1 antigen binding fragment can comprise a VH having an amino acid sequence of any one of SEQ ID NOS: 332, 334, 336, 338, 340, 346, 348, 350, 352, 354, 356, 358, 360, 362, 364, 366, 368, 370, 372, 374, 376, and 378. An anti-PD-1 antibody or an anti-PD-1 antigen binding fragment can comprise a VL having an amino acid sequence of any one of SEQ ID NOS: 333, 335, 337, 339, 341, 347, 349, 351, 353, 355, 357, 359, 361, 363, 365, 367, 369, 371, 373, 375, 377, and 379.

An anti-PD-1 antibody or an anti-PD-1 antigen binding fragment can comprise a heavy chain or VH having an amino acid sequence of any one of SEQ ID NOS: 332, 334, 336, 338, 340, 346, 348, 350, 352, 354, 356, 358, 360, 362, 364, 366, 368, 370, 372, 374, 376, and 378, or a portion corresponding to a VH thereof. An anti-PD-1 antibody or an anti-PD-1 antigen binding fragment can comprise a light chain or VL having an amino acid sequence of any one of SEQ ID NOS: 333, 335, 337, 339, 341, 347, 349, 351, 353, 355, 357, 359, 361, 363, 365, 367, 369, 371, 373, 375, 377, and 379, or a portion corresponding to a VL thereof.

In one instance, an anti-PD-1 antibody or an anti-PD-1 antigen binding fragment comprises a VH having an amino acid sequence shown in SEQ ID NO: 332, and a VL having an amino acid sequence shown in SEQ ID NO: 333. In another instance, an anti-PD-1 antibody or an anti-PD-1 antigen binding fragment comprises a VH having an amino acid sequence shown in SEQ ID NO: 334, and a VL having an amino acid sequence shown in SEQ ID NO: 335. In another instance, an anti-PD-1 antibody or an anti-PD-1 antigen binding fragment comprises a VH having an amino acid sequence shown in SEQ ID NO: 336, and a VL having an amino acid sequence shown in SEQ ID NO: 337. In another instance, an anti-PD-1 antibody or an anti-PD-1 antigen binding fragment comprises a VH having an amino acid sequence shown in SEQ ID NO: 338, and a VL having an amino acid sequence shown in SEQ ID NO: 339. In another instance, an anti-PD-1 antibody or an anti-PD-1 antigen binding fragment comprises a VH having an amino acid sequence shown in SEQ ID NO: 340, and a VL having an amino acid sequence shown in SEQ ID NO: 341. In another instance, an anti-PD-1 antibody or an anti-PD-1 antigen binding fragment comprises a VH having an amino acid sequence shown in SEQ ID NO: 346, and a VL having an amino acid sequence shown in SEQ ID NO: 347. In another instance, an anti-PD-1 antibody or an anti-PD-1 antigen binding fragment comprises a VH having an amino acid sequence shown in SEQ ID NO: 348, and a VL having an amino acid sequence shown in SEQ ID NO: 349. In another instance, an anti-PD-1 antibody or an anti-PD-1 antigen binding fragment comprises a VH having an amino acid sequence shown in SEQ ID NO: 350, and a VL having an amino acid sequence shown in SEQ ID NO: 351. In another instance, an anti-PD-1 antibody or an anti-PD-1 antigen binding fragment comprises a VH having an amino acid sequence shown in SEQ ID NO: 352, and a VL having an amino acid sequence shown in SEQ ID NO: 353. In another instance, an anti-PD-1 antibody or an anti-PD-1 antigen binding fragment comprises a VH having an amino acid sequence shown in SEQ ID NO: 354, and a VL having an amino acid sequence shown in SEQ ID NO: 355. In another instance, an anti-PD-1 antibody or an anti-PD-1 antigen binding fragment comprises a VH having an amino acid sequence shown in SEQ ID NO: 356, and a VL having an amino acid sequence shown in SEQ ID NO: 357. In another instance, an anti-PD-1 antibody or an anti-PD-1 antigen binding fragment comprises a VH having an amino acid sequence shown in SEQ ID NO: 358, and a VL having an amino acid sequence shown in SEQ ID NO: 359. In another instance, an anti-PD-1 antibody or an anti-PD-1 antigen binding fragment comprises a VH having an amino acid sequence shown in SEQ ID NO: 360, and a VL having an amino acid sequence shown in SEQ ID NO: 361. In another instance, an anti-PD-1 antibody or an anti-PD-1 antigen binding fragment comprises a VH having an amino acid sequence shown in SEQ ID NO: 362, and a VL having an amino acid sequence shown in SEQ ID NO: 363. In another instance, an anti-PD-1 antibody or an anti-PD-1 antigen binding fragment comprises a VH having an amino acid sequence shown in SEQ ID NO: 364, and a VL having an amino acid sequence shown in SEQ ID NO: 365. In another instance, an anti-PD-1 antibody or an anti-PD-1 antigen binding fragment comprises a VH having an amino acid sequence shown in SEQ ID NO: 366, and a VL having an amino acid sequence shown in SEQ ID NO: 367. In another instance, an anti-PD-1 antibody or an anti-PD-1 antigen binding fragment comprises a VH having an amino acid sequence shown in SEQ ID NO: 368, and a VL having an amino acid sequence shown in SEQ ID NO: 369. In another instance, an anti-PD-1 antibody or an anti-PD-1 antigen binding fragment comprises a VH having an amino acid sequence shown in SEQ ID NO: 370, and a VL having an amino acid sequence shown in SEQ ID NO: 371. In another instance, an anti-PD-1 antibody or an anti-PD-1 antigen binding fragment comprises a VH having an amino acid sequence shown in SEQ ID NO: 372, and a VL having an amino acid sequence shown in SEQ ID NO: 373. In another instance, an anti-PD-1 antibody or an anti-PD-1 antigen binding fragment comprises a VH having an amino acid sequence shown in SEQ ID NO: 374, and a VL having an amino acid sequence shown in SEQ ID NO: 375. In another instance, an anti-PD-1 antibody or an anti-PD-1 antigen binding fragment comprises a VH having an amino acid sequence shown in SEQ ID NO: 376, and a VL having an amino acid sequence shown in SEQ ID NO: 377. In another instance, an anti-PD-1 antibody or an anti-PD-1 antigen binding fragment comprises a VH having an amino acid sequence shown in SEQ ID NO: 378, and a VL having an amino acid sequence shown in SEQ ID NO: 379.

In one instance, an anti-PD-1 antibody or an anti-PD-1 antigen binding fragment comprises a VH having an amino acid sequence of SEQ ID NO: 332, and a VL having an amino acid sequence of SEQ ID NO: 333. In another instance, an anti-PD-1 antibody or an anti-PD-1 antigen binding fragment comprises a VH having an amino acid sequence of SEQ ID NO: 334, and a VL having an amino acid sequence of SEQ ID NO: 335. In another instance, an anti-PD-1 antibody or an anti-PD-1 antigen binding fragment comprises a VH having an amino acid sequence of SEQ ID NO: 336, and a VL having an amino acid sequence of SEQ ID NO: 337. In another instance, an anti-PD-1 antibody or an anti-PD-1 antigen binding fragment comprises a VH having an amino acid sequence of SEQ ID NO: 338, and a VL having an amino acid sequence of SEQ ID NO: 339. In another instance, an anti-PD-1 antibody or an anti-PD-1 antigen binding fragment comprises a VH having an amino acid sequence of SEQ ID NO: 340, and a VL having an amino acid sequence of SEQ ID NO: 341. In another instance, an anti-PD-1 antibody or an anti-PD-1 antigen binding fragment comprises a VH having an amino acid sequence of SEQ ID NO: 346, and a VL having an amino acid sequence of SEQ ID NO: 347. In another instance, an anti-PD-1 antibody or an anti-PD-1 antigen binding fragment comprises a VH having an amino acid sequence of SEQ ID NO: 348, and a VL having an amino acid sequence of SEQ ID NO: 349. In another instance, an anti-PD-1 antibody or an anti-PD-1 antigen binding fragment comprises a VH having an amino acid sequence of SEQ ID NO: 350, and a VL having an amino acid sequence of SEQ ID NO: 351. In another instance, an anti-PD-1 antibody or an anti-PD-1 antigen binding fragment comprises a VH having an amino acid sequence of SEQ ID NO: 352, and a VL having an amino acid sequence of SEQ ID NO: 353. In another instance, an anti-PD-1 antibody or an anti-PD-1 antigen binding fragment comprises a VH having an amino acid sequence of SEQ ID NO: 354, and a VL having an amino acid sequence of SEQ ID NO: 355. In another instance, an anti-PD-1 antibody or an anti-PD-1 antigen binding fragment comprises a VH having an amino acid sequence of SEQ ID NO: 356, and a VL having an amino acid sequence of SEQ ID NO: 357. In another instance, an anti-PD-1 antibody or an anti-PD-1 antigen binding fragment comprises a VH having an amino acid sequence of SEQ ID NO: 358, and a VL having an amino acid sequence of SEQ ID NO: 359. In another instance, an anti-PD-1 antibody or an anti-PD-1 antigen binding fragment comprises a VH having an amino acid sequence of SEQ ID NO: 360, and a VL having an amino acid sequence of SEQ ID NO: 361. In another instance, an anti-PD-1 antibody or an anti-PD-1 antigen binding fragment comprises a VH having an amino acid sequence of SEQ ID NO: 362, and a VL having an amino acid sequence of SEQ ID NO: 363. In another instance, an anti-PD-1 antibody or an anti-PD-1 antigen binding fragment comprises a VH having an amino acid sequence of SEQ ID NO: 364, and a VL having an amino acid sequence of SEQ ID NO: 365. In another instance, an anti-PD-1 antibody or an anti-PD-1 antigen binding fragment comprises a VH having an amino acid sequence of SEQ ID NO: 366, and a VL having an amino acid sequence of SEQ ID NO: 367. In another instance, an anti-PD-1 antibody or an anti-PD-1 antigen binding fragment comprises a VH having an amino acid sequence of SEQ ID NO: 368, and a VL having an amino acid sequence of SEQ ID NO: 369. In another instance, an anti-PD-1 antibody or an anti-PD-1 antigen binding fragment comprises a VH having an amino acid sequence of SEQ ID NO: 370, and a VL having an amino acid sequence of SEQ ID NO: 371. In another instance, an anti-PD-1 antibody or an anti-PD-1 antigen binding fragment comprises a VH having an amino acid sequence of SEQ ID NO: 372, and a VL having an amino acid sequence of SEQ ID NO: 373. In another instance, an anti-PD-1 antibody or an anti-PD-1 antigen binding fragment comprises a VH having an amino acid sequence of SEQ ID NO: 374, and a VL having an amino acid sequence of SEQ ID NO: 375. In another instance, an anti-PD-1 antibody or an anti-PD-1 antigen binding fragment comprises a VH having an amino acid sequence of SEQ ID NO: 376, and a VL having an amino acid sequence of SEQ ID NO: 377. In another instance, an anti-PD-1 antibody or an anti-PD-1 antigen binding fragment comprises a VH having an amino acid sequence of SEQ ID NO: 378, and a VL having an amino acid sequence of SEQ ID NO: 379.

In one instance, an anti-PD-1 antibody or an anti-PD-1 antigen binding fragment comprises a VH CDR1 having an amino acid sequence of SEQ ID NO: 380, a VH CDR2 having an amino acid sequence of SEQ ID NO: 381, a VH CDR3 having an amino acid sequence of SEQ ID NO: 382, VL CDR1 having an amino acid sequence of SEQ ID NO: 383, a VL CDR2 having an amino acid sequence of SEQ ID NO: 384, and a VL CDR3 having an amino acid sequence of SEQ ID NO: 385. In one instance, an anti-PD-1 antibody or an anti-PD-1 antigen binding fragment comprises a VH CDR1 having an amino acid sequence of SEQ ID NO: 386, a VH CDR2 having an amino acid sequence of SEQ ID NO: 387, a VH CDR3 having an amino acid sequence of SEQ ID NO: 388, VL CDR1 having an amino acid sequence of SEQ ID NO: 389, a VL CDR2 having an amino acid sequence of SEQ ID NO: 390, and a VL CDR3 having an amino acid sequence of SEQ ID NO: 391. In one instance, an anti-PD-1 antibody or an anti-PD-1 antigen binding fragment comprises a VH CDR1 having an amino acid sequence of SEQ ID NO: 392, a VH CDR2 having an amino acid sequence of SEQ ID NO: 393, a VH CDR3 having an amino acid sequence of SEQ ID NO: 394, VL CDR1 having an amino acid sequence of SEQ ID NO: 395, a VL CDR2 having an amino acid sequence of SEQ ID NO: 396, and a VL CDR3 having an amino acid sequence of SEQ ID NO: 397. In one instance, an anti-PD-1 antibody or an anti-PD-1 antigen binding fragment comprises a VH CDR1 having an amino acid sequence of SEQ ID NO: 398, a VH CDR2 having an amino acid sequence of SEQ ID NO: 399, a VH CDR3 having an amino acid sequence of SEQ ID NO: 400, VL CDR1 having an amino acid sequence of SEQ ID NO: 401, a VL CDR2 having an amino acid sequence of SEQ ID NO: 402, and a VL CDR3 having an amino acid sequence of SEQ ID NO: 403.

In some embodiments, the antibody or antigen binding fragment thereof is an anti-PD-L1 antibody or antigen binding fragment. Programmed death-ligand 1 (PD-L1) is a ligand for an immunosuppressive receptor “programmed death receptor 1 (PD-1)” that is predominantly expressed in activated T and B cells, which can negatively regulate antigen receptor signaling. The ligands (PD-L1 and PD-L2) for PD-1 may be constitutively expressed or may be derived into a number of cell types, including non-hematopoietic cell tissues and various tumor types. PD-L1 is expressed in B cells, T cells, bone marrow cells and dendritic cells (DCs), but also on non-lymphatic organs such as peripheral cells, pseudo-vascular endothelial cells and heart, lungs, etc. A non-limiting, exemplary, human PD-L1 amino acid sequence is MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTTTNSKREEKLFNVTSTLRINTTTNEIFYCTFRRLDPEENHTAEL VIPELPLAHPPNE RTHLVILGAILLCLGVALTFIFRLRKGRMMDVKKCGIQDTNSKKQSDTHLEET (SEQ ID NO: 330)

In one embodiment, an anti-PD-L1 antibody or an anti-PD-L1 antigen binding fragment of the disclosure comprises a combination of a heavy chain variable region (VH) and a light chain variable region (VL) described herein. In another embodiment, an anti-PD-L1 antibody or an anti-PD-L1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-L1 antibody or an anti-PD-L1 antigen binding fragment of the disclosure comprises a modified Avelumab (Bavencio, 451238, KXG2PJ551I, MSB-0010682, MSB-0010718C, PF-06834635, CAS 1537032-82-8: EMD Serono, Merck & Co., Merck KGaA, Merck Serono, National Cancer Institute (NCI), Pfizer), Durvalumab (Imfinzi, 28×28×90 KV (UNII code), MEDI-4736, CAS 1428935-60-7: AstraZeneca, Celgene, Children's Hospital Los Angeles (CHLA), City of Hope National Medical Center, MedImmune, Memorial Sloan-Kettering Cancer Center, Mirati Therapeutics, National Cancer Institute (NCI), Samsung Medical Center (SMC), Washington University), Atezolizumab (Tecentriq, 52CMIOWC3Y, MPDL-3280A, RG-7446, RO-5541267, CAS 1380723-44-3: Academisch Medisch Centrum (AMC), Chugai Pharmaceutical, EORTC, Genentech, Immune Design (Merck & Co.), Memorial Sloan-Kettering Cancer Center, National Cancer Institute (NCI), Roche, Roche Center for Medical Genomics), Sugemalimab (CS-1001, WBP-3155: CStone Pharmaceuticals, EQRx, Pfizer), KN-046 (CAS 2256084-03-2: Jiangsu Alphamab Biopharmaceuticals, Sinovent), APL-502 (CBT-502, TQB-2450: Apollomics, Jiangsu Chia Tai Tianqing Pharmaceutical), Envafolimab (3D-025, ASC-22, KN-035, hu56V1-Fc-m1, CAS 2102192-68-5: 3D Medicines, Ascletis, Jiangsu Alphamab Biopharmaceuticals, Suzhou Alphamab, Tracon Pharmaceuticals, Inc.), Bintrafusp alfa (M-7824, MSB-0011359C, NW9K8CIJN3, CAS 1918149-01-5: EMD Serono, GlaxoSmithKline, Merck KGaA, National Cancer Institute (NCI)), STI-1014 (STI-A1014, ZKAB-001: Lec's Pharmaceutical, Sorrento Therapeutics), PD-L1 t-haNK (ImmunityBio, NantKwest), A-167 (HBM-9167, KL-A167: Harbour BioMed, Sichuan Kelun-Biotech Biopharmaceutical), IMC-001 (STI-3031, STI-A-1015, STI-A1015, ImmuneOncia Therapeutics, Sorrento Therapeutics), HTI-1088 (SHR-1316: Atridia, Jiangsu Hengrui), IO-103 (IO Biotech), CX-072 (CytomX Therapeutics), AUPM-170 (CA-170: Aurigene, Curis), GS-4224 (Gilcad), ND-021 (NM21-1480, PRO-1480: CStone Pharmaceuticals, Numab Therapeutics), BNT-311 (DuoBody-PD-L1x4-1BB, GEN-1046: BioNTech, Genmab), BGB-A333 (BeiGene), IBI-322 (Innovent Biologics), NM-01 (Nanomab Technology, Shanghai First People's Hospital), LY-3434172 (Eli Lilly), LDP (Dragonboat Biopharmaceutical), CDX-527 (Celldex Therapeutics), IBI-318 (Innovent Biologics, Lilly), 89Zr-DFO-REGN3504 (Regencron), ALPN-202 (CD80 vlgD-Fc: Alpine Immune Sciences), INCB-086550 (Incytc), LY-3415244 (Eli Lilly), SHR-1701 (Jiangsu Hengrui), JS-003 (JS003-30, JS003-SD: Shanghai Junshi Biosciences), HLX-20 (PL2 #3: Henlix Biotech, Shanghai Henlius Biotech), ES-101 (INBRX-105, INBRX-105-1: Elpiscience BioPharma, Inhibrx), MSB-2311 (MabSpace Biosciences), PD-1-Fc-OX40L (SL-279252, TAK-252: Heat Biologics, Shattuck Labs, Takeda), FS-118, FS118 mAb2, LAG-3/PD-L1 mAb2: F-star Therapeutics, Merck & Co., Merck KGaA), FAZ-053 (LAE-005: Lackna Therapeutics, Novartis), Lodapolimab (LY-3300054, NR4MAD6PPB, CAS 2118349-31-6: Eli Lilly), MCLA-145 (Incyte, Merus), BMS-189 (BMS-986189, PD-L1-Milla from Bristol-Myers Squibb), Cosibelimab (CK-301, TG-1501, CAS 2216751-26-5: Checkpoint Therapeutics, Dana-Farber Cancer Institute, Samsung Biologics, TG Therapeutics), IL-15Ralpha-SD/IL-15 (KD-033: Kadmon), WP-1066 (CAS 857064-38-1: M.D. Anderson Cancer Center, Moleculin Biotech), BMS-936559 (MDX-1105: Bristol-Myers Squibb, Medarex, National Institute Allergy Infect Dis.), BMS-986192 (Bristol-Myers Squibb), RC-98 (RemeGen), CD-200AR-L (CD200AR-L: OX2 Therapeutics, University of Minnesota), ATA-3271 (Atara Biotherapeutics), IBC-Ab002 (ImmunoBrain Checkpoint), BMX-101 (Biomunex Pharmaceuticals), AVA-04-VbP (Avacta), ACE-1708 (Acepodia Biotech), KY-1043 (Kymab, Provenance Biopharmaceuticals), ACE-05 (YBL-013: Y-Biologics), ONC-0055 (ONC0055, PRS-344 S-095012: Picris Pharmaceuticals, Servier), TLJ-1-CK (I-Mab Biopharma), GR-1405 (Chinese Academy of Medical Sciences), PD-1ACR-T (Taipci Medical University), N-809 (N-IL15/PD-L1: ImmunityBio), CB-201 (Crescendo Biologics), MEDI-1109 (MedImmunc), AVA-004 (AVA-04: Avacta), CA-327 (Aurigene, Curis), ALN-PDL (Alnylam Pharmaceuticals), KY-1003 (Kymab), CD22 (aPD-L1) CAR-T cells (SL-22P: Hebci Senlang Biotechnology), ATA-2271 (M28z1XXPD-IDNR CAR T cells: Atara Biotherapeutics), and Zeushield cytotoxic T lymphocytes (Second Xiangya Hosp Central South Univ.).

In some embodiments, the anti-PD-L1 antibody is Avelumab, Durvalumab, Atczolizumab, Sugemalimab, Envafolimab, Lodapolimab, or Cosibelimab, or a modified version thereof. In some embodiments, the anti-PD-L1 antibody is Avelumab, Durvalumab, Atezolizumab, Sugemalimab, Envafolimab, Lodapolimab, or Cosibelimab. In some embodiments, the antibody is a biosimilar of Avclumab, Durvalumab, Atczolizumab, Sugemalimab, Envafolimab, Lodapolimab, or Cosibclimab.

TABLE 1 provides the sequences of exemplary anti-PD-L1 antibodies and anti-PD-L1 antigen binding fragments that can be modified to prepare anti-PD-L1 immunoconjugates. TABLE 1 also provides exemplary combinations of CDRs that can be utilized in a modified anti-PD-L1 immunoconjugate. Reference to an anti-PD-L1 antibody herein may alternatively refer to an anti-PD-L1 antigen binding fragment.

In some embodiments, an anti-PD-L1 antibody or an anti-PD-L1 antigen binding fragment comprises a heavy chain or VH having an amino acid sequence of any one of SEQ ID NOS: 232, 234, 236, 238, 242, 244, or 248. An anti-PD-L1 antibody or an anti-PD-L1 antigen binding fragment comprises a heavy chain or VH having an amino acid sequence of any one of SEQ ID NOS: 233, 235, 237, 239, 243, 245, or 249. In one instance, an anti-PD-L1 antibody or an anti-PD-L1 antigen binding fragment comprises a heavy chain or VH having an amino acid sequence of SEQ ID NO: 232, and a light chain or VL having an amino acid sequence of SEQ ID NO: 233. In another instance, an anti-PD-L1 antibody or an anti-PD-L1 antigen binding fragment comprises a heavy chain or VH having an amino acid sequence of SEQ ID NO: 234, and a light chain or VL having an amino acid sequence of SEQ ID NO: 235. In another instance, an anti-PD-L1 antibody or an anti-PD-L1 antigen binding fragment comprises a heavy chain or VH having an amino acid sequence of SEQ ID NO: 236, and a light chain or VL having an amino acid sequence of SEQ ID NO: 237. In another instance, an anti-PD-L1 antibody or an anti-PD-L1 antigen binding fragment comprises a heavy chain or VH having an amino acid sequence of SEQ ID NO: 238, and a light chain or VL having an amino acid sequence of SEQ ID NO: 239. In another instance, an anti-PD-L1 antibody or an anti-PD-L1 antigen binding fragment comprises a VH having an amino acid sequence of SEQ ID NO: 242, and a VL having an amino acid sequence of SEQ ID NO: 243. In another instance, an anti-PD-L1 antibody or an anti-PD-L1 antigen binding fragment comprises a VH having an amino acid sequence of SEQ ID NO: 244, and a VL having an amino acid sequence of SEQ ID NO: 245. In another instance, an anti-PD-L1 antibody or an anti-PD-L1 antigen binding fragment comprises a heavy chain or VH having an amino acid sequence of SEQ ID NO: 248, and a light chain or VL having an amino acid sequence of SEQ ID NO: 249.

In some embodiments, an anti-PD-L1 antibody or an anti-PD-L1 antigen binding fragment comprises a heavy chain or VH having an amino acid sequence of any one of SEQ ID NOS: 232, 234, 236, 238, 242, 244, or 248, or a portion corresponding to a VH thereof. An anti-PD-L1 antibody or an anti-PD-L1 antigen binding fragment comprises a light chain or VL having an amino acid sequence of any one of SEQ ID NOS: 233, 235, 237, 239, 243, 245, or 249, or a portion corresponding to a VL thereof. In one instance, an anti-PD-L1 antibody or an anti-PD-L1 antigen binding fragment comprises a heavy chain or VH having an amino acid sequence shown in SEQ ID NO: 232, and a light chain or VL having an amino acid sequence shown in SEQ ID NO: 233. In another instance, an anti-PD-L1 antibody or an anti-PD-L1 antigen binding fragment comprises a heavy chain or VH having an amino acid sequence shown in SEQ ID NO: 234, and a light chain or VL having an amino acid sequence shown in SEQ ID NO: 235. In another instance, an anti-PD-L1 antibody or an anti-PD-L1 antigen binding fragment comprises a heavy chain or VH having an amino acid sequence shown in SEQ ID NO: 236, and a light chain or VL having an amino acid sequence shown in SEQ ID NO: 237. In another instance, an anti-PD-L1 antibody or an anti-PD-L1 antigen binding fragment comprises a heavy chain or VH having an amino acid sequence shown in SEQ ID NO: 238, and a light chain or VL having an amino acid sequence shown in SEQ ID NO: 239. In another instance, an anti-PD-L1 antibody or an anti-PD-L1 antigen binding fragment comprises a VH having an amino acid sequence shown in SEQ ID NO: 242, and a VL having an amino acid sequence shown in SEQ ID NO: 243. In another instance, an anti-PD-L1 antibody or an anti-PD-L1 antigen binding fragment comprises a VH having an amino acid sequence shown in SEQ ID NO: 244, and a VL having an amino acid sequence shown in SEQ ID NO: 245. In another instance, an anti-PD-L1 antibody or an anti-PD-L1 antigen binding fragment comprises a heavy chain or VH having an amino acid sequence shown in SEQ ID NO: 248, and a light chain or VL having an amino acid sequence shown in SEQ ID NO: 249.

In one instance, an anti-PD-L1 antibody or an anti-PD-L1 antigen binding fragment comprises a VH CDR1 having an amino acid sequence of SEQ ID NO: 250, a VH CDR2 having an amino acid sequence of SEQ ID NO: 251, a VH CDR3 having an amino acid sequence of SEQ ID NO: 252, VL CDR1 having an amino acid sequence of SEQ ID NO: 253, a VL CDR2 having an amino acid sequence of SEQ ID NO: 254, and a VL CDR3 having an amino acid sequence of SEQ ID NO: 255.

In one instance, an anti-PD-L1 antibody comprises a single domain binding antibody having an amino acid sequence of SEQ ID NO: 256, a tri-specific fusion single chain antibody construct having an amino acid sequence of SEQ ID NO: 257, or a bispecific tetrameric antibody like engager having an amino acid sequence of SEQ ID NO: 258.

TABLE 1
Exemplary Antibodies for Immune Cell Associated Antigens

| Antibody | | | |
|---|---|---|---|
| or Ag- | | | SEQ |
| binding | Antigen | | ID |
| fragment | Bound | Sequence | NO |
| Tislelizumab, | PD-1 | QVQLQESGPGLVKPSETLSLTCTVSGFSLTSYGVHWIRQPPGK | 332 |
| Baizean, | | GLEWIGVIYADGSTNYNPSLKSRVTISKDTSKNQVSLKLSSVT | |
| 0KVO411B3N, | | AADTAVYYCARAYGNYWYIDVWGQGTTVTVSSASTKGPSVFP | |
| BGB- | | LAPCSRSTSESTAALGCLVKDYFPEPVTVSWNSGALTSGVHTFPA | |
| A317, | | VLQSSGLYSLSSVVTVPSSSLGTKTYTCNVDHKPSNTKVDKRVES | |
| hu317- | | KYGPPCPPCPAPPVAGGPSVFLFPPKPKDTLMISRTPEVTCVVVAV | |
| 1/IgG4mt2 | | SQEDPEVQFNWYVDGVEVHNAKTKPREEQFNSTYRVVSVLTVV | |
| Heavy | | HQDWLNGKEYKCKVSNKGLPSSIEKTISKAKGQPREPQVYTLPPS | |
| Chain (VH | | QEEMTKNQVSLTCLVKGFYPSDIAVEWESNGQPENNYKTTPPVL | |
| in Bold) | | DSDGSFFLYSKLTVDKSRWQEGNVFSCSVMHEALHNHYTQKSLS | |
| | | LSLGK | |
| Tislelizumab, | PD-1 | DIVMTQSPDSLAVSLGERATINCKSSESVSNDVAWYQQKPGQP | 333 |
| Baizean, | | PKLLINYAFHRFTGVPDRESGSGYGTDFTLTISSLQAEDVAVY | |
| 0KVO411B3N, | | YCHQAYSSPYTFGQGTKLEIKRTVAAPSVFIFPPSDEQLKSGTAS | |
| BGB- | | VVCLLNNFYPREAKVQWKVDNALQSGNSQESVTEQDSKDSTYS | |
| A317, | | LSSTLTLSKADYEKHKVYACEVTHQGLSSPVTKSFNRGEC | |
| hu317- | | | |
| 1/IgG4mt2 | | | |
| Light Chain | | | |
| (VL in | | | |
| Bold) | | | |
| Sintilimab, | PD-1 | QVQLVQSGAEVKKPGSSVKVSCKASGGTFSSYAISWVRQAPG | 334 |
| Tyvyt, IBI- | | QGLEWMGLIIPMEDTAGYAQKFQGRVAITVDESTSTAYMELS | |
| 308 Heavy | | SLRSEDTAVYYCARAEHSSTGTFDYWGQGTLVTVSSASTKGPS | |
| Chain (VH | | VFPLAPCSRSTSESTAALGCLVKDYFPEPVTVSWNSGALTSGVHT | |
| in Bold) | | FPAVLQSSGLYSLSSVVTVPSSSLGTKTYTCNVDHKPSNTKVDKR | |
| | | VESKYGPPCPPCPAPEFLGGPSVFLFPPKPKDTLMISRTPEVTCVV | |
| | | VDVSQEDPEVQFNWYVDGVEVHNAKTKPREEQFNSTYRVVSVL | |
| | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLGK | |
| Sintilimab, | PD-1 | DIQMTQSPSSVSASVGDRVTITCRASQGISSWLAWYQQKPGK | 335 |
| Tyvyt, IBI- | | APKLLISAASSLQSGVPSRFSGSGSGTDFTLTISSLQPEDFATYY | |
| 308 Light | | CQQANHLPFTFGGGTKVEIKRTVAAPSVFIFPPSDEQLKSGTAS | |
| Chain (VL | | VVCLLNNFYPREAKVQWKVDNALQSGNSQESVTEQDSKDSTYS | |
| in Bold) | | LSSTLTLSKADYEKHKVYACEVTHQGLSSPVTKSFNRGEC | |
| Toripalimab, | PD-1 | QGQLVQSGAEVKKPGASVKVSCKASGYTFTDYEMHWVRQA | 336 |
| TeRuiPuLi, | | PIHGLEWIGVIESETGGTAYNQKFKGRVTITADKSTSTAYMEL | |
| Terepril, | | SSLRSEDTAVYYCAREGITTVATTYYWYFDVWGQGTTVTVSS | |
| Tuoyi, JS- | | ASTKGPSVFPLAPCSRSTSESTAALGCLVKDYFPEPVTVSWNSGA | |
| 001, TAB- | | LTSGVHTFPAVLQSSGLYSLSSVVTVPSSSLGTKTYTCNVDHKPS | |
| 001 Heavy | | NTKVDKRVESKYGPPCPPCPAPEFLGGPSVFLFPPKPKDTLMISRT | |
| Chain (VH | | PEVTCVVVDVSQEDPEVQFNWYVDGVEVHNAKTKPREEQFNST | |
| in Bold) | | YRVVSVLTVLHQDWLNGKEYKCKVSNKGLPSSIEKTISKAKGQP | |
| | | REPQVYTLPPSQEEMTKNQVSLTCLVKGFYPSDIAVEWESNGQPE | |
| | | NNYKTTPPVLDSDGSFFLYSRLTVDKSRWQEGNVFSCSVMHEAL | |
| | | HNHYTQKSLSLSLGK | |
| Toripalimab, | PD-1 | DVVMTQSPLSLPVTLGQPASISCRSSQSIVHSNGNTYLEWYLQ | 337 |
| TeRuiPuLi, | | KPGQSPQLLIYKVSNRFSGVPDRFSGSGSGTDFTLKISRVEAED | |
| Terepril, | | VGVYYCFQGSHVPLTFGQGTKLEIKRTVAAPSVFIFPPSDEQLK | |
| Tuoyi, JS- | | SGTASVVCLLNNFYPREAKVQWKVDNALQSGNSQESVTEQDSK | |
| 001, TAB- | | DSTYSLSSTLTLSKADYEKHKVYACEVTHQGLSSPVTKSFNRGEC | |
| 001 Light | | | |
| Chain (VL | | | |
| in Bold) | | | |
| Camrelizumab, | PD-1 | EVOLVESGGGLVQPGGSLRLSCAASGFTFSSYMMSWVRQAP | 338 |
| HR- | | GKGLEWVATISGGGANTYYPDSVKGRFTISRDNAKNSLYLQM | |
| 301210, | | NSLRAEDTAVYYCARQLYYFDYWGQGTTVTVSSASTKGPSVF | |
| INCSHR- | | PLAPCSRSTSESTAALGCLVKDYFPEPVTVSWNSGALTSGVHTFP | |
| 01210, | | AVLQSSGLYSLSSVVTVPSSSLGTKTYTCNVDHKPSNTKVDKRVE | |
| SHR-1210 | | SKYGPPCPPCPAPEFLGGPSVFLFPPKPKDTLMISRTPEVTCVVVD | |
| Heavy | | VSQEDPEVQFNWYVDGVEVHNAKTKPREEQFNSTYRVVSVLTV | |
| Chain (VH | | LHQDWLNGKEYKCKVSNKGLPSSIEKTISKAKGQPREPQVYTLPP | |
| in Bold) | | SQEEMTKNQVSLTCLVKGFYPSDIAVEWESNGQPENNYKTTPPV | |
| | | LDSDGSFFLYSRLTVDKSRWQEGNVFSCSVMHEALHNHYTQKSL | |
| | | SLSLGK | |
| Camrelizumab, | PD-1 | DIQMTQSPSSLSASVGDRVTITCLASQTIGTWLTWYQQKPGK | 339 |
| HR- | | APKLLIYTATSLADGVPSRFSGSGSGTDFTLTISSLQPEDFATY | |
| 301210, | | YCQQVYSIPWTFGGGTKVEIKRTVAAPSVFIFPPSDEQLKSGTA | |
| INCSHR- | | SVVCLLNNFYPREAKVQWKVDNALQSGNSQESVTEQDSKDSTYS | |
| 01210, | | LSSTLTLSKADYEKHKVYACEVTHQGLSSPVTKSFNRGEC | |
| SHR-1210 | | | |
| Light Chain | | | |
| (Light | | | |
| Chain in | | | |
| Bold) | | | |
| Cemiplimab, | PD-1 | EVQLLESGGVLVQPGGSLRLSCAASGFTFSNFGMTWVRQAPG | 340 |
| Cemiplimab- | | KGLEWVSGISGGGRDTYFADSVKGRFTISRDNSKNTLYLQMN | |
| rwlc, | | SLKGEDTAVYYCVKWGNIYFDYWGQGTLVTVSSASTKGPSVF | |
| LIBTAYO ®, | | PLAPCSRSTSESTAALGCLVKDYFPEPVTVSWNSGALTSGVHTFP | |
| 6QVL057INT, | | AVLQSSGLYSLSSVVTVPSSSLGTKTYTCNVDHKPSNTKVDKRVE | |
| H4H7798N, | | SKYGPPCPPCPAPEFLGGPSVFLFPPKPKDTLMISRTPEVTCVVVD | |
| REGN- | | VSQEDPEVQFNWYVDGVEVHNAKTKPREEQFNSTYRVVSVLTV | |
| 2810, SAR- | | LHQDWLNGKEYKCKVSNKGLPSSIEKTISKAKGQPREPQVYTLPP | |
| 439684 | | SQEEMTKNQVSLTCLVKGFYPSDIAVEWESNGQPENNYKTTPPV | |
| Heavy | | LDSDGSFFLYSRLTVDKSRWQEGNVFSCSVMHEALHNHYTQKSL | |
| Chain (VH | | SLSLGK | |
| in Bold) | | | |
| Cemiplimab, | PD-1 | DIQMTQSPSSLSASVGDSITITCRASLSINTFLNWYQQKPGKAP | 341 |
| Cemiplimab- | | NLLIYAASSLHGGVPSRESGSGSGTDFTLTIRTLQPEDFATYYC | |
| rwlc, | | QQSSNTPFTFGPGTVVDFRRTVAAPSVFIFPPSDEQLKSGTASVV | |
| LIBTAYO ®, | | CLLNNFYPREAKVQWKVDNALQSGNSQESVTEQDSKDSTYSLSS | |
| 6QVL057INT, | | TLTLSKADYEKHKVYACEVTHQGLSSPVTKSFNRGEC | |
| H4H7798N, | | | |
| REGN- | | | |
| 2810, SAR- | | | |
| 439684 | | | |
| Light Chain | | | |
| (VL in | | | |
| Bold) | | | |
| Lambrolizumab, | PD-1 | QVQLVQSGVEVKKPGASVKVSCKASGYTFTNYYMYWVRQAP | 346 |
| Pembrolizumab, | | GQGLEWMGGINPSNGGTNFNEKFKNRVTLTTDSSTTTAYME | |
| KEYTRUDA ®, | | LKSLQFDDTAVYYCARRDYRFDMGFDYWGQGTTVTVSSAST | |
| MK- | | KGPSVFPLAPCSRSTSESTAALGCLVKDYFPEPVTVSWNSGALTS | |
| 3475, SCH- | | GVHTFPAVLQSSGLYSLSSVVTVPSSSLGTKTYTCNVDHKPSNTK | |
| 900475, | | VDKRVESKYGPPCPPCPAPEFLGGPSVFLFPPKPKDTLMISRTPEV | |
| h409A11 | | TCVVVDVSQEDPEVQFNWYVDGVEVHNAKTKPREEQFNSTYRV | |
| Heavy | | VSVLTVLHQDWLNGKEYKCKVSNKGLPSSIEKTISKAKGQPREPQ | |
| Chain (VH | | VYTLPPSQEEMTKNQVSLTCLVKGFYPSDIAVEWESNGQPENNY | |
| in Bold) | | KTTPPVLDSDGSFFLYSRLTVDKSRWQEGNVFSCSVMHEALHNH | |
| | | YTQKSLSLSLGK | |
| Lambrolizumab, | PD-1 | EIVLTQSPATLSLSPGERATLSCRASKGVSTSGYSYLHWYQQK | 347 |
| Pembrolizumab, | | PGQAPRLLIYLASYLESGVPARFSGSGSGTDFTLTISSLEPEDF | |
| KEYTRUDA ®, | | AVYYCQHSRDLPLTFGGGTKVEIKRTVAAPSVFIFPPSDEQLKS | |
| MK- | | GTASVVCLLNNFYPREAKVQWKVDNALQSGNSQESVTEQDSKD | |
| 3475, SCH- | | STYSLSSTLTLSKADYEKHKVYACEVTHQGLSSPVTKSFNRGEC | |
| 900475, | | | |
| h409A11 | | | |
| Light | | | |
| Chain (VL | | | |
| in Bold) | | | |
| Lambrolizumab, | PD-1 | QVQLVQSGVEVKKPGASVKVSCKASGYTFTNYYMYWVRQAPG | 348 |
| Pembrolizumab, | | QGLEWMGGFPSNGGTNFNEKFKNRVTLTTDSSTTTAYMELKSLQ | |
| KEYTRUDA ®, | | FDDTAVYYCARRDYRFDMGFDYWGQGTTVTVSS | |
| MK- | | | |
| 3475, SCH- | | | |
| 900475, | | | |
| h409A11 | | | |
| VH | | | |
| Lambrolizumab, | PD-1 | EIVLTQSPATLSLSPGERATLSCRASKGVSTSGYSYLHWYQQKPG | 349 |
| Pembrolizumab, | | QAPRLLIYLASYLESGVPARFSGSGSGTDFTLTISSLEPEDFAVY | |
| KEYTRUDA ®, | | YCQHSRDLPLTFGGGTKVEIK | |
| MK- | | | |
| 3475, SCH- | | | |
| 900475, | | | |
| h409A11 | | | |
| VL | | | |
| Nivolumab, | PD-1 | QVQLVESGGGVVQPGRSLRLDCKASGITFSNSGMHWVRQAPGK | 350 |
| Nivolumab | | GLEWVAVIWYDGSKRYYADSVKGRFTISRDNSKNTLFLQMNSLR | |
| BMS, | | AEDTAVYYCATNDDYWGQGTLVTVSS | |
| OPDIVO ®, | | | |
| BMS- | | | |
| 936558, | | | |
| MDX-1106, | | | |
| ONO-4538 | | | |
| VH | | | |
| Nivolumab, | PD-1 | EIVLTQSPATLSLSPGERATLSCRASQSVSSYLAWYQQKPGQAPR | 351 |
| Nivolumab | | LLIYDASNRATGIPARFSGSGSGTDFTLTISSLEPEDFAVYYCQQ | |
| BMS, | | SSNWPRTFGQGTKVEIK | |
| OPDIVO®, | | | |
| BMS- | | | |
| 936558, | | | |
| MDX-1106, | | | |
| ONO-4538 | | | |
| VL | | | |
| Prolgolimab, | PD-1 | QVQLVQSGGGLVQPGGSLRLSCAASGFTFSSYWMYWVRQVP | 352 |
| Forteca, | | GKGLEWVSAIDTGGGRTYYADSVKGRFAISRVNAKNTMYLQ | |
| BCD-100 | | MNSLRAEDTAVYYCARDEGGGTGWGVLKDWPYGLDAWGQ | |
| Heavy | | GTLVTVSSASTKGPSVFPLAPSSKSTSGGTAALGCLVKDYFPEPV | |
| Chain (VH | | TVSWNSGALTSGVHTFPAVLQSSGLYSLSSVVTVPSSSLGTQTYI | |
| in Bold) | | CNVNHKPSNTKVDKRVEPKSCDKTHTCPPCPAPEAAGGPSVFLFP | |
| | | PKPKDTLMISRTPEVTCVVVDVSHEDPEVKFNWYVDGVEVHNAK | |
| | | TKPREEQYNSTYRVVSVLTVLHQDWLNGKEYKCKVSNKALPAPI | |
| | | EKTISKAKGQPREPQVYTLPPSREEMTKNQVSLTCLVKGFYPSDI | |
| | | AVEWESNGQPENNYKTTPPVLDSDGSFFLYSKLTVDKSRWQQGN | |
| | | VFSCSVMHEALHNHYTQKSLSLSPGK | |
| Prolgolimab, | PD-1 | QPVLTQPLSVSVALGQTARITCGGNNIGSKNVHWYQQKPGQ | 353 |
| Forteca, | | APVLVIYRDSNRPSGIPERFSGSNSGNTATLTISRAQAGDEADY | |
| BCD-100 | | YCQVWDSSTAVFGTGTKLTVLQRTVAAPSVFIFPPSDEQLKSGT | |
| Light Chain | | ASVVCLLNNFYPREAKVQWKVDNALQSGNSQESVTEQDSKDST | |
| (VL in | | YSLSSTLTLSKADYEKHKVYACEVTHQGLSSPVTKSFNRGEC | |
| Bold) | | | |
| Balstilimab, | PD-1 | QVQLVESGGGVVQPGRSLRLSCAASGFTFSSYGMHWVRQAP | 354 |
| 1Q2QT5M7EO, | | GKGLEWVAVIWYDGSNKYYADSVKGRFTISRDNSKNTLYLQ | |
| AGEN- | | MNSLRAEDTAVYYCASNGDHWGQGTLVTVSSASTKGPSVFPL | |
| 2034, | | APCSRSTSESTAALGCLVKDYFPEPVTVSWNSGALTSGVHTFPAV | |
| AGEN- | | LQSSGLYSLSSVVTVPSSSLGTKTYTCNVDHKPSNTKVDKRVESK | |
| 2034w | | YGPPCPPCPAPEFLGGPSVFLFPPKPKDTLMISRTPEVTCVVVDV | |
| Heavy | | SQEDPEVQFNWYVDGVEVHNAKTKPREEQFNSTYRVVSVLTVLH | |
| Chain (VH | | QDWLNGKEYKCKVSNKGLPSSIEKTISKAKGQPREPQVYTLPPSQ | |
| in Bold) | | EEMTKNQVSLTCLVKGFYPSDIAVEWESNGQPENNYKTTPPVLD | |
| | | SDGSFFLYSRLTVDKSRWQEGNVFSCSVMHEALHNHYTQKSLSL | |
| | | SLG | |
| Balstilimab, | PD-1 | EIVMTQSPATLSVSPGERATLSCRASQSVSSNLAWYQQKPGQ | 355 |
| 1Q2QT5M7EO, | | APRLLIYGASTRATGIPARFSGSGSGTEFTLTISSLQSEDFAVYY | |
| AGEN- | | CQQYNNWPRTFGQGTKVEIKRTVAAPSVFIFPPSDEQLKSGTAS | |
| 2034, | | VVCLLNNFYPREAKVQWKVDNALQSGNSQESVTEQDSKDSTYS | |
| AGEN- | | LSSTLTLSKADYEKHKVYACEVTHQGLSSPVTKSFNRGEC | |
| 2034w | | | |
| Light Chain | | | |
| (VL in | | | |
| Bold) | | | |
| Dostarlimab, | PD-1 | EVQLLESGGGLVQPGGSLRLSCAASGFTFSSYDMSWVRQAPG | 356 |
| ANB-011, | | KGLEWVSTISGGGSYTYYQDSVKGRFTISRDNSKNTLYLQMN | |
| GSK- | | SLRAEDTAVYYCASPYYAMDYWGQGTTVTVSSASTKGPSVFP | |
| 4057190A, | | LAPCSRSTSESTAALGCLVKDYFPEPVTVSWNSGALTSGVHTFPA | |
| POGVQ9A4S5, | | VLQSSGLYSLSSVVTVPSSSLGTKTYTCNVDHKPSNTKVDKRVES | |
| TSR- | | KYGPPCPPCPAPEFLGGPSVFLFPPKPKDTLMISRTPEVTCVVVDV | |
| 042, WBP- | | SQEDPEVQFNWYVDGVEVHNAKTKPREEQFNSTYRVVSVLTVL | |
| 285 Heavy | | HQDWLNGKEYKCKVSNKGLPSSIEKTISKAKGQPREPQVYTLPPS | |
| Chain (VH | | QEEMTKNQVSLTCLVKGFYPSDIAVEWESNGQPENNYKTTPPVL | |
| in Bold) | | DSDGSFFLYSRLTVDKSRWQEGNVFSCSVMHEALHNHYTQKSLS | |
| | | LSLGK | |
| Dostarlimab, | PD-1 | DIQLTQSPSFLSAYVGDRVTITCKASQDVGTAVAWYQQKPGK | 357 |
| ANB-011, | | APKLLIYWASTLHTGVPSRFSGSGSGTEFTLTISSLQPEDFATY | |
| GSK- | | YCQHYSSYPWTFGQGTKLEIKRTVAAPSVFIFPPSDEQLKSGTA | |
| 4057190A, | | SVVCLLNNFYPREAKVQWKVDNALQSGNSQESVTEQDSKDSTYS | |
| POGVQ9A4 | | LSSTLTLSKADYEKHKVYACEVTHQGLSSPVTKSFNRGEC | |
| S5, TSR- | | | |
| 042, WBP- | | | |
| 285 Light | | | |
| Chain (VL | | | |
| in Bold) | | | |
| Serplulimab, | PD-1 | QVQLVESGGGLVKPGGSLRLSCAASGFTFSNYGMSWIRQAPG | 358 |
| HLX-10 | | KGLEWSTISGGGSNIYYADSVKGRFTISRDNAKNSLYLQMNSL | |
| Heavy | | RAEDTAVYYCVSYYYGIDFWGQGTSVTVSSASKYGPSVFPLAPC | |
| Chain (VH | | SRSTSESTAALGCLVKDYFPEPVTVSWNSGALTSGVHTFPAVLQS | |
| in Bold) | | SGLYSLSSVVTVPSSSLGTKTYTCNVDHKPSNTKVDKRVESKYGP | |
| | | PCPPCPAPEFLGGPSVFLFPPKPKDTLMISRTPEVTCVVVDVSQED | |
| | | PEVQFNWYVDGVEVHNAKTKPREEQFNSTYRVVSVVLTVLHQD | |
| | | WLNGKEYKCKVSNKGLPSSIEKTISKAKGQPREPQVYTLPPSQEE | |
| | | MTKNQVSLTCLVKGFYPSDIAVEWESNGQPENNYKTTPPVLDSD | |
| | | GSFFLYSRLTVDKSRWQEGNVFSCSVMHEALHNHYTQKSLSLSL | |
| | | GK | |
| Serplulimab, | PD-1 | DIQMTQSPSSLSASVGDRVTITCKASQDVTTAVAWYQQKPGK | 359 |
| HLX-10 | | APKLLIYWASTRHTGVPSRFSGSGSGTDFTLTISSLQPEDFATY | |
| Light Chain | | YCQQHYTIPWTFGGGTKLEIKRTVAAPSVFIFPPSDEQLKSGTA | |
| (VL in | | SVVCLLNNFYPREAKVQWKVDNALQSGNSQESVTEQDSKDSTYS | |
| Bold) | | LSSTLTLSKADYEKHKVYACEVTHQGLSSPVTKSFNRGEC | |
| Retifanlimab, | PD-1 | QVQLVQSGAEVKKPGASVKVSCKASGYSFTSYWMNWVRQAP | 360 |
| 2Y3T5IF01Z, | | GQGLEWIGVIHPSDSETWLDQKFKDRVTITVDKSTSTAYMEL | |
| INCMGA- | | SSLRSEDTAVYYCAREHYGTSPFAYWGQGTLVTVSSASTKGPS | |
| 00012, | | VFPLAPCSRSTSESTAALGCLVKDYFPEPVTVSWNSGALTSGVHT | |
| INCMGA- | | FPAVLQSSGLYSLSSVVTVPSSSLGTKTYTCNVDHKPSNTKVDKR | |
| 0012, MGA- | | VESKYGPPCPPCPAPEFLGGPSVFLFPPKPKDTLMISRTPEVTCVV | |
| 012 Heavy | | VDVSQEDPEVQFNWYVDGVEVHNAKTKPREEQFNSTYRVVSVL | |
| Chain (VH | | TVLHQDWLNGKEYKCKVSNKGLPSSIEKTISKAKGQPREPQVYT | |
| in Bold) | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Retifanlimab, | PD-1 | EIVLTQSPATLSLSPGERATLSCRASESVDNYGMSFMNWFQQ | 361 |
| 2Y3T5IF01Z, | | KPGQPPKLLIHAASNQGSGVPSRFSGSGSGTDFTLTISSLEPED | |
| INCMGA- | | FAVYFCQQSKEVPYTFGGGTKVEIKRTVAAPSVFIFPPSDEQLK | |
| 00012, | | SGTASVVCLLNNFYPREAKVQWKVDNALQSGNSQESVTEQDSK | |
| INCMGA- | | DSTYSLSSTLTLSKADYEKHKVYACEVTHQGLSSPVTKSFNRGEC | |
| 0012, MGA- | | | |
| 012 Light | | | |
| Chain (VL | | | |
| in Bold) | | | |
| Sasanlimab, | PD-1 | QVQLVQSGAEVKKPGASVKVSCKASGYTFTSYWINWVRQAP | 362 |
| LZZ0IC2EWP, | | GQGLEWMGNIYPGSSLTNYNEKFKNRVTMTRDTSTSTVYME | |
| PF- | | LSSLRSEDTAVYYCARLSTGTFAYWGQGTLVTVSSASTKGPSV | |
| 06801591, | | FPLAPCSRSTSESTAALGCLVKDYFPEPVTVSWNSGALTSGVHTF | |
| RN-888 | | PAVLQSSGLYSLSSVVTVPSSSLGTKTYTCNVDHKPSNTKVDKRV | |
| Heavy | | ESKYGPPCPPCPAPEFLGGPSVFLFPPKPKDTLMISRTPEVTCVVV | |
| Chain (VH | | DVSQEDPEVQFNWYVDGVEVHNAKTKPREEQFNSTYRVVSVLT | |
| in Bold) | | VLHQDWLNGKEYKCKVSNKGLPSSIEKTISKAKGQPREPQVYTLP | |
| | | PSQEEMTKNQVSLTCLVKGFYPSDIAVEWESNGQPENNYKTTPP | |
| | | VLDSDGSFFLYSRLTVDKSRWQEGNVFSCSVMHEALHNHYTQKS | |
| | | LSLSLGK | |
| Sasanlimab, | PD-1 | DIVMTQSPDSLAVSLGERATINCKSSQSLWDSGNQKNFLTWY | 363 |
| LZZOIC2EWP, | | QQKPGQPPKLLIYWTSYRESGVPDRFSGSGSGTDFTLTISSLQ | |
| PF- | | AEDVAVYYCONDYFYPHTFGGGTKVEIKRTVAAPSVFIFPPSDE | |
| 06801591, | | QLKSGTASVVCLLNNFYPREAKVQWKVDNALQSGNSQESVTEQ | |
| RN-888 | | DSKDSTYSLSSTLTLSKADYEKHKVYACEVTHQGLSSPVTKSFNR | |
| Light | | GEC | |
| Chain (VL | | | |
| in Bold) | | | |
| Spartalizumab, | PD-1 | EVQLVQSGAEVKKPGESLRISCKGSGYTFTTYWMHWVRQAT | 364 |
| NVP- | | GQGLEWMGNIYPGTGGSNFDEKFKNRVTITADKSTSTAYME | |
| LZV-184, | | LSSLRSEDTAVYYCTRWTTGTGAYWGQGTTVTVSSASTKGPS | |
| PDR-001, | | VFPLAPCSRSTSESTAALGCLVKDYFPEPVTVSWNSGALTSGVHT | |
| QOG25L6Z8Z | | FPAVLQSSGLYSLSSVVTVPSSSLGTKTYTCNVDHKPSNTKVDKR | |
| Heavy | | VESKYGPPCPPCPAPEFLGGPSVFLFPPKPKDTLMISRTPEVTCVV | |
| Chain (VH | | VDVSQEDPEVQFNWYVDGVEVHNAKTKPREEQFNSTYRVVSVL | |
| in Bold) | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Spartalizumab, | PD-1 | EIVLTQSPATLSLSPGERATLSCKSSQSLLDSGNQKNFLTWYQ | 365 |
| NVP- | | QKPGQAPRLLIYWASTRESGVPSRFSGSGSGTDFTFTISSLEAE | |
| LZV-184, | | DAATYYCONDYSYPYTFGQGTKVEIKRTVAAPSVFIFPPSDEQL | |
| PDR-001, | | KSGTASVVCLLNNFYPREAKVQWKVDNALQSGNSQESVTEQDS | |
| QOG25L6Z8Z | | KDSTYSLSSTLTLSKADYEKHKVYACEVTHQGLSSPVTKSFNRGE | |
| Light | | C | |
| Chain (VL | | | |
| in Bold) | | | |
| Cetrelimab, | PD-1 | QVQLVQSGAEVKKPGSSVKVSCKASGGTFSSYAISWVRQAPG | 366 |
| JNJ-3283, | | QGLEWMGGIIPIFDTANYAQKFQGRVTITADESTSTAYMELSS | |
| JNJ- | | LRSEDTAVYYCARPGLAAAYDTGSLDYWGQGTLVTVSSASTK | |
| 63723283, | | GPSVFPLAPCSRSTSESTAALGCLVKDYFPEPVTVSWNSGALTSG | |
| LYK98WP91F | | VHTFPAVLQSSGLYSLSSVVTVPSSSLGTKTYTCNVDHKPSNTKV | |
| Heavy | | DKRVESKYGPPCPPCPAPEFLGGPSVFLFPPKPKDTLMISRTPEVT | |
| Chain (VH | | CVVVDVSQEDPEVQFNWYVDGVEVHNAKTKPREEQFNSTYRVV | |
| in Bold) | | SVLTVLHQDWLNGKEYKCKVSNKGLPSSIEKTISKAKGQPREPQV | |
| | | YTLPPSQEEMTKNQVSLTCLVKGFYPSDIAVEWESNGQPENNYK | |
| | | TTPPVLDSDGSFFLYSRLTVDKSRWQEGNVFSCSVMHEALHNHY | |
| | | TQKSLSLSLGK | |
| Cetrelimab, | PD-1 | EIVLTQSPATLSLSPGERATLSCRASQSVRSYLAWYQQKPGQA | 367 |
| JNJ-3283, | | PRLLIYDASNRATGIPARFSGSGSGTDFTLTISSLEPEDFAVYYC | |
| JNJ- | | QQRNYWPLTFGQGTKVEIKRTVAAPSVFIFPPSDEQLKSGTASV | |
| 63723283, | | VCLLNNFYPREAKVQWKVDNALQSGNSQESVTEQDSKDSTYSLS | |
| LYK98WP91F | | STLTLSKADYEKHKVYACEVTHQGLSSPVTKSFNRGEC | |
| Light | | | |
| Chain (VL | | | |
| in Bold) | | | |
| Tebotelimab, | PD-1 | DIQMTQSPSSLSASVGDRVTITCRASQDVSSVVAWYQQKPGK | 368 |
| MGD-013 | | APKLLIYSASYRYTGVPSRFSGSGSGTDFTLTISSLQPEDFATY | |
| Heavy | | YCQQHYSTPWTFGGGTKLEIKGGGSGGGGQVQLVQSGAEVKK | |
| Chain (VL | | PGASVKVSCKASGYSFTSYWMNWVRQAPGQGLEWIGVIHPSDSE | |
| in Bold) | | TWLDQKFKDRVTITVDKSTSTAYMELSSLRSEDTAVYYCAREHY | |
| | | GTSPFAYWGQGTLVTVSSGGCGGGEVAACEKEVAALEKEVAAL | |
| | | EKEVAALEKESKYGPPCPPCPAPEFLGGPSVFLFPPKPKDTLYITR | |
| | | EPEVTCVVVDVSQEDPEVQFNWYVDGVEVHNAKTKPREEQFNS | |
| | | TYRVVSVLTVLHQDWLNGKEYKCKVSNKGLPSSIEKTISKAKGQ | |
| | | PREPQVYTLPPSQEEMTKNQVSLTCLVKGFYPSDIAVEWESNGQP | |
| | | ENNYKTTPPVLDSDGSFFLYSRLTVDKSRWQEGNVFSCSVMHEA | |
| | | LHNHYTQKSLSLSLG | |
| Tebotelimab | PD-1 | EIVLTQSPATLSLSPGERATLSCRASESVDNYGMSFMNWFQQ | 369 |
| MGD-013 | | KPGQPPKLLIHAASNQGSGVPSRFSGSGSGTDFTLTISSLEPED | |
| Light Chain | | FAVYFCQQSKEVPYTFGGGTKVEIKGGGSGGGGQVQLVQSGA | |
| (VL in | | EVKKPGASVKVSCKASGYTFTDYNMDWVRQAPGQGLEWMGDI | |
| Bold) | | NPDNGVTIYNQKFEGRVTMTTDTSTSTAYMELRSLRSDDTAVYY | |
| | | CAREADYFYFDYWGQGTTLTVSSGGCGGGKVAACKEKVAALKE | |
| | | KVAALKEKVAALKE | |
| Pidilizumab, | PD-1 | QVQLVQSGSELKKPGASVKISCKASGYTFTNYGMNWVRQAP | 370 |
| CT-011, | | GQGLQWMGWINTDSGESTYAEEFKGRFVFSLDTSVNTAYLQI | |
| MDV-9300 | | TSLTAEDTGMYFCVRVGYDALDYWGQGTLVTVSSASTKGPSV | |
| Heavy | | FPLAPSSKSTSGGTAALGCLVKDYFPEPVTVSWNSGALTSGVHTF | |
| Chain (VH | | PAVLQSSGLYSLSSVVTVPSSSLGTQTYICNVNHKPSNTKVDKRV | |
| in Bold) | | EPKSCDKTHTCPPCPAPELLGGPSVFLFPPKPKDTLMISRTPEVTC | |
| | | VVVDVSHEDPEVKFNWYVDGVEVHNAKTKPREEQYNSTYRVVS | |
| | | VLTVLHQDWLNGKEYKCKVSNKALPAPIEKTISKAKGQPREPQV | |
| | | YTLPPSREEMTKNQVSLTCLVKGFYPSDIAVEWESNGQPENNYK | |
| | | TTPPVLDSDGSFFLYSKLTVDKSRWQQGNVFSCSVMHEALHNHY | |
| | | TQKSLSLSPGK | |
| Pidilizumab, | PD-1 | EIVLTQSPSSLSASVGDRVTITCSARSSVSYMHWFQQKPGKAP | 371 |
| CT-011, | | KLWIYRTSNLASGVPSRFSGSGSGTSYCLTINSLQPEDFATYYC | |
| MDV-9300 | | QQRSSFPLTFGGGTKLEIKRTVAAPSVFIFPPSDEQLKSGTASVV | |
| Light Chain | | CLLNNFYPREAKVQWKVDNALQSGNSQESVTEQDSKDSTYSLSS | |
| (VL in | | TLTLSKADYEKHKVYACEVTHQGLSSPVTKSFNRGEC | |
| Bold) | | | |
| SG-001 VH | PD-1 | QVQLVESGGGVVQPGRSLRLTCKASGLTFSSSGMHWVRQAPGK | 372 |
| | | GLEWVAVIWYDGSKRYYADSVKGRFTISRDNSKNTLFLQMNSLR | |
| | | AEDTAVYYCATNNDYWGQGTLVTVSS | |
| SG-001 VL | PD-1 | EIVLTQSPATLSLSPGERATLSCRASQSVSSYLAWYQQKPGQAPR | 373 |
| | | LLIYTASNRATGIPARFSGSGSGTDFTLTISSLEPEDFAVYYCQQ | |
| | | YSNWPRTFGQGTKVEIK | |
| mpLZM- | PD-1 | EVQLQQSGPVLVKPGASVKMSCKASGYTFTSYYMYWVKQSHGK | 374 |
| 009 VH | | SLEWIGGVNPSNGGTNFNEKFKSKATLTVDKSSSTAYMELNSLTS | |
| (Murine | | EDSAVYYCARRDYRYDMGFDYWGQGTTLTVSS | |
| Precursor of | | | |
| LZM-009) | | | |
| mpLZM- | PD-1 | QIVLTQSPAIMSASPGEKVTMTCRASKGVSTSGYSYLHWYQQKPG | 375 |
| 009 VL | | SSPRLLIYLASYLESGVPVRFSGSGSGTSYSLTISRMEAEDAATY | |
| (Murine | | YCQHSRELPLTFGTGTRLEIK | |
| Precursor of | | | |
| LZM-009) | | | |
| LZM-009 | PD-1 | QVQLVQSGAEVKKPGASVKVSCKASGYTFTSYYMYWVRQAPG | 376 |
| VH | | QGLEWMGGVNPSNGGTNFNEKFKSRVTITADKSTSTAYMELSSL | |
| | | RSEDTAVYYCARRDYRYDMGFDYWGQGTTVTVSS | |
| LZM-009 | PD-1 | EIVLTQSPATLSLSPGERATISCRASKGVSTSGYSYLHWYQQKPG | 377 |
| VL | | QAPRLLIYLASYLESGVPARFSGSGSGTDFTLTISSLEPEDFATY | |
| | | YCQHSRELPLTFGTGTKVEIK | |
| Budigalimab, | PD-1 | EIQLVQSGAEVKKPGSSVKVSCKASGYTFTHYGMNWVRQAP | 378 |
| 6VDO4TY3OO, | | GQGLEWVGWVNTYTGEPTYADDFKGRLTFTLDTSTSTAYME | |
| ABBV- | | LSSLRSEDTAVYYCTREGEGLGFGDWGQGTTVTVSSASTKGP | |
| 181, PR- | | SVFPLAPSSKSTSGGTAALGCLVKDYFPEPVTVSWNSGALTSGVH | |
| 1648817 | | TFPAVLQSSGLYSLSSVVTVPSSSLGTQTYICNVNHKPSNTKVDK | |
| Heavy | | KVEPKSCDKTHTCPPCPAPEAAGGPSVFLFPPKPKDTLMISRTPEV | |
| Chain (VH | | TCVVVDVSHEDPEVKFNWYVDGVEVHNAKTKPREEQYNSTYRV | |
| in Bold) | | VSVLTVLHQDWLNGKEYKCKVSNKALPAPIEKTISKAKGQPREP | |
| | | QVYTLPPSREEMTKNQVSLTCLVKGFYPSDIAVEWESNGQPENN | |
| | | YKTTPPVLDSDGSFFLYSKLTVDKSRWQQGNVFSCSVMHEALHN | |
| | | HYTQKSLSLSPGK | |
| Budigalimab, | PD-1 | DVVMTQSPLSLPVTPGEPASISCRSSQSIVHSHGDTYLEWYLQ | 379 |
| 6VDO4TY3OO, | | KPGQSPQLLIYKVSNRFSGVPDRFSGSGSGTDFTLKISRVEAED | |
| ABBV- | | VGVYYCFQGSHIPVTFGQGTKLEIKRTVAAPSVFIFPPSDEQLKS | |
| 181, PR- | | GTASVVCLLNNFYPREAKVQWKVDNALQSGNSQESVTEQDSKD | |
| 1648817 | | STYSLSSTLTLSKADYEKHKVYACEVTHQGLSSPVTKSFNRGEC | |
| Light Chain | | | |
| (VL in | | | |
| Bold) | | | |
| Lambrolizumab, | PD-1 | NYYMY | 380 |
| Pembrolizumab, | | | |
| KEYTRUDA ®, | | | |
| MK- | | | |
| 3475, SCH- | | | |
| 900475, | | | |
| h409A11 | | | |
| VH CDR1 | | | |
| Lambrolizumab, | PD-1 | GINPSNGGTNFNEKFKN | 381 |
| Pembrolizumab, | | | |
| KEYTRUDA ®, | | | |
| MK- | | | |
| 3475, SCH- | | | |
| 900475, | | | |
| h409A11 | | | |
| VH CDR2 | | | |
| Lambrolizumab, | PD-1 | RDYRFDMGFDY | 382 |
| Pembrolizumab, | | | |
| KEYTRUDA ®, | | | |
| MK- | | | |
| 3475, SCH- | | | |
| 900475, | | | |
| h409A11 | | | |
| VH CDR3 | | | |
| Lambrolizumab, | PD-1 | RASKGVSTSGYSYLH | 383 |
| Pembrolizumab, | | | |
| KEYTRUDA ®, | | | |
| MK- | | | |
| 3475, SCH- | | | |
| 900475, | | | |
| h409A11 | | | |
| VL CDR1 | | | |
| Lambrolizumab, | PD-1 | LASYLES | 384 |
| Pembrolizumab, | | | |
| KEYTRUDA ®, | | | |
| MK- | | | |
| 3475, SCH- | | | |
| 900475, | | | |
| h409A11 | | | |
| VL CDR2 | | | |
| Lambrolizumab, | PD-1 | QHSRDLPLT | 385 |
| Pembrolizumab, | | | |
| KEYTRUDA ®, | | | |
| MK- | | | |
| 3475, SCH- | | | |
| 900475, | | | |
| h409A11 | | | |
| VL CDR3 | | | |
| Nivolumab, | PD-1 | NSGMH | 386 |
| Nivolumab | | | |
| BMS, | | | |
| OPDIVO ® | | | |
| BMS- | | | |
| 936558, | | | |
| MDX-1106, | | | |
| ONO-4538 | | | |
| VH CDR1 | | | |
| Nivolumab, | PD-1 | VIWYDGSKRYYADSVKG | 387 |
| Nivolumab | | | |
| BMS, | | | |
| OPDIVO ®, | | | |
| BMS- | | | |
| 936558, | | | |
| MDX-1106, | | | |
| ONO-4538 | | | |
| VH CDR2 | | | |
| Nivolumab, | PD-1 | NDDY | 388 |
| Nivolumab | | | |
| BMS, | | | |
| OPDIVO ®, | | | |
| BMS- | | | |
| 936558, | | | |
| MDX-1106, | | | |
| ONO-4538 | | | |
| VH CDR3 | | | |
| Nivolumab, | PD-1 | RASQSVSSYLA | 389 |
| Nivolumab | | | |
| BMS, | | | |
| OPDIVO ®, | | | |
| BMS- | | | |
| 936558, | | | |
| MDX-1106, | | | |
| ONO-4538 | | | |
| VL CDR1 | | | |
| Nivolumab, | PD-1 | DASNRAT | 390 |
| Nivolumab | | | |
| BMS, | | | |
| OPDIVO ®, | | | |
| BMS- | | | |
| 936558, | | | |
| MDX-1106, | | | |
| ONO-4538 | | | |
| VL CDR2 | | | |
| Nivolumab, | PD-1 | QQSSNWPRT | 391 |
| Nivolumab | | | |
| BMS, | | | |
| OPDIVO ®, | | | |
| BMS- | | | |
| 936558, | | | |
| MDX-1106, | | | |
| ONO-4538 | | | |
| VL CDR3 | | | |
| Serplulimab, | PD-1 | FTFSNYGMS | 392 |
| HLX-10 | | | |
| VH CDR1 | | | |
| Serplulimab, | PD-1 | TISGGGSNIY | 393 |
| HLX-10 | | | |
| VH CDR2 | | | |
| Serplulimab, | PD-1 | VSYYYGIDF | 394 |
| HLX-10 | | | |
| VH CDR3 | | | |
| Serplulimab, | PD-1 | KASQDVTTAVA | 395 |
| HLX-10 | | | |
| VL CDR1 | | | |
| Serplulimab, | PD-1 | WASTRHT | 396 |
| HLX-10 | | | |
| VL CDR2 | | | |
| Serplulimab, | PD-1 | QQHYTIPWT | 397 |
| HLX-10 | | | |
| VL CDR3 | | | |
| SG-001 VH | PD-1 | GLTFSSSG | 398 |
| CDR1 | | | |
| SG-001 VH | PD-1 | IWYDGSKR | 399 |
| CDR2 | | | |
| SG-001 VH | PD-1 | ATNNDY | 400 |
| CDR3 | | | |
| SG-001 VL | PD-1 | RASQSVSSYLA | 401 |
| CDR1 | | | |
| SG-001 VL | PD-1 | TASNRAT | 402 |
| CDR2 | | | |
| SG-001 VL | PD-1 | QQYSNWPRT | 403 |
| CDR3 | | | |
| Avelumab | PD-L1 | EVQLLESGGGLVQPGGSLRLSCAASGFTESSYIMMWVRQAPG | 232 |
| (Generic) | | KGLEWVSSIYPSGGITFYADTVKGRFTISRDNSKNTLYLQMNS | |
| Bavencio | | LRAEDTAVYYCARIKLGTVTTVDYWGQGTLVTVSSASTKGPS | |
| (Brand) | | VFPLAPSSKSTSGGTAALGCLVKDYFPEPVTVSWNSGALTSGVHT | |
| 451238 | | FPAVLQSSGLYSLSSVVTVPSSSLGTQTYICNVNHKPSNTKVDKK | |
| KXG2PJ551I | | VEPKSCDKTHTCPPCPAPELLGGPSVFLFPPKPKDTLMISRTPEVT | |
| MSB- | | CVVVDVSHEDPEVKFNWYVDGVEVHNAKTKPREEQYNSTYRVV | |
| 0010682 | | SVLTVLHQDWLNGKEYKCKVSNKALPAPIEKTISKAKGQPREPQ | |
| MSB- | | VYTLPPSRDELTKNQVSLTCLVKGFYPSDIAVEWESNGQPENNYK | |
| 0010718C | | TTPPVLDSDGSFFLYSKLTVDKSRWQQGNVFSCSVMHEALHNHY | |
| PF- | | TQKSLSLSPGK | |
| 06834635 | | | |
| Heavy | | | |
| Chain (VH | | | |
| in Bold) | | | |
| Avelumab | PD-L1 | QSALTQPASVSGSPGQSITISCTGTSSDVGGYNYVSWYQQHPG | 233 |
| (Generic) | | KAPKLMIYDVSNRPSGVSNRFSGSKSGNTASLTISGLQAEDEA | |
| Bavencio | | DYYCSSYTSSSTRVFGTGTKVTVLGQPKANPTVTLFPPSSEELQA | |
| (Brand) | | NKATLVCLISDFYPGAVTVAWKADGSPVKAGVETTKPSKQSNNK | |
| 451238 | | YAASSYLSLTPEQWKSHRSYSCQVTHEGSTVEKTVAPTECS | |
| KXG2PJ551I | | | |
| MSB- | | | |
| 0010682 | | | |
| MSB- | | | |
| 0010718C | | | |
| PF- | | | |
| 06834635 | | | |
| Light Chain | | | |
| (VL in | | | |
| Bold) | | | |
| Durvalumab | PD-L1 | EVQLVESGGGLVQPGGSLRLSCAASGFTFSRYWMSWVRQAP | 234 |
| (Generic) | | GKGLEWVANIKQDGSEKYYVDSVKGRFTISRDNAKNSLYLQ | |
| Imfinzi | | MNSLRAEDTAVYYCAREGGWFGELAFDYWGQGTLVTVSSAS | |
| (Brand) | | TKGPSVFPLAPSSKSTSGGTAALGCLVKDYFPEPVTVSWNSGALT | |
| 28X28X90 | | SGVHTFPAVLQSSGLYSLSSVVTVPSSSLGTQTYICNVNHKPSNTK | |
| KV (UNII | | VDKRVEPKSCDKTHTCPPCPAPEFEGGPSVFLFPPKPKDTLMISRT | |
| code) | | PEVTCVVVDVSHEDPEVKFNWYVDGVEVHNAKTKPREEQYNST | |
| MEDI-4736 | | YRVVSVLTVLHQDWLNGKEYKCKVSNKALPASIEKTISKAKGQP | |
| Heavy | | REPQVYTLPPSREEMTKNQVSLTCLVKGFYPSDIAVEWESNGQPE | |
| Chain (VH | | NNYKTTPPVLDSDGSFFLYSKLTVDKSRWQQGNVFSCSVMHEAL | |
| in Bold) | | HNHYTQKSLSLSPGK | |
| Durvalumab | PD-L1 | EIVLTQSPGTLSLSPGERATLSCRASQRVSSSYLAWYQQKPGQ | 235 |
| (Generic) | | APRLLIYDASSRATGIPDRESGSGSGTDFTLTISRLEPEDFAVYY | |
| Imfinzi | | CQQYGSLPWTFGQGTKVEIKRTVAAPSVFIFPPSDEQLKSGTAS | |
| (Brand) | | VVCLLNNFYPREAKVQWKVDNALQSGNSQESVTEQDSKDSTYS | |
| 28X28X90 | | LSSTLTLSKADYEKHKVYACEVTHQGLSSPVTKSFNRGEC | |
| KV (UNII | | | |
| code) | | | |
| MEDI-4736 | | | |
| Light Chain | | | |
| (VL in | | | |
| Bold) | | | |
| Atezolizumab | PD-L1 | EVQLVESGGGLVQPGGSLRLSCAASGFTESDSWIHWVRQAPG | 236 |
| (Generic) | | KGLEWVAWISPYGGSTYYADSVKGRFTISADTSKNTAYLQMN | |
| Tecentriq | | SLRAEDTAVYYCARRHWPGGFDYWGQGTLVTVSSASTKGPS | |
| (Brand) | | VFPLAPSSKSTSGGTAALGCLVKDYFPEPVTVSWNSGALTSGVHT | |
| 52CMIOWC3YMPDL- | | FPAVLQSSGLYSLSSVVTVPSSSLGTQTYICNVNHKPSNTKVDKK | |
| 3280ARG- | | VEPKSCDKTHTCPPCPAPELLGGPSVFLFPPKPKDTLMISRTPEVT | |
| 7446RO- | | CVVVDVSHEDPEVKFNWYVDGVEVHNAKTKPREEQYASTYRVV | |
| 5541267 | | SVLTVLHQDWLNGKEYKCKVSNKALPAPIEKTISKAKGQPREPQ | |
| Heavy | | VYTLPPSREEMTKNQVSLTCLVKGFYPSDIAVEWESNGQPENNY | |
| Chain (VH | | KTTPPVLDSDGSFFLYSKLTVDKSRWQQGNVFSCSVMHEALHNH | |
| in Bold) | | YTQKSLSLSPGK | |
| Atezolizumab | PD-L1 | DIQMTQSPSSLSASVGDRVTITCRASQDVSTAVAWYQQKPGK | 237 |
| (Generic) | | APKLLIYSASFLYSGVPSRESGSGSGTDFTLTISSLQPEDFATYY | |
| Tecentriq | | CQQYLYHPATFGQGTKVEIKRTVAAPSVFIFPPSDEQLKSGTAS | |
| (Brand) | | VVCLLNNFYPREAKVQWKVDNALQSGNSQESVTEQDSKDSTYS | |
| 52CMIOWC3YMPDL- | | LSSTLTLSKADYEKHKVYACEVTHQGLSSPVTKSFNRGEC | |
| 3280ARG- | | | |
| 7446RO- | | | |
| 5541267 | | | |
| Light Chain | | | |
| (VL in | | | |
| Bold) | | | |
| Sugemalimab | PD-L1 | EVQLLESGGGLVQPGGSLRLSCAASGFTFSSYAMSWVRQAPG | 238 |
| (Generic) | | KGLEWVSGISGSGGFTYYADSVKGRFTISRDNSKNTLYLQMN | |
| CS-1001 | | SLRAEDTAVYYCAKPPRGYNYGPFDYWGQGTLVTVSSASTKG | |
| WBP-3155 | | PSVFPLAPCSRSTSESTAALGCLVKDYFPEPVTVSWNSGALTSGV | |
| Heavy | | HTFPAVLQSSGLYSLSSVVTVPSSSLGTKTYTCNVDHKPSNTKVD | |
| Chain (VH | | KRVESKYGPPCPPCPAPEFLGGPSVFLFPPKPKDTLMISRTPEVTC | |
| in Bold) | | VVVDVSQEDPEVQFNWYVDGVEVHNAKTKPREEQFNSTYRVVS | |
| | | VLTVLHQDWLNGKEYKCKVSNKGLPSSIEKTISKAKGQPREPQV | |
| | | YTLPPSQEEMTKNQVSLTCLVKGFYPSDIAVEWESNGQPENNYK | |
| | | TTPPVLDSDGSFFLYSRLTVDKSRWQEGNVFSCSVMHEALHNHY | |
| | | TQKSLSLSLGK | |
| Sugemalimab | PD-L1 | SYVLTQPPSVSVAPGQTARITCGGNNIGSKSVHWYQQKPGQA | 239 |
| (Generic) | | PVLVVYDDSDRPSGIPERFSGSNSGNTATLTISRVEAGDEADYY | |
| CS-1001 | | CQVWDSSSDHVVFGGGTKLTVLGQPKAAPSVTLFPPSSEELQA | |
| WBP-3155 | | NKATLVCLISDFYPGAVTVAWKADSSPVKAGVETTTPSKQSNNK | |
| Light | | YAASSYLSLTPEQWKSHRSYSCQVTHEGSTVEKTVAPTECS | |
| Chain (VL | | | |
| in Bold) | | | |
| JS-003 | PD-L1 | QGQLQESGPSLVKPSQTLSLTCTVSGDSITRGYWNWIRKHPGKGL | 242 |
| JS003-30 | | EYIGYISYTGSTYSNLSLKSRVTISRDTSKNQYYLKLSSVTAADTA | |
| JS003-SD | | VYYCATSTGWLDPVDYWGQGTLVTVSS | |
| VH | | | |
| JS-003 | PD-L1 | DIVMTQSPDSLAVSLGERATINCKASQNVDTSVAWFQQKPGQPP | 243 |
| JS003-30 | | KALIYSASFRYSGVPDRFSGSGSGTDFTLTISSLQAEDVAVYFCQQ | |
| JS003-SD | | YYGYPFTFGQGTKLEIK | |
| VL | | | |
| HLX-20 | PD-L1 | EVQLVQSGGGLVKPGGSLRLSCAASGFTFSSYTMNWVRQAPGK | 244 |
| PL2#3 VH | | GLEWVSSISSGSDYLYYADSVKGRFTISRDNAKNSLYLQMNSLRA | |
| | | EDTAVYYCARNELRWYPQAGAFDRWGQGTMVTVSS | |
| HLX-20 | PD-L1 | QSVVTQPPSMSAAPGQRVTISCSGSSSYIESSYVGWYQQLPGTAP | 245 |
| PL2#3 VL | | RLLIYDDDMRPSGIPDRESGSKSGTSATLAITGLQTGDEADYYCEI | |
| | | WRSGLGGVFGGGTKLTVL | |
| Lodapolimab | PD-L1 | QVQLVQSGAEVKKPGSSVKVSCKASGGTFSSYAISWVRQAPG | 248 |
| (Generic) | | QGLEWMGGIIPIFGTANYAQKFQGRVTITADKSTSTAYMELSS | |
| LY-3300054 | | LRSEDTAVYYCARSPDYSPYYYYGMDVWGQGTTVTVSSASTK | |
| NR4MAD6 | | GPSVFPLAPSSKSTSGGTAALGCLVKDYFPEPVTVSWNSGALTSG | |
| PPB Heavy | | VHTFPAVLQSSGLYSLSSVVTVPSSSLGTQTYICNVNHKPSNTKV | |
| Chain (VH | | DKRVEPKSCDKTHTCPPCPAPEAEGAPSVFLFPPKPKDTLMISRTP | |
| in Bold) | | EVTCVVVDVSHEDPEVKFNWYVDGVEVHNAKTKPREEQYNSTY | |
| | | RVVSVLTVLHQDWLNGKEYKCKVSNKALPSSIEKTISKAKGQPR | |
| | | EPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALH | |
| | | NHYTQKSLSLSPGK | |
| Lodapolimab | PD-L1 | QSVLTQPPSASGTPGQRVTISCSGSSSNIGSNTVNWYQQLPGT | 249 |
| (Generic) | | APKLLIYGNSNRPSGVPDRFSGSKSGTSASLAISGLQSEDEADY | |
| LY-3300054 | | YCQSYDSSLSGSVFGGGIKLTVLGQPKAAPSVTLFPPSSEELQAN | |
| NR4MAD6 | | KATLVCLISDFYPGAVTVAWKADSSPVKAGVETTTPSKQSNNKY | |
| PPB Light | | AASSYLSLTPEQWKSHRSYSCQVTHEGSTVEKTVAPAECS | |
| Chain (VL | | | |
| in Bold) | | | |
| HLX-20 | PD-L1 | SYTMN | 250 |
| PL2#3 VH | | | |
| CDR1 | | | |
| HLX-20 | PD-L1 | SISSGSDYLYYADSVKG | 251 |
| PL2#3 VH | | | |
| CDR2 | | | |
| HLX-20 | PD-L1 | NELRWYPQAGAFDR | 252 |
| PL2#3 VH | | | |
| CDR3 | | | |
| HLX-20 | PD-L1 | SGSSSYIESSYVG | 253 |
| PL2#3 VL | | | |
| CDR1 | | | |
| HLX-20 | PD-L1 | DDDMRPS | 254 |
| PL2#3 VL | | | |
| CDR2 | | | |
| HLX-20 | PD-L1 | EIWRSGLGGV | 255 |
| PL2#3 VL | | | |
| CDR3 | | | |
| Envafolimab | PD-L1 | QVQLVESGGGLVQPGGSLRLSCAASGKMSSRRCMAWFRQAP | 256 |
| (Generic) | | GKERERVAKLLTTSGSTYLADSVKGRFTISRDNSKNTVYLQM | |
| 3D-025 | | NSLRAEDTAVYYCAADSFEDPTCTLVTSSGAFQYWGQGTLVTVS | |
| ASC-22 | | SEPKSSDKTHTCPPCPAPELLGGPSVFLFPPKPKDTLMISRTPEV | |
| KN-035 | | TCVVVAVSHEDPEVKFNWYVDGVEVHNAKTKPREEQYNSTYRV | |
| hu56V1-Fc- | | VSVLTVLHQDWLNGKEYKCKVSNKALPAGIEKTISKAKGQPREP | |
| m1 single- | | QVYTLPPSRDELTKNQVSLTCLVKGFYPSDIAVEWESNGQPENNY | |
| domain | | KTTPPVLDSDGSFFLYSKLTVDKSRWQQGNVFSCSVMHEALHNH | |
| antibody | | YTQKSLSLSPGK | |
| (VH in | | | |
| Bold) | | | |
| ND-021 | PD-L1 | DIQMTQSPASLSASVGDRVTITCQASQSIGTYLAWYQQKPGKPPK | 257 |
| NM21-1480 | | LLIYRAFILASGVPSRFSGSGSGTDFTLTISSLQPEDFATYYCQSN | |
| PRO-1480 | | FYSDSTTIGPNAFGTGTKVTVLGGGGGSEVQLVESGGGLVQPGGS | |
| Tri-specific | | LRLSCAASGFSFSANYYPCWVRQAPGKGLEWIGCIYGGSSDITYD | |
| fusion | | ANWTKGRFTISRDNSKNTVYLQMNSLRAEDTAVYYCARSAWYS | |
| single-chain | | GWGGDLWGQGTLVTVSSGGGGSGGGGSGGGGSGGGGSIQMTQ | |
| antibody | | SPSSLSASVGDRVTITCQASQSISNRLAWYQQKPGKAPKLLIYSAS | |
| construct | | TLASGVPSRFSGSGSGTDFTLTISSLQPEDFATYYCQSTYYGNDGN | |
| | | AFGTGTKVTVLGGGGGSEVQLVESGGGLVQPGGSLRLSCAASGF | |
| | | SFNSDYWIYWVRQAPGKGLEWIASIYGGSSGNTQYASWAQGRFT | |
| | | ISRDNSKNTVYLQMNSLRAEDTAVYFCARGYVDYGGATDLWGQ | |
| | | GTLVTVSSGGGGSGGGGSIQMTQSPSSLSASVGDRVTITCQSSESV | |
| | | YSNNQLSWYQQKPGQPPKLLIYDASDLASGVPSRFSGSGSGTDFT | |
| | | LTISSLQPEDFATYYCAGGFSSSSDTAFGGGTKLTVLGGGGGSGG | |
| | | GGSGGGGSGGGGSEVQLVESGGGLVQPGGSLRLSCAASGFSLSS | |
| | | NAMGWVRQAPGKGLEYIGIISVGGFTYYASWAKGRFTISRDNSK | |
| | | NTVYLQMNSLRAEDTATYFCARDRHGGDSSGAFYLWGQGTLVT | |
| | | VSS | |
| ACE-05 | PD-L1 | QMQLVQSGAEVKKPGSSVKVSCKASGGTFSSYAISWVRQAPGQG | 258 |
| YBL-013 | | LEWMGRIIPILGIANYAQKFQGRVTITADKSTSTAYMELSSLRSED | |
| Bispecific | | TAVYYCAKPRDGYNLVAFDIWGQGTMVTVSSASTKGPSVFPLAP | |
| tetrameric | | SSKSTSGGTAALGCLVKDYFPEPVTVSWNSGALTSGVHTFPAVL | |
| antibody- | | QSSGLYSLSSVVTVPSSSLGTQTYICNVNHKPSNTKVDKKVEPKS | |
| like cell | | CDKTHTCPPCPAPELLGGPGGGGSEVQLQQSGPELVKPGPSMKIS | |
| engager | | CKASGYSFTGYTMNWVKQSHGKNLEWMGLINPYKGVSTYNQK | |
| (ALICE) | | FKDKATLTVDKSSSTAYMELLSLTSEDSAVYYCARSGYYGDSDW | |
| comprising | | YFDVWGQGTTLTVFSQMQLVQSGAEVKKPGSSVKVSCKASGGT | |
| two identical | | FSSYAISWVRQAPGQGLEWMGRIIPILGIANYAQKFQGRVTITAD | |
| light chains | | KSTSTAYMELSSLRSEDTAVYYCAKPRDGYNLVAFDIWGQGTM | |
| (LC) | | VTVSSASTKGPSVFPLAPSSKSTSGGTAALGCLVKDYFPEPVTVS | |
| consisting of | | WNSGALTSGVHTFPAVLQSSGLYSLSSVVTVPSSSLGTQTYICNV | |
| antigen | | NHKPSNTKVDKKVEPKSCDKTHTCPPCPAPELLGGPGGGGSDIQ | |
| binding | | MTQTTSSLSASLGDRVTISCRASQDIRNYLNWYQQKPDGTVKLLI | |
| domains | | YYTSRLHSGVPSKFSGSGSGTDYSLTISNLEQEDIATYFCQQGNTL | |
| (ABDs) | | PWTFAGGTKLEIKRQLVLTQPPSVSGAPGQRVTISCTGSSSNIGAG | |
| targeting | | YDVHWYQQLPGAAPKLLIYGDINRPSGVPDRESGSKSGISASLAIT | |
| programmed | | GLQAEDEADYYCQSYDSSLSGGVFGGGTKLTVLRTVAAPSVFIFP | |
| cell death- | | PSDEQLKSGTASVVCLLNNFYPREAKVQWKVDNALQSGNSQESV | |
| ligand 1 | | TEQDSKDSTYSLSSTLTLSKADYEKHKVYACEVTHQGLSSPVTKS | |
| (PD-L1), | | FNRGEC | |
| and two | | | |
| different | | | |
| heavy chain | | | |
| (HC)-like | | | |
| chains | | | |
| (ACE-05- | | | |
| VH and | | | |
| ACE-05- | | | |
| VL) each | | | |
| consisting of | | | |
| an anti-PD- | | | |
| L1 ABD and | | | |
| an anti-CD3 | | | |
| ABD; | | | |
| wherein | | | |
| each HC | | | |
| comprises a | | | |
| G4S linker | | | |
| (SEQ ID | | | |
| NO: 691) | | | |
| between the | | | |
| hinge region | | | |
| and the | | | |
| second ABD | | | |

### Modification to Fc Region

Disclosed herein are antibodies or antigen binding fragments thereof that comprise an Fc region, wherein the Fc region comprises at least one covalently linked linker. In some embodiments, the linker is a chemical linker. In some embodiments, the chemical linker is covalently attached to a tyrosine, aspartic acid, glutamic acid, arginine, histidine, or lysine residue. In some embodiments, the chemical linker is covalently attached to a lysine, cysteine, or tyrosine residue. In some embodiments, the chemical linker is covalently attached to a cysteine residue. In some embodiments, the chemical linker is covalently attached to a lysine residue. In some embodiments, the chemical linker is covalently attached to a constant region of the antibody.

In some embodiments, the antibody comprises an Fc region. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S229P Fc mutation. In some instances, a modified Fc comprises a human IgG1 kappa where the heavy chain CH2 domain is engineered with a triple mutation such as, for example: (a) L238P, L239E, and P335S; or (2) K248; K288; and K317.

In some embodiments, the Fc region has an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 260 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid). In some embodiments, the Fc region comprises one or more mutations which make the Fc region susceptible to modification or conjugation at a particular residue, such as by incorporation of a cysteine residue at a position which does not contain a cysteine in SEQ ID NO: 260. Alternatively, the Fc region could be modified to incorporate a modified natural amino acid or an unnatural amino acid which comprises a conjugation handle, such as one connected to the modified natural amino acid or unnatural amino acid through a linker. In some embodiments, the Fc region does not comprise any mutations which facilitate the attachment of a linker to an additional cytokine (e.g., an IL-18 polypeptide). In some embodiments, the chemical linker is attached to a native residue as set forth in SEQ ID NO: 260. In some embodiments, the chemical linker is attached to a native lysine residue of SEQ ID NO: 260.

In some embodiments, the chemical linker is attached to the Fc region at an amino acid residue at one of positions 10-90 of SEQ ID NO: 260. In some embodiments, the chemical linker is attached to the Fc region at an amino acid residue at one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 260. In some embodiments, the chemical linker is attached to the Fc region at an amino acid residue at one of positions 10-30, 50-70, or 80-100 of SEQ ID NO: 260. In some embodiments, the chemical linker is attached to the Fc region at an amino acid residue at any one of positions 20-40, 65-85, or 90-110 of SEQ ID NO: 260. In some embodiments, the chemical linker is attached to the Fc region at an amino acid residue at one of positions 15-26, 55-65, or 85-90 of SEQ ID NO: 260. In some embodiments, the chemical linker is attached to the Fc region at an amino acid residue at any one of positions 25-35, 70-80, or 95-105 of SEQ ID NO: 260. In some embodiments, the chemical linker is attached to the Fc region at an amino acid residue at any one of positions 30, 32, 72, 74, or 101 of SEQ ID NO: 260. In some embodiments, the chemical linker is attached to the Fc region at an amino acid residue at any one of positions K30, K32, K72, K74, or K101 of SEQ ID NO: 260. In some embodiments, the chemical linker is attached to the Fc region at amino acid residue 30 of SEQ ID NO: 260. In some embodiments, the chemical linker is attached to the Fc region at amino acid residue 32 of SEQ ID NO: 260. In some embodiments, the chemical linker is attached to the Fc region at amino acid residue 72 of SEQ ID NO: 260. In some embodiments, the chemical linker is attached to the Fc region at amino acid residue 74 of SEQ ID NO: 260. In some embodiments, the chemical linker is attached to the Fc region at amino acid residue 101 of SEQ ID NO: 260.

The chemical linker can be covalently attached to one amino acid residue of an Fc region of the antibody. In some embodiments, the chemical linker is covalently attached to a non-terminal residue of the Fc region. In some embodiments, the non-terminal residue is in the CH1, CH2, or CH3 region of the antibody. In some embodiments, the non-terminal residue is in the CH2 region of the antibody.

In some embodiments, the chemical linker is covalently attached at an amino acid residue of the antibody or antigen binding fragment which selectively binds a immune associated antigen (e.g., an anti-PD-1 antibody) such that the function of the antibody or antigen binding fragment is maintained (e.g., without denaturing the polypeptide). For example, when the antibody or antigen binding fragment is a human IgG (e.g., human IgG1), exposed lysine residues and exposed tyrosine residues are present at the following positions (refer to web site www.imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Tyrosine Residues: CH2 domain (position 278, position 296, and position 300) CH3 domain (position 436).

The human IgG, such as human IgG1, may also be modified with a lysine or tyrosine residue at any one of the positions listed above in order provide a residue which is ideally surface exposed for subsequent modification.

In some embodiments, the chemical linker is covalently attached at an amino acid residue in the constant region of an antibody. In some embodiments, the chemical linker is covalently attached at an amino acid residue in the CH1, CH2, or CH3 region. In some embodiments, the chemical inker is covalently attached at an amino acid residue in the CH2 region. In some embodiments, the chemical linker may be covalently attached to one amino acid residue in the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the chemical linker is covalently attached to one lysine residue of a human IgG Fc region. In some embodiments, the chemical linker is covalently attached at Lys 246, Lys 248, Lys 288, Lys 290, or Lys 316 of an Fc region of the antibody, wherein amino acid residue position number is based on EU numbering. In some embodiments, the chemical linker is covalently attached at Lys 246 of an Fc region of the antibody, wherein amino acid residue position number is based on EU numbering. In some embodiments, the chemical linker is covalently attached at Lys 248 of an Fc region of the antibody, wherein amino acid residue position number is based on EU numbering. In some embodiments, the chemical linker is covalently attached at Lys 288 of an Fc region of the antibody, wherein amino acid residue position number is based on EU numbering. In some embodiments, the chemical linker is covalently attached at Lys 290 of an Fc region of the antibody, wherein amino acid residue position number is based on EU numbering. In some embodiments, the chemical linker is covalently attached at Lys 317 of the antibody, wherein amino acid residue position number is based on EU numbering.

The chemical linker can be covalently attached to an amino acid residue selected from a subset of amino acid residues. In some embodiments, the subset comprises two three, four, five, six, seven, eight, nine, or ten amino acid residues of an Fc region of the antibody. The chemical linker can be covalently attached to one of two lysine residues of an Fc region of the antibody.

In some embodiments, the antibody will comprise two linkers covalently attached to the Fc region of the antibody. In some embodiments, each of the two linkers will be covalently attached to a different heavy chain of the antibody. In some embodiments, each of the two linkers will be covalently attached to a different heavy chain of the antibody at a residue position which is the same. In some embodiments, each of the two linkers will be covalently attached to a different heavy chain of antibody at a residue position which is different. When the two linkers are covalently attached to residue positions which differ, any combination of the residue positions provided herein may be used in combination.

### Method of Modifying an Fc Region

Also provided herein are method of preparing a modified Fc region of an antibody or antigen binding fragment, such as for the attachment of a linker, a conjugation handle, the IL-18 polypeptide, or any combination thereof to the antibody or antigen binding fragment. A variety of methods for site-specific modification of Fc regions of antibodies are known in the art.

Modification with an Affinity Peptide Configured to Site-Specifically Attach Linker to the Antibody

In some embodiments, an Fc region is modified to incorporate a linker, a conjugation handle, or a combination thereof. In some embodiments, the modification is performed by contacting the Fc region with an affinity peptide bearing a payload configured to attach a linker or other group to the Fc region, such as at a specific residue of the Fc region. In some embodiments, the linker is attached using a reactive group which forms a bond with a residue of the Fc region. In some embodiments, the affinity peptide comprises a cleavable linker. The cleavable linker is configured on the affinity peptide such that after the linker or other group is attached to the Fc region, the affinity peptide can be removed, leaving behind only the desired linker or other group attached to the Fc region. The linker or other group can then be used further to add attach additional groups, such as a cytokine or a linker attached to a cytokine, to the Fc region.

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from (1) QETNPTENLYFQQKNMQCQRRFYEALHDPNLNEEQRNARIRSIRDDDC (SEQ ID NO: 261); (2) QTADNQKNMQCQRRFYEALHDPNLNEEQRNARIRSIRDDCSQSANLLAEAQQLNDAQA PQA (SEQ ID NO: 262); (3) QETKNMQCQRRFYEALHDPNLNEEQRNARIRSIRDDDC (SEQ ID NO: 263); (4) QETFNKQCQRRFYEALHDPNLNEEQRNARIRSIRDDDC (SEQ ID NO: 264); (5) QETFNMQCQRRFYEALHDPNLNKEQRNARIRSIRDDDC (SEQ ID NO: 265); (6) QETFNMQCQRRFYEALHDPNLNEEQRNARIRSIKDDC (SEQ ID NO: 266); (7) QETMQCQRRFYEALHDPNLNEEQRNARIRSIKDDC (SEQ ID NO: 267); (8) QETQCQRRFYEALHDPNLNEEQRNARIRSIKDDC (SEQ ID NO: 268); (9) QETCQRRFYEALHDPNLNEEQRNARIRSIKDDC (SEQ ID NO: 269); (10) QETRGNCAYHKGQLVWCTYH (SEQ ID NO: 270); and (11) QETRGNCA YHKGQIIWCTYH (SEQ ID NO: 271), or a corresponding peptide which has been truncated at the N-terminus by one, two, three, four, or five residues.

An exemplary affinity peptide with cleavable linker and conjugation handle payload capable of attaching the payload to residue K248 of an antibody as provided herein is shown below (as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066.

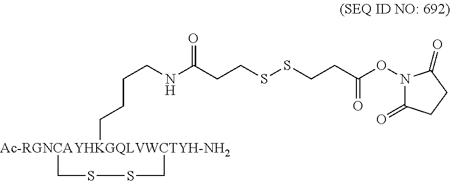

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the linking group (e.g., the relevant portion of the affinity peptide would have a structure of

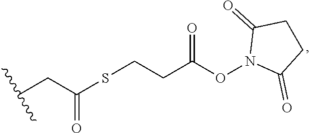 [SMILES: *CC(=O)SCCC(=O)ON1C(=O)CCC1=O]

or another of the cleavable linkers discussed below). Such alternative affinity peptides include those described in, for example “AJICAP Second Generation: Improved Chemical Site-Specific Conjugation Technology for Antibody-Drug Conjugation Technology for Antibody-Drug Conjugate Production” (Working Paper, Fujii et al., DOI: 10.26434/chemrxiv-2023-9p5p7, chemrxiv.org/engage/chemrxiv/article-details/63d5f7131125965a9e7df8a5 (Accessed 20 Feb. 2023, Version 1 published 30 Jan. 2023)). Exemplary affinity peptides provided therein include those shown below, wherein the left structure targets K248 of the Fc region and the right structure targets K288 of the Fc region (EU numbering).

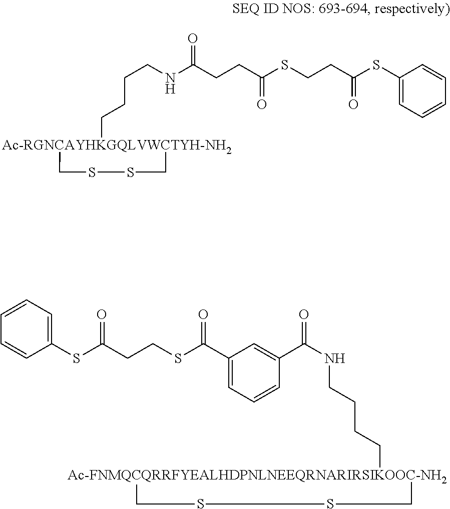

The affinity peptide of the disclosure can comprise a cleavable linker. In some embodiments, the cleavable linker of the affinity peptide connects the affinity peptide to the group which is to be attached to the Fc region and is configured such that the peptide can be cleaved after the group comprising the linker or conjugation handle has been attached. In some embodiments, the cleavable linker is a divalent group. In some embodiments, the cleavable linker can comprise a thioester group, an ester group, a sulfane group; a methanimine group; an oxyvinyl group; a thiopropanoate group; an ethane-1,2-diol group; an (imidazole-1-yl) methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linker is:

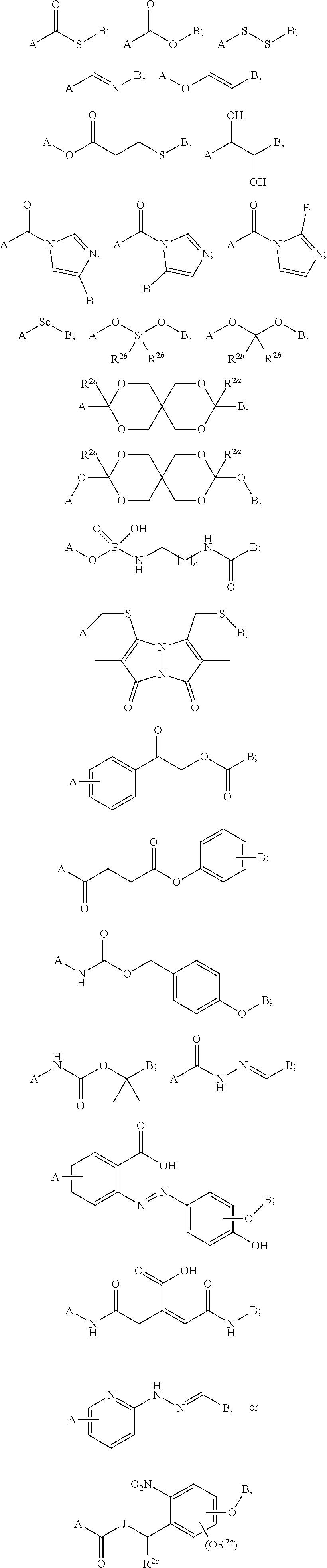

wherein:
one of A or B is a point of attachment the linker and the other of A or B is a point of attachment to the affinity peptide;
each R^{2a} is independently H or optionally substituted alkyl;
each R^{2b} is independently H or optionally substituted alkyl;
R^{2c} is a H or optionally substituted alkyl;
J is a methyl, a N, a S, a Si, or an O atom; and
r is 1, 2, 3, 4, 5, 6, 7, 8, 9, or 10.

The affinity peptide comprises a reactive group which is configured to enable the covalent attachment of the linker/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide). In some embodiments, the reactive group is:

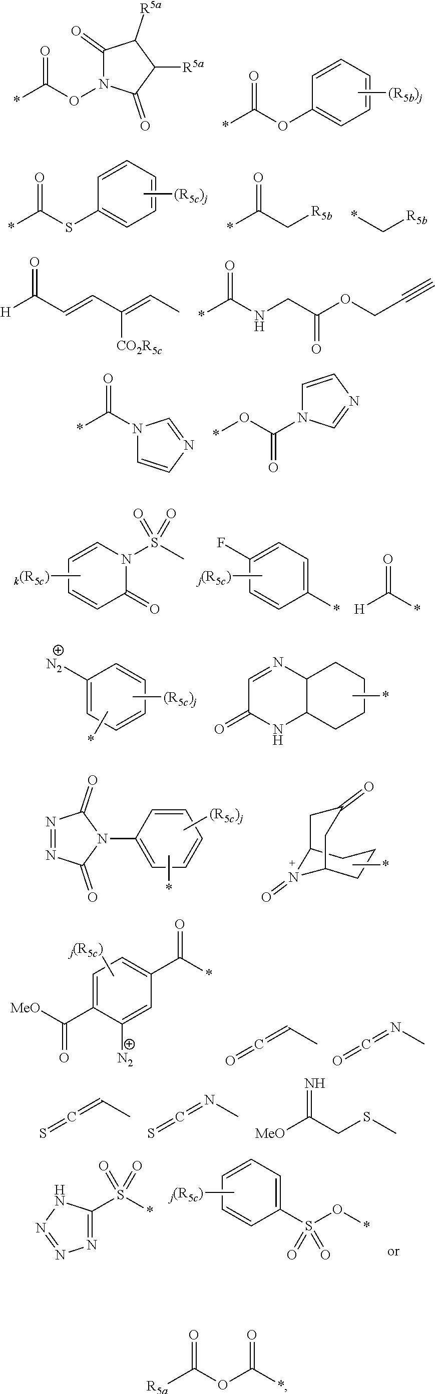

wherein:
each R₅ₐ, R_{5b}, and R_{5c} is independently H, halogen, or optionally substituted alkyl;
each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the antibody or antigen binding fragment due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linker). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B-C, wherein A is the sulfhydryl reactive conjugation handle (e.g., maleimide, α,β-unsaturated carbonyl, a-halogenated carbonyl), B is a linking group, and C is the new conjugation handle (e.g., an alkyne such as DBCO). Specific non-limiting examples of bifunctional linking reagents include

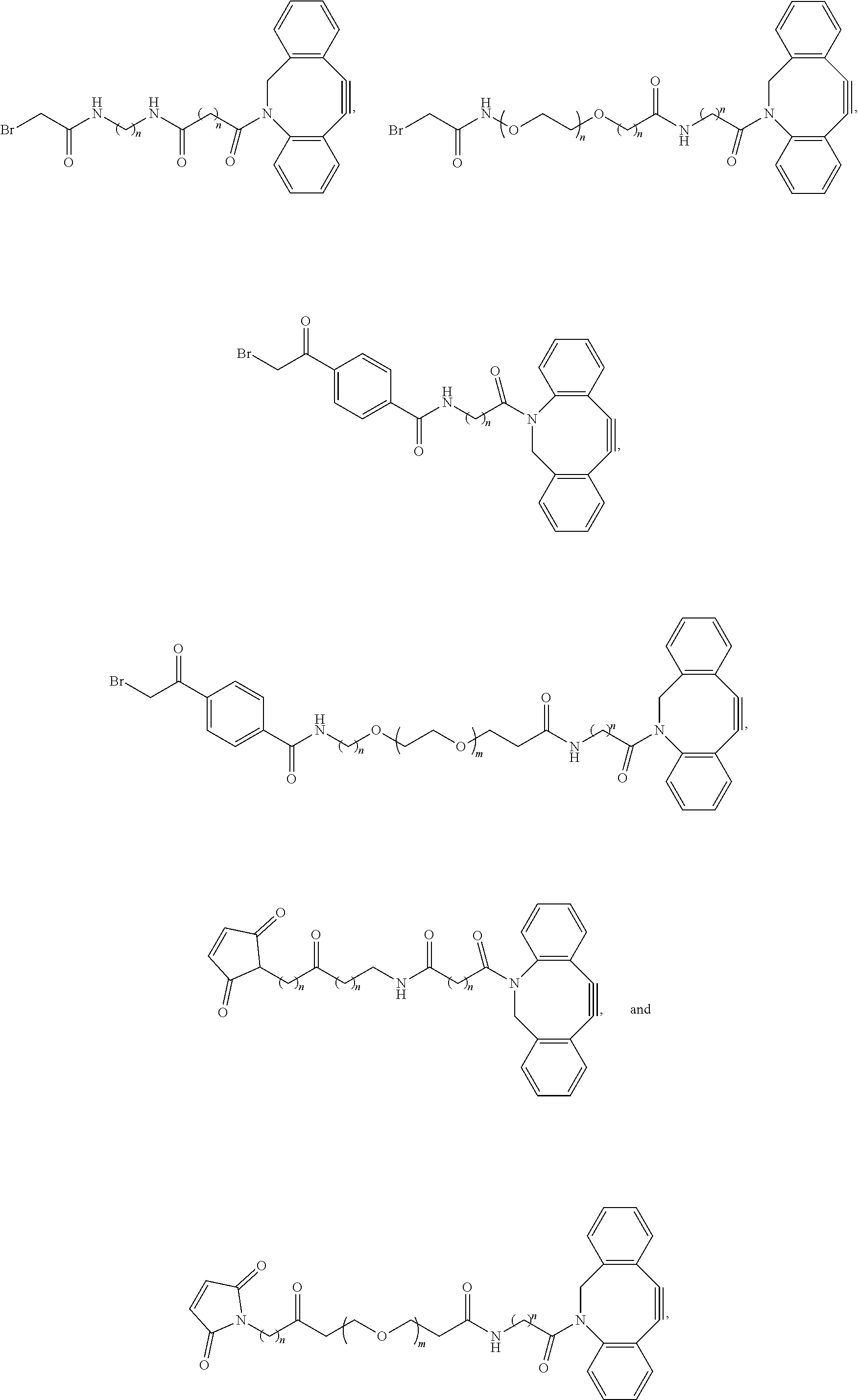

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-18 polypeptide.

### Alternative Methods of Modifying Fc Region

While the affinity peptide mediated modification of an Fc region of an antibody provide supra possesses many advantages over other methods which can be used to site-specifically modify the Fc region (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure.

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications PCT Publication No. WO2020188061, US Patent Publication No. US2019194641, US Patent Publication No. US2021128743, U.S. Pat. Nos. 9,764,038, and 10,434,180, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the immunocytokine composition comprises a full length antibody heavy chain and an antibody light chain, wherein the acyl donor glutamine-containing tag is located at the carboxyl terminus of a heavy chain, a light chain, or both the heavy chain and the light chain.

In some embodiments, the immunocytokine composition comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, provided herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, provided herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX, wherein X is A or P (SEQ ID NO: 272), or GGLLQGPP (SEQ ID NO: 273), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, provided herein is a method for conjugating a moiety of interest (Z) to an antibody, comprising the steps of: (a) providing an antibody having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, provided herein is a method for conjugating a moiety of interest (Z) to an antibody, comprising the steps of: (a) providing an antibody having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the C_{H}2 domain Optionally, the antibody is free of heavy chain N297-linked glycosylation. Optionally, the acceptor glutamine is at position 295 and the residue at the +2 position is the residue at position 297 (EU index numbering) of an antibody heavy chain. The antibody comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue of Formula II on a solid support to provide a sample comprising immobilized antibodies, reacting the sample comprising immobilized antibodies, optionally recovering any unreacted compound and re-introducing such recovered compound to the solid support for reaction with immobilized antibodies, and eluting the antibody conjugates to provide an antibody composition comprising a Z moiety.

In an alternative embodiment, an amino acid residue comprising a conjugation handle can be incorporated into the Fc region of the antibody (e.g., during expression of the antibody) at a desired location (e.g., any of the locations provided herein). In some embodiments, the amino acid residue comprising the conjugation handle is an unnatural amino acid.

### Conjugation Handle Chemistry

In some embodiments, the appropriately modified Fc region of the antibody or antigen binding fragment will comprise a conjugation handle which is used to conjugate the antibody or antigen binding fragment to an IL-18 polypeptide to produce an immunocytokine composition provided herein.

Any suitable reactive group capable of reacting with a complementary reactive group attached to the IL-18 polypeptide can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, potassium acyl trifluoroborate (KAT) ligation or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne, azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, potassium acyl trifluoroborate, hydroxylamine (e.g., O-substituted hydroxylamine) and hydrazide. In some embodiments, the IL-18 polypeptide comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

### Linker Structure

In some embodiments, the linker used to attach the antibody or antigen binding fragment and the IL-18 polypeptide comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein). In some embodiments, the linker is a peptide linker. In some embodiments, the linker is a non-peptide linker (e.g., does not consist of amino acid residues).

### Chemical Linkers

In some embodiments, the linker is a chemical linker. In some embodiments, the chemical linker comprises at least one portion which is not comprised of amino acid residues. In some embodiments, the linker comprises a polymer. In some embodiments, the linker comprises a water soluble polymer. In some embodiments, the linker comprises poly(alkylene oxide), polysaccharide, poly(vinyl poly(vinyl pyrrolidone), alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker is branched and comprises a linear portion of a chain of at least 10, 20, 50, 100, 500, 1000, 2000, 3000, or 5000 atoms. In some embodiments, the linker comprises a linear portion of at from 1 to 1000 atoms, 1 to 900 atoms, 1 to 800 atoms, 1 to 500 atoms, 1 to 400 atoms, 1 to 300 atoms, 1 to 200 atoms, 1 to 100 atoms, 1 to 50 atoms, 10 to 1000 atoms, 10 to 900 atoms, 10 to 800 atoms, 10 to 500 atoms, 10 to 400 atoms, 10 to 300 atoms, 10 to 200 atoms, 10 to 100 atoms, 10 to 50 atoms, 25 to 1000 atoms, 25 to 900 atoms, 25 to 800 atoms, 25 to 500 atoms, 25 to 400 atoms, 25 to 300 atoms, 25 to 200 atoms, 25 to 100 atoms, 25 to 50 atoms, 50 to 1000 atoms, 50 to 900 atoms, 50 to 800 atoms, 50 to 500 atoms, 50 to 400 atoms, 50 to 300 atoms, 50 to 200 atoms, or 50 to 100 atoms. In some embodiments, the linker has a linear length of from about 10 angstroms to about 200 angstroms. In some embodiments, the linker has a linear length of from about 10 to 500, 10 to 200, 10 to 150, 10 to 125, 10 to 100, 10 to 75, 10 to 50, 25 to 200, 25 to 150, 25 to 125, 25 to 100, 25 to 75, 25 to 50, 50 to 200, 50 to 150, 50 to 100, or 50 to 75 angstroms.

In some embodiments, the linker has a molecular weight of about 200 Daltons to about 2000 Daltons. In some embodiments, the linker has a molecular weight of about 200 Daltons to about 5000 Daltons. In some embodiments, the linker has a molecular weight of 200 Daltons to 100,000 Daltons. In some embodiments, the linker has a molecular weight of at least about 500 Daltons, at least about 1,000 Daltons, at least about 5,000 Daltons, at least about 10,000 Daltons, at least about 15,000 Daltons, at least about 20,000 Daltons, at least about 25,000 Daltons, or at least about 30,000 Daltons. In some embodiments, the linker as a molecular weight of at most about 100,000 Daltons, at most about 50,000 Daltons, at most about 40,000 Daltons, at most about 30,000 Daltons, at most about 25,000 Daltons, at most about 20,000 Daltons at most about 15,000 Daltons, at most about 10,000 Daltons, or at most about 5,000 Daltons.

In some embodiments, the linker comprises a reaction product of one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other is.

In some embodiments, the linker comprises a structure of Formula (X)

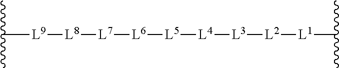

wherein each of L¹, L², L³, L⁴, L⁵, L⁶, L⁷. L⁸, and L⁹ is independently —O—, —NR^{L}—, —(C₁-C₆ alkylene) NR^{L}—, —NR^{L} (C₁-C₆ alkylene)-, —N(R^{L})₂_{⁺}—, —(C₁-C₆ alkylene) N(R^{L})₂_{⁺}—, —N(R^{L})₂_{⁺}—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene) C(═O)—, —C(═O) (C₁-C₆ alkylene)-, —C(═O)O—, —OC(═O)—, —OC(═O)O—, —C(═O) NR^{L}—, —C(═O) NR^{L}(C₁-C₆ alkylene)-, —(C₁-C₆ alkylene) C(═O) NR^{L}—, —NR^{L}C(═O)—, —(C₁-C₆ alkylene) NR^{L}C(═O)—, —NR^{L}C(═O) (C₁-C₆ alkylene)-, —OC(═O) NR^{L}—, —NR^{L}C(═O)O—, —NR^{L}C(═O) NR^{L}—, —NR^{L}C(═S) NR^{L}—, —CR^{L}N—, —N—CR^{L}, —NR^{L}S(═O)₂—, —S(═O)₂NR^{L}, —C(═O) NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each [symbol] is a point of attachment to the antibody or antigen binding fragment or the IL-18 polypeptide.

In some embodiments, the linker consists of a plurality of structures of Formula (X) to form the linkage between the antibody or antigen binding fragment and the IL-18 polypeptide (e.g., 1, 2, 3, 4, 5, 6, 7, 8, 9, 10, or more structures of Formula (X) appended from end to end, where: only the terminal [symbol] denote points of attachment to the antibody or antigen binding fragment or the IL-18 polypeptide).

In some embodiments, the polymer comprises a linker comprising a structure of Formula (X′)

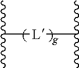

wherein each L′ is independently —O—, —NR^{L}—, —(C₁-C₆ alkylene) NR^{L}—, —NR^{L} (C₁-C₆ alkylene)-, —N(R^{L})₂_{⁺}—, —(C₁-C₆ alkylene)N(R^{L})₂_{⁺}—, —N(R^{L})₂_{⁺}—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene) C(═O)—, —C(═O) (C₁-C₆ alkylene)-, —C(═O)O—, —OC(═O)—, —OC(═O)O—, —C(═O) NR^{L}—, —C(═O) NR^{L} (C₁-C₆ alkylene)-, —(C₁-C₆ alkylene) C(═O) NR^{L}—, —NR^{L}C(═O)—, —(C₁-C₆ alkylene) NR^{L}C(═O)—, —NR^{L}C(═O) (C₁-C₆ alkylene)-, —OC(═O) NR^{L}—, —NR^{L}C(═O)O—, —NR^{L}C(═O) NR^{L}—, —NR^{L}C(=S) NR^{L}—, —CR^{L}═N—, —N═CR^{L}, —NR^{L}S(═O)₂—, —S(═O)₂NR^{L}—, —C(—O) NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene);
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
g is an integer from 1-100,
wherein each [symbol] is a point of attachment to the modified IL-18 polypeptide or the antibody or antigen binding fragment.

In some embodiments, the linker of Formula (X) or Formula (X′) comprises the structure:

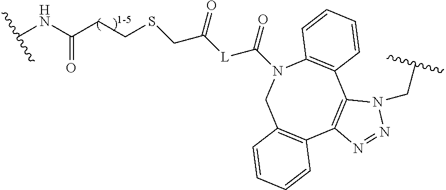

wherein

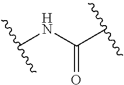 [SMILES: *NC(*)=O]

is the first point of attachment to a lysine residue of the antibody or antigen binding fragment;
L is a linking group; and

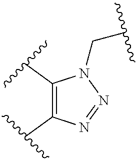 [SMILES: *Cn1nnc(*)c1*]

is a point of attachment to a linking group which connects to the first point of attachment,
or a regioisomer thereof.

In some embodiments, L has a structure

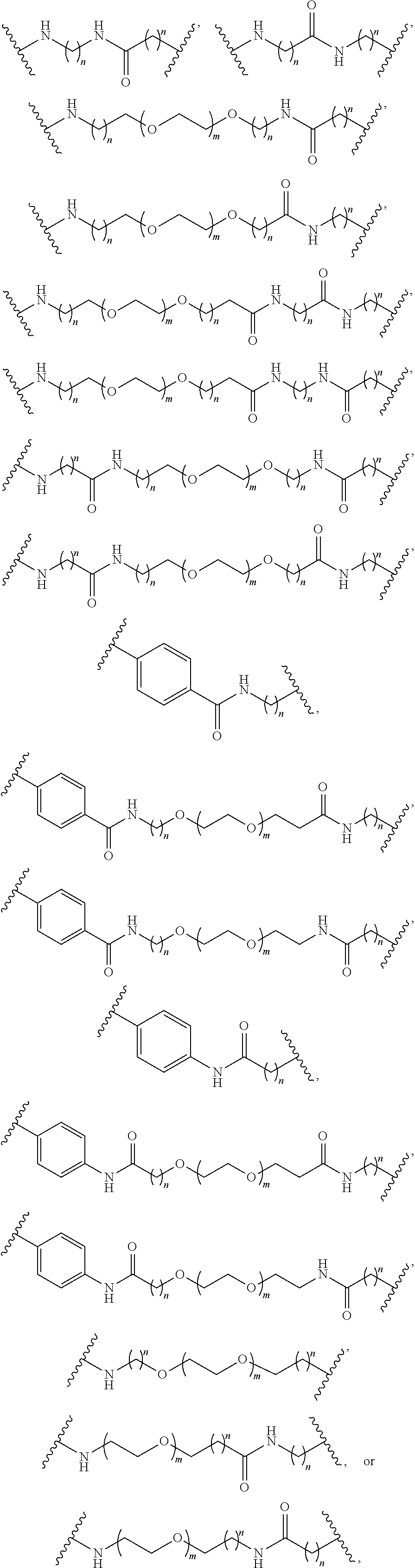

wherein each n is independently an integer from 1-6 and each m is an integer from 1-30. In some embodiments, each m is independently 2 or 3. In some embodiments, each m is an integer from 1-24, from 1-18, from 1-12, or from 1-6.

In some embodiments, the linker of Formula (X) or of Formula (X′) comprises the structure:

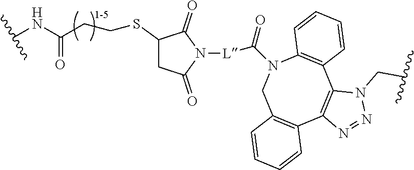

wherein

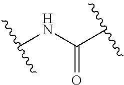 [SMILES: *NC(*)=O]

is the first point of attachment to a lysine residue of the polypeptide which selectively binds to PD-1;
L is a linking group; and

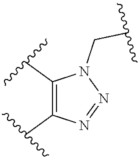 [SMILES: *Cn1nnc(*)c1*]

is a point of attachment to a linking group which connects to the first point of attachment,
or a regioisomer thereof.

In some embodiments, L″ has a structure

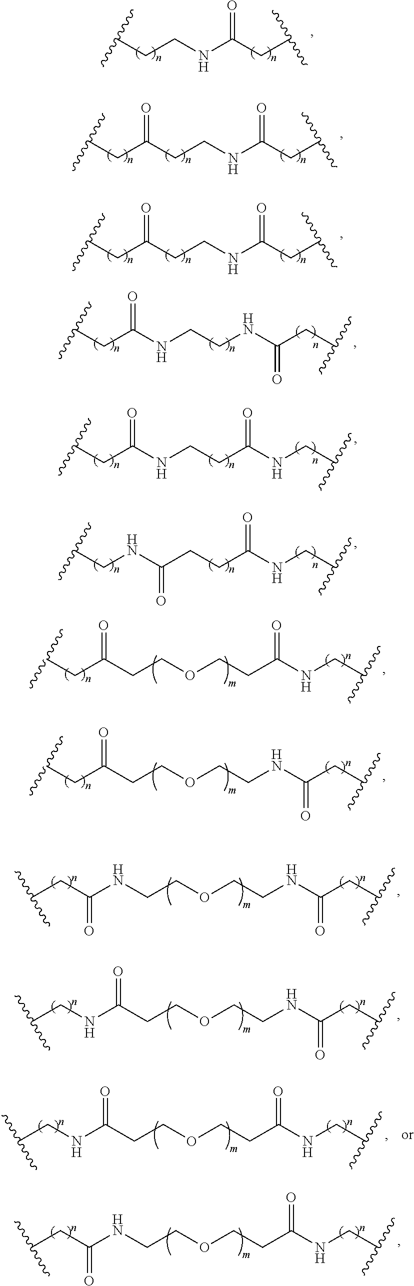

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

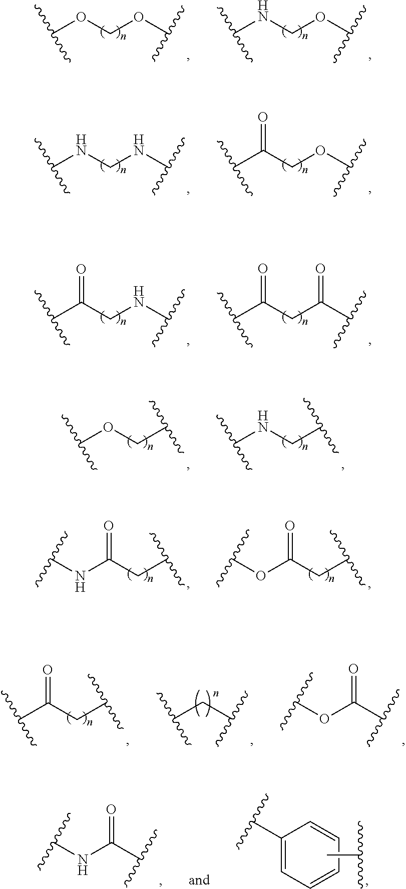

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

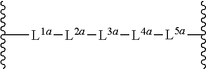

wherein each of L^{1a}, L^{2a}, L^{3a}, L^{4a}, L^{5a}, is independently-O—, —NR^{La}—, —(C₁-C₆ alkylene) NR^{La}—, —NR^{La} (C₁-C₆ alkylene)-, —N(R^{L})₂_{⁺}, —(C₁-C₆ alkylene)N(R^{La})₂_{⁺}(C₁-C₆ alkylene)-, —N(R^{L})₂_{⁺}—, —OP(═O)(OR^{La})O—, —S—, —(C₁-C₆ alkylene)S—, —S(C₁-C₆ alkylene)-, —S(═O)—, —S(═O)₂—, —C(═O)—, (C₁-C₆ alkylene) C(═O)—, —C(═O) (C₁-C₆ alkylene)-, —C(═O)O—, —OC(═O)—, —OC(═O)O—, —C(═O) NR^{La}—, —C(═O) NR^{La} (C₁-C₆ alkylene)-, —(C₁-C₆ alkylene) C(═O) NR^{La}—, —NR^{La}C(═O)—, —(C₁-C₆ alkylene) NR^{La}C(═O)—, —NR^{La}C(═O) (C₁-C₆ alkylene)-, —OC(═O) NR^{La}—, —NR^{La}C(═O)O—, —NR^{La}C(═O) NR^{La}—, —NR^{La}C(═S) NR^{La}—, —CR^{La}—N—, —N═CR^{La}, —NR^{La}S(═O)₂-, —S(═O)₂NR^{La}, —C(═O) NR^{La}S(═O)₂-, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}-, —(O—CH₂—CH₂)_{qf}-, —(CH₂—CH(CH₃)—O)_{qg}-, —(O—CH(CH₃)—CH₂)_{qh}-, a reaction product of a conjugation handle and a complementary conjugation handle, or absent; (C₁-C₆ alkylene)
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the linking group which connects to the first point of attachment (e.g., the point of attachment to the IL-18 polypeptide) comprises poly(ethylene glycol). In some embodiments, the linking group comprises about 2 to about 30 poly(ethylene glycol) units. In some embodiments, the linking group which connects to the first point of attachment (e.g., the point of attachment to the IL-18 polypeptide) is a functionality attached to a cytokine provided herein which comprises an azide (e.g., the triazole is the reaction product of the azide).

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle comprise a structure of

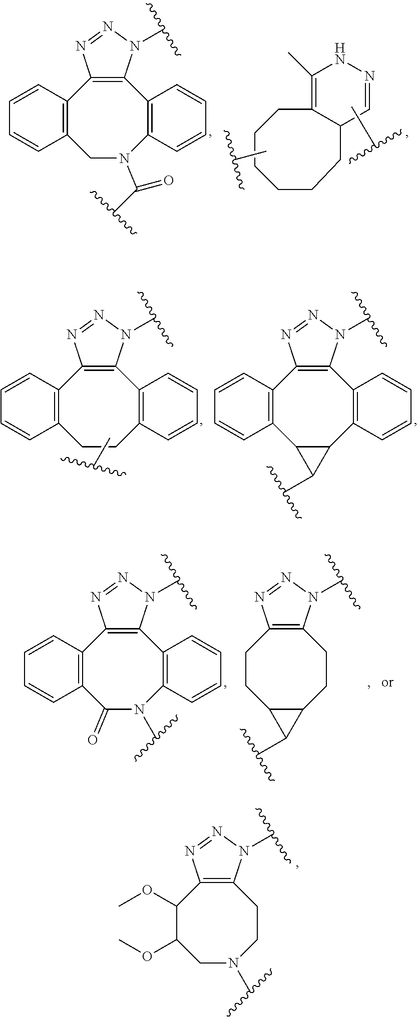

or a regioisomer or derivative thereof.

### Peptide Linkers

In some embodiments, the antibody or antigen binding fragment is linked to the IL-18 polypeptide through a peptide linker. In some embodiments, the antibody or antigen binding fragment is linked to the IL-18 polypeptide as a fusion protein. In such instances, the linker comprises one or more peptide bonds between the antibody or antigen binding fragment and the IL-18 polypeptide. In some embodiments, the linker between the fusion protein of the antibody or antigen binding fragment and the IL-18 polypeptide is a bond. In some embodiments, the linker between the fusion protein of the antibody or antigen binding fragment and the IL-18 polypeptide is a linking peptide. Non-limiting examples of linking peptides include, but are not limited to (GS) n (SEQ ID NO: 424), (GGS)ₙ (SEQ ID NO: 425), (GGGS)ₙ (SEQ ID NO: 426), (GGSG)ₙ (SEQ ID NO: 427), or (GGSGG)ₙ (SEQ ID NO: 428), (GGGGS)ₙ (SEQ ID NO: 429), wherein n is 1, 2, 3, 4, 5, 6, 7, 8, 9, or 10. For example, a linking peptide can be (GGGGS) 3 (SEQ ID NO: 430) or (GGGGS)ₙ (SEQ ID NO: 431). In some embodiments, the IL-18 polypeptide is fused to the C-terminal end of the antibody or antigen binding fragment (optionally through a linking peptide). In some embodiments, the IL-18 polypeptide is fused to the N-terminal end of the antibody or antigen binding fragment (optionally through a linking peptide).

### Cleavable Linkers

In some embodiments, the linker (e.g., a chemical or peptide linker as provided herein) is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the cleavable linker is cleaved by a tumor metabolite at, near, or in the tumor microenvironment. In some embodiments, the cleavable linker is cleaved by a protease at, near, or in the tumor microenvironment.

### Act-IL-18 Polypeptides

The present disclosure relates to activatable immunocytokine compositions containing activatable IL-18 (Act-IL-18) polypeptides that are useful as therapeutic agents. The Activatable IL-18 polypeptides provided herein attached to antibodies can be used as immunotherapies or as parts of other immunotherapy regimens. According to one aspect of the instant disclosure are Act-IL-18 polypeptides themselves which can be useful either as part of activatable immunocytokine compositions described herein, or for other uses. Thus, the instant disclosure expressly regards the Act-IL-18 polypeptides themselves as described herein as being a distinct aspect of the invention (i.e., in certain aspects the invention can include an Act-IL-18 polypeptide described herein independently of an activatable immunocytokine composition).

In one aspect, the Act-IL-18 polypeptides of activatable immunocytokines of the disclosure comprise artificial polypeptides which are attached to an IL-18 polypeptide. An artificial polypeptide can comprise a group such as polymer or spacer used to link the artificial moiety to the IL-18 polypeptide. The artificial polypeptide comprises a cleavable group which, when cleaved, releases all or a portion of the artificial polypeptide. Upon cleavage, the Act-IL-18 is converted into an active form of the IL-18 polypeptide which is capable of performing IL-18 activity.

In one aspect, provided herein, is an Act-IL-18 polypeptide of an activatable immunocytokine comprising an artificial polypeptide attached to an IL-18 polypeptide. In some embodiments, the artificial polypeptide inhibits the Act-IL-18 polypeptide from interacting with and/or signaling through an IL-18 receptor. In some embodiments, the artificial polypeptide is capable of undergoing a change in response to a condition or stimulus which results in a conversion of the Act-IL-18 polypeptide into an active IL-18 polypeptide. In some embodiments, the change is a cleavage of at least a portion of the artificial polypeptide from the IL-18 polypeptide.

In one aspect, an Act-IL-18 of an activatable immunocytokine of the instant disclosure is activated in or near a target tissue of a subject. In some embodiments, the Act-IL-18 is preferentially activated in or near the target tissue of a subject (e.g., activated at a higher rate in or near the target tissue compared to other tissue). In some embodiments, the Act-IL-18 is activated preferentially at or near a target tissue of the subject such that the area at or near the target tissue comprises at least 2-fold, at least 4-fold, at least 8-fold, at least 10-fold, at least 15-fold, at least 20-fold, at least 50-fold, at least 100-fold, at least 200-fold, at least 500-fold, or at least 1000-fold the concentration of the active form of the IL-18 polypeptide compared to non-target tissues. In some embodiments, the concentrations compared are the peak concentrations of the active form of the IL-18 polypeptide after administration of the Act-IL-18. In some embodiments, the Act-IL-18 is preferentially activated in or near disease tissue of the subject. In some embodiments, the Act-IL-18 polypeptide is preferentially activated in or near cancer tissue of the subject. In some embodiments, the Act-IL-18 polypeptide is preferentially activated in or near a tumor microenvironment. In some embodiments, the Act-IL-18 is preferentially activated in the tumor microenvironment compared to non-tumor tissue.

In one aspect, provided herein, is an activatable interleukin-18 (Act-IL-18) polypeptide of an activatable immunocytokine comprising: an artificial polypeptide attached to an interleukin-18 (IL-18) polypeptide, wherein the artificial polypeptide comprises a protease cleavage site, and wherein cleavage at the protease cleavage site converts the Act-IL-18 into an active form of the IL-18 polypeptide.

### Artificial Polypeptides

In one aspect, provided herein, are artificial polypeptides attached to IL-18 polypeptides incorporated into activatable immunocytokines. Artificial polypeptides as provided herein serve to detune the activity of the IL-18 polypeptide while they are attached in an intact form. In some embodiments, cleavage of the artificial polypeptide serves to activate the IL-18 polypeptide (e.g., allowing the IL-18 polypeptide to signal through IL-18Rab). An artificial polypeptide provided herein can be attached to any residue. In some embodiments, the artificial polypeptide is an artificial polypeptide (e.g., attached to a terminal residue of the IL-18 polypeptide).

In some embodiments, the artificial polypeptide can have other functionalities attached (e.g., in addition to being attached to the IL-18 polypeptide, the artificial polypeptide is also attached to another group, such as an additional polypeptide (e.g., antibody, dummy receptor, another cytokine), a half-life extension polymer (e.g., poly(ethylene glycol) (PEG)), or another desired functionality). In some embodiments, cleavage of the artificial polypeptide serves also to cleave this additional group from the IL-18 polypeptide.

In one aspect, the Act-IL-18 polypeptides provided herein comprise an artificial polypeptide attached to a terminal residue of the IL-18 polypeptide. In some embodiments, the artificial polypeptide is covalently attached to the IL-18 polypeptide. In some embodiments, the artificial polypeptide is a group which is not naturally attached to the terminus of a WT IL-18 polypeptide, such as the natural precursor 36 amino acid propeptide directly attached to the WT IL-18. When the IL-18 polypeptide is a variant IL-18 polypeptide (e.g., having at least one amino acid substitution relative to WT IL-18), then the artificial polypeptide can be the natural propeptide. In some embodiments, the artificial polypeptide is engineered to possess the properties provided herein. In some embodiments, the artificial polypeptide is fused to an IL-18 polypeptide (e.g., as a fusion protein). In some embodiments, the artificial polypeptide is chemically attached to an IL-18 polypeptide, or is incorporated into an IL-18 polypeptide by synthetic means (e.g., during synthesis of an IL-18 polypeptide).

In some embodiments, an artificial polypeptide provided herein inhibits at least one activity associated with an IL-18 polypeptide, such as the ability to bind to an IL-18 receptor or effectuate signaling through the IL-18 receptor (IL-18Rab) (e.g., inducing production of IFNγ in an immune cell). In some embodiments, when the artificial polypeptide is intact, the Act-IL-18 polypeptide is in an inactive state (e.g., lacks or has a substantially diminished ability to bind IL-18Rab or signal through IL-18Rab).

In some embodiments, the presence of the intact artificial polypeptide on the IL-18 polypeptide results in the Act-IL-18 polypeptide displaying a binding affinity to IL-18Rab or an IL-18R subunit which is at least 10-fold lower, at least 100-fold lower, at least 200-fold lower, at least 500-fold lower, or at least 1000-fold lower than WT IL-18. In some embodiments, the presence of the intact artificial polypeptide on the IL-18 polypeptide results in the Act-IL-18 polypeptide displaying a binding affinity to IL-18Rab or an IL-18R subunit which is at least 10-fold lower, at least 100-fold lower, at least 200-fold lower, at least 500-fold lower, or at least 1000-fold lower than the IL-18 polypeptide without the artificial polypeptide.

In some embodiments, the presence of the intact artificial polypeptide on the IL-18 polypeptide results in the Act-IL-18 polypeptide displaying an ability to induce IFNγ production in a cell (e.g., an immune cell such as an NK cell) which is at least 10-fold lower, at least 100-fold lower, at least 200-fold lower, at least 500-fold lower, or at least 1000-fold lower than WT IL-18. In some embodiments, the presence of the intact artificial polypeptide on the IL-18 polypeptide results in the Act-IL-18 polypeptide displaying an ability to induce IFNγ production in a cell (e.g., an immune cell such as an NK cell) which is at least 10-fold lower, at least 100-fold lower, at least 200-fold lower, at least 500-fold lower, or at least 1000-fold lower than the IL-18 polypeptide without the artificial polypeptide.

In some embodiments, the artificial polypeptide comprises a protease cleavage site. In some embodiments, the protease cleavage site is a site which is amenable to cleavage under certain specified or known conditions.

In some embodiments, the protease cleavage site is selected such that it is preferentially cleaved (e.g., cleaved at a faster rate or cleaved in more abundance) at a designated target tissue of a subject. In some embodiments, the protease cleavage site is preferentially cleaved at or near a target tissue of the subject such that the protease cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the target tissue is diseased tissue of the subject. In some embodiments, the target tissue is cancer tissue of the subject. In some embodiments, the target tissue is a tumor microenvironment. In some embodiments, the target tissue is a tumor.

In some embodiments, the protease cleavage site is positioned such that all or only a portion of the artificial polypeptide is removed from the IL-18 polypeptide after cleavage. In some embodiments, none of the artificial polypeptide is present on the IL-18 polypeptide after cleavage (e.g., only residues corresponding to residues in SEQ ID NO: 1 are present after cleavage). In some embodiments, a portion of the artificial polypeptide remains attached to the IL-18 polypeptide after cleavage.

The artificial polypeptide can be attached to either the N-terminal residue or the C-terminal residue of the IL-18 polypeptide. In some embodiments, the artificial polypeptide is attached to the N-terminal residue. In some embodiments, the artificial polypeptide is attached to the N-terminal amine of the IL-18 polypeptide. In some embodiments, the artificial polypeptide is attached to the C-terminal residue. In some embodiments, the artificial polypeptide is attached to the C-terminal carboxyl of the IL-18 polypeptide. In some embodiments, the N-terminal residue is the residue closest to residue position 1 of SEQ ID NO: 1 which is present on an IL-18 polypeptide as provided herein (e.g., the first residue of SEQ ID NO: 1 which has not been truncated). In some embodiments, the N-terminal residue of the IL-18 polypeptide is the residue at a position corresponding to position 1 in SEQ ID NO: 1. In some embodiments, the N-terminal residue of the IL-18 polypeptide is Y1. In some embodiments, the N-terminal residue of the IL-18 polypeptide is Y1G. In some embodiments, the N-terminal residue of the IL-18 polypeptide is YIM. In some embodiments, the C-terminal residue is the residue at position 157 of SEQ ID NO: 1. In some embodiments, the C-terminal residue is D157.

In some embodiments, terminal residues of the IL-18 polypeptide are substituted such that the artificial polypeptide is positioned such that the entirety of the artificial polypeptide is cleaved from the IL-18 polypeptide. For example, if it is intended to introduce a cleavage site at a position corresponding to residue 1 of SEQ ID NO: 1, a protease cleavage sequence P1-P2-P3-P′1-P′2-P′3 can be selected (where the cleavage site is between P3 and P′1), and residues 1, 2, and 3 of SEQ ID NO: 1 can be substituted for P′1, P′2, and P′3 respectively, with P1-P2-P3-appended thereon. In this case, the artificial polypeptide would be considered to comprise P1-P2-P3-, with P′1, P′2, and P′3 as part of the IL-18 polypeptide (substituted residues).

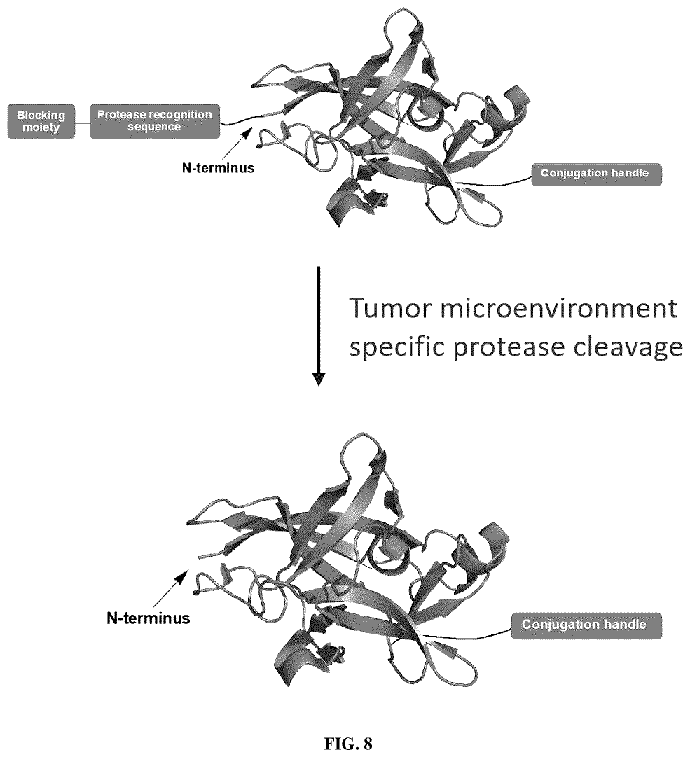

Examples of artificial polypeptides and their mechanisms of action are shown in FIGS. 8-13. FIG. 8 depicts an artificial polypeptide affixed to a terminal residue of the IL-18 polypeptide. Also attached to the artificial polypeptide is a blocking moiety which facilitates keeping the IL-18 polypeptide in an inactive state. When the artificial polypeptide is cleaved (e.g., by a tumor microenvironment protease), the entire artificial polypeptide is cleaved (revealing the terminal residue of the IL-18 polypeptide, as is the blocking moiety. The IL-18 in this example has a conjugation handle attached elsewhere to the IL-18 polypeptide, which can serve to attach an additional group (e.g., half-life extension polymer or an additional polypeptide), and it can be attached either before or after cleavage of the artificial polypeptide.

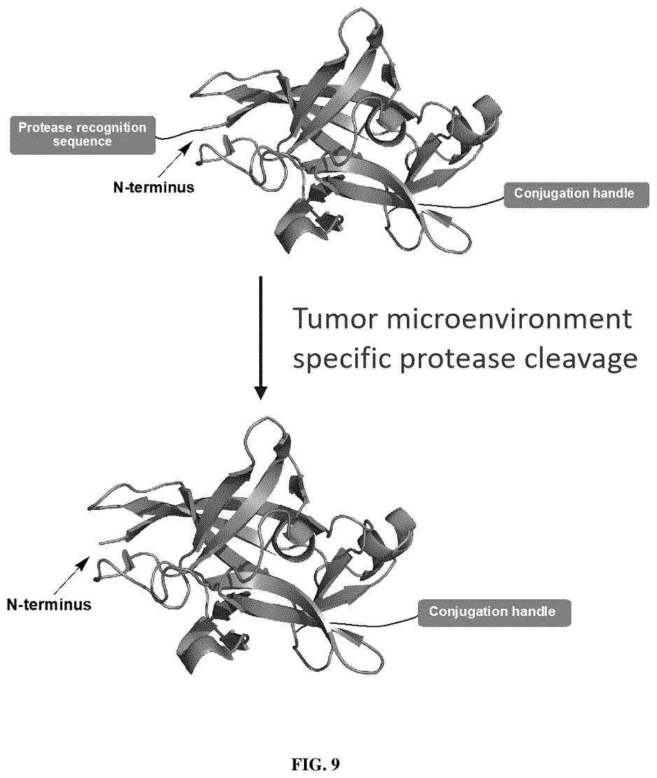

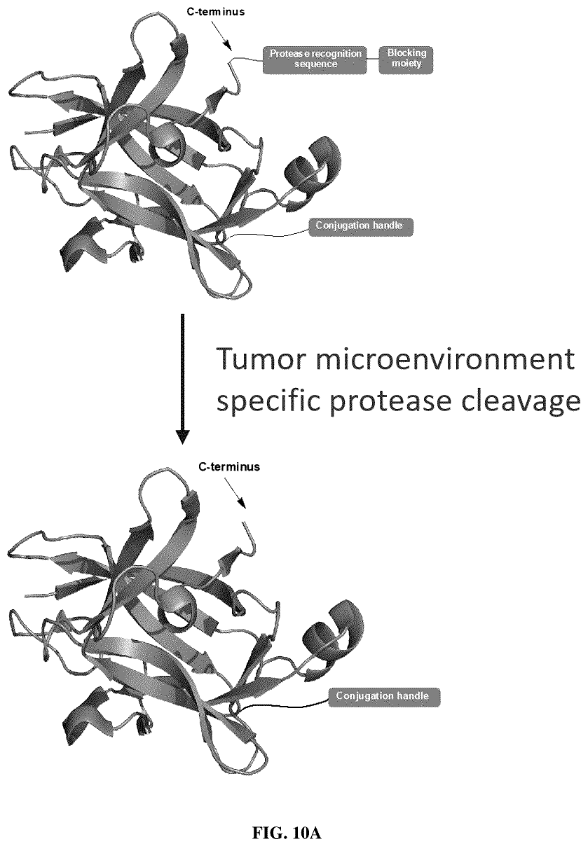

FIG. 9 shows a similar Act-IL-18 construct shown in in the previous figure but lacks the blocking moiety. In some instances, the presence of only a short peptide on the N-terminus can sufficiently detune the activity of the IL-18 polypeptide, and the activity can be restored upon cleavage.

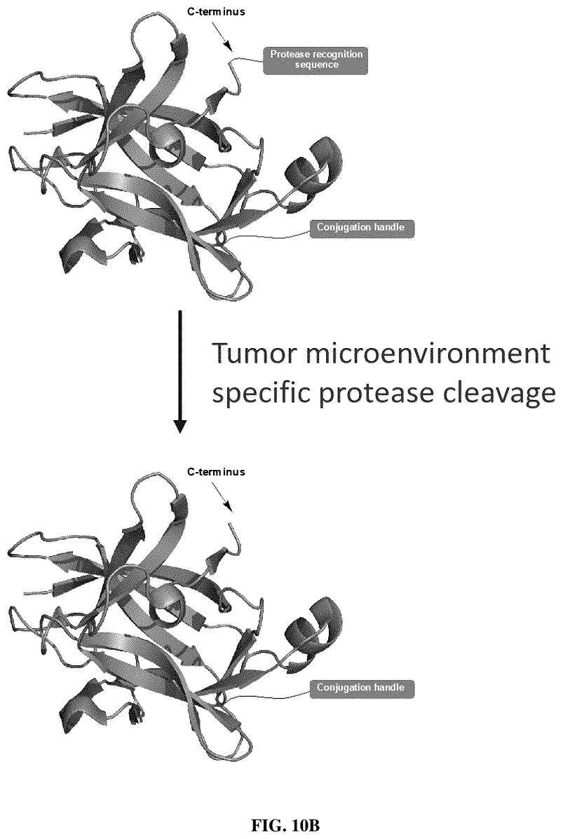

FIG. 10A shows a similar Act-IL-18 construct to that shown in FIG. 8, but the artificial polypeptide is attached to the C-terminus.

FIG. 10B shows a similar Act-IL-18 construct to that shown in FIG. 9, but the artificial polypeptide is attached to the C-terminus.

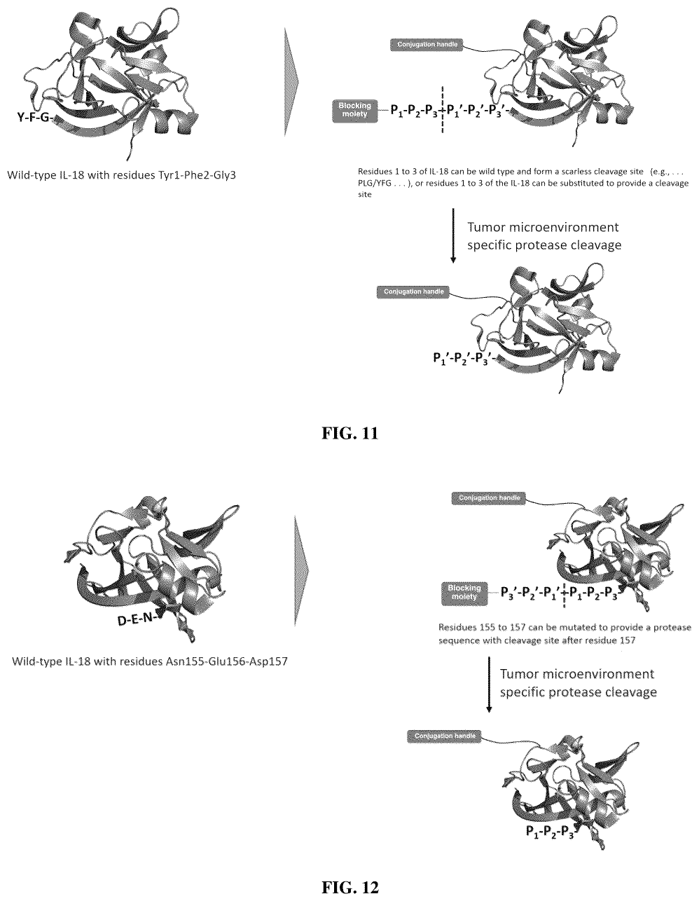

FIG. 11 depicts an analogous construct to that shown in FIG. 8, but residues 1, 2, and 3 of the IL-18 polypeptide are substituted such that the artificial polypeptide is cleaved entirely (residues 1, 2, and 3, though substituted, still correspond to positions 1, 2 and 3 of SEQ ID NO: 1). FIG. 12 is analogous to FIG. 11, but the artificial polypeptide is at the C-terminus. Alternatively to substituting residues 1, 2, and 3 of the IL-18 as depicted in FIG. 11, a cleavable peptide could be selected such that natural residues 1, 2, and 3 of the IL-18 polypeptide (e.g., Y, F and G, respectively), or a subset of the natural residues, form part of the cleavage recognition site of the relevant protease such that the entire artificial polypeptide is cleaved, thus leaving an unmodified N-terminus of the IL-18 polypeptide after cleavage. For example, a sequence of -PLG- can be appended the N-terminal Y of the IL-18 polypeptide to allow complete cleavage of the artificial polypeptide by a matrix metalloprotease. Additionally, in such a case, the N-terminal residue or set of residues (e.g., residues 1, 2, and/or 3) can be substituted relative to SEQ ID NO: 1 in order to optimize cleavage efficiency of the cleavage site. Such an approach could also be adopted at the C-terminus of the IL-18 polypeptide.

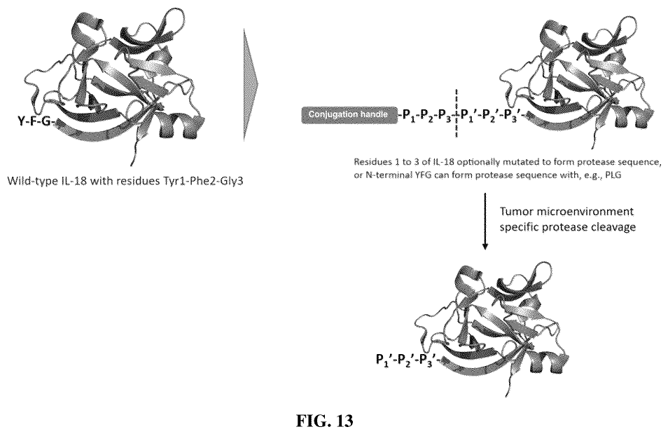

FIG. 13 depicts an Act-IL-18 polypeptide comprising an artificial polypeptide which has a conjugation handle attached to it such that cleavage of the artificial polypeptide will also cleave the conjugation handle (or anything which has been attached to the conjugation handle) from the IL-18 polypeptide. By way of non-limiting example, such a construct could be used to conjugate an antibody to the IL-18 polypeptide. Such a construct could be used to tissue specifically deliver an IL-18 polypeptide, then also activate it at the desired site through cleavage at the target tissue.

In some embodiments, cleavage at the protease cleavage site leaves no amino acid residues attached to the IL-18 polypeptide. In some embodiments, cleavage at the protease cleavage site leaves at least 1 amino acid residue attached to the IL-18 polypeptide. In some embodiments, cleavage at the protease cleavage site leaves at most 1, 2, 3, 4, or 5 amino acid residues attached to the IL-18 polypeptide. In some embodiments, cleavage at the protease cleavage site leaves 1, 2, 3, 4, or 5 amino acid residues attached to the IL-18 polypeptide. In some embodiments, cleavage at the protease cleavage site leaves 1, 2, 3, 4, 5, 6, 7, 8, 9, or 10 or more amino acid residues attached to the IL-18 polypeptide.

In some embodiments, an Act-IL-18 can optionally contain a tag used for expression and/or purification of a recombinant Act-IL-18 as provided herein. The tag can be situated upstream of an N-terminal artificial polypeptide or downstream of a C-terminal artificial polypeptide. The tag may be removed (e.g., enzymatically) prior to final formulation and/or administration to a subject. Exemplary tags include a HIS6 (HHHHHH, SEQ ID NO: 687), a Strep Tag (WSHPQFEK (SEQ ID NO: 688) or AWAHPQPGG (SEQ ID NO: 689), or a chitin binding tag (TTNPGVSAWQVNTAYTAGQLVTYNGKTYKCLQPHTSLAGWEPSNVPALWQLQ, SEQ ID NO: 87).

The artificial polypeptide can be cleaved by a protease. In some embodiments, the artificial polypeptide contains a protease cleavage site that can be cleaved specifically by one or more proteases. In some embodiments, the protease cleavage site can be cleaved at a site preferred by one or more proteases.

In some embodiments, the protease is found at higher concentrations and/or demonstrates higher proteolytic activity at or near a target tissue of a subject. In some embodiments, the target tissue is disease tissue. In some embodiments, the target tissue is a cancer. In some embodiments, the target tissue is a tumor microenvironment.

In some embodiments, the protease is found at higher concentrations and/or demonstrates higher proteolytic activity at or near the tumor microenvironment relative to non-tumor tissue. In some embodiments, the protease is found at higher concentrations at or near the tumor microenvironment relative to non-tumor tissue. In some embodiments, the protease demonstrates higher proteolytic activity at or near the tumor microenvironment relative to non-tumor tissue. In some embodiments, the protease is selected from kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, and a tumor cell surface protease. In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the protease is selected from kallikrein, thrombin, chymase, carboxypeptidase A, an elastase, proteinase 3 (PR-3), granzyme M, urokinase plasminogen activator (uPA), a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, and a tumor cell surface protease. In some embodiments, the cleavable peptide is cleavable by a protease selected from a kallikrein, thrombin, chymase, carboxypeptidase A, an elastase, proteinase 3 (PR-3), urokinase plasminogen activator (uPA), granzyme M, a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, a matriptase, and a tumor cell surface protease, or any combination thereof. In some embodiments, the protease is urokinase plasminogen activator (uPA) or matriptase. In some embodiments, the cleavable peptide is cleavable by urokinase plasminogen activator (uPA) or matriptase. In some embodiments, the protease is a protease selected from the Table below.

Tissue/Tumor specific proteases

| | | |
|---|---|---|
| Protease | Specificity | Other Aspects |

Secreted by CD8⁺ Cytotoxic T cells

| | | |
|---|---|---|
| Granzyme B (grB) | Cleaves after Asp | Type of serine protease; Strongly |
| | | implicated in inducing perforin-dependent |
| | | target cell apoptosis |
| Granzyme A (grA) | trypsin-like, cleaves after basic | Type of serine protease. |
| | residues | |
| Granzyme H (grH) | Unknown substrate specificity | Type of serine protease; |
| | | Other granzymes are also secreted by killer |
| | | T cells, but not all are present in humans |
| Caspase-8 | Cleaves after Asp residue | Type of cysteine protease; plays and |
| | | essential role in TCR-induced cellular |
| | | expansion. |
| Mucosa-associated | Cleaves after Arg residues | Type of cysteine protease; likely acts both |
| lymphoid tissue | | as a scaffold and proteolytically active |
| (MALT1) | | enzyme in the CBM-dependent signaling |
| | | pathway. |
| Tryptase | Target: angiotensin I, | Type of mast cell-specific serine protease; |
| | fibrinogen, prourokinase, | trypsin-like; resistant to inhibition by |
| | TGFβ; preferentially cleaves | macromolecular protease inhibitors |
| | proteins after lysine or | expressed in mammals due to their |
| | arginine residues. | tetrameric structure, with all sites facing |
| | | narrow central pore; also associated with |
| | | inflammation. |

Associated with Inflammation:

| | | |
|---|---|---|
| Thrombin | Targets: FGF-2 | Type of serine protease; modulates activity |
| | HB-EGF, Osteo-pontin, | of vascular growth factors, chemokines |
| | PDGF, VEGF | and extracellular proteins; strengthens |
| | | VEGF-induced proliferation; induces cell |
| | | migration; angiogenic factor; regulates |
| | | hemostasis |
| Chymase | Exhibit chymotrypsin-like | Type of mast cell-specific serine protease. |
| | specificity, cleaving proteins | |
| | after aromatic amino acid | |
| | residues. | |
| Carboxypeptidase A | Cleaves amino acid residues | Type of zinc-dependent metalloproteinase |
| (MC-CPA) | from C-terminal end of | |
| | peptides and proteins | |
| Kallikreins | Targets: high molecular | Type of serine protease; modulate |
| | weight kininogen, pro- | relaxation response; contribute to |
| | urokinase | inflammatory response; activates pro- |
| | | apoptotic signaling |
| Elastase | Targets: E-cadherin, GM-CSF, | Type of neutrophil serine protease; |
| | IL-1, IL-2, IL-6, IL-8, | degrades ECM components; regulates |
| | p38^{MAPK}, TNFα, VE-cadherin. | inflammatory response; activates pro- |
| | | apoptotic signaling. |
| Cathepsin G | Targets: ENA-78, IL-8, MCP- | Type of serine protease; degrades ECM |
| | 1, MMP-2, MT1-MMP, PAI- | components; chemo-attractant of |
| | 1, RANTES, TGFβ, TNFα | leukocytes; regulates inflammatory |
| | | response; promotes apoptosis. |
| PR-3 | Targets:ENA-78, IL-8, IL-18, | Type of serine protease; promotes |
| | JNK, p38^{MAPK}, TNFα | inflammatory response; activates pro- |
| | | apoptotic signaling. |
| Granzyme M (GrM) | Cleaves after Met and other | Type of serine protease; only expressed in |
| | long, unbranched hydrophobic | NK cells. |
| | residue. | |
| Calpains | Cleave between Arg and Gly | Family of cysteine proteases; calcium- |
| | | dependent; activation is involved in the |
| | | process of numerous inflammation- |
| | | associated diseases. |

In some embodiments, the protease cleavage site is comprised within a protease recognition sequence. The protease recognition sequence can be recognized and cleaved by the protease. In some embodiments, the protease recognition sequence is a natural peptide sequence which has been incorporated into the artificial polypeptide. In some embodiments, the protease recognition sequence is a synthetic (e.g., man-made, designed, or engineered) sequence. In some embodiments, the protease recognition sequence comprises an amino acid sequence having at least about 80%, at least about 85%, at least about 90%, at least about 95%, at least about 96%, at least about 97%, at least about 98%, at least about 99%, or 100% identical to a sequence set forth in Table 2A.

TABLE 2A
Protease specific protease recognition sequences

| | | Protease recognition |
|---|---|---|
| SEQ ID NO: | Protease | sequences |
| 501 | MMP7 | KRALGLPG |
| 502 | MMP9 | PR(S/T)(L/I)(S/T) |
| 503 | MMP9 | LEATA |
| 504 | MMP11 | GGAANLVRGG |
| 505 | MMP14 | SGRIGFLRTA |
| 506 | MMP | PLGLAG |
| 507 | MMP | PLGLAX |
| 508 | MMP | ESPAYYTA |
| 509 | MMP | RLQLKL |
| 510 | MMP | RLQLKAC |
| 511 | Urokinase | SGRSA |
| | plasminogen activator | |
| | (uPA) | |
| 512 | uPA | DAFK |
| 513 | uPA | GGGRR |
| 514 | Lysosomal Enzyme | GFLG |
| 515 | Lysosomal Enzyme | ALAL |
| 516 | Lysosomal Enzyme | FK |
| 517 | CathepsinB | NLL |
| 518 | CathepsinD | GLLYA |
| 519 | Cathepsin K | GGPRGLPG |
| 520 | Prostate Specific | HSSKLQ |
| | Antigen | |
| 521 | Prostate Specific | HSSKLQL |
| | Antigen | |
| 522 | Prostate Specific | HSSKLQEDA |
| | Antigen | |
| 523 | Thrombin | DPRSFL |
| 524 | Thrombin | PPRSFL |
| 525 | Caspase-3 | DEVD |
| 526 | Caspase-3 | DEVDP |
| 527 | Caspase-3 | KGSGDVEG |
| 528 | Interleukin 1P | GWEHDG |
| | converting enzyme | |
| 529 | FAP | KQEQNPGST |
| 530 | Kallikrein 2 | GKAFRR |
| 531 | Plasmin | DAFK |
| 532 | Plasmin | DVLK |
| 533 | Plasmin | DAFK |
| 534 | TOP | ALLLALL |
| 100 | MMP9 (also referred | SGGPGPAGMKGLPG |
| | to herein as Linker X) | |
| 101 | MMP9 | SGGPGPAGMKGLPGS |
| 102 | MMP2-7-9-14 + | EAGRSANHEPLGLVAT |
| | matriptase + uPA + | |
| | Legumain | |
| 103 | MMP9 + matriptase + | PQASTGRSGG |
| | uPA | |
| 104 | MMP9 + matriptase + | PQGSTGRAAG |
| | uPA | |
| 105 | MMP9 + matriptase + | PPASSGRAGG |
| | uPA | |
| 106 | MMP7-9-10 | RSGVPLSLYSGSGGGK |
| 107 | MMP7-9-10 | RSGMPYDLYHPSGK |
| 108 | MMP7-9-10 | RGPDSGGFMLTSGK |
| 109 | MMP7-9-10 | RGSGHEQLTVSGGSK |
| 110 | MMP7-9-10 | RSGRAAAVKSPSGK |
| 111 | MMP7-9-10 | RGSGISSGLLSGRSDNHSGK |
| 112 | MMP7-9-10 | RGDLLAVVAASGGK |
| 113 | MMP7-9-10 | RGGISSGLLSGRSGK |
| 114 | Meprin A/B | SGGGKKLADEPEGGS |
| 115 | Meprin A/B (High | GGGKFLADEPEGG |
| | Efficiency) | |
| 116 | Cathepsin S, K, L | ARLQSAAP |
| 117 | Cathepsin S + MMP9 + | ARLQSAAPAGLKGA |
| | Meprin A | |
| 118 | MMP9 (High | GSGGPGPAGMHGLPGGS |
| | Efficiency) | |
| 119 | uPA | GGGSHTGRSAYFGGGS |
| 120 | MMP9-2 | SGGPGPAGLKGAPGS |
| 121 | MMP2-7-9 | VPLSLYSG |
| 122 | uPA + Matriptase + | SGLLSGRSDNH |
| | legumain | |
| 123 | uPA | SGRSDNIGGGS |
| 124 | MMP-14 + uPA | LQESLRSKESGRSDI |
| 125 | uPA | ISSGLLSGRSDNH |
| 126 | uPA | LSGRSDDH |
| 127 | uPA | ISSGLLSGRSDQH |
| 128 | uPA | ISSGLLSGRSDNI |
| 129 | MMP2-7-9 | PLGLAG |
| 130 | MMP | GPAGMKGL |
| 131 | MMP | LSGRSDQH |
| 132 | MMP | LSGRSDNI |
| 133 | MMP | ISSGLLSGRSDNH |
| 134 | MMP | GPLGVRG |
| 135 | MMP | GPLGLAR |
| 136 | MMP | GPAALVGA |
| 137 | MMP | GPAALIGG |
| 138 | MMP | GPLNLVGR |
| 139 | MMP | GPAGLVAD |
| 140 | MMP | GPANLVAP |
| 141 | MMP | VPLSLYSG |
| 142 | MMP | SGESPAYYTA |
| 143 | MMP | GGPRGLG |
| 144 | MMP | HSSKLQ |
| 145 | MMP | HSSKLQL |
| 146 | MMP7 | KRALGLPG |
| 147 | MMP9 | LEATA |
| 148 | MMP11 | GGAANLVRGG |
| 149 | MMP14 | SGRIGFLRTA |
| 150 | MMP | PLGLA |
| 151 | MMP | ESPAYYTA |
| 152 | MMP | RLQLKL |
| 153 | MMP | RLQLKAC |
| 154 | uPA | SGRSA |
| 155 | uPA | (D-Ala)FK |
| 156 | uPA | GGGRR |
| 157 | Lysosomal Enzyme | GFLG |
| 158 | Prostate Specific | HSSKLQEDA |
| | Antigen | |
| 159 | HSV Protease | LVLASSSFGY |
| 160 | HIV Protease | GVSQNYPIVG |
| 161 | CMV Protease | GVVQASCRLA |
| 162 | Thrombin | DPRSFL |
| 163 | Thrombin | PPRSFL |
| 164 | Caspase-3 | DEVD |
| 165 | Caspase-3 | DEVDP |
| 166 | Caspase-3 | KGSGDVEG |
| 167 | Interleukin 1β | GWEHDG |
| | converting enzyme | |
| 168 | Enterokinase | EDDDDKA |
| 169 | FAP | KQEQNPGST |
| 170 | Kallikrein 2 | GKAFRR |
| 171 | Plasmin | DAFK |
| 172 | Plasmin | DVLK |
| 173 | Plasmin | DAFK |
| 174 | TOP | ALLLALL |
| 175 | MMP A3 | SGAKPRALTA |
| 176 | MMP B49 | SGLRLAAITA |
| 177 | MMP B74 | SGESLAYYTA |
| 178 | MMP B74P | SGESPAYYTA |
| 179 | MMP B74R | SGESLRYYTA |
| 180 | MMP C9 | SGRSLSRLTA |
| 181 | MMP C9R | SGRSLRRLTA |
| 182 | MMP A13 | SGAVSWLLTA |
| 183 | MMP A13P | SGAPSWLLTA |
| 184 | MMP A13R | SGAVRWLLTA |
| 185 | MMP B37 | SGANISDLTA |
| 186 | MMP A34 | SGNRYSSLTA |
| 187 | MMP A21/ | SGHMHKALTA |
| | MMPA21A | |
| 190 | MMP | GEEGEEPLGLAG |
| 191 | MMP | GPLGLAG |
| 192 | | EAGRSANHTPAGLTGP |
| 193 | | GEAGRSANHTPAGLTGP |
| 194 | MMP (partial | PLG |
| | sequence) | |

In some embodiments, the protease recognition sequence comprises an amino acid sequence having at least about 80%, at least about 85%, at least about 90%, at least about 95%, at least about 96%, at least about 97%, at least about 98%, at least about 99%, or 100% identical to a sequence set forth in Table 2B.

TABLE 2B
Protease Cleavage Sequences

| SEQ ID NO | Sequence |
|---|---|
| 101 | SGGPGPAGMKGLPGS |
| 601 | SSRGPASNRRLPLGLAG |
| 602 | SSRAVFRKNLGPLGLAG |
| 603 | SGRVLRKAGPQPLVD |
| 604 | SGRVLTLRKAPWGLLE |
| 605 | SGRVLGPAGLVG |
| 606 | SGRVLGPANLVG |
| 607 | SGRVLPAGLVG |
| 608 | SGRVLGPLNLSG |
| 609 | RQRRSAPLGLAG |
| 610 | RQRRSPLGLAG |
| 611 | SGRVLTLRKAGPAGLVG |
| 612 | SGRVLTLRKAGPANLVG |
| 613 | SGRVLRKAGPAGLVG |
| 614 | SGRVLRKAGPANLVG |
| 615 | SGRVLPANLVG |
| 616 | SGRVAGLVG |
| 617 | SGRVANLVG |
| 618 | PASNRRLPLGLAG |
| 619 | SSRVFRKPANLAG |
| 620 | SGRVLTLRKAALPLAM |
| 621 | SSRGRRGPYMLQG |
| 622 | SSRGPYMLQG |
| 623 | SGRVLPLGMRA |
| 624 | SGRVLPYAMTA |
| 625 | KWGKSAPLGLAG |
| 626 | RYGKSAPLGLAG |
| 627 | RQRRSAAPLGLAG |
| 628 | RQRRSVVGG |
| 629 | SPLGLAGS |
| 630 | RGRKVANLVG |
| 631 | RQRKVANLVG |
| 632 | RGRRVANLVG |
| 633 | RGRKSPANLVG |
| 634 | RGRKPYMLQG |
| 635 | RGRKSPYMLQG |
| 636 | RGRKPQPLVD |
| 637 | RGRKSPQPLVD |
| 638 | RGRKSQPLVD |
| 639 | SGRVAPYMLQG |
| 640 | SGRVYMLQG |
| 641 | SGRVAPQPLVD |
| 642 | SGRVQPLVD |
| 643 | RGRRGP |
| 644 | PLGLAG |
| 645 | PLG |
| 646 | SGRVANLVG |
| 647 | SGRVANLVGGSG |
| 648 | RGRRVANLVG |
| 649 | RGRRVANLVGGSG |
| 650 | SGRVAN |
| 651 | RGRRVAN |
| 652 | SGRPLG |
| 653 | RGRRPLG |
| 654 | SGRPLGLAG |
| 655 | RGRRPLGLAG |
| 656 | RGRRVANPLGLAG |
| 657 | RGRRVANPLGLAGSG |
| 658 | RGRRPLGLAGGSG |
| 659 | RGRRVANPLG |
| 660 | RGRRVANPLGGSG |
| 661 | RGRRSGRVANPLG |
| 662 | RGRRSGRAANLVRPLGLAG |
| 663 | AANLVR |
| 664 | RGRRSGRAANLVRPLG |
| 665 | RGRRAANLVRPLG |
| 666 | RGRRAANLVRPLGLAG |
| 667 | RGRRAANLVR |
| 668 | RGRRSGRAANLVR |
| 669 | HSSKLQ |
| 670 | RGRRHSSKLQ |
| 671 | RGRRSGRHSSKLQ |
| 672 | RGRRHSSKLQPLG |
| 673 | RGRRSGRHSSKLQPLG |
| 674 | RGRRHSSKLQVAN |
| 675 | RGRRSGRHSSKLQVAN |
| 676 | SGRVANPLGLAG |
| 677 | SGRVANPLGLAGSG |
| 678 | SGRPLGLAGGSG |
| 679 | SGRVANPLG |
| 680 | SGRVANPLGGSG |
| 681 | SGRAANLVRPLG |
| 682 | SGRAANLVRPLGLAG |
| 683 | SGRAANLVR |
| 684 | SGRHSSKLQ |
| 685 | SGRHSSKLQPLG |
| 686 | SGRHSSKLQVAN |

In some embodiments, cleavage of the protease cleavage site leaves no amino acid residues attached to the IL-18 polypeptide. In such cases, the protease recognition sequence can be selected such that portions of the IL-18 polypeptide make up part of the recognition sequence, or are compatible therewith. For example, in some embodiments, the sequence PLG is appended to the N-terminus of the IL-18 polypeptide (e.g., residue 1 of SEQ ID NO: 1), which results in the specific cleavage site being between the G of the PLG and the N-terminus of the IL-18 polypeptide, thereby resulting in a “scarless” activated IL-18 polypeptide after cleavage.

In some embodiments, a portion of the protease recognition sequence which defines the protease cleavage site will be comprised in the sequence of the IL-18 polypeptide (e.g., part of the protease recognition sequence will be comprised at positions which correspond to positions of SEQ ID NO: 1). In some embodiments, the portion of the protease recognition sequence comprised in the sequence of the IL-18 polypeptide will be substituted relative to the sequence set forth in SEQ ID NO: 1. For example, in some embodiments, the last three amino acids of SEQ ID NO: 1 are substituted with -PLG in order to form part of a protease recognition site with the artificial polypeptide.

In some embodiments, cleavage of the protease cleavage site leaves at least 1 amino acid residue attached to the IL-18 polypeptide. In some embodiments, cleavage of the protease cleavage site leaves at most 1, 2, 3, 4, or 5 amino acid residues attached to the IL-18 polypeptide. In some embodiments, cleavage of the protease cleavage site leaves 1, 2, 3, 4, or 5 amino acid residues attached to the IL-18 polypeptide. In some embodiments, cleavage of the protease cleavage site leaves 1, 2, 3, 4, 5, 6, 7, 8, 9, or 10 or more amino acid residues attached to the IL-18 polypeptide. In some embodiments, cleavage of the protease cleavage site leaves at most 10, 9, 8, 7, 6, 5, 4, 3, 2, or 1 amino acid residues attached to the IL-18 polypeptide.

Further exemplary protease recognition sequence, such as cleavable peptide sequences which can be incorporated into an Act-IL-18 polypeptide as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, WO2021/202678, and/or Chen et. al., J Bio Chem, 277, V6 P4485-4491 (2002). Further examples of cleavable peptide sequences compatible with the instant disclosure are described in Patent Cooperation Treaty Publication No. WO2024/150172.

### Blocking Moieties

In some embodiments, the artificial polypeptide comprises a blocking moiety. In some embodiments, the blocking moiety is a group which, when attached to the IL-18 polypeptide in the Act-IL-18 polypeptide, acts to disrupt or inhibit binding of the IL-18 polypeptide with the IL-18 receptor or a subunit thereof (e.g., as measured by experiments designed to detect binding, or by in vitro or in vivo activity analysis of the Act-IL-18 polypeptide).

In some embodiments, the blocking moiety is a steric blocking group or a specific blocking group. In some embodiments, the blocking moiety is a steric blocking group. In some embodiments, a steric blocking group has no specific interaction with the IL-18 polypeptide, but its presence hinders the interaction of the Act-IL-18 polypeptide with the receptor owing to its bulk. In some embodiments, the steric blocking group is a polymer (e.g., polyethylene glycol) or a polypeptide (e.g., albumin, an Fc region, etc.).

In some embodiments, the blocking moiety is a specific blocking group. In some embodiments, the specific blocking group has a specific binding or other interaction to IL-18. Non-limiting examples of specific blocking groups can include IL-18 propeptides, antibodies or antigen binding fragments which bind IL-18, IL-18 receptor subunits or domains or other fragments thereof, IL-18 binding proteins or fragments thereof, or other groups capable of specific binding to IL-18.

In some embodiments, the blocking moiety is a propeptide of IL-18, or a variant thereof. In some embodiments, the blocking moiety is a propeptide of IL-18. Endogenously, human IL-18 is expressed as an immature, inactive 193 amino acid having the sequence MAAEPVEDNCINFVAMKFIDNTLYFIAEDDENLESDYFGKLESKLSVIRNLNDQVLFIDQ GNRPLFEDMTDSDCRDNAPRTIFIISMYKDSQPRGMA VTISVKCEKISTLSCENKIISFKE MNPPDNIKDTKSDIIFFQRSVPGHDNKMQFESSSYEGYFLACEKERDLFKLILKKEDELG DRSIMFTVQNED (SEQ ID NO: 88), the first 36 amino acids of which are a propeptide which is cleaved by caspases to yield the mature, active form of IL-18 (SEQ ID NO: 1). In some embodiments, the blocking moiety is attached to the N-terminus of the IL-18 polypeptide (e.g., through a cleavable peptide comprising the specific cleavage site and any optional linker peptides), and the blocking moiety is a propeptide of IL-18. In some embodiments, the blocking moiety is a human IL-18 propeptide or a variant thereof. In some embodiments, the blocking moiety is an IL-18 propeptide an amino acid sequence having at least 80%, 85%, 90%, 95%, 96%, 97%, 98%, 99%, or 100% sequence identity to the sequence set forth in SEQ ID NO: 89. In some embodiments, the blocking moiety is an IL-18 propeptide having a sequence at least 95% identical to the sequence set forth in SEQ ID NO: 89. In some embodiments, the blocking moiety is an IL-18 propeptide comprising the sequence set forth in SEQ ID NO: 89. In some embodiments, the blocking moiety is an IL-18 propeptide comprising the sequence set forth in SEQ ID NO: 89 with a substitution for residue C10 (e.g., a C10S substitution (SEQ ID NO: 90) or a C10A substitution (SEQ ID NO: 91). In some embodiments, the IL-18 propeptide comprises one or more modifications (e.g., amino acid substitutions) which reduce the affinity of the IL-18 propeptide for the IL-18 polypeptide in the Act-IL-18 polypeptide. In some embodiments, the blocking moiety is an IL-18 propeptide, and the protease cleavage site of the Act-IL-18 polypeptide is different from that of the endogenous propeptide (i.e., the specific cleavage site is not the bond between D36 and Y37 of SEQ ID NO: 88). In some embodiments, the IL-18 propeptide acting as a blocking moiety is connected to the N-terminus of the IL-18 polypeptide in the Act-IL-18 polypeptide through another protease recognition sequence (and optionally one or more linking peptides).

In some embodiments, the blocking moiety is a modified IL-18 propeptide (e.g., human IL-18 propeptide) directly attached to the N-terminus of the IL-18 polypeptide. In some embodiments, the modified IL-18 propeptide comprises modifications which change the natural caspase cleavage site of SEQ ID NO: 88 to a site cleaved by another protease (e.g., any of the proteases provided herein, such as a matrix metalloprotease). In some embodiments, the three C-terminal amino acids of the IL-18 propeptide are substituted to -PLG (e.g., SEQ ID NOs: 91, 96, and 97). In some embodiments, the IL-18 propeptide comprises the -PLG substitution and at least one of the first 3 residues of the IL-18 polypeptide is substituted relative to SEQ ID NO: 1 to make a complete protease recognition sequences.

### Exemplary IL-18 Propeptide Sequences

| SEQ ID | Blocking Moiety Sequence |
|---|---|
| 89 | MAAEPVEDNCINFVAMKFIDNTLYFIAEDDENLESD |
| 90 | MAAEPVEDNSINFVAMKFIDNTLYFIAEDDENLESD |
| 91 | MAAEPVEDNAINFVAMKFIDNTLYFIAEDDENLESD |
| 92 | MAAEPVEDNCINFVAMKFIDNTLYFIAEDDENLPLG |
| 96 | MAAEPVEDNSINFVAMKFIDNTLYFIAEDDENLPLG |
| 97 | MAAEPVEDNAINFVAMKFIDNTLYFIAEDDENLPLG |

In some embodiments, an Act-IL-18 polypeptide comprising a human IL-18 propeptide or variant thereof as a blocking moiety exhibits substantially reduced activity compared to the IL-18 polypeptide by itself or the activated form of the IL-18 polypeptide (e.g., substantially no activity, or activity which is reduced by more than 1000-fold). In some embodiments, it may be desirable for the Act-IL-18 with an IL-18 propeptide attached to retain a greater activity prior to activation. In order to accomplish this, in some embodiments, it may be advantageous to use an IL-18 propeptide from a non-human species. In some embodiments, an Act-IL-18 polypeptide provided herein comprises an IL-18 propeptide from a non-human species. In some embodiments, the non-human species is a mammal. In some embodiments, the non-human species is a primate, a rodent, an equine, a bovine, an ursine, a porcine, an equine, a chiroptera, a camelid, or other animal. In some embodiments, the non-human IL-18 propeptide has an amino acid sequence which is at least 50%, 60%, 70%, or 75% identical to that of SEQ ID NO: 89.

In some embodiments, the blocking moiety comprises a portion (e.g., a domain or portion thereof) of an IL-18 receptor subunit, or a variant thereof. In some embodiments, the blocking moiety comprises a portion (e.g., a domain or portion thereof) of an IL-18 receptor subunit, or a variant thereof, and the blocking moiety is connected to the C-terminus of the IL-18 polypeptide in the Act-IL-18 polypeptide. In some embodiments, the portion of the IL-18 receptor subunit or variant thereof is attached to the C-terminus of the IL-18 polypeptide in the Act-IL-18 polypeptide (e.g., through a cleavable peptide comprising the specific cleavage site and any optional linker peptides). In some embodiments, the blocking moiety comprises a portion of the IL-18 receptor alpha subunit or the IL-18 receptor beta subunit, or a variant thereof. In some embodiments, the blocking moiety comprises a portion of the IL-18 receptor alpha subunit or a variant thereof. In some embodiments, the blocking moiety comprises a domain of the IL-18 receptor alpha subunit or a variant thereof. In some embodiments, the blocking moiety comprises an extracellular domain, or a variant thereof, of the IL-18 receptor alpha subunit. In some embodiments, the blocking moiety comprises the D1, D2, or D3 domain, or a variant thereof, of the IL-18 receptor alpha subunit.

In some embodiments, the blocking moiety comprises the D3 domain, or a variant thereof, of the IL-18 receptor alpha subunit. The sequence of the human D3 domain of the IL-18 receptor alpha subunit is shown in SEQ ID NO: 93 below. In some embodiments, the blocking moiety comprises an amino acid sequence having at least about 80%, 85%, 90%, 95%, 96%, 97%, 98%, 99%, or 100% sequence identity to the sequence set forth in SEQ ID NO: 93. In some embodiments, the blocking moiety comprises an amino acid sequence having at least 95% sequence identity to the sequence set forth in SEQ ID NO: 93. In some embodiments, the blocking moiety comprises the sequence set forth in SEQ ID NO: 93. In some embodiments, the blocking moiety D3 domain of the IL-18 receptor alpha subunit comprises substitutions which remove glycosylation sites from the D3 domain (e.g., N to D substitutions, as in SEQ ID NO: 94). In some embodiments, the blocking moiety comprises the amino acid sequence set forth in SEQ ID NO: 94 below. In some embodiments, the cysteines are substituted for another amino acid (e.g., A or S) in the blocking moiety (e.g., in SEQ ID NO: 93 or 94). In some embodiments, the blocking moiety comprises an amino acid sequence set forth in the table below (e.g., 98 or 99, which contain substitutions to remove cysteine residues to facilitate site specific conjugation to the antibody or antigen binding fragment).

| SEQ ID | Blocking Moiety Sequence |
|---|---|
| 93 | SNIVPVLLGPKLNHVAVELGKNVRLNCSALLNEEDVIYW |
| | MFGEENGSDPNIHEEKEMRIMTPEGKWHASKVLRIENIGE |
| | SNLNVLYNCTVASTGGTDTKSFILVRKAD |
| 94 | SNIVPVLLGPKLNHVAVELGKNVRLDCSALLNEEDVIYW |
| | MFGEEDGSDPNIHEEKEMRIMTPEGKWHASKVLRIENIGE |
| | SNLNVLYDCTVASTGGTDTKSFILVRKAD |
| 98 | SNIVPVLLGPKLNHVAVELGKNVRLNSSALLNEEDVIYW |
| | MFGEENGSDPNIHEEKEMRIMTPEGKWHASKVLRIENIGE |
| | SNLNVLYNSTVASTGGTDTKSFILVRKAD |
| 99 | SNIVPVLLGPKLNHVAVELGKNVRLDASALLNEEDVIYW |
| | MFGEEDGSDPNIHEEKEMRIMTPEGKWHASKVLRIENIGE |
| | SNLNVLYDATVASTGGTDTKSFILVRKAD |

In some embodiments, it may be desirable to introduce one or more substitutions of amino acids to the sequence of the D3 domain of the IL-18 receptor alpha subunit when used as a blocking moiety in order to enhance or detune the binding of the D3 domain to the IL-18 polypeptide in the Act-IL-18 polypeptide. In some embodiments, the substitutions can act to improve the affinity to IL-18 (enhanced attenuation of IL-18 activity) or decrease the affinity to IL-18 (greater attenuation of IL-18 activity).

The full length human IL-18 receptor alpha subunit has the sequence MNCRELPLTLWVLISVSTAESCTSRPHITVVEGEPFYLKHCSCSLAHEIETTTKSWYKSSG SQEHVELNPRSSSRIALHDCVLEFWPVELNDTGSYFFQMKNYTQKWKLNVIRRNKHSCF TERQVTSKIVEVKKFFQITCENSYYQTLVNSTSLYKNCKKLLLENNKNPTIKKNAEFEDQ GYYSCVHFLHHNGKLFNITKTFNITIVEDRSNIVPVLLGPKLNHVA VELGKNVRLNCSAL LNEEDVIYWMFGEENGSDPNIHEEKEMRIMTPEGKWHASKVLRIENIGESNLNVLYNCT VASTGGTDTKSFILVRKADMADIPGHVFTRGMIIAVLILVAVVCLVTVCVIYRVDLVLFY RHLTRRDETLTDGKTYDAFVSYLKECRPENGEEHTFA VEILPRVLEKHFGYKLCIFERDV VPGGAVVDEIHSLIEKSRRLIIVLSKSYMSNEVRYELESGLHEALVERKIKIILIEFTPVTDF TFLPQSLKLLKSHRVLKWKADKSLSYNSRFWKNLLYLMPAKTVKPGRDEPEVLPVLSES (SEQ ID NO: 95) (UniProt ID: Q13478).

### Linking Peptides

In some embodiments, the artificial polypeptide comprises one or more linking peptides. In some embodiments, the linking peptide of an artificial polypeptide is positioned between the protease cleavage site and the IL-18 polypeptide (i.e., the linking peptide remains attached to the IL-18 polypeptide after cleavage) or is positions between the protease cleavage site and a blocking moiety, or both (e.g., the Act-IL-18 polypeptide has two linking peptides). In some embodiments, a linking peptide comprises from 1 to 50 amino acid, from 1 to 40 amino acids, from 1 to 30 amino acids, from 1 to 25 amino acids, from 1 to 20 amino acids, from 1 to 15 amino acids, from 1 to 10 amino acids, or from 1 to 5 amino acids. In some embodiments, the linking peptide comprises from 1 to 15 amino acids. In some embodiments, the linking peptide consists of amino acids glycine and serine. In some embodiments, the linking peptide consists of glycines. Non-limiting examples of a linking peptide include, but are not limited to (GS)ₙ (SEQ ID NO: 424), (GGS)ₙ (SEQ ID NO: 425), (GGGS)ₙ (SEQ ID NO: 426), (GGSG)ₙ (SEQ ID NO: 427), or (GGSGG)ₙ (SEQ ID NO: 428), (GGGGS)ₙ (SEQ ID NO: 429), wherein n is 1, 2, 3, 4, 5, 6, 7, 8, 9, or 10. For example, a linking peptide can be (GGGGS) 3 (SEQ ID NO: 430) or (GGGGS) 4 (SEQ ID NO: 431). Additional linking peptides can include GGGGSGGGGSGGGG (SEQ ID NO: 432). In embodiments where the artificial polypeptide comprises multiple linking peptides, each linking peptide can be the same or different.

### Molecule Orientations

Act-IL-18 polypeptides of activatable immunocytokines provided herein can have a variety of orientations. In some embodiments, an Act-IL-18 polypeptide provided herein comprises an orientation according to any one of the below formulas:
(a) IL-18-CS;
(b) IL-18-LP₁-CS;
(c) IL-18-LP₁-CS-LP₂-BM;
(d) IL-18-CS-LP₁-BM;
(e) IL-18-CS-BM;
(f) IL-18-LP₁-CP-BM;
(g) BM-CS-IL-18;
(h) BM-LP₁-CS-IL-18;
(i) BM-LP₁-CS-LP₂-IL-18;
(j) BM-CS-LP₁-IL-18;
(k) CS-LP₁-IL-18; and
(l) CS-IL-18;
   wherein IL-18 is the IL-18 polypeptide, CS is a moiety comprising the specific cleavage site (e.g., a protease recognition sequence), LP₁ is a first linking peptide, LP₂ is a second linking peptide, and BM is a blocking moiety, and wherein the orientations are shown in an N-terminal to C-terminal order.

In some embodiments, the Act-IL-18 polypeptide comprises the orientation of formula (a). In some embodiments, the Act-IL-18 polypeptide comprises the orientation of formula (b). In some embodiments, the Act-IL-18 polypeptide comprises the orientation of formula (c). In some embodiments, the Act-IL-18 polypeptide comprises the orientation of formula (d). In some embodiments, the Act-IL-18 polypeptide comprises the orientation of formula (e). In some embodiments, the Act-IL-18 polypeptide comprises the orientation of formula (f). In some embodiments, the Act-IL-18 polypeptide comprises the orientation of formula (g). In some embodiments, the Act-IL-18 polypeptide comprises the orientation of formula (h). In some embodiments, the Act-IL-18 polypeptide comprises the orientation of formula (i). In some embodiments, the Act-IL-18 polypeptide comprises the orientation of formula (j). In some embodiments, the Act-IL-18 polypeptide comprises the orientation of formula (k). In some embodiments, the Act-IL-18 polypeptide comprises the orientation of formula (l).

In the orientations described above, it is expressly contemplated herein that the IL-18 polypeptide can be any of the IL-18 polypeptides described herein, the CS moiety can be any of the cleavage sequences described herein (e.g., any of those in Table 2A or Table 2B, or one having a specific sequence identity thereto, or another cleavage sequence as described herein), the blocking moieties can be any of the blocking moieties described herein (e.g., any of the IL-18 propeptides as described herein), and the linking peptides can be any of the linking peptides described herein, independently of any of the other components.

In certain preferred embodiments, the Act-IL-18 polypeptides as described herein are of the formula (g) BM-CS-IL-18. In some embodiments, the blocking moiety is an IL-18 propeptide as described herein (e.g., any of the IL-18 propeptides described herein, including any of the variants described herein). In some embodiments, the CS group is one of the cleavage sequence described herein (e.g., any of those in Table 2A or Table 2B, or any variant thereof described herein). In some embodiments, the IL-18 polypeptide is any on of those described herein (e.g., any one of those set forth in SEQ ID NOs: 1-86, or another IL-18 variant polypeptide described herein).

### IL-18 Polypeptides

The Act-IL-18 polypeptides of activatable immunocytokines herein comprise IL-18 polypeptides. An IL-18 polypeptide of the constructs of the instant disclosure can contain a number of modifications to WT IL-18 (SEQ ID NO: 1), including without limitation amino acid substitutions, deletions, additions, or attachment of polymer moieties.

In some embodiments, the IL-18 polypeptide comprises an amino acid sequence having at least about 80%, 85%, 90%, 95%, 96%, 97%, 98%, or 99% sequence identity to SEQ ID NO: 1. In some embodiments, the IL-18 polypeptide comprises an amino acid sequence having at least about 80%, 85%, 90%, 95%, 96%, 97%, 98%, 99%, or 100% identity to a sequence set forth in any one of SEQ ID NOs: 2-67. In some embodiments, the IL-18 polypeptide comprises an amino acid sequence having at least about 80%, 85%, 90%, 95%, 96%, 97%, 98%, 99%, or 100% identity to a sequence set forth in any one of SEQ ID NOs: 79-83. In some embodiments, the IL-18 polypeptide comprises an amino acid sequence having at least about 80%, 85%, 90%, 95%, 96%, 97%, 98%, 99%, or 100% identity to a sequence set forth in any one of SEQ ID NOs: 84-86. In some embodiments, the IL-18 polypeptide comprises the amino acid sequence set forth in SEQ ID NO: 30. In some embodiments, the IL-18 polypeptide comprises the amino acid sequence set forth in SEQ ID NO: 79. In some embodiments, the IL-18 polypeptide comprises the amino acid sequence set forth in SEQ ID NO: 80. In some embodiments, the IL-18 polypeptide comprises the amino acid sequence set forth in SEQ ID NO: 81. In some embodiments, the IL-18 polypeptide comprises the amino acid sequence set forth in SEQ ID NO: 82. In some embodiments, the IL-18 polypeptide comprises the amino acid sequence set forth in SEQ ID NO: 83. In some embodiments, the IL-18 polypeptide comprises the amino acid sequence set forth in SEQ ID NO: 84. In some embodiments, the IL-18 polypeptide comprises the amino acid sequence set forth in SEQ ID NO: 85. In some embodiments, the IL-18 polypeptide comprises the amino acid sequence set forth in SEQ ID NO: 86.

### Amino Acid Substitutions of IL-18 Polypeptides

In some embodiments, the IL-18 polypeptide of an Act-IL-18 polypeptide described herein comprises a polypeptide of at least 1, at least 2, at least 3, at least 4, at least 5, at least 6, at least 7, at least 8 at least 9, or more substitutions at one or more amino acid residues, whereas the positions of the substitutions are relative to positions in IL-18 of SEQ ID NO:1. In some embodiments, the IL-18 polypeptide comprises 1 to 9 amino acid substitutions. In some embodiments, the Act-IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-18 polypeptide comprises up to 20, up to 19, up to 18, up to 17, up to 16, up to 15, up to 14, up to 13, up to 12, up to 11, up to 10, up to 9, or up to 8 amino acid substitutions to the sequence set forth in SEQ ID NO: 1.

Unless specifically mentioned otherwise, the amino acid substitutions provided in this paragraph, and elsewhere in this disclosure, is with respect to SEQ ID NO: 1, as a reference sequence. In certain embodiments, the IL-18 polypeptide of the Act-IL-18 polypeptide comprises a substitution at residue Y1. In certain embodiments, the IL-18 polypeptide of the Act-IL-18 polypeptide can comprises Y1M substitution. In certain embodiments, the IL-18 polypeptide of the Act-IL-18 polypeptide comprises a substitution at residue F2. In certain embodiments, the IL-18 polypeptide of the Act-IL-18 polypeptide can comprises F2A substitution. In certain embodiments, the IL-18 polypeptide of the Act-IL-18 polypeptide comprises a substitution at residue E6. In certain embodiments, the IL-18 polypeptide of the Act-IL-18 polypeptide comprises E6K substitution. In certain embodiments, the IL-18 polypeptide of the Act-IL-18 polypeptide comprises E6R substitution. In certain embodiments, the IL-18 polypeptide of the Act-IL-18 polypeptide comprises a substitution at residue K8. In certain embodiments, the IL-18 polypeptide of the Act-IL-18 polypeptide comprises K8L substitution. In certain embodiments, the IL-18 polypeptide of the Act-IL-18 polypeptide comprises K8E substitution. In certain embodiments, the IL-18 polypeptide of the Act-IL-18 polypeptide e comprises K8R substitution. In certain embodiments, the IL-18 polypeptide of the Act-IL-18 polypeptide comprises a substitution at residue V11. In certain embodiments, the IL-18 polypeptide of the Act-IL-18 polypeptide comprises V11I substitution. In certain embodiments, the modified IL-18 polypeptide comprises a substitution at residue E31. In certain embodiments, the IL-18 polypeptide of the Act-IL-18 polypeptide comprises E31A substitution. In certain embodiments, the IL-18 polypeptide of the Act-IL-18 polypeptide comprises a substitution at residue T34. In certain embodiments, the IL-18 polypeptide of the Act-IL-18 polypeptide comprises T34A substitution. In certain embodiments, the IL-18 polypeptide of the Act-IL-18 polypeptide comprises a substitution at residue D35. In certain embodiments, the IL-18 polypeptide of the Act-IL-18 polypeptide comprises D35A substitution. In certain embodiments, the IL-18 polypeptide of the Act-IL-18 polypeptide comprises a substitution at residue S36. In certain embodiments, the IL-18 polypeptide of the Act-IL-18 polypeptide comprises S36A substitution. In certain embodiments, the IL-18 polypeptide of the Act-IL-18 polypeptide comprises a substitution at residue D37. In certain embodiments, the IL-18 polypeptide of the Act-IL-18 polypeptide comprises D37A substitution. In certain embodiments, the IL-18 polypeptide of the Act-IL-18 polypeptide comprises a substitution at residue D40. In certain embodiments, the IL-18 polypeptide of the Act-IL-18 polypeptide comprises D40A substitution. In certain embodiments, the IL-18 polypeptide of the Act-IL-18 polypeptide comprises a substitution at residue N41. In certain embodiments, the IL-18 polypeptide of the Act-IL-18 polypeptide comprises N41A substitution. In certain embodiments, the IL-18 polypeptide of the Act-IL-18 polypeptide comprises a substitution at residue 149. In certain embodiments, the IL-18 polypeptide of the Act-IL-18 polypeptide comprises 149E substitution. In certain embodiments, the IL-18 polypeptide of the Act-IL-18 polypeptide comprises 149M substitution. In certain embodiments, the IL-18 polypeptide of the Act-IL-18 polypeptide comprises 149R substitution. In certain embodiments, the IL-18 polypeptide of the Act-IL-18 polypeptide comprises a substitution at residue K53. In certain embodiments, the IL-18 polypeptide of the Act-IL-18 polypeptide comprises K53A substitution. In certain embodiments, the IL-18 polypeptide of the Act-IL-18 polypeptide comprises a substitution at residue D54. In certain embodiments, the IL-18 polypeptide of the Act-IL-18 polypeptide comprises D54A substitution. In certain embodiments, the IL-18 polypeptide of the Act-IL-18 polypeptide comprises a substitution at residue S55. In certain embodiments, the IL-18 polypeptide of the Act-IL-18 polypeptide comprises S55A substitution. In certain embodiments, the IL-18 polypeptide of the Act-IL-18 polypeptide comprises S55T substitution. In certain embodiments, the IL-18 polypeptide of the Act-IL-18 polypeptide comprises S55H substitution. In certain embodiments, the IL-18 polypeptide of the Act-IL-18 polypeptide comprises S55R substitution. In certain embodiments, the IL-18 polypeptide of the Act-IL-18 polypeptide comprises a substitution at residue T63. In certain embodiments, the IL-18 polypeptide of the Act-IL-18 polypeptide comprises T63A substitution. In certain embodiments, the IL-18 polypeptide of the Act-IL-18 polypeptide comprises a substitution at residue Q103. In certain embodiments, the IL-18 polypeptide of the Act-IL-18 polypeptide comprises Q103R substitution. In certain embodiments, the IL-18 polypeptide of the Act-IL-18 polypeptide comprises Q103E substitution. In certain embodiments, the IL-18 polypeptide of the Act-IL-18 polypeptide comprises Q103K substitution. In certain embodiments, the IL-18 polypeptide of the Act-IL-18 polypeptide comprises a substitution at residue G108. In certain embodiments, the IL-18 polypeptide of the Act-IL-18 polypeptide comprises G108A substitution. In certain embodiments, the IL-18 polypeptide of the Act-IL-18 polypeptide comprises a substitution at residue H109. In certain embodiments, the IL-18 polypeptide of the Act-IL-18 polypeptide comprises H109A substitution. In certain embodiments, the IL-18 polypeptide of the Act-IL-18 polypeptide comprises a substitution at residue D110. In certain embodiments, the IL-18 polypeptide of the Act-IL-18 polypeptide comprises D110A substitution. In certain embodiments, the IL-18 polypeptide of the Act-IL-18 polypeptide comprises a substitution at residue D132. In certain embodiments, the IL-18 polypeptide of the Act-IL-18 polypeptide comprises D132A substitution. In certain embodiments, the IL-18 polypeptide of the Act-IL-18 polypeptide comprises a substitution at residue V153. In certain embodiments, the IL-18 polypeptide of the Act-IL-18 polypeptide comprises V153R substitution. In certain embodiments, the IL-18 polypeptide of the Act-IL-18 polypeptide comprises V153E substitution. In certain embodiments, the IL-18 polypeptide of the Act-IL-18 polypeptide comprises V153Y substitution. In certain embodiments, the IL-18 polypeptide of the Act-IL-18 polypeptide comprises a substitution at residue C38. In certain embodiments, the IL-18 polypeptide of the Act-IL-18 polypeptide comprises C38A substitution. In certain embodiments, the IL-18 polypeptide of the Act-IL-18 polypeptide comprises C38S substitution. In certain embodiments, the IL-18 polypeptide of the Act-IL-18 polypeptide comprises a substitution at residue C68. In certain embodiments, the IL-18 polypeptide of the Act-IL-18 polypeptide comprises C68A substitution. In certain embodiments, the IL-18 polypeptide of the Act-IL-18 polypeptide comprises C68S substitution. In certain embodiments, the IL-18 polypeptide of the Act-IL-18 polypeptide comprises a substitution at residue C76. In certain embodiments, the IL-18 polypeptide of the Act-IL-18 polypeptide comprises C76A substitution. In certain embodiments, the IL-18 polypeptide of the Act-IL-18 polypeptide comprises C76S substitution. In certain embodiments, the modified IL-18 polypeptide comprises a substitution at residue C127. In certain embodiments, the IL-18 polypeptide of the Act-IL-18 polypeptide comprises C127A substitution. In certain embodiments, the IL-18 polypeptide of the Act-IL-18 polypeptide comprises C127S substitution. In certain embodiments, the IL-18 polypeptide of the Act-IL-18 polypeptide comprises a substitution at residue C38, C68, C76, and/or C127. In certain embodiments, the IL-18 polypeptide of the Act-IL-18 polypeptide comprises a C38A, C38S, C68A, C68S, C76A, C76S, C127A, and/or C127S substitution. In certain embodiments, the IL-18 polypeptide of the Act-IL-18 polypeptide comprises C38A, C76A, and C127A substitutions. In certain embodiments, the IL-18 polypeptide of the Act-IL-18 polypeptide comprises C38S, C76S and C127S substitutions.

In some embodiments, the modified IL-18 polypeptide comprises at least one additional modification to the amino acid sequence of SEQ ID NO: 1 selected from: Y01X, F02X, E06X, S10X, V11X, D17X, C38X, M51X, K53X, D54X, S55X, T63X, C68X, E69X, K70X, C76X, E85X, M86X, T95X, D98X, AND C127X, wherein each X is independently a natural or non-natural amino acid. In some embodiments, the modified IL-18 polypeptide comprises at least one additional modification to the amino acid sequence of SEQ ID NO: 1 selected from: Y01G, F02A, E06K, S10T, V11I, D17N, C38S, C38A, C38Q, M51G, K53A, D54A, S55A, T63A, C68S, C68A, E69C, K70C, C76S, C76A, E85C, M86C, T95C, D98C, C127A, and C127S.

In some embodiments, the Act-IL-18 polypeptide of an activatable immunocytokine comprises a modified IL-18 polypeptide comprising E06K and K53A, wherein residue position numbering of the modified IL-18 polypeptide is based on SEQ ID NO: 1 as a reference sequence. In some embodiments, the modified IL-18 polypeptide further comprises T63A. In some embodiments, the modified IL-18 polypeptide further comprises at least one of Y01X, S55X, F02X, D54X, C38X, C68X, E69X, K70X, C76X, or C127X, wherein each X is independently an amino acid or an amino acid derivative. In some embodiments, the modified IL-18 polypeptide further comprises at least one of Y01G, S55A, F02A, D54A, C38S, C38A, C68S, C68A, K70C, C76S, C76A, C127S, or C127A.

In some embodiments, the modified IL-18 peptide comprises at least one modification to the amino acid sequence of SEQ ID NO: 1, wherein the modification is E06X, K53X, S55X, or T63X, wherein X is a natural or non-natural amino acid. In some embodiments, the modified IL-18 peptide comprises at least two additional modifications to the amino acid sequence of SEQ ID NO: 1, wherein the modifications comprise E06X and K53X; E06X and S55X; K53X and S55X; E06X and T63X; or K53X and T63X, wherein X is a natural or non-natural amino acid. In some embodiments, the modified IL-18 peptide comprises at least three additional modifications to the amino acid sequence of SEQ ID NO: 1, wherein the modifications comprise E06X, K53X, and S55X; or E06X, K53X, and T63X, wherein X is a natural or non-natural amino acid. In some embodiments, the modified IL-18 peptide comprises at least four additional modifications to the amino acid sequence of SEQ ID NO: 1, wherein the modifications comprise E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In each embodiment wherein a plurality of amino acids residues are replaced with a natural or non-natural amino acid X, each X is independently the same or a different amino acid.

In some embodiments, the modified IL-18 peptide comprises at least one additional modification to the amino acid sequence of SEQ ID NO: 1, wherein the modification is E06K, V11I, K53A, S55A, or T63A. In some embodiments, the modified IL-18 peptide comprises at least two additional modifications to the amino acid sequence of SEQ ID NO: 1, wherein the modifications comprise E06K and K53A; E06K and S55A; K53A and S55A; E06K and T63A; or K53A and T63A. In some embodiments, the modified IL-18 peptide comprises at least three additional modifications to the amino acid sequence of SEQ ID NO: 1, wherein the modifications comprise E06K, K53A, and S55A; E06K, V11I, and K53A; E06K, C38A, and K53A; or E06K, K53A, and T63A. In some embodiments, the modified IL-18 peptide comprises at least four additional modifications to the amino acid sequence of SEQ ID NO: 1, wherein the modifications comprise E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the modified IL-18 peptide comprises at least six modifications to the amino acid sequence of SEQ ID NO: 1, wherein the modifications comprise E06K, K53A, C38S, C68S, C76S, and C127S; or K53A, T63A, C38S, C68S, C76S, and C127S. In some embodiments, the modified IL-18 polypeptide comprises at least seven modifications to the sequence of SEQ ID NO: 1, wherein the seven modifications comprise E6K, V11I, C38A, K53A, T63A, C76A, C127A. In some embodiments, the modified IL-18 peptide comprises at least eight modifications to the amino acid sequence of SEQ ID NO: 1, wherein the modifications comprise Y01G, F02A, E06K, M51G, K53A, D54A, S55A, and T63A. In some embodiments, the modified IL-18 peptide comprises at least eight additional modifications to the amino acid sequence of SEQ ID NO: 1, wherein the modifications comprise Y01G, F02A, E06K, M51G, K53A, D54A, S55A, and T63A.

In one aspect, provided herein, is a modified IL-18 polypeptide with a polymer as provided herein (e.g., a polymer attached to a residue as provided herein), further comprising E06K and K53A, wherein residue position numbering of the modified IL-18 polypeptide is based on SEQ ID NO: 1 as a reference sequence. In some embodiments, the modified IL-18 polypeptide comprises an amino acid sequence at least 80%, at least 85%, at least 90%, at least 95%, or at least 98% identical to the amino acid sequence of SEQ ID NO: 30. In some embodiments, the modified IL-18 polypeptide comprises an amino acid sequence at least 80%, at least 85%, at least 90%, at least 95%, or at least 98% identical to the amino acid sequence of SEQ ID NO: 59. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue of SEQ ID NO: 1. In some embodiments, each cysteine residue is substituted with serine or alanine. In some embodiments, the modified IL-18 polypeptide comprises a polymer covalently attached to an amino acid residue. In some embodiments, the modified IL-18 polypeptide comprises amino acid substitutions at 1, 2, 3, 4, 5, or 6 methionine residues.

An IL-18 polypeptide of an Act-IL-18 polypeptide as described herein can comprise one or more non-canonical amino acids. “Non-canonical” amino acids can refer to amino acid residues in D- or L-form that are not among the 20 canonical amino acids generally incorporated into naturally occurring proteins. In some embodiments, one or more amino acids of the active form of the Act-IL-18 polypeptides are substituted with one or more non-canonical amino acids. Non-canonical amino acids include, but are not limited to N-alpha-(9-Fluorenylmethyloxycarbonyl)-L-azidolysine (Fmoc-L-Lys (N3)-OH), N-alpha-(9-Fluorenylmethyloxycarbonyl)-L-biphenylalanine (Fmoc-L-Bip-OH), and N-alpha-(9-Fluorenylmethyloxycarbonyl)-O-benzyl-L-tyrosine (Fmoc-L-Tyr (Bzl)-OH.

Exemplary non-canonical amino acids include azido-lysine (AzK), hydroxylysine, allo-hydroxylysine, ε-N,N,N-trimethyllysine, —N-acetyllysine, 5-hydroxylysine, Fmoc-Lys (Me, Boc), Fmoc-Lys (Me) 3, Fmoc-Lys (palmitoyl), Fmoc-L-photo-lysine, DL-5-hydroxylysine, H-L-photo-lysine, and/or other similar amino acids. Example non-canonical amino acids also include D-methionine, selenocysteine, and/or other similar amino acids.

Exemplary non-canonical amino acids also include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, Na-ethylglycine, Na-ethylaspargine, isodesmosine, allo-isoleucine, @-methylarginine, Na-methylglycine, Na-methylisoleucine, Na-methylvaline, γ-carboxyglutamate, O-phosphoserine, Na-acetylserine, Na-formylmethionine, 3-methylhistidine, and/or other similar amino acids.

In some embodiments, amino acid residues of the Act-IL-18 polypeptides are substituted with modified lysine residues. In some embodiments, the modified lysine residues comprise an amino, azide, allyl, ester, and/or amide functional groups. In some embodiments, the modified lysine residues contain conjugation handles which can serve as useful anchor points to attach additional moieties to the active form of the Act-IL-18 polypeptides. In some embodiments, the modified lysine residues have a structure built from precursors Structure 1, Structure 2, Structure 3, or Structure 4:

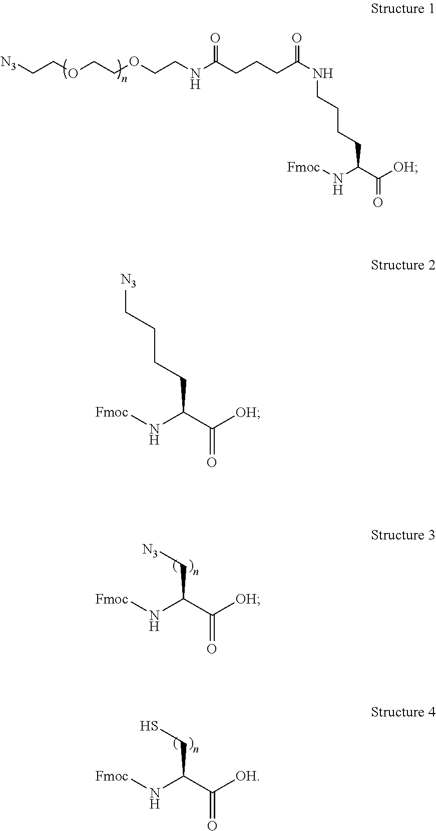

In some embodiments, the Act-IL-18 polypeptide contains a substitution for modified amino acid residues which can be used for attachment of additional functional groups which can be used to facilitate conjugation reaction or attachment of various payloads to the Act-IL-18 polypeptide (e.g., polymers). The substitution can be for a naturally occurring amino acid which is more amenable to attachment of additional functional groups (e.g., aspartic acid, cysteine, glutamic acid, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of such modified amino acid residues include the modified lysine, glutamic acid, aspartic acid, and cysteine provided below:

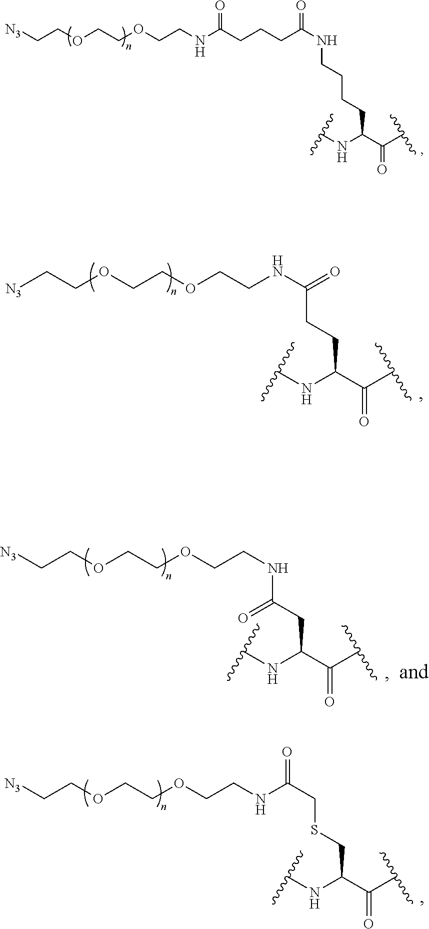

wherein each n is an integer from 1-30. These non-limiting examples of modified amino acid residues can be used at any location at which it is desirable to add an additional functionality (e.g., a polymer) to the Act-IL-18 polypeptide.

In some embodiments, any of structures 1-4, the modified lysine, the modified glutamic acid, the modified aspartic acid, or the modified cysteine provided above can be substituted for a different residue of the Act-IL-18 polypeptide (e.g., any of residues 68-70 or residues 80-100 using SEQ ID NO: 1 as a reference sequence) to allow for conjugation at a different site of the IL-18 polypeptide. The azide functionality may also be replaced with another suitable conjugation handle.

The conjugation handles provided herein can be any suitable reactive group capable of reacting with a complementary reactive group. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, potassium acyl trifluoroborate (KAT) ligations, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

In some embodiments, a group attached to the Act-IL-18 polypeptide (e.g., a polymer moiety or an additional polypeptide) comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-18 polypeptide (e.g., the polymer or the additional polypeptide) will comprise a reaction product of a conjugation handle with a complementary conjugation handle which was used to attach the group to the Act-IL-18 polypeptide.

The Active form of the IL-18 Polypeptide

In some embodiments, cleavage of the protease cleavage site of the artificial polypeptide converts the Act-IL-18 into an active form of the IL-18 polypeptide, which preferably remains attached to the antibody or antigen binding fragment after cleavage. As used herein, the active form of the IL-18 polypeptide refers to the cleaved version of the IL-18 polypeptide which possesses some or all of the activity associated with the IL-18 polypeptide which is inactivated by the artificial polypeptide. Additionally, unless context clearly indicates otherwise, reference to simply “the IL-18 polypeptide” refers to an IL-18 polypeptide which was never prepared in an activatable form (E.g., it refers to the base IL-18 polypeptide on which an Act-IL-18 polypeptide is based). In some embodiments, the active form of the IL-18 polypeptide comprises a portion the artificial polypeptide still attached to the IL-18 polypeptide (e.g., a subset of amino acid residues of the artificial polypeptide). In some embodiments, the active form of the IL-18 polypeptide is the same as the IL-18 polypeptide (e.g., has the same amino acid sequence as the IL-18 polypeptide because the entire artificial polypeptide has been cleaved). It is expected that activities associated with the IL-18 polypeptide (activated or non-activated form) will be substantially the same when the molecule is attached to the antibody or antigen binding fragment. For any of the activities described below, it is similarly contemplated herein that such activities can also apply to an activated immunocytokine provided herein (i.e., for each of the activities described below, the activities can also refer to the activity of the activated immunocytokine instead of the free activated IL-18 polypeptide).

In some embodiments, the active form of the IL-18 polypeptide provided herein display reduced binding to the IL-18 binding protein (IL-18BP) relative to WT-IL-18. The active form of the IL-18 polypeptides may also display binding characteristics for the IL-18Rab that differ from wild-type IL-18 (e.g., a higher affinity for the IL-18Rab heterodimer or a lower affinity for the IL-18Rab heterodimer). In preferred embodiments, the affinity for IL-18Rab of the active form of the IL-18 polypeptide is not substantially lower than the affinity of WT IL-18 for IL-18Rab (e.g., the active form of the Act-IL-18 polypeptide's affinity for IL-18Rab is no less than about 500× lower than wild type IL-18).

In some embodiments, the active form of the IL-18 polypeptides displays increased affinity for an IL-18 receptor alpha subunit (IL-18Ra) or an IL-18 receptor beta subunit (IL-18Rb) relative to wild type IL-18. In some embodiments, the active form of the IL-18 polypeptides have an increased affinity for the IL-18Ra/b heterodimer relative to IL-18 WT. In one aspect, the active form of the IL-18 polypeptides described herein have decreased affinity for the IL-18Ra/b heterodimer relative to wild type IL-18.

In some embodiments, the binding affinity between the active form of the IL-18 polypeptides and IL-18Rα is the same as or lower than the binding affinity between a wild-type IL-18 and IL-18Ra. In some embodiments, the binding affinity between the active form of the IL-18 polypeptides and IL-18Rα is the same as or higher than the binding affinity between a wild-type IL-18 and IL-18Ra. In some embodiments, the binding affinity between the active form of the IL-18 polypeptides and IL-18Rb is the same as or lower than the binding affinity between a wild-type IL-18 and IL-18Rb. In some embodiments, the binding affinity between the active form of the IL-18 polypeptides and IL-18Rb is the same as or higher than the binding affinity between a wild-type IL-18 and IL-18Rb. In some embodiments, the binding affinity between the active form of the IL-18 polypeptides and the IL-18Ra/b heterodimer is the same as or lower than the binding affinity between a wild-type IL-18 and the IL-18Ra/b heterodimer. In some embodiments, the binding affinity between the active form of the IL-18 polypeptides and the IL-18Ra/b heterodimer is the same as or higher than the binding affinity between a wild-type IL-18 and the IL-18Ra/b heterodimer.

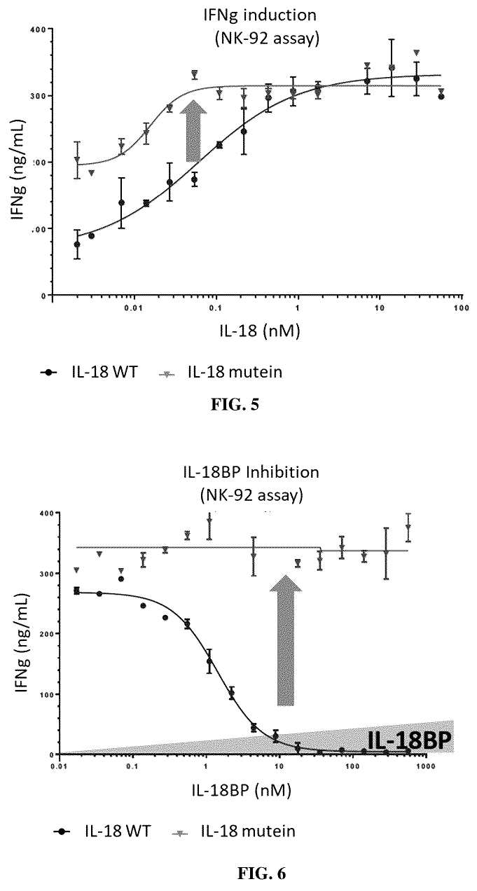

In some embodiments, an active form of the IL-18 polypeptide provided herein displays an ability to induce interferon gamma (IFNγ) production after administration to a subject. In some embodiments, the ability to induce IFNγ is comparable to that of a wild type IL-18 (e.g., displays an EC50 for IFNγ induction that is within about 10-fold of that of a wild type IL-18). An exemplary IL-18 polypeptide displaying this characteristic is shown in FIG. 5, which shows a comparison of IFNγ production (ng/mL, y-axis) as a function of concentration of a wild type versus an IL-18 polypeptide (mutein) (nM, x-axis).

In some embodiments, an active form of the Act-IL-18 polypeptide provided herein also display a reduced binding IL-18 binding protein (IL-18BP). In some embodiments, the active form of the IL-18 polypeptide provided herein can induce IFNγ even in the presence of IL-18BP (e.g., the ability of the active form of the Act-IL-18 polypeptide to induce IFNγ is not substantially inhibited by the presence of IL-18BP) (nM, x-axis). An example of an IL-18 polypeptide with this property compared to wild type IL-18 is shown in FIG. 6, which shows IFNγ production (ng/mL, y-axis) as a function of IL-18BP concentration (nM, x-axis) in a sample treated with the same level of wild type IL-18 (circles) or the IL-18 polypeptide (inverted triangles). In some embodiments, an active form of IL-18 polypeptide provided herein displays a similar or only slightly reduced ability to induce IFNγ production compared to wild type IL-18.

In some embodiments, an active form of IL-18 polypeptide provided herein displays a significant reduction in inhibition of the ability to induce IFNγ production in the presence of IL-18BP compared to wild type IL-18. In some embodiments, an active form of IL-18 polypeptide provided herein displays a similar or only slightly reduced ability to induce IFNγ production compared to wild type IL-18, and a significant reduction in inhibition of the ability to induce IFNγ production in the presence of IL-18BP compared to wild type IL-18.

### Points of Attachment of Chemical Linkers to IL-18 Polypeptides

The activatable immunocytokines provided herein comprise linkers which have a point of attachment to the Act-IL-18 polypeptide. In some embodiments, the linker is a chemical linker. As discussed supra, the linker has another point of attachment to the antibody or antigen binding fragment at any residue as provided herein. The point of attachment to the Act-IL-18 polypeptide is to a residue as provided herein. In some preferred embodiments, the linker is attached to the IL-18 polypeptide such that the IL-18 polypeptide remains tethered to the antibody or antigen binding fragment after cleavage of the artificial polypeptide attached (i.e., the active form of the IL-18 polypeptide remains attached to the antibody or antigen binding fragment thereof).

In some embodiments, the linker is attached to an amino acid residue of the IL-18 polypeptide of the Act-IL-18 polypeptide. In some embodiments, the linker is attached to any amino acid residue of the IL-18 polypeptide (e.g., at a position corresponding to any one of positions 1-157 of SEQ ID NO: 1). In some embodiments, the linker is attached at a non-terminal residue of the IL-18 polypeptide (e.g., a residue at position corresponding to any one of positions 2-156 of SEQ ID NO: 1). In some embodiments, the linker is attached at a non-terminal residue of the Act-IL-18 polypeptide, wherein the IL-18 polypeptide has been extended or truncated by one or more amino acids relative to SEQ ID NO: 1.

In some embodiments, the linker is attached to the IL-18 polypeptide of the Act-IL-18 polypeptide at a residue in a region comprising residues 2-156, wherein residue position numbering is based on SEQ ID NO: 1 as a reference sequence. In some embodiments, the linker is attached to the IL-18 polypeptide at a residue in a region comprising residues 30-150. In some embodiments, the linker is attached to the IL-18 polypeptide at a residue in a region comprising residues 33-43, residues 60-100, residues 65-75, residues 80-90, residues 85-100, residues 90-110, residues 115-130, residues 120-130, or residues 140-150. In some embodiments, the linker is attached to the IL-18 polypeptide at a residue selected from residue 38, 68, 69, 70, 76, 78, 85, 86, 95, 98, 121, 127, and 144. In some embodiments, the linker is attached to the IL-18 polypeptide at a residue selected from 68, 69, 70, 85, 86, and 98. In some embodiments, the linker is attached to the IL-18 polypeptide at residue 68, 69, or 70. In some embodiments, the linker is attached to the IL-18 polypeptide at residue 85, 86, 95, or 98. In some embodiments, the linker is attached to the IL-18 polypeptide at residue 68. In some embodiments, the linker is attached to the IL-18 polypeptide at residue 69. In some embodiments, the linker is attached to the IL-18 polypeptide at residue 70. In some embodiments, the linker is attached to the IL-18 polypeptide at residue 85. In some embodiments, the linker is attached to the IL-18 polypeptide at residue 86. In some embodiments, the linker is attached to the IL-18 polypeptide at residue 95. In some embodiments, the linker is attached to the IL-18 polypeptide at residue 98.

In some embodiments, the linker is attached to the IL-18 polypeptide of the Act-IL-18 polypeptide at a residue which is known in the art to be compatible with attachment of a polymer to the IL-18 polypeptide without having a profound impact on the bioactivity of the IL-18 polypeptide. Examples of these residues include residues 38, 76, 78, 121, 127, and 144, based on SEQ ID NO: 1 as a reference sequence, as described in PCT Pub. No. WO2004091517A2, which is hereby incorporated by reference as if set forth in its entirety.

In some embodiments, the residue to which the linker is attached is a natural amino acid residue. In some embodiments, the residue to which the linker is covalently attached is selected from cysteine, aspartate, asparagine, glutamate, glutamine, serine, threonine, lysine, and tyrosine. In some embodiments, the residue to which the linker is covalently attached is selected from asparagine, aspartic acid, cysteine, glutamic acid, glutamine, lysine, and tyrosine. In some embodiments, the linker is covalently attached to a cysteine. In some embodiments, the linker is covalently attached to a lysine. In some embodiments, the linker is covalently attached to a glutamine. In some embodiments, the linker is covalently attached to an asparagine. In some embodiments, the residue to which the linker is attached is a tyrosine. In some embodiments, the residue to which the linker is attached is the natural amino acid in that position in SEQ ID NO: 1.

In some embodiments, the linker is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired conjugation handle, such as a CLICK chemistry reagent such as an azide, alkyne, etc.). In some embodiments, the linker is covalently attached to site-specifically to a natural amino acid.

In some embodiments, the linker is attached at an unnatural amino acid residue. In some embodiments, the unnatural amino acid residue comprises a conjugation handle. In some embodiments, the conjugation handle facilitates the addition of the linker to the modified Act-IL-18 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, WO2021133839A1 WO2006069246A2, and WO2007079130A2, each of which is incorporated by reference as if set forth in its entirety.

In some embodiments, the linker is covalently attached at residue 68. In some embodiments, the linker is covalently attached at residue C68, C68E, C68D, C68Q, C68K, C68N, or C68Y. In some embodiments, the linker is covalently attached at residue C68. In some embodiments, the linker is covalently attached to an unnatural amino acid at residue 68.

In some embodiments, the linker is covalently attached at residue 69. In some embodiments, the linker is covalently attached at residue E69, E69C, E69D, E69Q, E69K, E69N, or E69Y. In some embodiments, the linker is covalently attached at residue E69. In some embodiments, the linker is covalently attached residue E69C. In some embodiments, the linker is covalently attached to an unnatural amino acid at residue 69.

In some embodiments, the linker is covalently attached at residue 70. In some embodiments, the linker is covalently attached at residue K70, K70C, K70D, K70Q, K70E, K70N, or K70Y. In some embodiments, the linker is covalently attached at residue K70. In some embodiments, the linker is covalently attached residue K70C. In some embodiments, the linker is covalently attached to an unnatural amino acid at residue 70.

In some embodiments, the linker is covalently attached at residue 85. In some embodiments, the linker is covalently attached at residue E85, E85C, E85D, E85Q, E85K, E85N, or E85Y. In some embodiments, the linker is covalently attached at residue E85. In some embodiments, the linker is covalently attached residue E85C. In some embodiments, the linker is covalently attached to an unnatural amino acid at residue 85.

In some embodiments, the linker is covalently attached at residue 86. In some embodiments, the linker is covalently attached at residue M86C, M86D, M86Q, M86K, M86N, M86E, or M86Y. In some embodiments, the linker is covalently attached M86C. In some embodiments, the linker is covalently attached to an unnatural amino acid at residue 86.

In some embodiments, the polymer is covalently attached at residue 95. In some embodiments, the polymer is covalently attached at residue T95, T95C, T95D, T95Q, T95K, T95N, T95E, or T95Y. In some embodiments, the polymer is covalently attached at residue T95C, T95D, T95Q, T95K, T95N, T95E, or T95Y. In some embodiments, the polymer is covalently attached at residue T95C. In some embodiments, the polymer is covalently attached to an unnatural amino acid at residue 95.

In some embodiments, the linker is covalently attached at residue 98. In some embodiments, the linker is covalently attached at residue D98, D98C, D98Q, D98K, D98N, D98E, or D98Y. In some embodiments, the linker is covalently attached at residue D98C. In some embodiments, the linker is covalently attached to an unnatural amino acid at residue 98.

In some embodiments, the linker is covalently attached through a modified natural amino acid. In some embodiments, the modified natural amino acid comprises a conjugation handle. In some embodiments, the linker is covalently attached through a modified amino acid a. In some embodiments, the modified amino acid a is an amino-acid-PEG-azide group. In some embodiments, the modified amino acid a is a glutamate, aspartate, lysine, cysteine, or tyrosine modified to incorporate an azide group linked to the amino acid through a PEG spacer. In some embodiments, the modified amino acid a has a structure selected from:

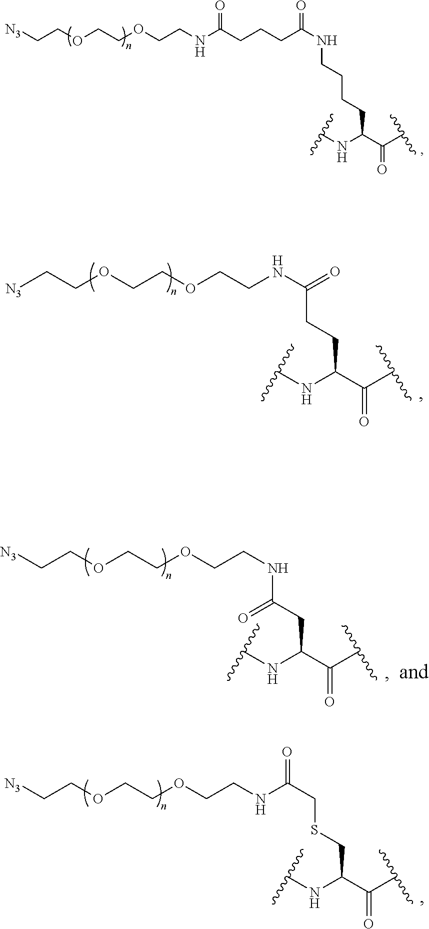

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. The modified amino acid a can be incorporated at any point of attachment of the IL-18 polypeptide of an Act-IL-18 polypeptide as provided herein. In some embodiments, the modified amino acid a is located at a position on the modified IL-18 polypeptide selected from residue 68, residue 69, residue 70, residue 85, residue 86, residue 95, or residue 98.

Where IL-18 polypeptides of Act-IL-18 polypeptides contain unnatural amino acids or modified natural amino acids (e.g., those provided herein for purposes of conjugation), these amino acids may be incorporated into the IL-18 polypeptides using many techniques known in the art for introduction such modifications. For example, recombinant proteins with unnatural amino acids can be made using methods as described in Patent Cooperation Treaty Publication Nos. WO2016115168, WO2002085923, WO2005019415, and WO2005003294. Alternatively, or in combination, unnatural or modified natural amino acids can be incorporated into chemically synthesized proteins during synthesis.

In some embodiments, the linker is attached to the C-terminus of the IL-18 polypeptide, optionally by a linking peptide (e.g., any one of the linking peptides described herein). In some embodiments, the linker is attached using a sortase mediated reaction. For example, an IL-18 polypeptide described herein can have a sortase recognition sequence (e.g., LPETGGH) appended to the C-terminus of the IL-18 polypeptide, optionally via a linking peptide, such as those described herein. The IL-18 polypeptide (e.g., as incorporated into an Act-IL-18 polypeptide) can then be reacted with a suitable donor group (e.g., an additional group containing an N-terminal glycine, preferably a polyglycine). The donor group can be a polypeptide (e.g., a full length protein desired to be attached to the Act-IL-18 polypeptide, such as an antibody as described herien to directly form an activatable immunocytokine), or can be a small peptide comprising a conjugation handle (e.g., a short peptide of, e.g., 2-10 amino acids, which comprises at least 1, preferably 2-3 N-terminal glycine residue(s) linked to a suitable conjugation handle). Exemplary linkers which can be attached via sortase include the GGK-PEG9-N3 and GGE-PEG9-N3 reagents shown below, though other similar such linkers can also be added (e.g., ones which have variable PEG sizes, alternative amino acids attached to the GG group, alternative conjugation handles, etc.). As is apparent to one of ordinary skill in the art, in the sortase region, the “GGH” portion of the sortase recognition sequence is replaced by the glycine (or polyglycine) containing sequence of the reagent (or additional group polypeptide as the case may be). Descriptions of sortase mediated conjugation to proteins can be found in, for example, Patent Cooperation Treaty Publication Nos. WO2013/003555A1, WO2014/140317A2, and WO2020/007899A1.

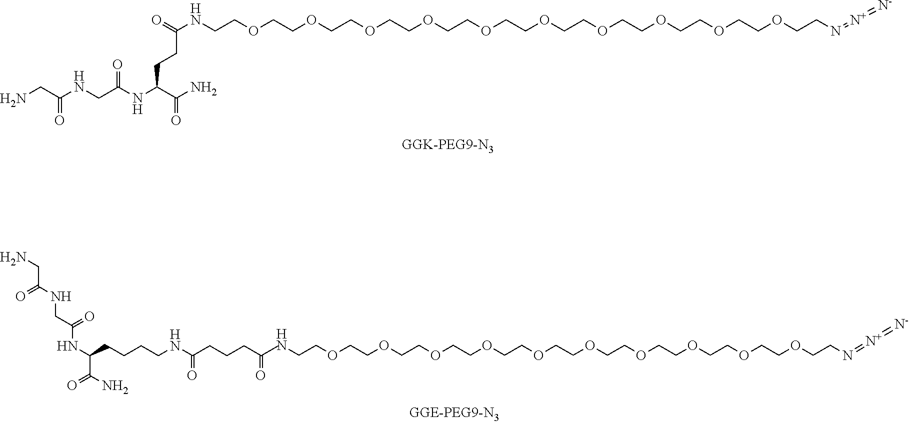

Biological activity of Act-IL-18 and the Active Form of the IL-18 Polypeptide Binding Affinity

In some embodiments the Act-IL-18 (either free or incorporated into an activatable immunocytokine) exhibits a decreased affinity for the IL-18 receptor of at least 10-fold, at least 100 fold, at least 500 fold, at least 1000 fold lower in comparison to wild type IL-18 or the active form of the IL-18 polypeptide (or a corresponding activatable immunocytokine comprising the same). In some embodiments, the Act-IL-18 exhibits an increased EC50 for the production of IFNγ that is at least 5 fold, at least 10-fold, at least 100 fold, at least 500 fold, at least 1000 fold higher in comparison to wild type IL-18 or the active from of the IL-18 polypeptide. In some embodiments, the Act-IL-18 exhibits an increased EC50 for the production of IFNγ that is at least 5 fold, at least 10-fold, at least 100 fold, at least 500 fold, at least 1000 fold higher in comparison to the active form of the IL-18 polypeptide. In some embodiments, the Act-IL-18 exhibits an increased EC50 for the production of IFN-g that is at least 5 fold, at least 10-fold, at least 100 fold, at least 500 fold, at least 1000 fold higher in comparison to the IL-18 polypeptide.

In some embodiments, the active form of the IL-18 polypeptide (either free or incorporated into an activatable immunocytokine) provided herein exhibits reduced affinity for IL-18 binding protein (IL-18BP) compared to WT IL-18 (SEQ ID NO: 1). In some embodiments, the active form of the IL-18 polypeptide exhibits at least 2-fold, at least 5-fold, at least 10-fold, at least 20-fold, at least 30-fold, at least 40-fold, at least 50-fold, at least 60-fold, at least 70-fold, at least 80-fold, at least 90-fold, or at least 100-fold lower affinity for IL-18BP compared to WT IL-18. In some embodiments, the active form of the IL-18 polypeptide exhibits at least a 10-fold lower affinity for IL-18BP compared to WT IL-18. In some embodiments, the active form of the IL-18 polypeptide exhibits at least a 20-fold lower affinity for IL-18BP compared to WT IL-18. In some embodiments, the active form of the IL-18 polypeptide exhibits at least a 50-fold lower affinity for IL-18BP compared to WT IL-18. In some embodiments, the active form of the IL-18 polypeptide exhibits at least an 80-fold lower affinity for IL-18BP compared to WT IL-18. In some embodiments, the active form of the IL-18 polypeptide exhibits at least a 100-fold lower affinity for IL-18BP compared to WT IL-18.

In some embodiments, the active form of the IL-18 polypeptide provided herein (either free or incorporated into an activatable immunocytokine) exhibits a reduced binding to IL-18BP as measured by KD. In some embodiments, the active form of the IL-18 polypeptide exhibits a KD with IL-18BP of at least about 1 nM, at least about 5 nM, at least about 10 nM, at least about 15 nM, at least about 20 nM, at least about 25 nM, at least about 50 nM, at least about 100 nM, at least about 200 nM, at least about 300 nM, at least about 400 nM, or at least about 500 nM. In some embodiments, the active form of the IL-18 polypeptide exhibits a KD with IL-18BP of at least about 1 nM. In some embodiments, the active form of the IL-18 polypeptide exhibits a KD with IL-18BP of at least about 5 nM. In some embodiments, the active form of the IL-18 polypeptide exhibits a KD with IL-18BP of at least about 50 nM. In some embodiments, the active form of the IL-18 polypeptide exhibits a KD with IL-18BP of at least about 100 nM. In some embodiments, the active form of the IL-18 polypeptide exhibits a KD with IL-18BP of at least about 500 nM.

In some embodiments, the active form of the IL-18 polypeptide (either free or incorporated into an activatable immunocytokine) displays at most an only slightly diminished affinity for IL-18Rab compared to WT IL-18 (SEQ ID NO: 1). In some embodiments, the active form of the IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 Rab as compared to the affinity of WT IL-18 for IL-18Rab. In some embodiments, the active form of the IL-18 polypeptide exhibits at most a 10-fold lower affinity for IL-18Rab as compared to the affinity of WT IL-18 for IL-18Rab. In some embodiments, the active form of the IL-18 polypeptide exhibits at most a 20-fold lower affinity for IL-18Rab as compared to the affinity of WT IL-18 for IL-18Rab. In some embodiments, the active form of the IL-18 polypeptide exhibits at most a 50-fold lower affinity for IL-18Rab as compared to the affinity of WT IL-18 for IL-18Rab. In some embodiments, the active form of the IL-18 polypeptide exhibits at most a 100-fold lower affinity for IL-18 Rab as compared to the affinity of WT IL-18 for IL-18Rab. In some embodiments, the active form of the IL-18 polypeptide exhibits an increased affinity for IL-18Rab compared to WT IL-18. In some embodiments, the affinity is increased by at least 2-fold, at least 4-fold, at least 6-fold, at least 8-fold, or at least 10-fold compared to WT IL-18.

In some embodiments, the active form of the IL-18 polypeptide (either free or incorporated into an activatable immunocytokine) displays at most an only slightly diminished affinity for IL-18Rab compared to the corresponding IL-18 polypeptide without the artificial polypeptide. In some embodiments, the active form of the IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 Rab as compared to the affinity of the corresponding IL-18 polypeptide without the artificial polypeptide for IL-18Rab. In some embodiments, the active form of the IL-18 polypeptide exhibits at most a 2-fold lower affinity for IL-18Rab as compared to the affinity of the corresponding IL-18 polypeptide without the artificial polypeptide for IL-18Rab. In some embodiments, the active form of the IL-18 polypeptide exhibits at most a 3-fold lower affinity for IL-18Rab as compared to the affinity of the corresponding IL-18 polypeptide without the artificial polypeptide for IL-18Rab. In some embodiments, the active form of the IL-18 polypeptide exhibits at most a 4-fold lower affinity for IL-18Rab as compared to the affinity of the corresponding wild type IL-18 polypeptide without the N-terminal domain for IL-18Rab. In some embodiments, the active form of the IL-18 polypeptide exhibits at most a 5-fold lower affinity for IL-18 Rab as compared to the affinity of the corresponding IL-18 polypeptide without the artificial polypeptide for IL-18Rab. In some embodiments, the active form of the IL-18 polypeptide exhibits at most a 10-fold lower affinity for IL-18Rab as compared to the affinity of the corresponding IL-18 polypeptide without the artificial polypeptide for IL-18Rab.

In some embodiments, the active form of the IL-18 polypeptide provided herein (either free or incorporated into an activatable immunocytokine) exhibits at most only a slight reduction in binding to IL-18Rab as measured by KD. In some embodiments, the active form of the IL-18 polypeptide exhibits a KD with IL-18Rab of at most about 10 nM, at most about 20 nM, at most about 30 nM, at most about 50 nM, at most about 75 nM, at most about 100 nM, or at most about 200 nM. In some embodiments, the active form of the IL-18 polypeptide exhibits a KD with IL-18Rab of at most about 20 nM. In some embodiments, the active form of the Act-IL-18 polypeptide exhibits a KD with IL-18Rab of at most about 30 nM. In some embodiments, the active form of the IL-18 polypeptide exhibits a KD with IL-18Rab of at most about 40 nM. In some embodiments, the active form of the IL-18 polypeptide exhibits a KD with IL-18Rab of at most about 50 nM. In some embodiments, the active form of the IL-18 polypeptide exhibits an increase in binding to IL-18Rab compared to WT IL-18 as measured by KD. In some embodiments, the active form of the IL-18 polypeptide has a KD with IL-18Rα of at most about 2 nM, at most about 1 nM, at most about 0.5 nM, or at most about 0.2 nM.

In some embodiments, the active form of the IL-18 polypeptide (either free or incorporated into an activatable immunocytokine) exhibits a wide window in which the active form of the IL-18 polypeptide will bind to IL-18Rab even in the presence of IL-18BP. In some embodiments, this window can be measured by a ratio of KD of the Act-IL-18/IL-18BP interaction over KD of the IL-18/IL-18Rab interaction, where a larger number indicates a larger window in which the active form of the Act-IL-18 polypeptide is expected to be active in vivo. In some embodiments, the active form of the IL-18 polypeptide exhibits a ratio of KD of the IL-18/IL-18BP interaction over KD of the IL-18/IL-18Rab interaction of at least about 1, at least about 2, at least about 3, at least about 4, at least about 5, at least about 10, at least about 15, at least about 20, at least about 25, at least about 30, at least about 35, at least about 40, at least about 45, or at least about 50. In some embodiments, the active form of the IL-18 polypeptide exhibits a ratio of KD of the IL-18/IL-18BP interaction over KD of the IL-18/IL-18Rab interaction of at least about 2. In some embodiments, the active form of the IL-18 polypeptide exhibits a ratio of KD of the IL-18/IL-18BP interaction over KD of the IL-18/IL-18Rab interaction of at least about 5. In some embodiments, the active form of the IL-18 polypeptide exhibits a ratio of KD of the IL-18/IL-18BP interaction over KD of the IL-18/IL-18Rab interaction of at least about 10 . . . . In some embodiments, the active form of the IL-18 polypeptide exhibits a ratio of KD of the IL-18/IL-18BP interaction over KD of the IL-18/IL-18Rab interaction of at least about 25 . . . . In some embodiments, the active form of the IL-18 polypeptide exhibits a ratio of KD of the IL-18/IL-18BP interaction over KD of the IL-18/IL-18Rab interaction of at least about 30. In some embodiments, the active form of the IL-18 polypeptide exhibits a ratio of KD of the IL-18/IL-18BP interaction over KD of the IL-18/IL-18Rab interaction of at least about 40. In some embodiments, the active form of the IL-18 polypeptide exhibits a ratio of KD of the IL-18/IL-18BP interaction over KD of the IL-18/IL-18Rab interaction of at least about 45. In some embodiments, the active form of the IL-18 polypeptide exhibits a ratio of KD of the IL-18/IL-18BP interaction over KD of the IL-18/IL-18Rab interaction of at least about 50.

Functional Activity of the activated form of the IL-18 Polypeptide

In some embodiments, the active form of the IL-18 polypeptides provided herein (either free or incorporated into an activatable immunocytokine) display one or more activities associated with WT IL-18. In some embodiments, the active form of the IL-18 polypeptide exhibits a similar ability to signal through the IL-18 receptor (IL-18Rab) but lacks the ability or displays a reduced ability to be inhibited by IL-18BP. In some embodiments, the active form of the IL-18 polypeptide's ability to signal through IL-18Rab is reduced compared to WT IL-18 by only a small amount.

In some embodiments, the active form of the IL-18 polypeptide (either free or incorporated into an activatable immunocytokine) modulates IFNγ production when in contact with a cell (e.g., an immune cell, such as an NK cell). In some embodiments, the active form of the IL-18 polypeptide's ability to modulate IFNγ production is measured as a half-maximal effective concentration (EC50). In some embodiments, an EC50 (nM) of the active form of the Act-IL-18 polypeptide's ability to induce IFNγ is less than 10-fold higher than, less than 5-fold higher than, or less than an EC50 (nM) of an IL-18 polypeptide of SEQ ID NO: 1. In some embodiments, the EC50 of the active form of the Act-IL-18 polypeptide's ability to induce IFNγ is less than 10-fold higher than an EC50 (nM) of an IL-18 polypeptide of SEQ ID NO: 1. In some embodiments, the EC50 of the active form of the Act-IL-18 polypeptide's ability to induce IFNγ is less than 5-fold higher than an EC50 (nM) of an IL-18 polypeptide of SEQ ID NO: 1. In some embodiments, the EC50 of the active form of the IL-18 polypeptide's ability to induce IFNγ is less than an EC50 (nM) of an IL-18 polypeptide of SEQ ID NO: 1. In some embodiments, the EC50 of the active form of the IL-18 polypeptide's ability to induce IFNγ is less than 10-fold higher than, less than 8-fold higher than, less than 6-fold higher than, less than 5-fold higher than, less than 4-fold higher than, less than 3-fold higher than, or less than 2-fold higher than an EC50 (nM) of an IL-18 polypeptide of SEQ ID NO: 1. In some embodiments, the EC50 of the active form of the IL-18 polypeptide's ability to induce IFNγ is measured by an IFNγ induction cellular assay.

In some embodiments, an EC50 of the active form of the IL-18 polypeptide's ability to induce IFNγ production (either free or incorporated into an activatable immunocytokine) is less than about 100 nM, less than about 75 nM, less than about 50 nM, less than about 40 nM, less than about 30 nM, less than about 20 nM, less than about 15 nM, or less than about 10 nM. In some embodiments, an EC50 of the active form of the IL-18 polypeptide's ability to induce IFNγ production is less than about 100 nM. In some embodiments, an EC50 of the active form of the IL-18 polypeptide's ability to induce IFNγ production is less than about 50 nM. In some embodiments, an EC50 of the active form of the IL-18 polypeptide's ability to induce IFNγ production is less than about 10 nM.

In some embodiments, the active form of the IL-18 polypeptide (either free or incorporated into an activatable immunocytokine) exhibits a reduced ability to have its IFNγ induction activity inhibited by IL-18BP compared to WT IL-18. In some embodiments, the active form of the IL-18 displays a half-maximal inhibitory concentration (IC50) by IL-18BP which is at least about 10-fold higher than, at least about 20-fold higher than, at least about 50-fold higher than, at least about 75-fold higher than, at least about 100-fold higher than, at least about 200-fold higher than, at least about 300-fold higher than, at least about 400-fold higher than, at least about 500-fold higher than, at least about 600-fold higher than, at least about 700-fold higher than, at least about 800-fold higher than, at least about 900-fold higher than, or at least about 1000-fold higher than an IC50 of WT IL-18's inhibition by IL-18BP. In some embodiments, the active form of the IL-18 displays a half-maximal inhibitory concentration (IC50) by IL-18BP which is at least about 100-fold higher than an IC50 of WT IL-18's inhibition by IL-18BP. In some embodiments, the active form of the IL-18 displays a half-maximal inhibitory concentration (IC50) by IL-18BP which is at least about 500-fold higher than an IC50 of WT IL-18's inhibition by IL-18BP. In some embodiments, the active form of the IL-18 displays a half-maximal inhibitory concentration (IC50) by IL-18BP which is at least about 1000-fold higher than an IC50 of WT IL-18's inhibition by IL-18BP.

In some embodiments, the active form of the IL-18 polypeptide (either free or incorporated into an activatable immunocytokine) exhibits a favorable ratio of half-maximal inhibitory concentration (IC50) by IL-18BP over a half-maximal effective concentration (EC50) of IFNγ induction (IC50/EC50 ratio). In some embodiments, the IC50/EC50 ratio is increased compared to WT IL-18. In some embodiments, the IC50/EC50 ratio is increased by at least about 2-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold compared to WT IL-18. In some embodiments, the IC50/EC50 ratio is increased by at least about 10-fold compared to WT IL-18. In some embodiments, the IC50/EC50 ratio is increased by at least about 100-fold compared to WT IL-18. In some embodiments, the IC50/EC50 ratio is increased by at least about 500-fold compared to WT IL-18. In some embodiments, the IC50/EC50 ratio of the active form of the Act-IL-18 polypeptide is at least about 2, at least about 5, at least about 10, at least about 50, at least about 100, at least about 250, or at least about 500.

In some embodiments, the active form of the IL-18 polypeptide (either free or incorporated into an activatable immunocytokine) modulates IFNγ production, and wherein an EC50 (nM) of the active form of the Act-IL-18 polypeptide against IFNγ is less than an EC50 (nM) of an IL-18 polypeptide of SEQ ID NO: 1. In some embodiments, the EC50 (nM) of the active form of the Act-IL-18 polypeptide against IFNγ is at least 10-fold less than the EC50 (nM) of an IL-18 polypeptide of SEQ ID NO: 1. In some embodiments, the EC50 (nM) of the active form of the Act-IL-18 polypeptide against IFNγ is about 10-fold less than the EC50 (nM) of an IL-18 polypeptide of SEQ ID NO: 1. In some embodiments, the EC50 (nM) of the active form of the Act-IL-18 polypeptide against IFNγ is about 15-fold less than the EC50 (nM) of a n IL-18 polypeptide of SEQ ID NO: 1.

Relative Activity of the Activatable and Active form of IL-18

In some embodiments, the activated form of the IL-18 polypeptide (e.g., after cleavage of the specific cleavage site) (either free or incorporated into an activatable immunocytokine) exhibits an enhanced activity associated with IL-18 compared to the Act-IL-18 polypeptide with the specific cleavage site intact. In some embodiments, the active form of the IL-18 polypeptide exhibits an activity which is enhanced by at least 5-fold, 10-fold, 20-fold, 50-fold, 100-fold, 200-fold, 500-fold, 1000-fold, 2000-fold, 5000-fold, 10000-fold, 15000-fold, or 20,000-fold higher than the Act-IL-18 polypeptide. Such activities can include induction of production of IFNγ in a cell (e.g., an immune cell such as an NK cell), activation of signaling through the IL-18 receptor (e.g., in a reporter assay), or another in vitro or in vivo activity. In some embodiments, the activated form of the IL-18 polypeptide exhibits enhanced binding to the IL-18 receptor or a subunit thereof (e.g., the IL-18 receptor alpha subunit) compared to the Act-IL-18 polypeptide (e.g., has a KD which is at least 10-fold, 20-fold, 50-fold, or 100-fold lower).

In some embodiments, the Act IL-18 polypeptide (either free or incorporated into an activatable immunocytokine) exhibits a half-maximal effective concentration (EC50) for IL-18 receptor signaling activity (e.g., in a HEK-Blue reporter assay) which is higher than that of the activated form of the IL-18 polypeptide. In some embodiments, the Act IL-18 polypeptide exhibits an EC50 for IL-18 receptor signaling activity which is at least 1,000-fold higher, 2,000-fold higher, 5,000-fold higher, 10,000-fold higher, 15,000-fold-higher, or 20,000-fold higher than the activated form of the IL-18 polypeptide. In some embodiments, the Act IL-18 polypeptide exhibits an EC50 for IL-18 receptor signaling activity which is at least 1,000-fold higher than the activated form of the IL-18 polypeptide. In some embodiments, the Act IL-18 polypeptide exhibits an EC50 for IL-18 receptor signaling activity which is at least 5,000-fold higher than the activated form of the IL-18 polypeptide. In some embodiments, the Act IL-18 polypeptide exhibits an EC50 for IL-18 receptor signaling activity which is at least 10,000-fold higher than the activated form of the IL-18 polypeptide. In some embodiments, the Act IL-18 polypeptide exhibits an EC50 for IL-18 receptor signaling activity which is at least 20,000-fold higher than the activated form of the IL-18 polypeptide.

In some embodiments, the Act-IL-18 polypeptide (either free or incorporated into an activatable immunocytokine) exhibits only a modest reduction in activity compared to the activated form of the IL-18 polypeptide. In some embodiments, the Act IL-18 polypeptide exhibits a half-maximal effective concentration (EC50) for IL-18 receptor signaling activity which is from about 10-fold higher to about 100-fold higher than the activated form of the IL-18 polypeptide. In some embodiments, the Act IL-18 polypeptide exhibits an EC50 for IL-18 receptor signaling activity which is from about 10-fold higher to about 50-fold higher than the activated form of the IL-18 polypeptide.

In some embodiments, the activated form of the IL-18 polypeptide (either free or incorporated into an activatable immunocytokine) has a comparable activity compared to that of the IL-18 polypeptide from which the Act-IL-18 polypeptide is derived. In some embodiments, the activated form of the IL-18 polypeptide exhibits a half-maximal effective concentration (EC50) for IL-18 receptor signaling activity which is within about 10-fold of the IL-18 polypeptide.

### Biological Activity of the Immunocytokines.

In some embodiments, an immunocytokine described herein exhibits one or more activities associated with the antibody or antigen binding fragment and/or an IL-18 polypeptide after cleavage of the Act-IL-18.

In some embodiments, the immunocytokine exhibits an ability to bind to the IL-18 receptor. In some embodiments, the immunocytokine exhibits an ability to bind to the IL-18 receptor after cleavage of the Act-IL-18 which is comparable to WT IL-18. In some embodiments, immunocytokine exhibits an ability to bind to the IL-18 receptor (IL-18Rαβ) which is reduced by at most 2-fold, at most 5-fold, at most 10-fold, at most 20-fold, at most 50-fold, at most 100-fold, at most 200-fold, at most 300-fold, at most 400-fold, or at most 1000-fold compared to WT IL-18. In some embodiments, the immunocytokine exhibits an enhanced ability to bind the IL-18Rαβ. In some embodiments, the immunocytokine exhibits an ability to bind to the IL-18Rαβ which is increased by at least 2-fold, at least 3-fold, at least 5-fold, or at least 10-fold compared to WT IL-18.

In some embodiments, the immunocytokine exhibits an ability to stimulate production of IFNγ upon contact with a cell (e.g., an immune cell, such as an NK cell) after cleavage of the Act-IL-18. In some embodiments, the ability of the immunocytokine to stimulate IFNγ production is somewhat reduced compared to WT IL-18. In some embodiments, a half-maximal effective concentration (EC50) of the ability of the immunocytokine to stimulate production of IFNγ is at most 100-fold higher than, at most 50-fold higher than, at most 20-fold higher than, at most 10-fold higher than, at most 5-fold higher than, or at most 2-fold higher than that of a WT IL-18. In some embodiments, the ability of the immunocytokine to stimulate IFNγ production is enhanced compared to WT IL-18. In some embodiments, a half-maximal effective concentration (EC50) of the ability of the immunocytokine to stimulate production of IFNγ is at least 5-fold lower than, at least 10-fold lower than, at least 20-fold lower than, at least 50-fold lower than, at least 75-fold lower than, or at least 100-fold higher than that of a WT IL-18.

In some embodiments, the immunocytokine exhibits an ability to stimulate production of IFNγ upon contact with a cell (e.g., an immune cell, such as an NK cell) after cleavage of the Act-IL-18 which is only somewhat reduced as compared to the IL-18 polypeptide not comprised in the immunocytokine (e.g., unconjugated IL-18 polypeptide). In some embodiments, the EC50 of IFNγ stimulation is at most 5-fold greater than, at most 10-fold greater than, at most 50-fold greater than, or at most 100-fold greater than that that of the IL-18 polypeptide not comprised in the immunocytokine. In some embodiments, the immunocytokine exhibits an ability to induce IFNγ production in a cell as measured by half-maximal effective concentration (EC₅₀) which is within about 100-fold of the corresponding IL-18 polypeptide not comprised in the immunocytokine. In some embodiments, the immunocytokine exhibits a lower EC₅₀ than WT IL-18. In some embodiments, the immunocytokine exhibits a lower EC₅₀ than WT IL-18 by at least 2-fold, 5-fold, 10-fold, 20-fold, 30-fold, 50-fold, or 100-fold.

In some embodiments, the immunocytokine exhibits a reduced ability to bind IL-18 binding protein (IL-18BP) before and after cleavage of the Act-IL-18. In some embodiments, the ability of immunocytokine to bind IL-18BP is reduced by at least 2-fold, at least 5-fold, at least 10-fold, at least 20-fold, at least 30-fold, at least 50-fold, or at least 100-fold compared to WT IL-18. In some embodiments, the immunocytokine does not display any substantial ability to bind IL-18 BP.

In some embodiments, the immunocytokine exhibits a reduced ability to have its IFNγ production stimulatory activity inhibited by IL-18BP. In some embodiments, the ability of the immunocytokine to be inhibited by IL-18BP is measured as a half maximal inhibitory concentration (IC₅₀). In some embodiments, the immunocytokine exhibits an IC₅₀ by IL-18BP that is at least 2-fold higher than, at least 5-fold higher than, at least 10-fold higher than, at least 15-fold higher than, at least 20-fold higher than, at least 25-fold higher than, at least 30-fold higher than, at least 40-fold higher than, or at least 50-fold higher than an IC₅₀ of WT IL-18's inhibition by IL-18BP. In some embodiments, the immunocytokine exhibits an IC₅₀ by IL-18BP that is at least 100-fold higher than an IC₅₀ of WT IL-18's inhibition by IL-18BP. In some embodiments, the immunocytokine exhibits an IC₅₀ by IL-18BP that is at least 200-fold higher than an IC₅₀ of WT IL-18's inhibition by IL-18BP. In some embodiments, the immunocytokine exhibits an IC₅₀ by IL-18BP that is at least 500-fold higher than an IC₅₀ of WT IL-18's inhibition by IL-18BP. In some embodiments, the immunocytokine exhibits an IC₅₀ by IL-18BP that is at least 1000-fold higher than an IC₅₀ of WT IL-18's inhibition by IL-18BP.

In some embodiments, the immunocytokine retains binding associated with the antibody or antigen binding fragment. In some embodiments, the immunocytokine retains binding to the antigen of the antibody or antigen binding fragment. In some embodiments, the immunocytokine exhibits binding affinity (K_{D}) to the antigen of the antibody which is within 5-fold of the binding affinity of the antibody not attached to the IL-18 polypeptide. In some embodiments, the immunocytokine exhibits binding affinity (K_{D}) to the antigen of the antibody which is within 2.5-fold of the binding affinity of the antibody not attached to the IL-18 polypeptide. In some embodiments, the binding is determined by ELISA. In some embodiments, the binding is determined by BLI.

In some embodiments, the immunocytokine retains binding to one or more Fc receptors associated with the antibody or antigen binding fragment. In some embodiments, the Fc receptor is selected from FcRn, CD64, CD32a, CD16, and CD32b, or any combination thereof. In some embodiments, the immunocytokine exhibits a binding affinity (K_{D}) to at least one Fc receptor which is within 10-fold of the binding affinity of the antibody not attached to the IL-18 polypeptide. In some embodiments, the immunocytokine exhibits a binding affinity (K_{D}) to at least one Fc receptor which is less than 10-fold higher, less than 5-fold higher, less than 4-fold higher, less than 3-fold higher, less than 2-fold higher, or less than the binding affinity of the antibody not attached to the IL-18 polypeptide. In some embodiments, the immunocytokine exhibits a binding affinity (K_{D}) to each of FcRn, CD64, CD32a, CD16, and CD32B which is less than 10-fold higher, less than 5-fold higher, less than 4-fold higher, less than 3-fold higher, less than 2-fold higher, or less than the binding affinity of the antibody not attached to the IL-18 polypeptide. In some embodiments, the immunocytokine exhibits a binding affinity (K_{D}) to each of FcRn, CD64, CD32a, CD16, and CD32B which is within 10-fold of the binding affinity of the antibody not attached to the IL-18 polypeptide. In some embodiments, the immunocytokine exhibits a binding affinity (K_{D}) to each of FcRn, CD64, CD32a, CD16, and CD32B which is within 20-fold of the binding affinity of the antibody not attached to the IL-18 polypeptide. In some embodiments, the immunocytokine exhibits a binding affinity (K_{D}) to each of FcRn, CD64, CD32a, CD16, and CD32B which is within 50-fold of the binding affinity of the antibody not attached to the IL-18 polypeptide. In some embodiments, the immunocytokine exhibits a binding affinity (K_{D}) to each of FcRn, CD64, CD32a, CD16, and CD32B which is within 100-fold of the binding affinity of the antibody not attached to the IL-18 polypeptide.

In some embodiments, the immunocytokine exhibits synergistic efficacy owing to the presence of both molecules in one molecule. In some embodiments, the immunocytokine exhibits enhanced activity compared to cither molecule alone. In some embodiments, the immunocytokine exhibits enhanced anti-tumor growth inhibition compared to the antibody alone. In some embodiments, the immunocytokine exhibits enhanced anti-tumor growth inhibition compared to the antibody and the IL-18 polypeptide administered in combination. In some embodiments, the IL-18 polypeptide is administered as a half-life extended version (e.g., PEGylated, attached to an Fc region (e.g., an Fc fusion), or attached to a negative control antibody). In some embodiments, the immunocytokine exhibits similar or enhanced antitumor activity at the same concentration as the antibody administered alone. In some embodiments, the immunocytokine exhibits similar or enhanced antitumor activity when administered at a dose which is less than 0.5-fold, 0.25-fold, or 0.1-fold the dose of the antibody alone.

### Compositions

In one aspect, provided herein is a pharmaceutical composition comprising an antibody linked to a modified IL-18 polypeptide described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition further comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

In one aspect, described herein is a liquid or lyophilized composition that comprises a described antibody or antigen binding fragment linked to a modified IL-18 polypeptide. In some embodiments, the antibody or antigen binding fragment linked to the IL-18 polypeptide modified IL-18 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The activatable immunocytokines described herein can be in a variety of dosage forms. In some embodiments, the activatable immunocytokine is dosed as a lyophilized powder. In some embodiments, the activatable immunocytokine is dosed as a suspension. In some embodiments, the activatable immunocytokine is dosed as a solution. In some embodiments, the activatable immunocytokine is dosed as an injectable solution. In some embodiments, the activatable immunocytokine is dosed as an IV solution.

### Methods of Treatment

In one aspect, described herein, is a method of treating cancer in a subject in need thereof, comprising: administering to the subject an effective amount of an activatable immunocytokine or a pharmaceutical composition as described herein. In some embodiments, the cancer is a solid cancer. A cancer or tumor can be, for example, a primary cancer or tumor or a metastatic cancer or tumor. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

Combination therapies with one or more additional active agents are contemplated herein.

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the activatable immunocytokine is administered in a single dose of the effective amount of activatable immunocytokine, including further embodiments in which (i) the activatable immunocytokine is administered once a day; or (ii) the activatable immunocytokine is administered once a day; or (ii) the activatable immunocytokine is administered to the subject multiple times over the span of one day. In some embodiments, the conjugate is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

### Methods of Manufacturing

In one aspect, described herein, is a method of making an activatable immunocytokine, comprising providing an antibody or antigen binding fragment thereof (e.g., an antibody or antigen binding fragment provided herein), wherein the antibody comprises a reactive group (e.g., a conjugation handle), contacting the reactive group with a complementary reactive group attached to an Act-IL-18 polypeptide, and forming the activatable immunocytokine. The resulting composition is any of the compositions provided herein.

In some embodiments, providing the antibody comprising the reactive group comprises attaching the reactive group to the antibody. In some embodiments, the reactive group is added site-specifically. In some embodiments, attaching the reactive group to the antibody comprises contacting the antibody with an affinity group comprising a reactive functionality which forms a bond with a specific residue of the antibody. In some embodiments, attaching the reactive group to the antibody comprises contacting the antibody with an enzyme. In some embodiments, the enzyme is configured to site-specifically attach the reactive group to a specific residue of the antibody. In some embodiments, the enzyme is glycosylation enzyme or a transglutaminase enzyme.

In some embodiments, the method further comprises attaching the complementary reactive group to the Act-IL-18 polypeptide.

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Throughout the instant description, certain numerical or other similar values may be described as, for example, “at least” or “at most” a set of values indicated in a list form (e.g., “at least 2, 3, 4, 5, or 6”). In such cases, unless context clearly indicates otherwise, it is intended that the phrase “at least,” “at most,” or other similar term is applied individually to each value in the list. For example, the phrase “at least 2, 3, 4, 5, or 6” is to be interpreted as “at least 2, at least 3, at least 4, at least 5, or at least 6.”

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of IL-18 polypeptides or other polypeptides. As used herein, “attached” or “covalently attached” means that the group is tethered to the indicated reside, and such tethering can include a linking group (i.e., a linker). Thus, for a group “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (Kp) between the two relevant molecules. When comparing Kp values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ L P ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

Referred to herein are amino acid or amino acid sequences which appear “upstream” of another referenced amino acid sequence. The term “upstream” in this context means the indicated amino acid or amino acid sequence is affixed to the N-terminal residue of the referenced amino acid sequence (e.g., a methionine residue positioned upstream of a sequence “SDGTK” would have a sequence of “MSDGTK”). In some cases, residue numbering of “upstream” amino acids or amino acid sequences uses a negative numbered numbering system (e.g., reference to positions at a −1, −2, or −3 position relative to a reference sequence). When such a numbering system is used, the −1 position corresponds to the amino acid affixed to the N-terminus of the reference sequence, the −2 position corresponds to the amino acid affixed at the N-terminus of the −1 position, and so on and so forth. For example, a sequence of AM positioned upstream of a reference sequence SDGTK would result in a full sequence of AMSDGTK, where M is at the −1 position and A is at the −2 position relative to the reference sequence.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

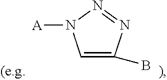

it is intended that the other regioisomer

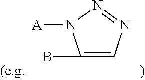

is also encompassed.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted | triazole |
| | cyclooctyne (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyoclooctene | dihydropyridazine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₈alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methyl ethyl (i-propyl), n-butyl, i-butyl, 5-butyl, n-pentyl, 1,1-dimethyl ethyl (i-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH—CH—, —CH₂CH═CH—, or —CH—CHCH₂—. In some embodiments, the alkenylene is —CH—CH—. In some embodiments, the alkenylene is —CH₂CH═CH—. In some embodiments, the alkenylene is —CH—CHCH₂—.

The term “alkynyl” refers to a type of alkyl group in which at least one carbon-carbon triple bond is present. In one embodiment, an alkenyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH3, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1 (2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quatemized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 0 atoms, 0-1 P atoms, and 0-1 S atoms in the ring. In some embodiments, a heteroaryl contains 1-4 N atoms, 0-1 0 atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(=O)NH₂, —C(=0)NH(alkyl), —C(=0)N(alkyl)₂, —S(=0)₂NH₂, —S(═O)₂NH(alkyl), —S(═O)₂N (alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —C0₂(Ci-C₄alkyl), —C(=0) NH₂, —C(=O)NH(C₁-C₄alkyl), —C(═O) N(C₁-C₄alkyl)₂, —S(═O)₂NH₂, —S(═O)₂NH(Ci-C₄alkyl), —S(=0)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(=0) C₁-C₄alkyl, and —S(=0)₂C₁-C₄alkyl. In some embodiments, optional substituents are independently selected from D, halogen, —CN, —NH₂, —OH, —NH(CH₃), —N(CH₃)₂, —NH (cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (=0).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18^{th} residue in a human IgG C_{H}2 region (EU numbering).

### Sequences (SEQ ID NOS) of IL-18 Polypeptides

TABLE 3

| SEQ | | |
|---|---|---|
| ID NO: | Modification | Sequence |
| 1 | Native | YFGKLESKLS VIRNLNDQVL FIDQGNRPLF EDMTDSDCRD |
| | sequence | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 2 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | E85C | ISFKCMNPPD NIKDTKSDII FFQRSVPGHD NKMQFESSSY |
| | | EGYFLACEKE RDLFKLILKK EDELGDRSIM FTVQNED |
| 3 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKCMNPPD NIKDTKSDII FFQRSVPGHD NKMQFESSSY |
| | C76A, E85C, | EGYFLAAEKE RDLFKLILKK EDELGDRSIM FTVQNED |
| | C127A | |
| 4 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | M86C | ISFKECNPPD NIKDTKSDII FFQRSVPGHD NKMQFESSSY |
| | | EGYFLACEKE RDLFKLILKK EDELGDRSIM FTVQNED |
| 5 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKECNPPD NIKDTKSDII FFQRSVPGHD NKMQFESSSY |
| | C76A, M86C, | EGYFLAAEKE RDLFKLILKK EDELGDRSIM FTVQNED |
| | C127A | |
| 6 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDTKSCII FFQRSVPGHD NKMQFESSSY |
| | C76A, D98C, | EGYFLAAEKE RDLFKLILKK EDELGDRSIM FTVQNED |
| | C127A | |
| 7 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSAENKI |
| | C76A, M86C, | ISFKECNPPD NIKDTKSDII FFQRSVPGHD NKMQFESSSY |
| | C127A | EGYFLAAEKE RDLFKLILKK EDELGDRSIM FTVQNED |
| 8 | E6K, V11I, | YFGKLKSKLS IIRNLNDQVL FIDQGNRPLF EDMTDSDARD |
| | C38A, K53A, | NAPRTIFIIS MYADSQPRGM AVAISVKAEK ISTLSAENKI |
| | T63A, C68A, | ISFKEMNPPD NIKDCKSDII FFQRSVPGHD NKMQFESSSY |
| | C76A, T95C, | EGYFLAAEKE RDLFKLILKK EDELGDRSIM FTVQNED |
| | C127A | |
| 9 | E6K, C38A, | YFGKLKSKLS VIRNLNDQVL FIDQGNRPLF EDMTDSDARD |
| | K53A, C68A, | NAPRTIFIIS MYADSQPRGM AVTISVKAEK ISTLSCENKI |
| | D98C | ISFKEMNPPD NIKDTKSCII FFQRSVPGHD NKMQFESSSY |
| | | EGYFLACEKE RDLFKLILKK EDELGDRSIM FTVQNED |
| 10 | E6K, C38A, | YFGKLKSKLS VIRNLNDQVL FIDQGNRPLF EDMTDSDCRD |
| | K53A, C68A, | NAPRTIFIIS MYADSQPRGM AVTISVKAEK NKMQFESSSY |
| | C76A, D98C, | ISFKEMNPPD NIKDTKSCII FFQRSVPGHD FTVQNED |
| | C127A | EGYFLAAEKE RDLFKLILKK EDELGDRSIM |
| 11 | E6K, V11I, | YFGKLKSKLS IIRNLNDQVL FIDQGNRPLF DEMTSDARD |
| | C38A, K53A, | NAPRTIFIIS MYADSQPRGM AVTISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 12 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 13 | E6K, K53A, | YFGKLKSKLS VIRNLNDQVL FIDQGNRPLF EDMTDSDCRD |
| | T63N | NAPRTIFIIS MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 14 | E6K, K53A, | YFGKLKSKLS VIRNLNDQVL FIDQGNRPLF EDMTDSDCRD |
| | S50A, T63N | NAPRTIFIIA MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 15 | E6K, K53A, | YFGKLKSKLS VIRNLNDQVL FIDQGNRPLF EDMTDSDCRD |
| | S50H, T63N | NAPRTIFIIH MYADSQPRGM AVNISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 16 | E6K, K53A, | YFGKLKSKLS VIRNLNDQVL FIDQGNRPLF EDMTDSDCRD |
| | T63N, S65A | NAPRTIFIIS MYADSQPRGM AVNIAVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 17 | E6K, K53A, | YFGKLKSKLS VIRNLNDQVL FIDQGNRPLF EDMTDSDCRD |
| | S50H | NAPRTIFIIH MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 18 | E6K, C38A, | YFGKLKSKLS VIRNLNDQVL FIDQGNRPLF EDMTDSDARD |
| | | NAPRTIFIIS MYADSQPRGM AVTISVKAEK ISTLSCENKI |
| | K53A, C68A | ISFKEMNPPD NIKDTKSDII FFQRSVPGHD NKMQFESSSY |
| | | EGYFLACEKE RDLFKLILKK EDELGDRSIM FTVQNED |
| 19 | E6K, K53A, | YFGKLKSKLS VIRNLNDQVL FIDQGNRPLF EDMTDSDCRD |
| | K79A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 20 | E6K, K53A, | YFGKLKSKLS VIRNLNDQVL FIDQGNRPLF EDMTDSDCRD |
| | R104A | NAPRTIFIIS MYADSQPRGM AVTISVKCEK ISTLSCENAI |
| | | ISFKEMNPPD NIKDTKSDII FFQRSVPGHD NKMQFESSSY |
| | | EGYFLACEKE RDLFKLILKK EDELGDRSIM FTVQNED |
| 21 | E6K, K53A, | YFGKLKSKLS VIRNLNDQVL FIDQGNRPLF EDMTDSDCRD |
| | G108A | NAPRTIFIIS MYADSQPRGM AVTISVKCEK ISTLSCENKI |
| | | ISFKEMNPPD NIKDTKSDII FFQRSVPAHD NKMQFESSSY |
| | | EGYFLACEKE RDLFKLILKK EDELGDRSIM FTVQNED |
| 22 | E6K, K53A, | YFGKLKSKLS VIRNLNDQVL FIDQGNRPLF EDMTDSDCRD |
| | H109A | NAPRTIFIIS MYADSQPRGM AVTISVKCEK ISTLSCENKI |
| | | ISFKEMNPPD NIKDTKSDII FFQRSVPGAD NKMQFESSSY |
| | | EGYFLACEKE RDLFKLILKK EDELGDRSIM FTVQNED |
| 23 | E6K, K53A, | YFGKLKSKLS VIRNLNDQVL FIDQGNRPLF EDMTDSDCRD |
| | K112A | NAPRTIFIIS MYADSQPRGM AVTISVKCEK ISTLSCENKI |
| | | ISFKEMNPPD NIKDTKSDII FFQRSVPGHD NAMQFESSSY |
| | | EGYFLACEKE RDLFKLILKK EDELGDRSIM FTVQNED |
| 24 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A | ISFKEMNPPD NIKDTKSDII FFQRSVPGHD NKMQFESSSY |
| | | EGYFLACEKE RDLFKLILKK EDELGDRSIM FTVQNED |
| 25 | E6K, C38Q, | YFGKLKSKLS VIRNLNDQVL FIDQGNRPLF EDMTDSDQRD |
| | K53A, T63A, | NAPRTIFIIS MYADSQPRGM AVAISVKCEK ISTLSAENKI |
| | C76A | ISFKEMNPPD NIKDTKSDII FFQRSVPGHD NKMQFESSSY |
| | | EGYFLACEKE RDLFKLILKK EDELGDRSIM FTVQNED |
| 26 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSCENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 27 | E6K, C38Q, | YFGKLKSKLS VIRNLNDQVL FIDQGNRPLF EDMTDSDQRD |
| | K53A, T63A, | NAPRTIFIIS MYADSQPRGM AVAISVKCEK ISTLSCENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 28 | E6K, C38A, | YFGKLKSKLS VIRNLNDQVL FIDQGNRPLF EDMTDSDARD |
| | K53A, T63A, | NAPRTIFIIS MYADSQPRGM AVAISVKCEK ISTLSAENKI |
| | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 29 | E6K, V11I, | YFGKLKSKLS IIRNLNDQVL FIDQGNRPLF EDMTDSDARD |
| | C38A, K53A, | NAPRTIFIIS MYADSQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 30 | E6K, V11I, | YFGKLKSKLS IIRNLNDQVL FIDQGNRPLF EDMTDSDARD |
| | C38A, K53A, | NAPRTIFIIS MYADSQPRGM AVAISVKCEK ISTLSAENKI |
| | T63A, C76A, | ISFKEMNPPD NIKDTKSDII FFQRSVPGHD NKMQFESSSY |
| | C127A | EGYFLAAEKE RDLFKLILKK EDELGDRSIM FTVQNED |
| 31 | C38A, C76A, | YFGKLESKLS VIRNLNDQVL FIDQGNRPLF EDMTDSDARD |
| | C127A | NAPRTIFIIS MYKDSQPRGM AVTISVKCEK ISTLSAENKI |
| | | ISFKEMNPPD NIKDTKSDII FFQRSVPGHD NKMQFESSSY |
| | | EGYFLAAEKE RDLFKLILKK EDELGDRSIM FTVQNED |
| 32 | C38A | YFGKLESKLS VIRNLNDQVL FIDQGNRPLF EDMTDSDARD |
| | | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 33 | E6K, C38A, | YFGKLKSKLS VIRNLNDQVL FIDQGNRPLF EDMTDSDARD |
| | K53A, T63A | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 34 | E06K, K53A, | YFGKLKSKLS VIRNLNDQVL FIDQGNRPLF EDMTDSDCRD |
| | S55A | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 35 | Y01G, F02A, | GAGKLKSKLS VIRNLNDQVL FIDQGNRPLF EDMTDSDCRD |
| | E06K, M51G, | NAPRTIFIIS GYAAAQPRGM AVAISVKCEK ISTLSCENKI |
| | K53A, D54A, | ISFKEMNPPD NIKDTKSDII FFQRSVPGHD NKMQFESSSY |
| | S55A, T63A | EGYFLACEKE RDLFKLILKK EDELGDRSIM FTVQNED |
| 36 | K53A | YFGKLESKLS VIRNLNDQVL FIDQGNRPLF EDMTDSDCRD |
| | | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 37 | S55A | YFGKLESKLS VIRNLNDQVL FIDQGNRPLF EDMTDSDCRD |
| | | NAPRTIFIIS MYKDAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 38 | E06K | YFGKLKSKLS VIRNLNDQVL FIDQGNRPLF EDMTDSDCRD |
| | | NAPRTIFIIS MYKDSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 39 | E06K, K53A | YFGKLKSKLS VIRNLNDQVL FIDQGNRPLF EDMTDSDCRD |
| | | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 40 | E06K, S55A | YFGKLKSKLS VIRNLNDQVL FIDQGNRPLF EDMTDSDCRD |
| | | NAPRTIFIIS MYKDAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 41 | K53A, S55A | YFGKLESKLS VIRNLNDQVL FIDQGNRPLF EDMTDSDCRD |
| | | NAPRTIFIIS MYADAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 42 | E06K, K53A, | YFGKLKSKLS VIRNLNDQVL FIDQGNRPLF EDMTDSDCRD |
| | S55A, T63A | NAPRTIFIIS MYADAQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 43 | E06K, K53A, | GFGKLKSKLS VIRNLNDQVL FIDQGNRPLF EDMTDSDCRD |
| | | NAPRTIFIIS MYADAQPRGM AVTISVKCEK ISTLSCENKI |
| | S55A, Y01G | ISFKEMNPPD NIKDTKSDII FFQRSVPGHD NKMQFESSSY |
| | | EGYFLACEKE RDLFKLILKK EDELGDRSIM FTVQNED |
| 44 | E06K, K53A, | YAGKLKSKLS VIRNLNDQVL FIDQGNRPLF EDMTDSDCRD |
| | | NAPRTIFIIS MYADAQPRGM AVTISVKCEK ISTLSCENKI |
| | S55A, F02A | ISFKEMNPPD NIKDTKSDII FFQRSVPGHD NKMQFESSSY |
| | | EGYFLACEKE RDLFKLILKK EDELGDRSIM FTVQNED |
| 45 | E06K, K53A, | YFGKLKSKLS VIRNLNDQVL FIDQGNRPLF EDMTDSDCRD |
| | S55A, D54A | NAPRTIFIIS MYAAAQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 46 | E06K, K53A, | YFGKLKSKLS VIRNLNDQVL FIDQGNRPLF EDMTDSDCRD |
| | | NAPRTIFIIS GYADAQPRGM AVTISVKCEK ISTLSCENKI |
| | S55A, M51G | ISFKEMNPPD NIKDTKSDII FFQRSVPGHD NKMQFESSSY |
| | | EGYFLACEKE RDLFKLILKK EDELGDRSIM FTVQNED |
| 47 | C38S, C68S, | YFGKLESKLS VIRNLNDQVL FIDQGNRPLF EDMTDSDSRD |
| | | NAPRTIFIIS MYKDSQPRGM AVTISVKSEK ISTLSSENKI |
| | C76S, C127S | ISFKEMNPPD NIKDTKSDII FFQRSVPGHD NKMQFESSSY |
| | | EGYFLASEKE RDLFKLILKK EDELGDRSIM FTVQNED |
| 48 | C38S, C68S, | YFGKLESKLS VIRNLNDQVL FIDQGNRPLF EDMTDSDSRD |
| | C76S, C127S, | NAPRTIFIIS MYKDSQPRGM AVTISVKSEC ISTLSSENKI |
| | K70C | ISFKEMNPPD NIKDTKSDII FFQRSVPGHD NKMQFESSSY |
| | | EGYFLASEKE RDLFKLILKK EDELGDRSIM FTVQNED |
| 49 | E06K, K53A, | YFGKLKSKLS VIRNLNDQVL FIDQGNRPLF EDMTDSDSRD |
| | S55A, C38S, | NAPRTIFIIS MYADAQPRGM AVTISVKSEC ISTLSSENKI |
| | C68S, C76S, | ISFKEMNPPD NIKDTKSDII FFQRSVPGHD NKMQFESSSY |
| | C127S, K70C | EGYFLASEKE RDLFKLILKK EDELGDRSIM FTVQNED |
| 50 | E06K, K53A, | YFGKLKSKLS VIRNLNDQVL FIDQGNRPLF EDMTDSDCRD |
| | T63A | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 51 | T63A | YFGKLESKLS VIRNLNDQVL FIDQGNRPLF EDMTDSDCRD |
| | | NAPRTIFIIS MYKDSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 52 | E06K, T63A | YFGKLKSKLS VIRNLNDQVL FIDQGNRPLF EDMTDSDCRD |
| | | NAPRTIFIIS MYKDSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 53 | K53A, T63A | YFGKLESKLS VIRNLNDQVL FIDQGNRPLF EDMTDSDCRD |
| | | NAPRTIFIIS MYADSQPRGM AVAISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 54 | E06K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 55 | K53A, T63A, | YFGKLESKLS VIRNLNDQVL FIDQGNRPLF EDMTDSDSRD |
| | C38S, C68S, | NAPRTIFIIS MYADSQPRGM AVAISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 56 | E6K, K53A, | YFGKLKSKLS VIRNLNDQVL FIDQGNRPLF EDMTDSDSRD |
| | C38S, C76S, | NAPRTIFIIS MYADSQPRGM AVTISVKCEK ISTLSSENKI |
| | C127S | ISFKEMNPPD NIKDTKSDII FFQRSVPGHD NKMQFESSSY |
| | | EGYFLASEKE RDLFKLILKK EDELGDRSIM FTVQNED |
| 57 | E6K, C38S, | YFGKLKSKLS VIRNLNDQVL FIDQGNRPLF EDMTDSDSRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 58 | E6K, K53A, | YFGKLKSKLS VIRNLNDQVL FIDQGNRPLF EDMTDSDSRD |
| | C38S, C68S, | NAPRTIFIIS MYADSQPRGM AVTISVKSEC ISTLSSENKI |
| | C76S, C127S, | ISFKEMNPPD NIKDTKSDII FFQRSVPGHD NKMQFESSSY |
| | K70C | EGYFLASEKE RDLFKLILKK EDELGDRSIM FTVQNED |
| 59 | E6K, C38A, | YFGKLKSKLS VIRNLNDQVL FIDQGNRPLF EDMTDSDARD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 60 | E6K, C38Q, | YFGKLKSKLS VIRNLNDQVL FIDQGNRPLF EDMTDSDQRD |
| | K53A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLACEKE RDLFKLILKK EDELGDRSIM FTVQNED |
| 61 | E6K, C38A, | YFGKLKSKLS VIRNLNDQVL FIDQGNRPLF EDMTDSDARD |
| | K53A, C76A | NAPRTIFIIS MYADSQPRGM AVTISVKCEK ISTLSAENKI |
| | | ISFKEMNPPD NIKDTKSDII FFQRSVPGHD NKMQFESSSY |
| | | EGYFLACEKE RDLFKLILKK EDELGDRSIM FTVQNED |
| 62 | E6K, C38A, | YFGKLKSKLS VIRNLNDQVL FIDQGNRPLF EDMTDSDARD |
| | K53A, C127A | NAPRTIFIIS MYADSQPRGM AVTISVKCEK ISTLSCENKI |
| | | ISFKEMNPPD NIKDTKSDII FFQRSVPGHD NKMQFESSSY |
| | | EGYFLAAEKE RDLFKLILKK EDELGDRSIM FTVQNED |
| 63 | E6K, C38A, | YFGKLKSKLS VIRNLNDQVL FIDQGNRPLF EDMTDSDARD |
| | K53A, C76A, | NAPRTIFIIS MYADSQPRGM AVTISVKCEK ISTLSAENKI |
| | C127A | ISFKEMNPPD NIKDTKSDII FFQRSVPGHD NKMQFESSSY |
| | | EGYFLAAEKE RDLFKLILKK EDELGDRSIM FTVQNED |
| 64 | E6K, K53A, | YFGKLKSKLS VIRNLNDQVL FIDQGNRPLF EDMTDSDARD |
| | C38A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 65 | E6K, C38Q, | YFGKLKSKLS VIRNLNDQVL FIDQGNRPLF EDMTDSDQRD |
| | K53A, S55A, | NAPRTIFIIS MYADAQPRGM AVAISVKCEK ISTLSCENKI |
| | T63A | ISFKEMNPPD NIKDTKSDII FFQRSVPGHD NKMQFESSSY |
| | | EGYFLACEKE RDLFKLILKK EDELGDRSIM FTVQNED |
| 66 | E6K, K53A, | YFGKLKSKLS VIRNLNDQVL FIDQGNRPLF EDMTDSDCRD |
| | K84A | NAPRTIFIIS MYADSQPRGM AVTISVKCEK ISTLSCENKI |
| | | ISFAEMNPPD NIKDTKSDII FFQRSVPGHD NKMQFESSSY |
| | | EGYFLACEKE RDLFKLILKK EDELGDRSIM FTVQNED |
| 67 | E6K, K53A, | YFGKLKSKLS VIRNLNDQVL FIDQGNRPLF EDMTDSDCRD |
| | D98A | NAPRTIFIIS MYADSQPRGM AVTISVKCEK ISTLSCENKI |
| | | ISFKEMNPPD NIKDTKSAII FFQRSVPGHD NKMQFESSSY |
| | | EGYFLACEKE RDLFKLILKK EDELGDRSIM FTVQNED |
| 79 | V11I, C38A, | YFGKLESKLS IIRNLNDQVL FIDQGNRPLF EDMTDSDARD |
| (a.k.a. | M51G, K53A, | NAPRTIFIIS GYADSQPRGM AVTISVKCEK ISTLSAENKI |
| C146) | C76A, C127A | ISFKEMNPPD NIKDTKSDII FFQRSVPGHD NKMQFESSSY |
| | | EGYFLAAEKE RDLFKLILKK EDELGDRSIM FTVQNED |
| 80 | E6K, V11I, | YFGKLKSKLS IIRNLNDQVL FIDQGNRPLF EDMTDSDARD |
| (a.k.a. | C38A, M51G, | NAPRTIFIIS GYADSQPRGM AVAISVKCEK ISTLSAENKI |
| C183) | K53A, T63A, | ISFKEMNPPD NIKDTKSDII FFQRSVPGHD NKMQFESSSY |
| | C76A, C127A | EGYFLAAEKE RDLFKLILKK EDELGDRSIM FTVQNED |
| 81 | N-terminal G, | GYFGKLKSKL SIIRNLNDQV LFIDQGNRPL FEDMTDSDAR |
| (a.k.a. | E6K, V11I, | DNAPRTIFII SGYADSQPRG MAVAISVKCE KISTLSAENK |
| C192) | C38A, M51G, | IISFKEMNPP DNIKDTKSDI IFFQRSVPGH DNKMQFESSS |
| | K53A, T63A, | YEGYFLAAEK ERDLFKLILK KEDELGDRSI MFTVQNED |
| | C76A, C127A | |
| 82 | N-terminal G, | GYFGKLKSKL SIIRNLNDQV LFIDQGNRPL FEDMTDSDAR |
| (a.k.a. | E6K, V11I, | DNAPRTIFII SMYADSQPRG MAVAISVKCE KISTLSAENK |
| C141) | C38A, K53A, | IISFKEMNPP DNIKDTKSDI IFFQRSVPGH DNKMQFESSS |
| | T63A, C76A, | YEGYFLAAEK ERDLFKLILK KEDELGDRSI MFTVQNED |
| | C127A | |
| 83 | N-terminal | GGGGYFGKLK SKLSIIRNLN DQVLFIDQGN RPLFEDMTDS |
| (a.k.a. | 4xG, E6K, | DARDNAPRTI FIISMYADSQ PRGMAVAISV KCEKISTLSA |
| C140) | V11I, C38A, | ENKIISFKEM NPPDNIKDTK SDIIFFQRSV PGHDNKMQFE |
| | K53A, T63A, | SSSYEGYFLA AEKERDLFKL ILKKEDELGD RSIMFTVQNE D |
| | C76A, C127A | |
| 84 | V11I, C38A, | YFGKLESKLS IIRNLNDQVL FIDQGNRPLF EDMTDSDARD |
| | M51G, K53A, | NAPRTIFIIS GYADSQPRGM AVAISVKCEK ISTLSAENKI |
| | T63A, C76A, | ISFKEMNPPD NIKDTKSDII FFQRSVPGHD NKMQFESSS |
| | C127A | YEGYFLAAEKE RDLFKLILKK EDELGDRSIM FTVQNED |
| 85 | E6K, C38A, | YFGKLKSKLS VIRNLNDQVL FIDQGNRPLF EDMTDSDARD |
| | M51G, K53A, | NAPRTIFIIS GYADSQPRGM AVTISVKCEK ISTLSAENKI |
| | C76A, C127A | ISFKEMNPPD NIKDTKSDII FFQRSVPGHD NKMQFESSS |
| | | YEGYFLAAEKE RDLFKLILKK EDELGDRSIM FTVQNED |
| 86 | C38A, M51G, | YFGKLESKLS VIRNLNDQVL FIDQGNRPLF EDMTDSDARD |
| | K53A, C76A, | NAPRTIFIIS GYADSQPRGM AVTISVKCEK ISTLSAENKI |
| | C127A | ISFKEMNPPD NIKDTKSDII FFQRSVPGHD NKMQFESSS |
| | | YEGYFLAAEKE RDLFKLILKK EDELGDRSIM FTVQNED |

### Additional Exemplary IL-18 Constructs

Also provided herein are Act-IL-18 polypeptides which comprise the modifications to SEQ ID NO: 1 listed in the table below as part of the IL-18 polypeptide portion of the Act-IL-18, each of which is assigned a Composition ID, which can be incorporated into an activatable immunocytokine as provided herein. In some embodiments, the Act-IL-18 polypeptide of an activatable immunocytokine comprises the set of amino acid substitutions shown for any one of the constructs depicted below. In the constructs depicted below, each of the substitutions is listed using SEQ ID NO: 1 as a reference sequence. In some embodiments, the IL-18 polypeptide an activatable immunocytokine comprises only the substitutions shown for a construct below relative to SEQ ID NO: 1 (i.e., the IL-18 polypeptide has only the indicated set of substitutions and the remaining residues are those set forth in SEQ ID NO: 1).

TABLE 4
Additional IL-18 Polypeptide

| Composition ID / | | Composition ID / | | Composition ID / | |
|---|---|---|---|---|---|
| Substitutions to | | Substitutions to | | Substitutions to | |
| SEQ ID NO: 1 | | SEQ ID NO: 1 | | SEQ ID NO: 1 | |
| C143 | V11I, C38A, | C156 | V11I, C38A, N41A, | C168 | V11I, C38A, C76A, |
| | K53A, C76A, | | K53A, C76A, | | S105K, C127A |
| | C127A | | C127A | | |
| C144 | V11I, C38A, | C157 | V11I, C38A, K53A, | C174 | K8L, E6K, V11I, |
| | K53A, T63A, | | C76A, C127A, | | C38A, K53A, T63A, |
| | C76A, C127A | | D132A | | C76A, C127A |
| C145 | V11I, C38A, | C158 | V11I, C38A, K53A, | C175 | E6K, V11I, C38A, |
| | K53A, S55A, | | C76A, G108A, | | I49E, K53A, T63A, |
| | C76A, C127A | | C127A | | C76A, C127A |
| C147 | V11I, C38A, | C159 | V11I, C38A, K53A, | C176 | E6K, V11I, C38A, |
| | K53A, D54A, | | C76A, H109A, | | I49M, K53A, T63A, |
| | C76A, C127A | | C127A | | C76A, C127A |
| C148 | F2A, V11I, C38A, | C160 | V11I, C38A, K53A, | C177 | E6K, V11I, C38A, |
| | K53A, C76A, | | C76A, D110A, | | I49R, K53A, T63A, |
| | C127A | | C127A | | C76A, C127A |
| C149 | V11I, E31A, | C161 | K8R, V11I, C38A, | C178 | E6K, V11I, C38A, |
| | C38A, K53A, | | C76A, Q103E, | | K53A, T63A, C76A, |
| | C76A, C127A | | C127A | | Q103R, C127A |
| C150 | V11I, T34A, | C162 | K8E, V11I, C38A, | C179 | E6K, K8E, V11I, |
| | C38A, K53A, | | C76A, Q103R, | | C38A, K53A, T63A, |
| | C76A, C127A | | C127A | | C76A, Q103R, |
| | | | | | C127A |
| C151 | V11I, D35A, | C163 | V11I, C38A, C76A, | C180 | E6K, V11I, C38A, |
| | C38A, K53A, | | Q103K, C127A | | K53A, T63A, C76A, |
| | C76A, C127A | | | | C127A, V153R |
| C152 | V11I, S36A, | C164 | V11I, C38A, S55H, | C181 | E6K, V11I, C38A, |
| | C38A, K53A, | | C76A, C127A | | K53A, T63A, C76A, |
| | C76A, C127A | | | | C127A, V153E |
| C153 | V11I, D37A, | C165 | V11I, C38A, S55R, | C182 | E6K, V11I, C38A, |
| | C38A, K53A, | | C76A, C127A | | K53A, T63A, C76A, |
| | C76A, C127A | | | | C127A, V153Y |
| C154 | V11I, E31A, | C166 | V11I, C38A, S55T, | C184 | E6R, V11I, C38A, |
| | D37A, C38A, | | C76A, C127A | | K53A, T63A, C76A, |
| | K53A, C76A, | | | | C127A |
| | C127A | | | | |
| C155 | V11I, C38A, | C167 | V11I, C38A, C76A, | C142 | Y1M, E6K, V11I, |
| | D40A, K53A, | | S105I, C127A | | C38A, K53A, T63A, |
| | C76A, C127A | | | | C76A, C127A |
| CX90 | M51K, K53S, | CX91 | C38S, M51K, K53S, | CX92 | M3L, C38S, C68S, |
| | Q56L, P57A, | | Q56L, P57A, M60L, | | C76S, C127S, E6A, |
| | M60L, S105D, | | C68D, S105D, | | K53A |
| | D110S, N111R | | D110S, N111R | | |
| CX93 | C38M, C68S, | CX94 | C38S, C68S, C76S, | CX95 | M51A, K53G, Q56R, |
| | C76S, C127S, | | C127S, E6Q, S10C, | | P57A, M60K |
| | E6A, K53A | | K53D, N111T, | | |
| | | | N155C | | |
| CX96 | C38M, C76S, | CX97 | C38S, C76S, | CX98 | M3L, C38S, C76S, |
| | C127S, E6A, | | C127S, E6Q, S10C, | | C127S, E6A, K53A |
| | K53A | | K53D, N111T, | | |
| | | | N155C | | |

### Exemplary Act-IL-18 Sequences and Controls

| | | Blocking | Cleavage | |
|---|---|---|---|---|
| Construct | IL-18 sequence | Moiety | and/or Linker | |
| number | modifications | Sequence | sequence | Full construct sequence |
| C190 | E6K, V11I, C38A, | MAAEPVEDN | N/A | MAAEPVEDNCINFVAMKFIDNT |
| | K53A, T63A, | CINFVAMKF | | LYFIAEDDENLESDYFGKLKSK |
| | C76A, C127A | IDNTLYFIA | | LSIIRNLNDQVLFIDQGNRPLF |
| | (SEQ ID NO: 30) | EDDENLESD | | EDMTDSDARDNAPRTIFIISMY |
| | | (SEQ ID | | ADSQPRGMAVAISVKCEKISTL |
| | | NO: 89) | | SAENKIISFKEMNPPDNIKDTK |
| | | | | SDIIFFQRSVPGHDNKMQFESS |
| | | | | SYEGYFLAAEKERDLFKLILKK |
| | | | | EDELGDRSIMFTVQNED (SEQ |
| | | | | ID NO: 68) |
| C127 | E6K, V11I, C38A, | MAAEPVEDN | SGGPGPAG | MAAEPVEDNCINFVAMKFIDNT |
| | K53A, T63A, | CINFVAMKF | MKGLPGS | LYFIAEDDENLESDSGGPGPAG |
| | C76A, C127A | IDNTLYFIA | (SEQ ID | MKGLPGSYFGKLKSKLSIIRNL |
| | (SEQ ID NO: 30) | EDDENLESD | NO: 101, | NDQVLFIDQGNRPLFEDMTDSD |
| | | (SEQ ID | Linker | ARDNAPRTIFIISMYADSQPRG |
| | | NO: 89) | X | MAVAISVKCEKISTLSAENKII |
| | | | | SFKEMNPPDNIKDTKSDIIFFQ |
| | | | | RSVPGHDNKMQFESSSYEGYFL |
| | | | | AAEKERDLFKLILKKEDELGDR |
| | | | | SIMFTVQNED (SEQ ID NO: |
| | | | | 69) |
| C185 | E6K, V11I, C38A, | MAAEPVEDN | PLG | MAAEPVEDNCINFVAMKFIDNT |
| | K53A, T63A, | CINFVAMKE | | LYFIAEDDENLESDPLGYFGKL |
| | C76A, C127A | IDNTLYFIA | | KSKLSIIRNLNDQVLFIDQGNR |
| | (SEQ ID NO: 30) | EDDENLESD | | PLFEDMTDSDARDNAPRTIFII |
| | | (SEQ ID | | SMYADSQPRGMAVAISVKCEKI |
| | | NO: 89) | | STLSAENKIISFKEMNPPDNIK |
| | | | | DTKSDIIFFQRSVPGHDNKMQF |
| | | | | ESSSYEGYFLAAEKERDLFKLI |
| | | | | LKKEDELGDRSIMFTVQNED |
| | | | | (SEQ ID NO: 70) |
| C186 | Y1M, E6K, V11I, | MAAEPVEDN | PLG | MAAEPVEDNCINFVAMKFIDNT |
| | C38A, K53A, | CINFVAMKF | | LYFIAEDDENLESDPLGMEGKL |
| | T63A, C76A, | IDNTLYFIA | | KSKLSIIRNLNDQVLFIDQGNR |
| | C127A | EDDENLESD | | PLFEDMTDSDARDNAPRTIFII |
| | | (SEQ ID | | SMYADSQPRGMAVAISVKCEKI |
| | | NO: 89) | | STLSAENKIISFKEMNPPDNIK |
| | | | | DTKSDI IFFQRSVPGHDNKMQF |
| | | | | ESSSYEGYFLAAEKERDLFKLI |
| | | | | LKKEDELGDRSIMFTVQNED |
| | | | | (SEQ ID NO: 71) |
| C187 | E6K, V11I, C38A, | MAAEPVEDN | PLGLAG | MAAEPVEDNCINFVAMKFIDNT |
| | K53A, T63A, | CINFVAMKE | (SEQ ID | LYFIAEDDENLESDPLGLAGYF |
| | C76A, C127A | IDNTLYFIA | NO: 506) | GKLKSKLSIIRNLNDQVLFIDQ |
| | (SEQ ID NO: 30) | EDDENLESD | | GNRPLFEDMTDSDARDNAPRTI |
| | | (SEQ ID | | FIISMYADSQPRGMAVAISVKC |
| | | NO: 89) | | EKISTLSAENKIISFKEMNPPD |
| | | | | NIKDTKSDIIFFQRSVPGHDNK |
| | | | | MQFESSSYEGYFLAAEKERDLF |
| | | | | KLILKKEDELGDRSIMFTVQNE |
| | | | | D (SEQ ID NO: 72) |
| C172 | E6K, V11I, C38A, | SNIVPVLLG | SGGPGPAG | YFGKLKSKLSIIRNLNDQVLFI |
| | K53A, T63A, | PKLNHVAVE | MKGLPG | DQGNRPLFEDMTDSDARDNAPR |
| | C76A, C127A | LGKNVRLNC | (SEQ ID | TIFIISMYADSQPRGMAVAISV |
| | (SEQ ID NO: 30) | SALLNEEDV | NO: 100, | KCEKISTLSAENKIISFKEMNP |
| | | IYWMFGEEN | Linker | PDNIKDTKSDIIFFQRSVPGHD |
| | | GSDPNIHEE | X) | NKMQFESSSYEGYFLAAEKERD |
| | | KEMRIMTPE | | LFKLILKKEDELGDRSIMFTVQ |
| | | GKWHASKVL | | NESGGPGPAGMKGLPGSNIVPV |
| | | RIENIGESN | | LLGPKLNHVAVELGKNVRLNCS |
| | | LNVLYNCTV | | ALLNEEDVIYWMFGEENGSDPN |
| | | ASTGGTDTK | | IHEEKEMRIMTPEGKWHASKVL |
| | | SFILVRKAD | | RIENIGESNLNVLYNCTVASTG |
| | | (SEQ ID | | GTDTKSFILVRKADHHHHHH |
| | | NO: 93) | | (SEQ ID NO: 73) |
| C173 | E6K, V11I, C38A, | SNIVPVLLG | PLGLAG | YFGKLKSKLSIIRNLNDQVLFI |
| | K53A, T63A, | PKLNHVAVE | (SEQ ID | DQGNRPLFEDMTDSDARDNAPR |
| | C76A, C127A | LGKNVRLNC | NO: 506) | TIFIISMYADSQPRGMAVAISV |
| | (SEQ ID NO: 30) | SALLNEEDV | | KCEKISTLSAENKIISFKEMNP |
| | | IYWMFGEEN | | PDNIKDTKSDIIFFQRSVPGHD |
| | | GSDPNIHEE | | NKMQFESSSYEGYFLAAEKERD |
| | | KEMRIMTPE | | LFKLILKKEDELGDRSIMFTVQ |
| | | GKWHASKVL | | NEPLGLAGSNIVPVLLGPKINH |
| | | RIENIGESN | | VAVELGKNVRLNCSALLNEEDV |
| | | LNVLYNCTV | | IYWMFGEENGSDPNIHEEKEMR |
| | | ASTGGTDTK | | IMTPEGKWHASKVLRIENIGES |
| | | SFILVRKAD | | NLNVLYNCTVASTGGTDTKSFI |
| | | (SEQ ID | | LVRKADHHHHHH (SEQ ID |
| | | NO: 93) | | NO: 74) |
| C189 | E6K, V11I, C38A, | SNIVPVLLG | SGGPGPAG | YFGKLKSKLSIIRNLNDQVLFI |
| | K53A, T63A, | PKLNHVAVE | MKGLPG | DQGNRPLFEDMTDSDARDNAPR |
| | C76A, C127A | LGKNVRLDC | (SEQ ID | TIFIISMYADSQPRGMAVAISV |
| | (SEQ ID NO: 30) | SALLNEEDV | NO: 100, | KCEKISTLSAENKIISFKEMNP |
| | | IYWMFGEED | Linker | PDNIKDTKSDIIFFQRSVPGHD |
| | | GSDPNIHEE | X) | NKMQFESSSYEGYFLAAEKERD |
| | | KEMRIMTPE | | LFKLILKKEDELGDRSIMFTVQ |
| | | GKWHASKVL | | NESGGPGPAGMKGLPGSNIVPV |
| | | RIENIGESN | | LLGPKLNHVAVELGKNVRLDCS |
| | | LNVLYDCTV | | ALLNEEDVIYWMFGEEDGSDPN |
| | | ASTGGTDTK | | IHEEKEMRIMTPEGKWHASKVL |
| | | SFILVRKAD | | RIENIGESNLNVLYDCTVASTG |
| | | (SEQ ID | | GTDTKSFILVRKADHHHHHH |
| | | NO: 93) | | (SEQ ID NO: 75) |
| C191 | E6K, V11I, C38A, | SNIVPVLLG | PLGLAG | YFGKLKSKLSIIRNLNDQVLFI |
| | K53A, T63A, | PKLNHVAVE | (SEQ ID | DQGNRPLFEDMTDSDARDNAPR |
| | C76A, C127A | LGKNVRLNC | NO: 506) | TIFIISMYADSQPRGMAVAISV |
| | (SEQ ID NO: 30) | SALLNEEDV | | KCEKISTLSAENKIISFKEMNP |
| | | IYWMFGEEN | | PDNIKDTKSDIIFFQRSVPGHD |
| | | GSDPNIHEE | | NKMQFESSSYEGYFLAAEKERD |
| | | KEMRIMTPE | | LFKLILKKEDELGDRSIMFTVQ |
| | | GKWHASKVL | | NESGGPLGLAGGSNIVPVLLGP |
| | | RIENIGESN | | KLNHVAVELGKNVRLNCSALLN |
| | | LNVLYNCTV | | EEDVIYWMFGEENGSDPNIHEE |
| | | ASTGGTDTK | | KEMRIMTPEGKWHASKVLRIEN |
| | | SFILVRKAD | | IGESNLNVLYNCTVASTGGTDT |
| | | (SEQ ID | | KSFILVRKADHHHHHH (SEQ |
| | | NO: 93) | | ID NO: 76) |
| C136 | E6K, V11I, C38A, | SNIVPVLLG | GGSGG | YFGKLKSKLSIIRNLNDQVLFI |
| | K53A, T63A, | PKLNHVAVE | (SEQ ID | DQGNRPLFEDMTDSDARDNAPR |
| | C76A, C127A | LGKNVRLNC | NO: 690) | TIFIISMYADSQPRGMAVAISV |
| | (SEQ ID NO: 30) | SALLNEEDV | | KCEKISTLSAENKIISFKEMNP |
| | | IYWMFGEEN | | PDNIKDTKSDIIFFQRSVPGHD |
| | | GSDPNIHEE | | NKMQFESSSYEGYFLAAEKERD |
| | | KEMRIMTPE | | LFKLILKKEDELGDRSIMFTVQ |
| | | GKWHASKVL | | NEGGSGGSNIVPVLLGPKLNHV |
| | | RIENIGESN | | AVELGKNVRLNCSALLNEEDVI |
| | | LNVLYNCTV | | YWMFGEENGSDPNIHEEKEMRI |
| | | ASTGGTDTK | | MTPEGKWHASKVLRIENIGESN |
| | | SFILVRKAD | | LNVLYNCTVASTGGTDTKSFIL |
| | | (SEQ ID | | VRKADHHHHHH (SEQ ID |
| | | NO: 93) | | NO: 77) |
| C137 | E6K, V11I, C38A, | SNIVPVLLG | GGGGSGGG | YFGKLKSKLSIIRNLNDQVLFI |
| | K53A, T63A, | PKLNHVAVE | GSGGGG | DQGNRPLFEDMTDSDARDNAPR |
| | C76A, C127A | LGKNVRLNC | (SEQ ID | TIFIISMYADSQPRGMAVAISV |
| | (SEQ ID NO: 30) | SALLNEEDV | NO: 432) | KCEKISTLSAENKIISFKEMNP |
| | | IYWMFGEEN | | PDNIKDTKSDIIFFQRSVPGHD |
| | | GSDPNIHEE | | NKMQFESSSYEGYFLAAEKERD |
| | | KEMRIMTPE | | LFKLILKKEDELGDRSIMFTVQ |
| | | GKWHASKVL | | NEGGGGSGGGGSGGGGSNIVPV |
| | | RIENIGESN | | LLGPKLNHVAVELGKNVRLNCS |
| | | LNVLYNCTV | | ALLNEEDVIYWMFGEENGSDPN |
| | | ASTGGTDTK | | IHEEKEMRIMTPEGKWHASKVL |
| | | SFILVRKAD | | RIENIGESNLNVLYNCTVASTG |
| | | (SEQ ID | | GTDTKSFILVRKADHHHHHH |
| | | NO: 93) | | (SEQ ID NO: 78) |
| C314 | E6K, V11I, C38A, | MAAEPVE | PLG | MAAEPVEDNAINFVAMKFID |
| | K53A, T63A, | DNAINFVA | | NTLYFIAEDDENLESDPLGYF |
| | C76A, C127A | MKFIDNTL | | GKLKSKLSIIRNLNDQVLFID |
| | (SEQ ID NO: 30) | YFIAEDDE | | QGNRPLFEDMTDSDARDNAP |
| | | NLESD | | RTIFIISMYADSQPRGMAVAIS |
| | | (SEQ ID | | VKCEKISTLSAENKIISFKEMN |
| | | NO: 91) | | PPDNIKDTKSDIIFFQRSVPGH |
| | | | | DNKMQFESSSYEGYFLAAEK |
| | | | | ERDLFKLILKKEDELGDRSIM |
| | | | | FTVQNED (SEQ ID NO: 579) |
| C315 | E6K, V11I, C38A, | MAAEPVE | PLG | MAAEPVEDNSINFVAMKFID |
| | K53A, T63A, | DNSINFVA | | NTLYFIAEDDENLESDPLGYF |
| | C76A, C127A | MKFIDNTL | | GKLKSKLSIIRNLNDQVLFID |
| | (SEQ ID NO: 30) | YFIAEDDE | | QGNRPLFEDMTDSDARDNAP |
| | | NLESD | | RTIFIISMYADSQPRGMAVAIS |
| | | (SEQ ID | | VKCEKISTLSAENKIISFKEMN |
| | | NO: 90) | | PPDNIKDTKSDIIFFQRSVPGH |
| | | | | DNKMQFESSSYEGYFLAAEK |
| | | | | ERDLFKLILKKEDELGDRSIM |
| | | | | FTVQNED (SEQ ID NO: 580) |
| C316 | E6K, V11I, C38A, | MAAEPVE | SGRVANLV | MAAEPVEDNSINFVAMKFID |
| | K53A, T63A, | DNSINFVA | G (SEQ | NTLYFIAEDDENLESDSGRVA |
| | C76A, C127A | MKFIDNTL | ID NO: | NLVGYFGKLKSKLSIIRNLND |
| | (SEQ ID NO: 30) | YFIAEDDE | 646) | QVLFIDQGNRPLFEDMTDSD |
| | | NLESD | | ARDNAPRTIFIISMYADSQPR |
| | | (SEQ ID | | GMAVAISVKCEKISTLSAENK |
| | | NO: 90) | | IISFKEMNPPDNIKDTKSDIIFF |
| | | | | QRSVPGHDNKMQFESSSYEG |
| | | | | YFLAAEKERDLFKLILKKEDE |
| | | | | LGDRSIMFTVQNED (SEQ ID |
| | | | | NO: 581) |
| C317 | E6K, V11I, C38A, | MAAEPVE | RGRRVANL | MAAEPVEDNSINFVAMKFID |
| | K53A, T63A, | DNSINFVA | VG (SEQ | NTLYFIAEDDENLESDRGRRV |
| | C76A, C127A | MKFIDNTL | ID NO: | ANLVGYFGKLKSKLSIIRNLN |
| | (SEQ ID NO: 30) | YFIAEDDE | 648) | DQVLFIDQGNRPLFEDMTDS |
| | | NLESD | | DARDNAPRTIFIISMYADSQP |
| | | (SEQ ID | | RGMAVAISVKCEKISTLSAEN |
| | | NO: 90) | | KIISFKEMNPPDNIKDTKSDIIF |
| | | | | FQRSVPGHDNKMQFESSSYE |
| | | | | GYFLAAEKERDLFKLILKKED |
| | | | | ELGDRSIMFTVQNED (SEQ ID |
| | | | | NO: 582) |
| C318 | E6K, V11I, C38A, | MAAEPVE | SGRVAN | MAAEPVEDNSINFVAMKFID |
| | K53A, T63A, | DNSINFVA | (SEQ ID | NTLYFIAEDDENLESDSGRVA |
| | C76A, C127A | MKFIDNTL | NO: 650) | NYFGKLKSKLSIIRNLNDQVL |
| | (SEQ ID NO: 30) | YFIAEDDE | | FIDQGNRPLFEDMTDSDARD |
| | | NLESD | | NAPRTIFIISMYADSQPRGMA |
| | | (SEQ ID | | VAISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 583) |
| C319 | E6K, V11I, C38A, | MAAEPVE | RGRRVAN | MAAEPVEDNSINFVAMKFID |
| | K53A, T63A, | DNSINFVA | (SEQ | NTLYFIAEDDENLESDRGRRV |
| | C76A, C127A | MKFIDNTL | ID NO: | ANYFGKLKSKLSIIRNLNDQV |
| | (SEQ ID NO: 30) | YFIAEDDE | 651) | LFIDQGNRPLFEDMTDSDAR |
| | | NLESD | | DNAPRTIFIISMYADSQPRGM |
| | | (SEQ ID | | AVAISVKCEKISTLSAENKIIS |
| | | NO: 90) | | FKEMNPPDNIKDTKSDIIFFQR |
| | | | | SVPGHDNKMQFESSSYEGYF |
| | | | | LAAEKERDLFKLILKKEDELG |
| | | | | DRSIMFTVQNED (SEQ ID NO: |
| | | | | 584) |
| C320 | E6K, V11I, C38A, | MAAEPVE | SGRPLG | MAAEPVEDNSINFVAMKFID |
| | K53A, T63A, | DNSINFVA | (SEQ ID | NTLYFIAEDDENLESDSGRPL |
| | C76A, C127A | MKFIDNTL | NO: 652) | GYFGKLKSKLSIIRNLNDQVL |
| | (SEQ ID NO: 30) | YFIAEDDE | | FIDQGNRPLFEDMTDSDARD |
| | | NLESD | | NAPRTIFIISMYADSQPRGMA |
| | | (SEQ ID | | VAISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 585) |
| C321 | E6K, V11I, C38A, | MAAEPVE | RGRRPLG | MAAEPVEDNSINFVAMKFID |
| | K53A, T63A, | DNSINFVA | (SEQ ID | NTLYFIAEDDENLESDRGRRP |
| | C76A, C127A | MKFIDNTL | NO: 653) | LGYFGKLKSKLSIIRNLNDQV |
| | (SEQ ID NO: 30) | YFIAEDDE | | LFIDQGNRPLFEDMTDSDAR |
| | | NLESD | | DNAPRTIFIISMYADSQPRGM |
| | | (SEQ ID | | AVAISVKCEKISTLSAENKIIS |
| | | NO: 90) | | FKEMNPPDNIKDTKSDIIFFQR |
| | | | | SVPGHDNKMQFESSSYEGYF |
| | | | | LAAEKERDLFKLILKKEDELG |
| | | | | DRSIMFTVQNED (SEQ ID NO: |
| | | | | 586) |
| C322 | E6K, V11I, C38A, | MAAEPVE | SGRVANLV | MAAEPVEDNSINFVAMKFID |
| | K53A, T63A, | DNSINFVA | GGSG | NTLYFIAEDDENLESDSGRVA |
| | C76A, C127A | MKFIDNTL | (SEQ ID | NLVGGSGYFGKLKSKLSIIRN |
| | (SEQ ID NO: 30) | YFIAEDDE | NO: 647) | LNDQVLFIDQGNRPLFEDMT |
| | | NLESD | | DSDARDNAPRTIFIISMYADS |
| | | (SEQ ID | | QPRGMAVAISVKCEKISTLSA |
| | | NO: 90) | | ENKIISFKEMNPPDNIKDTKS |
| | | | | DIIFFQRSVPGHDNKMQFESS |
| | | | | SYEGYFLAAEKERDLFKLILK |
| | | | | KEDELGDRSIMFTVQNED |
| | | | | (SEQ ID NO: 587) |
| C323 | E6K, V11I, C38A, | MAAEPVE | RGRRVANL | MAAEPVEDNSINFVAMKFID |
| | K53A, T63A, | DNSINFVA | VGGSG | NTLYFIAEDDENLESDRGRRV |
| | C76A, C127A | MKFIDNTL | (SEQ ID | ANLVGGSGYFGKLKSKLSIIR |
| | (SEQ ID NO: 30) | YFIAEDDE | NO: 649) | NLNDQVLFIDQGNRPLFEDM |
| | | NLESD | | TDSDARDNAPRTIFIISMYAD |
| | | (SEQ ID | | SQPRGMAVAISVKCEKISTLS |
| | | NO: 90) | | AENKIISFKEMNPPDNIKDTK |
| | | | | SDIIFFQRSVPGHDNKMQFES |
| | | | | SSYEGYFLAAEKERDLFKLIL |
| | | | | KKEDELGDRSIMFTVQNED |
| | | | | (SEQ ID NO: 588) |
| C324 | E6K, V11I, C38A, | MAAEPVE | RGRRVANP | MAAEPVEDNSINFVAMKFID |
| | K53A, T63A, | DNSINFVA | LG (SEQ | NTLYFIAEDDENLESDRGRRV |
| | C76A, C127A | MKFIDNTL | ID NO: | ANPLGYFGKLKSKLSIIRNLN |
| | (SEQ ID NO: 30) | YFIAEDDE | 659) | DQVLFIDQGNRPLFEDMTDS |
| | | NLESD | | DARDNAPRTIFIISMYADSQP |
| | | (SEQ ID | | RGMAVAISVKCEKISTLSAEN |
| | | NO: 90) | | KIISFKEMNPPDNIKDTKSDIIF |
| | | | | FQRSVPGHDNKMQFESSSYE |
| | | | | GYFLAAEKERDLFKLILKKED |
| | | | | ELGDRSIMFTVQNED (SEQ ID |
| | | | | NO: 589) |
| C325 | E6K, V11I, C38A, | MAAEPVE | RGRRPLGL | MAAEPVEDNSINFVAMKFID |
| | K53A, T63A, | DNSINFVA | AGGSG | NTLYFIAEDDENLESDRGRRP |
| | C76A, C127A | MKFIDNTL | (SEQ ID | LGLAGGSGYFGKLKSKLSIIR |
| | (SEQ ID NO: 30) | YFIAEDDE | NO: 658) | NLNDQVLFIDQGNRPLFEDM |
| | | NLESD | | TDSDARDNAPRTIFIISMYAD |
| | | (SEQ ID | | SQPRGMAVAISVKCEKISTLS |
| | | NO: 90) | | AENKIISFKEMNPPDNIKDTK |
| | | | | SDIIFFQRSVPGHDNKMQFES |
| | | | | SSYEGYFLAAEKERDLFKLIL |
| | | | | KKEDELGDRSIMFTVQNED |
| | | | | (SEQ ID NO: 590) |
| C326 | E6K, V11I, C38A, | MAAEPVE | RGRRVANP | MAAEPVEDNSINFVAMKFID |
| | K53A, T63A, | DNSINFVA | LGLAGSG | NTLYFIAEDDENLESDRGRRV |
| | C76A, C127A | MKFIDNTL | (SEQ ID | ANPLGLAGSGYFGKLKSKLSI |
| | (SEQ ID NO: 30) | YFIAEDDE | NO: 657) | IRNLNDQVLFIDQGNRPLFED |
| | | NLESD | | MTDSDARDNAPRTIFIISMYA |
| | | (SEQ ID | | DSQPRGMAVAISVKCEKISTL |
| | | NO: 90) | | SAENKIISFKEMNPPDNIKDT |
| | | | | KSDIIFFQRSVPGHDNKMQFE |
| | | | | SSSYEGYFLAAEKERDLFKLI |
| | | | | LKKEDELGDRSIMFTVQNED |
| | | | | (SEQ ID NO: 591) |
| C327 | E6K, C38A, | MAAEPVE | SGRPLG | MAAEPVEDNSINFVAMKFID |
| | K53A, C76A, | DNSINFVA | (SEQ ID | NTLYFIAEDDENLESDSGRPL |
| | C127A (SEQ ID | MKFIDNTL | NO: 652) | GYFGKLKSKLSVIRNLNDQV |
| | NO: 63) | YFIAEDDE | | LFIDQGNRPLFEDMTDSDAR |
| | | NLESD | | DNAPRTIFIISMYADSQPRGM |
| | | (SEQ ID | | AVTISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 592) |
| C328 | E6K, V11I, C38A, | MAAEPVE | SGRPLG | MAAEPVEDNSINFVAMKFID |
| | M51G, K53A, | DNSINFVA | (SEQ ID | NTLYFIAEDDENLESDSGRPL |
| | T63A, C76A, | MKFIDNTL | NO: 652) | GYFGKLKSKLSIIRNLNDQVL |
| | C127A (SEQ ID | YFIAEDDE | | FIDQGNRPLFEDMTDSDARD |
| | NO: 80, a.k.a. | NLESD | | NAPRTIFIISGYADSQPRGMA |
| | C183) | (SEQ ID | | VAISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 593) |
| C329 | V11I, C38A, | MAAEPVE | SGRPLG | MAAEPVEDNSINFVAMKFID |
| | M51G, K53A, | DNSINFVA | (SEQ ID | NTLYFIAEDDENLESDSGRPL |
| | T63A, C76A, | MKFIDNTL | NO: 652) | GYFGKLESKLSIIRNLNDQVL |
| | C127A (SEQ ID | YFIAEDDE | | FIDQGNRPLFEDMTDSDARD |
| | NO: 84) | NLESD | | NAPRTIFIISGYADSQPRGMA |
| | | (SEQ ID | | VAISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 594) |
| C330 | E6K, C38A, | MAAEPVE | SGRPLG | MAAEPVEDNSINFVAMKFID |
| | M51G, K53A, | DNSINFVA | (SEQ ID | NTLYFIAEDDENLESDSGRPL |
| | C76A, C127A | MKFIDNTL | NO: 652) | GYFGKLKSKLSVIRNLNDQV |
| | (SEQ ID NO: 85) | YFIAEDDE | | LFIDQGNRPLFEDMTDSDAR |
| | | NLESD | | DNAPRTIFIISGYADSQPRGM |
| | | (SEQ ID | | AVTISVKCEKISTLSAENKIISF |
| | | NO: 90) | | KEMNPPDNIKDTKSDIIFFQRS |
| | | | | VPGHDNKMQFESSSYEGYFL |
| | | | | AAEKERDLFKLILKKEDELGD |
| | | | | RSIMFTVQNED (SEQ ID NO: |
| | | | | 595) |
| C331 | C38A, M51G, | MAAEPVE | SGRPLG | MAAEPVEDNSINFVAMKFID |
| | K53A, C76A, | DNSINFVA | (SEQ ID | NTLYFIAEDDENLESDSGRPL |
| | C127A (SEQ ID | MKFIDNTL | NO: 652) | GYFGKLESKLSVIRNLNDQVL |
| | NO: 86) | YFIAEDDE | | FIDQGNRPLFEDMTDSDARD |
| | | NLESD | | NAPRTIFIISGYADSQPRGMA |
| | | (SEQ ID | | VTISVKCEKISTLSAENKIISFK |
| | | NO: 90) | | EMNPPDNIKDTKSDIIFFQRSV |
| | | | | PGHDNKMQFESSSYEGYFLA |
| | | | | AEKERDLFKLILKKEDELGDR |
| | | | | SIMFTVQNED (SEQ ID NO: |
| | | | | 596) |
| C332 | E6K, V11I, C38A, | MAAEPVE | None | MAAEPVEDNSINFVAMKFID |
| | K53A, T63A, | DNSINFVA | | NTLYFIAEDDENLESDYFGKL |
| | C76A, C127A | MKFIDNTL | | KSKLSIIRNLNDQVLFIDQGN |
| | (SEQ ID NO: 30) | YFIAEDDE | | RPLFEDMTDSDARDNAPRTIF |
| | | NLESD | | IISMYADSQPRGMAVAISVKC |
| | | (SEQ ID | | EKISTLSAENKIISFKEMNPPD |
| | | NO: 90) | | NIKDTKSDIIFFQRSVPGHDN |
| | | | | KMQFESSSYEGYFLAAEKER |
| | | | | DLFKLILKKEDELGDRSIMFT |
| | | | | VQNED (SEQ ID NO: 597) |
| C333 | E6K, V11I, C38A, | MAAEPVE | RGRRPLG | MAAEPVEDNSINFVAMKFID |
| | K53A, T63A, | DNSINFVA | (SEQ ID | NTLYFIAEDDENLESDRGRRP |
| | C76A, C127A, C- | MKFIDNTL | NO: 653) | LGYFGKLKSKLSIIRNLNDQV |
| | terminal sortase | YFIAEDDE | | LFIDQGNRPLFEDMTDSDAR |
| | tag (SEQ ID NO: | NLESD | | DNAPRTIFIISMYADSQPRGM |
| | 30 + C-terminal | (SEQ ID | | AVAISVKCEKISTLSAENKIIS |
| | sortase tage) | NO: 90) | | FKEMNPPDNIKDTKSDIIFFQR |
| | | | | SVPGHDNKMQFESSSYEGYF |
| | | | | LAAEKERDLFKLILKKEDELG |
| | | | | DRSIMFTVQNEDGGGGSGGG |
| | | | | GSLPETGGH**** (SEQ ID NO: |
| | | | | 598) |

****In SEQ ID NO: 598, prior to conjugation to an antibody to form an activatable immunocytokine, the “GGH” positiond at the C-terminus of the sequence is replaced by a suitable glycine containing reagent with a suitable conjugation handle, such as a GGK-PEG₉-N₃ group described herein, via a sortase mediated transfer.

### Exemplary Activatable Immunocytokines and Controls

Provided below are exemplary activatable immunocytokines according to the instant disclosure, as well as corresponding control molecules (e.g., those having a non-activatable IL-18 polypeptide, such as those having an IL-18 propeptide attached via a non-cleavable linker, or IL-18 polypeptides without an artificial terminal moiety attached).

| | | Ab point of | IL-18 | IL-18 Point of | |
|---|---|---|---|---|---|
| Composition | Antibody | attachment | Polypeptide | Attachment | Linker |
| A | LZM- | K248 (EU) | C086 (Non- | C68 | Br-PEG5- |
| | 009 | | activatable | | Azide |
| | | | control) | | |
| E | LZM- | K248 (EU) | C190 | C68 | Br-PEG5- |
| | 009 | | | | Azide |
| F | LZM- | K248 (EU) | C318 | C68 | Br-PEG5- |
| | 009 | | | | Azide |
| G | LZM- | K248 (EU) | C320 | C68 | Br-PEG5- |
| | 009 | | | | Azide |
| H | LZM- | K248 (EU) | C324 | C68 | Br-PEG5- |
| | 009 | | | | Azide |
| I | LZM- | K248 (EU) | C326 | C68 | Br-PEG5- |
| | 009 | | | | Azide |
| J | LZM- | K248 (EU) | C327 | C68 | Br-PEG5- |
| | 009 | | | | Azide |
| K | LZM- | K248 (EU) | C328 | C68 | Br-PEG5- |
| | 009 | | | | Azide |
| L | LZM- | K248 (EU) | C329 | C68 | Br-PEG5- |
| | 009 | | | | Azide |
| M | LZM- | K248 (EU) | C330 | C68 | Br-PEG5- |
| | 009 | | | | Azide |
| N | LZM- | K248 (EU) | C331 | C68 | Br-PEG5- |
| | 009 | | | | Azide |
| O | LZM- | K248 (EU) | C332 | C68 | Br-PEG5- |
| | 009 | | | | Azide |
| P | LZM- | K248 (EU) | C321 | C68 | Br-PEG5- |
| | 009 | | | | Azide |
| Q | LZM- | K248 (EU) | C321 | C68 | Br-PEG11- |
| | 009 | | | | Azide |
| R | LZM- | K248 (EU) | C321 | C68 | Br-PEG24- |
| | 009 | | | | Azide |
| S | LZM- | K248 (EU) | C086 (Non- | C68 | Br-PEG24- |
| | 009 | | activatable | | Azide |
| | | | control) | | |
| T | LZM- | K248 (EU) | C333 | C-terminus | GGK- |
| | 009 | | | (Sortase | PEG9- |
| | | | | mediated) | Azide |

For each of the immunocyotines described in the table above, the conjugation to the antibody is accomplished using AJICAP™ technology to covalently attach a reactive sulfide group at residue K248 of the Fc region of the antibody (EU numbering) (i.e., a structure of —C(O)—(CH₂)ₙ—SH is added to the side chain amine of the K248 residue, wherein n is 1-6). This reactive sulfide is then reacted with a heterobifunctional linking group of the structure

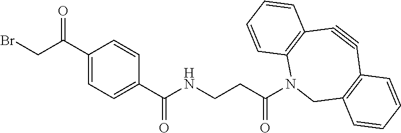 [SMILES: O=C(CBr)c1ccc(C(=O)NCCC(=O)N2Cc3ccccc3C#Cc3ccccc32)cc1]

in order to add a reactive alkyne conjugation handle. Subsequently, this alkyne bearing antibody is reacted with the IL-18 polypeptide conjugated to the linker indicated in the table above at the indicated residue of the IL-18 polypeptide. In the table above, Br-PEG5-Azide has the structure

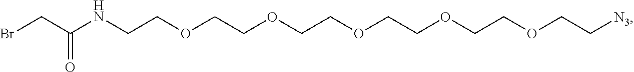 [SMILES: [N-]=[N+]=NCCOCCOCCOCCOCCOCCNC(=O)CBr]

whereas Br-PEG11-Azide and Br-PEG24-Azide having comparable structure with longer PEG chains as indicated in the molecule name (i.e., 11 and 24 ethylene glycol units respectively). For Composition T, the GGK-PEG9-Azide heterobifunctional linking group has the structure

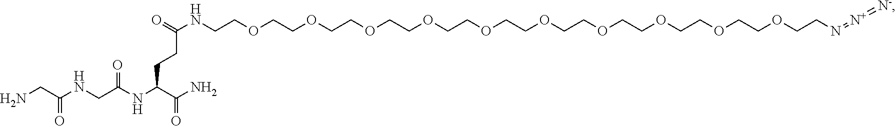

which is added to the C-terminus of the IL-18 polypeptide via a sortase mediate reaction (i.e., the GGH at the C-terminus of IL-18 polypeptide C333 is replaced with the heterobifunciontal linking group shown). As will be apparent to those of skill in the art, the GGK-PEG9-Azide linker described above could be readily substituted with another similar linker. All activatable immunocytokines described in the table above were prepared as DAR1 conjugates (i.e., a single activatable IL-18 polypeptide was added to the antibody).

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1A—Expression and Purification of Selected Activatable IL-18 Polypeptides

### Expression of IL-18 Polypeptides in Bacterial Cells

In some instances, Act-IL-18 polypeptides were produced as an N-terminal fusion to N-His-SUMO-IL18. The gene was synthesized and cloned by a commercial vendor. Plasmids were transformed into E. coli BL21 (DE3). Expression was performed in shake flasks with TB medium. The cells were grown at 37° C. until an OD600 of approximately 1.2 was reached, after which they were induced by 0.1 mM IPTG and cultured for another 20 hours at 18° C. Cells were harvested by centrifugation.

### Expression of IL-18 Polypeptides in Mammalian Cells

In some instances, Act-IL-18 polypeptides described herein were produced as a C-terminal fusion to a 6-his tag (SEQ ID NO: 687). The gene was synthesized and cloned into a proprietary vector by Genscript. Briefly, plasmids were amplified by Maxi-prep and used to transfect CHO cells. Cells were grown in serum-free Expression Medium at 37° C. with 5% CO₂ under shaking conditions. One day prior transfection, the cells were seeded at an appropriate density. On the day of transfection, DNA and transfection reagent were mixed at an optimal ratio and then added to the cells ready for transfection. Cells were harvested when the cell viability was less than 50%.

### Purification of IL18 Polypeptides Expressed in Bacterial Cells

Cell lysis-Cells were resuspended in lysis buffer (20 mM Tris/HCl, pH 8.0, 0.15 M NaCl, 10 mM Imidazole, 1 tablet of EDTA-free complete protease inhibitor (Roche, COEDTAF-RO) per liter production) at 100 mL buffer/L culture and disrupted by sonication for 15 minutes (5 sec ON/5 sec OFF/30%). The lysate was cleared of debris by centrifugation at 40′000 g for 2×45 minutes, changing flask in between, and subsequent filtration through a 0.22 μm filter.

Affinity Purification and Endotoxin Removal—The lysate was loaded on Ni NTA resin (Cytiva, 17524802) pre-equilibrated with 20 mM Tris/HCl, pH 8.0, 0.15 M NaCl, 10 mM Imidazole, at 5 mL/min and washed with the same buffer for 5 CV. To remove endotoxins, the column was washed with 20 mM Tris/HCl, pH 8.0, 0.15 M NaCl, 10 mM Imidazole, 0.1% Tryton X-114 at 10 mL/min for 30 CV. The column was washed with 20 mM Tris/HCl, pH 8.0, 0.15 M NaCl, 10 mM Imidazole, for 5 CV at 5 mL/min and the protein of interest eluted by linear increase of imidazole concentration. The column was then regenerated by 0.5M NaOH.

SUMO digestion and dialysis—To cleave the SUMO tag, SUMO protease was added to the elution pool at a w/w ratio of 1:250 (protein: SUMO enzyme) and incubated for 18 hours at 4° C. At the same time, the protein was dialysed (20 mM Tris, pH 8.0, 150 mM NaCl), to reduce the imidazole concentration.

Purification by reverse IMAC—In order to remove the cleaved tag and the SUMO protease, the digested protein was flown through a Ni NTA resin column pre-equilibrated with 20 mM Tris/HCl, pH 8.0, 0.15 M NaCl, 10 mM Imidazole, at 5 mL/min. The flow-through was collected.

Buffer Exchange—The flow-through was concentrated to approximately 2 mg/mL and buffer exchanged into either 20 mM HEPES, 150 mM NaCl, 0.5 mM TCEP, 10% glycerol, pH7.5 or PBS, 10% glycerol, pH7.4. Proteins were stored at −70° C.

### Purification of IL18 Polypeptides Expressed in Mammalian Cells

Supernatant of transfected CHO cells were clarified by centrifugation filtration. Supernatant was loaded onto an Ni NTA (Cytiva, 17524802) column at an appropriate flowrate. After washing, the protein was eluted by Imidazole gradient and buffer exchanged to the final formulation buffer (PBS pH 7.4 with 10% Glycerol). The proteins were concentrated to the desired concentration and stored at −70° C. The protein was characterized by SDS-PAGE, in reducing and non-reducing conditions, and SEC-HPLC analysis to determine the purity. The endotoxin level of protein was also detected. The concentration was determined by A280 method.

Identity of desired products is confirmed by SEC and Q-TOF Mass spectrometry.

### Example 2A—Cleavage of Activatable IL-18 Polypeptides

IL18 candidates (Tab. 1) were incubated with either MMP2 (SIGMA, PF023), MMP7 (SIGMA, CC1059), MMP9 (SIGMA, PF024), matriptasc (R&Dsystems, 3946-SEB-010) or uPA (R&Dsystems, 1310-SE-010) diluted in MMP buffer (25 mM TRIS, 10 mM CaCl2, 0.05% Brij25, pH 7.5) for the MMPs and matriptase and uPA buffer (50 mM Tris, 0.01% (v/v) Tween® 20, pH 8.5) for uPA at a final concentration of 1 μg/ml for MMP2, MMP7, MMP9, 0.1 ug/ml for matriptase and 5 ug/ml for uPA. Controls of proteins without enzymes were incubated in the same conditions. After cither 20 hours or 48 hours incubations, samples were collected for SDS gel analysis. For the candidates that presented a C-terminal masking domain, a further step of cleaning by Nickel beads was performed to remove the cleaved, histidine tagged masking domain and the non-cleaved proteins. Briefly, protein and enzyme solutions were incubated with an excess of Nickel beads (at least 10 uL dry beads for every expected 40 ug of protein) for 30 minutes in shaking conditions. The flow-through was collected and bounded residues were eluted from the beads by incubation with 20 mM TRIS, 150 mM NaCl, 500 mM Imidazole, pH 8. The digested samples were further analysed by SDS gel.

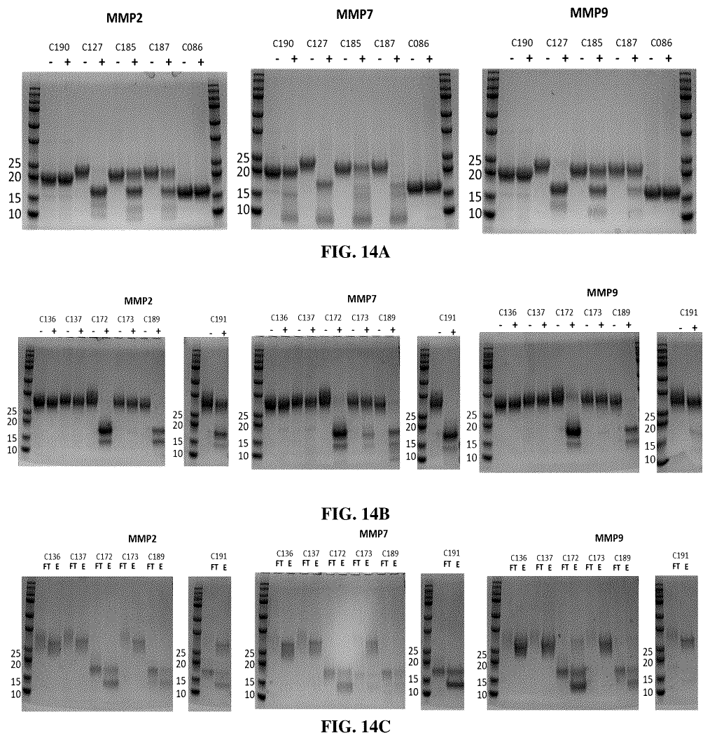

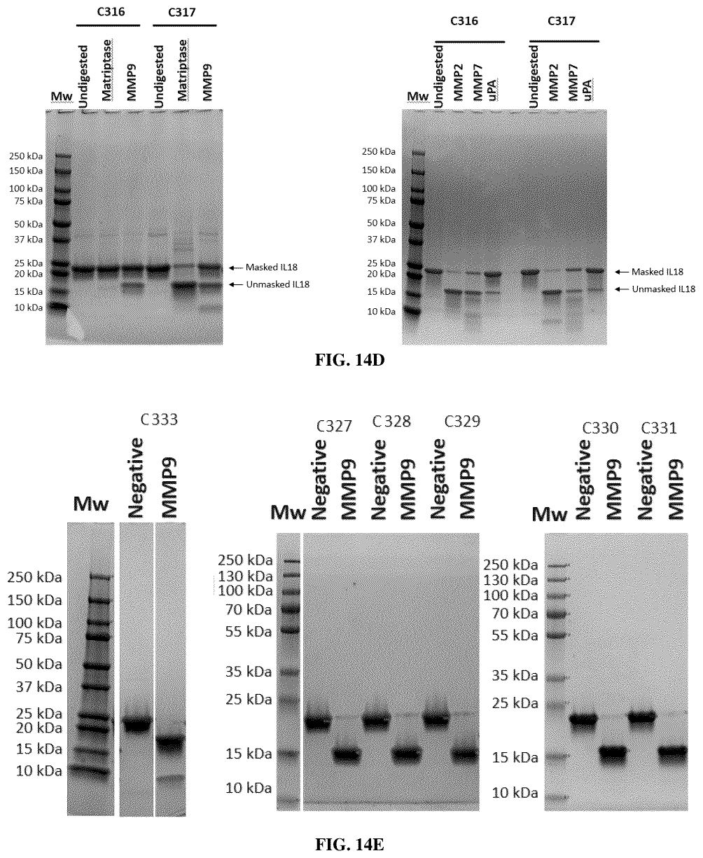

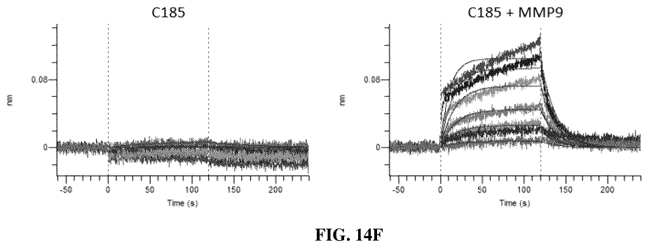

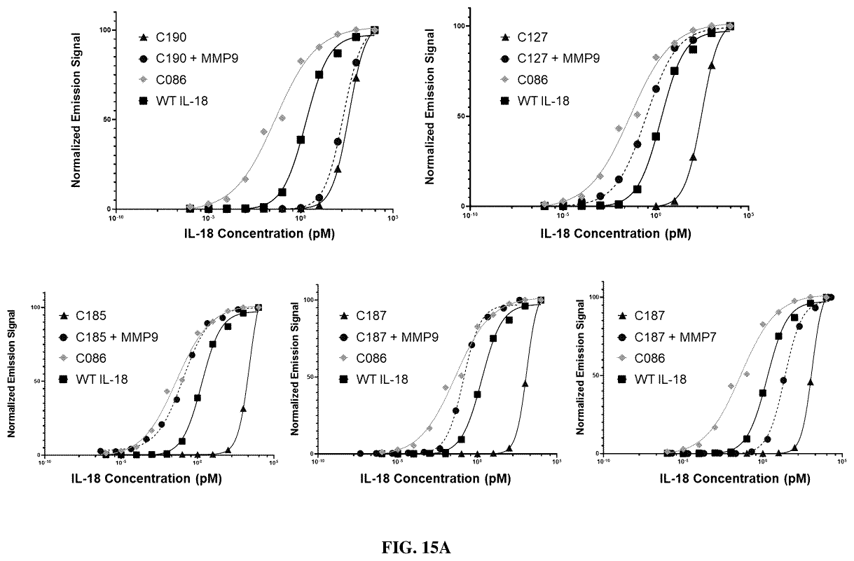

Images of gels were collected on a ChemiDoc Imaging System (BioRad) and digested protein was quantified by pixel count on ImageLab 6.1 (BioRad). Digestion percentages are reported (Tab. 3). FIGS. 14A-C show resulting SDS-PAGE gels from cleavage experiments performed on the indicated IL-18 molecules. FIG. 14A shows resulting digestion of N-terminal masked candidates by MMP2, MMP7, and MMP9, with protease treated samples indicated with a (+) and untreated samples indicated with (−). Variant C127 showed efficient cleavage by each enzyme, C185 showed moderate cleavage by each enzyme, and C187 showed moderate cleavage by MMP2 and MMP7 but moderate cleavage by MMP9, whereas base IL-18 C086 and non-cleavable control C190 remained intact in all conditions. FIG. 14B shows resulting digestion (unpurified) of C-terminal masked candidates by MMP2, MMP7, and MMP9, with protease treated samples indicated with a (+) and untreated samples indicated with (−). Variant C136 showed no cleavage by any enzyme, C137 showed no cleavage by any enzyme, C172 showed efficient cleavage by all enzymes, C173 showed some cleavage by MMP7, C189 showed efficient cleavage by all enzymes, and C191 showed moderate cleavage by MMP2, efficient cleavage by mMP7, and no/minimal cleavage by MMP9. FIG. 14C shows resulting digestion of nickel purified flow through (FT) and eluate (E) of the C-terminal masked candidates (same sample as FIG. 14B).

FIG. 14D shows a representative SDS-PAGE gel characterization of additional Act-IL-18 variants described herein, specifically C316 and C317 cleavage by matriptase, MMP9, MMP2, MMP7, and uPA depicted. Samples digested for 48 h had their cleavage percentage measured by analyzing intensities of −PAGE bands under reducing conditions, quantified using ImageLab 6.1 (BioRad) and the percentage corresponding to the cleaved species reported in the table below for additional variants tested.

| | MMP-2 | MMP-7 | MMP-9 | Matriptase | uPA |
|---|---|---|---|---|---|
| | (cleavage | (cleavage | (cleavage | (cleavage | (cleavage |
| Composition | %) | %) | %) | %) | %) |
| C190 | 0 | 0 | 0 | Not | Not |
| | | | | measured | measured |
| C127 | 100 | 100 | 100 | Not | Not |
| | | | | measured | measured |
| C185 | 57 | 6 | 60 | Not | Not |
| | | | | measured | measured |
| C187 | 59 | 100 | 20 | Not | Not |
| | | | | measured | measured |
| C136 | 0 | 0 | 0 | Not | Not |
| | | | | measured | measured |
| C137 | 0 | 0 | 0 | Not | Not |
| | | | | measured | measured |
| C172 | 100 | 100 | 93 | Not | Not |
| | | | | measured | measured |
| C173 | 0 | 27 | 0 | Not | Not |
| | | | | measured | measured |
| C189 | 100 | 100 | 100 | Not | Not |
| | | | | measured | measured |
| C191 | 43 | 100 | 0 | Not | Not |
| | | | | measured | measured |
| C314 | Not | Not | 70 | Not | Not |
| | measured | measured | | measured | measured |
| C315 | Not | Not | 65 | Not | Not |
| | measured | measured | | measured | measured |
| C316 | 80 | 34 | 23 | 0 | 12 |
| C317 | 74 | 25 | 41 | 80 | 14 |
| C318 | 90 | 0 | 38 | 0 | 49 |
| C319 | 100 | 0 | 61 | 89 | 8 |
| C320 | 100 | 11 | 100 | 0 | 0 |
| C321 | 92 | 0 | 100 | 36 | 4 |
| C322 | 95 | 90 | 58 | 4 | 25 |
| C323 | 95 | 78 | 76 | 85 | 7 |
| C324 | 88 | 0 | 100 | 76 | 10 |
| C325 | 100 | 81 | 100 | 19 | 0 |
| C326 | 100 | 50 | 100 | 81 | 0 |
| C327 | 100 | 0 | 100 | 22 | 0 |

### Example 2B

Act-IL18 candidates C185, C314, and C315 were digested as described above and characterized by BLI. Briefly, undigested and digested samples were compared for their binding against IL18 receptor on an Octet R8 (Sartorius). An Octet® High Precision Streptavidin 2.0 (SAX2) Biosensors (#18-5136) was coated with IL18 receptor alpha (Acro, IL1-H82E7) at 1 ng/ml and samples were characterized at different concentrations (from 1000 nM to 1.25 nM in 1:3 serial dilutions). A representative chromatogram of C185 before MMP treatment (left) and after MMP9 treatment (right) is shown in FIG. 14F.

### Example 3A-HEK-Blue IL18R Reporter Assay

An IL-18Rαβ positive HEK-Blue reporter cell line is used to determine binding of IL-18 variants to IL-18Rαβ and subsequent downstream signaling. The general protocol is outlined below.

5×104 cells HEK-Blue IL18R reporter cells (InvivoGen, #hkb-hmil18) are seeded into each well of a 96 well plate and stimulated with 0-100 nM of IL-18 polypeptide variants at 37° C. and 5% CO₂. After 20 h incubation, 20 μL of cell culture supernatant is then taken from each well and mixed with 180 μL QUANTI-Blue media in a 96 well plate, incubated for 1 hour at 37° C. and 5% CO₂. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Act-IL-18 polypeptides provided herein display reduced or eliminated binding ability to stimulate IFNγ compared to WT IL-18 or the IL-18 polypeptide without the artificial polypeptide. After cleavage, ability to stimulate IFNγ is restored, though may be altered relative to WT IL-18.

### Example 3B—HEK-Blue IL18R Reporter Assay—Results

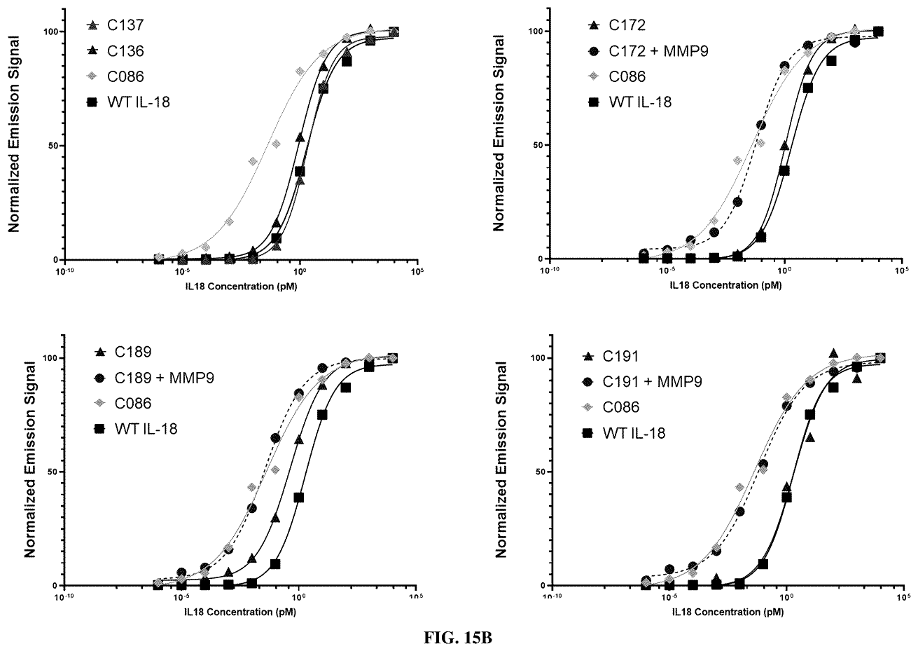

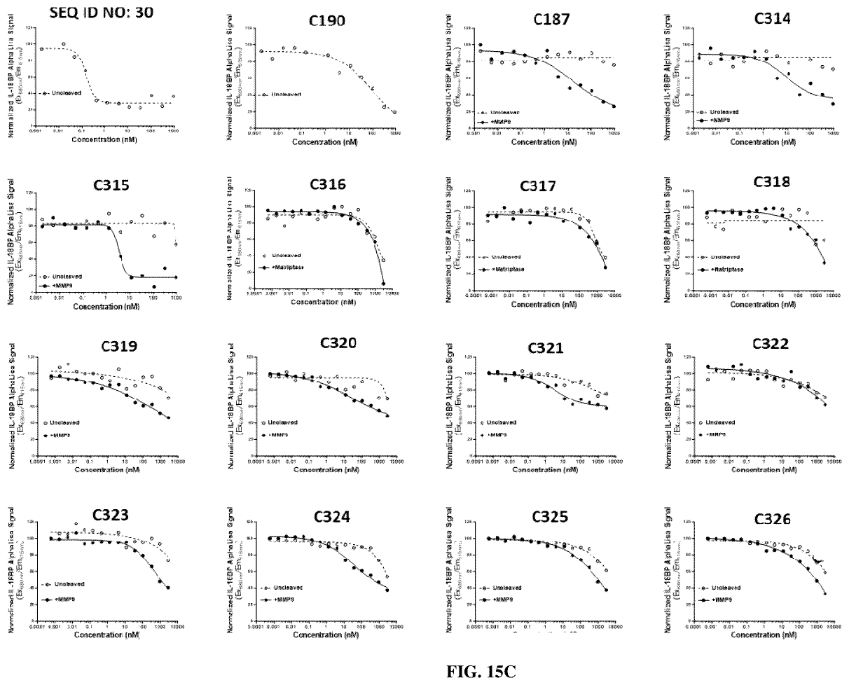

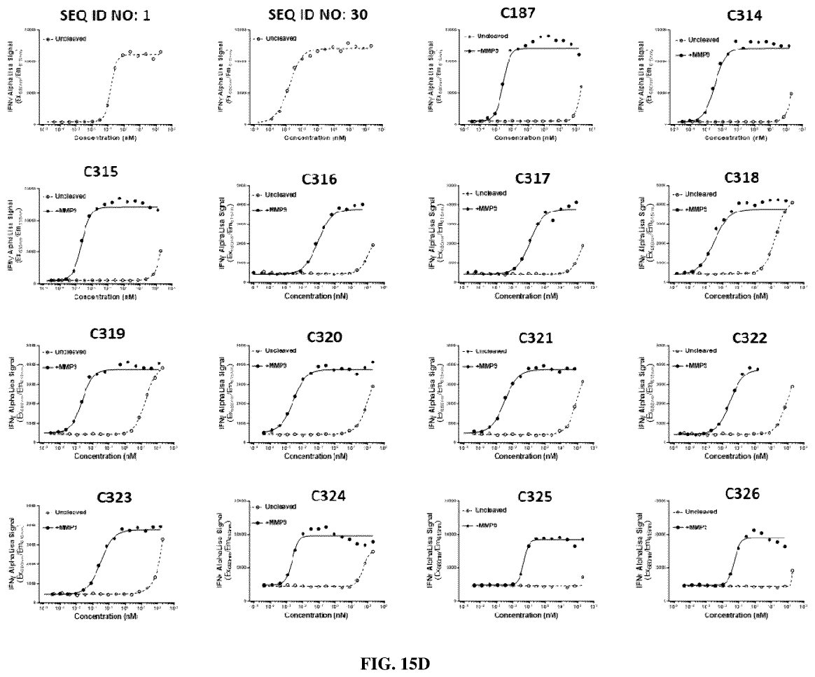

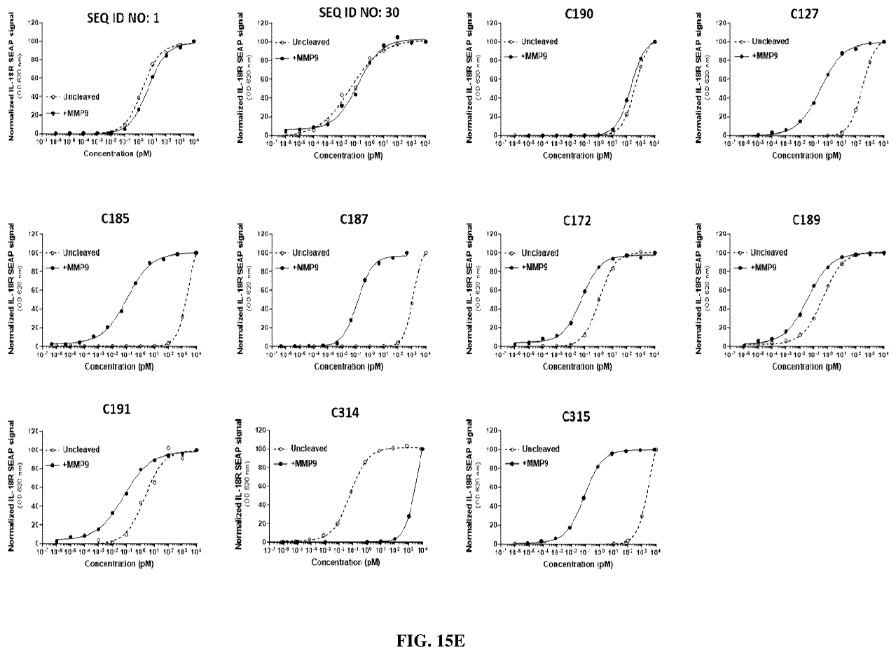

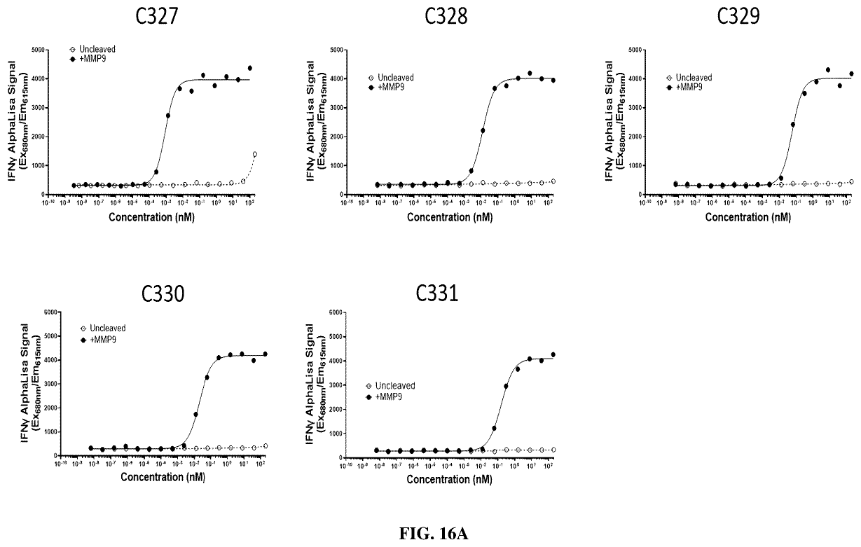

The HEK-Blue IL-18R reporter assay described above was performed on activatable and control IL-18 polypeptides before and after treatment with indicated MMPs. The activity in the HEK-Blue IL18R assays is provided in the table below. Dose response curves for these experiments for individual Act-IL-18s are shown in FIG. 15A and FIG. 15B.

| | HEK-blue | Fold potency |
|---|---|---|
| Molecule | EC50 (nM) | loss vs C086 |
| IL-18 WT | 1.89 | 45 |
| C086 (SEQ | 0.04 | — |
| ID NO: 30) | | |
| C190 | 403.5 | 9580 |
| C190 + MMP9 | 197.5 | 4689 |
| C127 | 294.7 | 6997 |
| C127 + MMP9 | 0.3007 | 7 |
| C185 | 2565 | 60897 |
| C185 + MMP9 | 0.1131 | 3 |
| C187 | 1239 | 29416 |
| C187 + MMP9 | 0.1357 | 3 |
| C187 + MMP7 | 24.27 | 576 |
| C136 | 0.8837 | 21 |
| C137 | 2.084 | 49 |
| C172 | 1.093 | 26 |
| C172 + MMP9 | 0.06126 | 1 |
| C189 | 0.4298 | 10 |
| C189 + MMP9 | 0.03635 | 1 |
| C191 | 2.015 | 48 |
| C191 + MMP9 | 0.06723 | 2 |

### Example 3C IL-18BP Binding alphaLISA Assay

A human IL-18BP AlphaLISA Assay Kit is used to determine the binding affinity of each IL-18 variant for IL-18BP, which detected the presence of free form IL-18BP.

Sixteen three-fold serial dilutions of IL-18 analytes are prepared in aMEM medium supplemented with 20% FCS, Glutamax, and 25 UM β-mercaptoethanol in the presence of 5 ng/ml of His-tagged human IL-18BP. Final IL-18 analytes concentration range from 2778 nM to 0.2 pM.

After 1 hr incubation at room temperature, free IL-18BP levels are measured using a Human IFNγ AlphaLISA Assay Kit. In a 384 well OPTIplate, 5 μL of 5× Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (KD) is calculated based on a variable slope, four parameter analysis using GraphPad PRISM software.

Act-IL-18 polypeptides provided herein may display reduced or eliminated binding to IL-18BP with the artificial polypeptide.

Binding of IL-18BP monomer determined by alphaLISA

| | | KD |
|---|---|---|
| Construct | Protease | (nM) |
| WT IL-18 | | 0.403 |
| (SEQ ID | | |
| NO: 1) | | |
| SEQ ID NO: 30 | | 22.48 |
| C190 | — | 4.33 |
| | MMP2 | |
| | MMP7 | |
| | MMP9 | |
| | Matriptase | |
| | uPA | |
| C127 | — | NT |
| | MMP2 | |
| | MMP7 | |
| | MMP9 | |
| | Matriptase | |
| | uPA | |
| C185 | — | >10000 |
| | MMP2 | |
| | MMP7 | |
| | MMP9 | 11.93 |
| | Matriptase | |
| | uPA | |
| C186 | — | NT |
| | MMP2 | |
| | MMP7 | |
| | MMP9 | |
| | Matriptase | |
| | uPA | |
| C187 | — | NT |
| | MMP2 | |
| | MMP7 | |
| | MMP9 | |
| | Matriptase | |
| | uPA | |
| C172 | — | NT |
| | MMP2 | |
| | MMP7 | |
| | MMP9 | |
| | Matriptase | |
| | uPA | |
| C173 | — | NT |
| | MMP2 | |
| | MMP7 | |
| | MMP9 | |
| | Matriptase | |
| | uPA | |
| C189 | — | NT |
| | MMP2 | |
| | MMP7 | |
| | MMP9 | |
| | Matriptase | |
| | uPA | |
| C191 | — | NT |
| | MMP2 | |
| | MMP7 | |
| | MMP9 | |
| | Matriptase | |
| | uPA | |
| C136 | — | NT |
| | MMP2 | |
| | MMP7 | |
| | MMP9 | |
| | Matriptase | |
| | uPA | |
| C137 | — | NT |
| | MMP2 | |
| | MMP7 | |
| | MMP9 | |
| | Matriptase | |
| | uPA | |
| C314 | — | >10000 |
| | MMP2 | |
| | MMP7 | |
| | MMP9 | 5.75 |
| | Matriptase | |
| | uPA | |
| C315 | — | >10000 |
| | MMP2 | |
| | MMP7 | |
| | MMP9 | 2.32 |
| | Matriptase | |
| | uPA | |
| C316 | — | 1059.85 |
| | MMP2 | |
| | MMP7 | |
| | MMP9 | 127.85 |
| | Matriptase | 722.55 |
| | uPA | |
| C317 | — | 434.80 |
| | MMP2 | |
| | MMP7 | |
| | MMP9 | 182.15 |
| | Matriptase | 287.10 |
| | uPA | |
| C318 | — | >10000 |
| | MMP2 | |
| | MMP7 | |
| | MMP9 | 14.49 |
| | Matriptase | 585.75 |
| | uPA | |
| C319 | — | 1157.00 |
| | MMP2 | |
| | MMP7 | |
| | MMP9 | 14.70 |
| | Matriptase | 435.3 |
| | uPA | |
| C320 | — | >10000 |
| | MMP2 | |
| | MMP7 | |
| | MMP9 | 25.95 |
| | Matriptase | 622.8 |
| | uPA | |
| C321 | — | >10000 |
| | MMP2 | |
| | MMP7 | |
| | MMP9 | 8.12 |
| | Matriptase | 517.95 |
| | uPA | |
| C322 | — | >10000 |
| | MMP2 | |
| | MMP7 | |
| | MMP9 | 390.65 |
| | Matriptase | 233.6 |
| | uPA | |
| C323 | — | 8550.0 |
| | MMP2 | |
| | MMP7 | |
| | MMP9 | 120.11 |
| | Matriptase | 283.59 |
| | uPA | |
| C324 | — | 1716 |
| | MMP2 | |
| | MMP7 | |
| | MMP9 | 36.55 |
| | Matriptase | |
| | uPA | |
| C325 | — | 1885 |
| | MMP2 | |
| | MMP7 | |
| | MMP9 | 157.6 |
| | Matriptase | |
| | uPA | |
| C326 | — | 2100 |
| | MMP2 | |
| | MMP7 | |
| | MMP9 | 139.0 |
| | Matriptase | 887.9 |
| | uPA | |

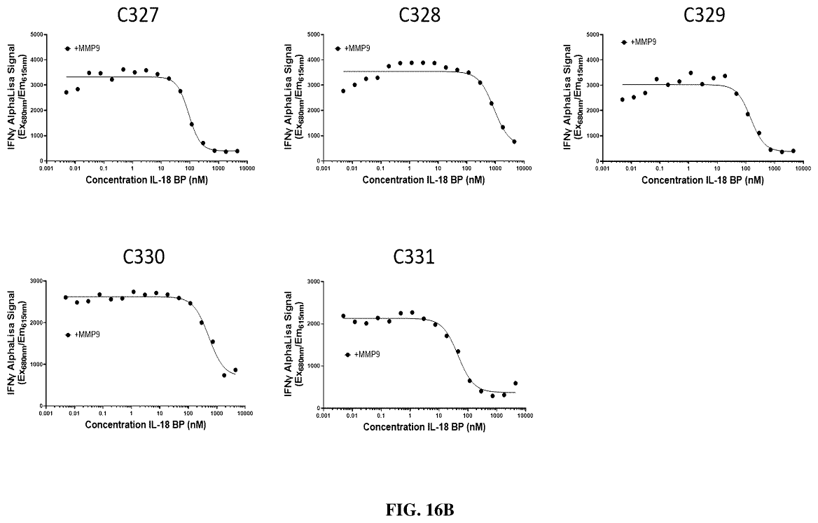

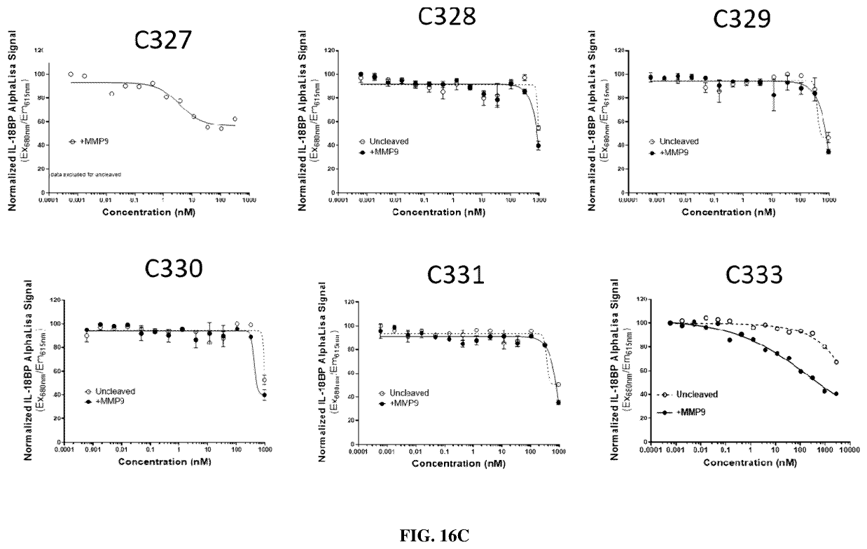

FIG. 15C shows plots measuring the ability of the MMP9 cleaved (solid black round symbols and black lines) and uncleaved (open round symbols and dotted black lines) masked IL-18 polypeptides to bind to the human IL-18 Binding Protein (IL-18BP). The figure shows free IL-18BP AlphaLISA signal on the y-axis, and dosage of IL-18 polypeptides on the x-axis. FIG. 16C shows similar plots for additional variants.

### Example 3D—IFNγ Induction Cellular Assay

The ability of IL-18 polypeptides provided herein are assessed for ability to induce IFN in a cellular assay according to the protocol below.

The NK cell line NK-92 derived from a patient with lymphoma (ATCC® CR^{L}-2407™) is cultured in aMEM medium supplemented with 20% FCS, Glutamax, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, cells are seeded at 100,000 cells/well in a 384 well titer plate and incubated at 37° C./5% CO₂. Sixteen 4-fold serial dilutions of IL-18 analytes are prepared in aMEM medium, and 1 ng/mL of IL-12 were added to the NK-92 cells. Final IL-18 analyte concentrations range from 56 nM to 5×10-5 pM.

After incubating the cells for 16-20 hr at 37° C./5% CO2, 5 μL of supernatant is carefully transferred to a 384 microwell OptiPlate. IFNγ levels are measured using a human IFNγ AlphaLISA Assay Kit. Briefly, 10 μL of 2.5× AlphaLISA Anti-IFNγ acceptor beads and biotinylated antibody anti-IFNγ mix are added to the 5 μL of NK-92 supernatants. The mixtures are incubated for 1 hr at room temperature with shaking. Under subdued light, 2.5 μL of 2× streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC50) are calculated based on a variable slope and four parameter analysis using GraphPad PRISM software.

Act-IL-18 polypeptides provided herein display reduced or eliminated binding ability to stimulate IFNγ compared to WT IL-18 or the IL-18 polypeptide without the artificial polypeptide. After cleavage, ability to stimulate IFNγ is restored, though may be altered relative to WT IL-18.

| | | IFNγ release |
|---|---|---|
| | | NK-92 assay |
| Construct | Protease | EC₅₀ (nM) |
| SEQ ID NO: 1 | | 0.3307 |
| SEQ ID NO: 30 | | 0.0018 |
| C190 | — | NT |
| | MMP2 | |
| | MMP7 | |
| | MMP9 | |
| | Matriptase | |
| | uPA | |
| C127 | — | NT |
| | MMP2 | |
| | MMP7 | |
| | MMP9 | |
| | Matriptase | |
| | uPA | |
| C185 | — | 210.1 |
| | MMP2 | |
| | MMP7 | |
| | MMP9 | 0.0026 |
| | Matriptase | |
| | uPA | |
| C186 | — | NT |
| | MMP2 | |
| | MMP7 | |
| | MMP9 | |
| | Matriptase | |
| | uPA | |
| C187 | — | NT |
| | MMP2 | |
| | MMP7 | |
| | MMP9 | |
| | Matriptase | |
| | uPA | |
| C172 | — | NT |
| | MMP2 | |
| | MMP7 | |
| | MMP9 | |
| | Matriptase | |
| | uPA | |
| C173 | — | NT |
| | MMP2 | |
| | MMP7 | |
| | MMP9 | |
| | Matriptase | |
| | uPA | |
| C189 | — | NT |
| | MMP2 | |
| | MMP7 | |
| | MMP9 | |
| | Matriptase | |
| | uPA | |
| C191 | — | NT |
| | MMP2 | |
| | MMP7 | |
| | MMP9 | |
| | Matriptase | |
| | uPA | |
| C136 | — | NT |
| | MMP2 | |
| | MMP7 | |
| | MMP9 | |
| | Matriptase | |
| | uPA | |
| C137 | — | NT |
| | MMP2 | |
| | MMP7 | |
| | MMP9 | |
| | Matriptase | |
| | uPA | |
| C314 | — | 255 |
| | MMP2 | |
| | MMP7 | |
| | MMP9 | 0.0032 |
| | Matriptase | |
| | uPA | |
| C315 | — | 251.9 |
| | MMP2 | |
| | MMP7 | |
| | MMP9 | 0.0022 |
| | Matriptase | |
| | uPA | |
| C316 | — | 531.90 |
| | MMP2 | |
| | MMP7 | |
| | MMP9 | 0.1972 |
| | Matriptase | 366.20 |
| | uPA | |
| C317 | — | 526.50 |
| | MMP2 | |
| | MMP7 | |
| | MMP9 | 0.1929 |
| | Matriptase | 6.338 |
| | uPA | |
| C318 | — | 21.93 |
| | MMP2 | |
| | MMP7 | |
| | MMP9 | 0.0053 |
| | Matriptase | 22.055 |
| | uPA | |
| C319 | — | 21.285 |
| | MMP2 | |
| | MMP7 | |
| | MMP9 | 0.0032 |
| | Matriptase | 5.911 |
| | uPA | |
| C320 | — | 157.60 |
| | MMP2 | |
| | MMP7 | |
| | MMP9 | 0.0034 |
| | Matriptase | 292.35 |
| | uPA | |
| C321 | — | 111.59 |
| | MMP2 | |
| | MMP7 | |
| | MMP9 | 0.0041 |
| | Matriptase | 1.748 |
| | uPA | |
| C322 | — | 204.60 |
| | MMP2 | |
| | MMP7 | |
| | MMP9 | 0.0577 |
| | Matriptase | 13.775 |
| | uPA | |
| C323 | — | 102.20 |
| | MMP2 | |
| | MMP7 | |
| | MMP9 | 0.0636 |
| | Matriptase | 3.444 |
| | uPA | |
| C324 | — | 51.67 |
| | MMP2 | |
| | MMP7 | |
| | MMP9 | 0.0022 |
| | Matriptase | |
| | uPA | |
| C325 | — | 201.2 |
| | MMP2 | |
| | MMP7 | |
| | MMP9 | 0.0467 |
| | Matriptase | 6.924 |
| | uPA | |
| C326 | — | 217.3 |
| | MMP2 | |
| | MMP7 | |
| | MMP9 | 0.0514 |
| | Matriptase | 2.834 |
| | uPA | |

FIG. 15D shows plots measuring the ability of the MMP9 cleaved (solid black round symbols and black lines) and uncleaved (open round symbols and dotted black lines) masked IL-18 polypeptides to stimulate the secretion of IFNgamma by NK-92 cells. The figure shows mean IFNg signal on the y-axis and dosage of the IL-18 polypeptides on the x-axis. FIG. 16A shows similar plats for additional variants.

### Example 3E-IL-18 Binding Protein Inhibition Cellular Assay

The NK cell line NK-92 derived from a patient with lymphoma (ATCC® CR^{L}-2407™) is cultured in aMEM medium supplemented with 20% FCS-Glutamax, 25 UM B-mercaptoethanol, and 100 IU/mL of recombinant human IL-2.

On the day of experiment, cells are harvested and washed with aMEM medium without IL-2 and containing 1 ng/ml of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in aMEM medium. 1 ng/ml of IL-12 containing 2 nM of each Act-IL-18 polypeptide variant is added to the NK-92 cells. The final IL-18 analyte concentration is 1 nM, and the final IL-18BPa concentration ranged from 566 nM to 17 pM.

After incubating the cells for 16-20 hr at 37° C./5% CO2, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2×SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC50) are calculated based on a variable slope and four parameter analysis using GraphPad PRISM software.

Act-IL-18 variants of the disclosure are active and able to induce IFNγ secretion in vitro after cleavage of the artificial polypeptide, but display reduces or no ability to induce IFNγ without cleavage.

| | | IL-18BP-mediated |
|---|---|---|
| | | IFNγ inhibition |
| | | NK-92 assay |
| | | IC₅₀ |
| Construct | Protease | (nM) |
| SEQ ID NO: 1 | | 0.403 |
| SEQ ID NO: 30 | | 22.48 |
| C190 | — | 4.33 |
| | MMP2 | |
| | MMP7 | |
| | MMP9 | |
| | Matriptase | |
| | uPA | |
| C127 | — | NT |
| | MMP2 | |
| | MMP7 | |
| | MMP9 | |
| | Matriptase | |
| | uPA | |
| C185 | — | NT |
| | MMP2 | |
| | MMP7 | |
| | MMP9 | 186.7 |
| | Matriptase | |
| | uPA | |
| C186 | — | NT |
| | MMP2 | |
| | MMP7 | |
| | MMP9 | |
| | Matriptase | |
| | uPA | |
| C187 | — | NT |
| | MMP2 | |
| | MMP7 | |
| | MMP9 | |
| | Matriptase | |
| | uPA | |
| C172 | — | NT |
| | MMP2 | |
| | MMP7 | |
| | MMP9 | |
| | Matriptase | |
| | uPA | |
| C173 | — | NT |
| | MMP2 | |
| | MMP7 | |
| | MMP9 | |
| | Matriptase | |
| | uPA | |
| C189 | — | NT |
| | MMP2 | |
| | MMP7 | |
| | MMP9 | |
| | Matriptase | |
| | uPA | |
| C191 | — | NT |
| | MMP2 | |
| | MMP7 | |
| | MMP9 | |
| | Matriptase | |
| | uPA | |
| C136 | — | NT |
| | MMP2 | |
| | MMP7 | |
| | MMP9 | |
| | Matriptase | |
| | uPA | |
| C137 | — | NT |
| | MMP2 | |
| | MMP7 | |
| | MMP9 | |
| | Matriptase | |
| | uPA | |
| C314 | — | NT |
| | MMP2 | |
| | MMP7 | |
| | MMP9 | 120.0 |
| | Matriptase | |
| | uPA | |
| C315 | — | NT |
| | MMP2 | |
| | MMP7 | |
| | MMP9 | 117.8 |
| | Matriptase | |
| | uPA | |
| C316 | — | NT |
| | MMP2 | |
| | MMP7 | |
| | MMP9 | 0.5546 |
| | Matriptase | |
| | uPA | |
| C317 | — | NA |
| | MMP2 | |
| | MMP7 | |
| | MMP9 | 0.4255 |
| | Matriptase | |
| | uPA | |
| C318 | — | NT |
| | MMP2 | |
| | MMP7 | |
| | MMP9 | 29.36 |
| | Matriptase | |
| | uPA | |
| C319 | — | NT |
| | MMP2 | |
| | MMP7 | |
| | MMP9 | 62.96 |
| | Matriptase | |
| | uPA | |
| C320 | — | NT |
| | MMP2 | |
| | MMP7 | |
| | MMP9 | 110.9 |
| | Matriptase | |
| | uPA | |
| C321 | — | NT |
| | MMP2 | |
| | MMP7 | |
| | MMP9 | 83.28 |
| | Matriptase | |
| | uPA | |
| C322 | — | NT |
| | MMP2 | |
| | MMP7 | |
| | MMP9 | 3.710 |
| | Matriptase | |
| | uPA | |
| C323 | — | NT |
| | MMP2 | |
| | MMP7 | |
| | MMP9 | 2.648 |
| | Matriptase | |
| | uPA | |
| C324 | — | NT |
| | MMP2 | |
| | MMP7 | |
| | MMP9 | |
| | Matriptase | |
| | uPA | |
| C325 | — | NT |
| | MMP2 | |
| | MMP7 | |
| | MMP9 | |
| | Matriptase | |
| | uPA | |
| C326 | — | NT |
| | MMP2 | |
| | MMP7 | |
| | MMP9 | |
| | Matriptase | |
| | uPA | |

FIG. 16B shows plots measuring the ability of increasing concentrations of human recombinant IL-18 binding protein (IL-18BP) to inhibit the secretion of IFNgamma by NK-92 cells stimulated with 1 nM of the MMP9 cleaved (solid black round symbols and black lines) and uncleaved (open round symbols and dotted black lines) masked IL-18 polypeptides. The figure shows mean IFNg signal on the y-axis and dosage of IL-18BP on the x-axis.

### Example 3F—HEK-Blue IL18R Reporter Assay

An IL-18Rαβ positive HEK-Blue reporter cell line is used to determine binding of IL-18 variants to IL-18Rαβ and subsequent downstream signaling. The general protocol is outlined below.

5×10⁴ cells HEK-Blue IL18R reporter cells (InvivoGen, #hkb-hmil18) are seeded into each well of a 96 well plate and stimulated with 0-100 nM of IL-18 polypeptide variants at 37° C. and 5% CO2. After 20 h incubation, 20 μL of cell culture supernatant is then taken from each well and mixed with 180 μL QUANTI-Blue media in a 96 well plate, incubated for 1 hour at 37° C. and 5% CO2. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Act-IL-18 polypeptides provided herein display reduced or eliminated binding ability to stimulate IFNγ compared to WT IL-18 or the IL-18 polypeptide without the artificial polypeptide. After cleavage, ability to stimulate IFNγ is restored, though may be altered relative to WT IL-18.

TABLE
EC₅₀ in HEK-Blue IL18R Reporter Assay Data

| | | Hek Blue reporter |
|---|---|---|
| | | assay |
| | | EC₅₀ |
| Construct | Protease | (pM) |
| SEQ ID NO: 1 | | 2.35 |
| SEQ ID NO: 30 | | 0.057 |
| C190 | — | 403.5 |
| | MMP2 | |
| | MMP7 | |
| | MMP9 | 197.5 |
| | Matriptase | |
| | uPA | |
| C127 | — | 294.7 |
| | MMP2 | |
| | MMP7 | |
| | MMP9 | 0.3007 |
| | Matriptase | |
| | uPA | |
| C185 | — | 2789 |
| | MMP2 | |
| | MMP7 | |
| | MMP9 | 0.0941 |
| | Matriptase | |
| | uPA | |
| C186 | — | NT |
| | MMP2 | |
| | MMP7 | |
| | MMP9 | |
| | Matriptase | |
| | uPA | |
| C187 | — | 1239 |
| | MMP2 | |
| | MMP7 | 24.57 |
| | MMP9 | 0.1357 |
| | Matriptase | |
| | uPA | |
| C172 | — | 1.093 |
| | MMP2 | |
| | MMP7 | |
| | MMP9 | 0.0570 |
| | Matriptase | |
| | uPA | |
| C173 | — | 0.1301 |
| | MMP2 | |
| | MMP7 | |
| | MMP9 | |
| | Matriptase | |
| | uPA | |
| C189 | — | 0.4298 |
| | MMP2 | |
| | MMP7 | |
| | MMP9 | 0.0364 |
| | Matriptase | |
| | uPA | |
| C191 | — | 2.015 |
| | MMP2 | |
| | MMP7 | |
| | MMP9 | 0.0672 |
| | Matriptase | |
| | uPA | |
| C136 | — | 0.8837 |
| | MMP2 | |
| | MMP7 | |
| | MMP9 | |
| | Matriptase | |
| | uPA | |
| C137 | — | 2.084 |
| | MMP2 | |
| | MMP7 | |
| | MMP9 | |
| | Matriptase | |
| | uPA | |
| C314 | — | 3997 |
| | MMP2 | |
| | MMP7 | |
| | MMP9 | 0.0627 |
| | Matriptase | |
| | uPA | |
| C315 | — | 3360 |
| | MMP2 | |
| | MMP7 | |
| | MMP9 | 0.0827 |
| | Matriptase | |
| | uPA | |
| C316 | — | NT |
| | MMP2 | |
| | MMP7 | |
| | MMP9 | |
| | Matriptase | |
| | uPA | |
| C317 | — | NT |
| | MMP2 | |
| | MMP7 | |
| | MMP9 | |
| | Matriptase | |
| | uPA | |
| C318 | — | NT |
| | MMP2 | |
| | MMP7 | |
| | MMP9 | |
| | Matriptase | |
| | uPA | |
| C319 | — | NT |
| | MMP2 | |
| | MMP7 | |
| | MMP9 | |
| | Matriptase | |
| | uPA | |
| C320 | — | NT |
| | MMP2 | |
| | MMP7 | |
| | MMP9 | |
| | Matriptase | |
| | uPA | |
| C321 | — | NT |
| | MMP2 | |
| | MMP7 | |
| | MMP9 | |
| | Matriptase | |
| | uPA | |
| C322 | — | NT |
| | MMP2 | |
| | MMP7 | |
| | MMP9 | |
| | Matriptase | |
| | uPA | |
| C323 | — | NT |
| | MMP2 | |
| | MMP7 | |
| | MMP9 | |
| | Matriptase | |
| | uPA | |
| C324 | — | NT |
| | MMP2 | |
| | MMP7 | |
| | MMP9 | |
| | Matriptase | |
| | uPA | |
| C325 | — | |
| | MMP2 | |
| | MMP7 | |
| | MMP9 | |
| | Matriptase | |
| | uPA | |
| C326 | — | |
| | MMP2 | |
| | MMP7 | |
| | MMP9 | |
| | Matriptase | |
| | uPA | |

FIG. 15E shows plots measuring the ability of the MMP9 cleaved (solid black round symbols and black lines) and uncleaved (open round symbols and dotted black lines) masked IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (OD 620 nm) on the y-axis, and dosage of the masked and unmasked IL-18 polypeptides on the x-axis.

### Example 3G: IFNγ Induction on Primary Human Cells

Ability of IL-18 variants to stimulate Human peripheral blood mononuclear cells (PBMCs) was assessed according to the following protocol.

Isolation of lymphocytes: Blood from Buffy Coats of healthy volunteers was diluted with equal volume of PBS and slowly poured on top of SepMate tube prefilled with 15 mL Histopaque-1077. Tubes were centrifuged for 10 minutes at 1200 g, the top layer was collected and washed 3 times with PBS containing 2% of Fetal Bovine Serum. PBMCs were counted and cryopreserved as aliquots of 20×10⁶ cells.

Cryopreserved PBMCs were thawed and seeded at 150 000 cells/well in a 96w round bottom 96 well plate. PBMCs were stimulated with a gradient of human IL-18 variants ranging from 0.2 pg/mL to 3600 ng/mL. All stimulations were performed in the presence of hIL-12 (1 ng/ml, Sino Biological, #CT011-H08H) for 24 hrs in RPMI containing 10% Fetal Bovine Serum.

Cytokine production after 24 hr stimulation were measured using Legendplex bead-based cytokine assay (Biolegend #740930) according to manufacturer protocol. Half maximal effective concentrations (EC₅₀) of IFNg released in culture supernatant were calculated based on a variable slope and four parameter analysis using GraphPad PRISM software.

TABLE
EC₅₀ in PBMC IFNg secretion Assay$

| | | IFNg release |
|---|---|---|
| | | PBMCs assay |
| | | EC₅₀ |
| Construct | Protease | (nM) |
| SEQ ID NO: 1 | | 0.698 |
| SEQ ID NO: 30 | | 0.0022 |
| C190 | — | |
| | MMP2 | |
| | MMP7 | |
| | MMP9 | |
| | Matriptase | |
| | uPA | |
| C127 | — | NT |
| | MMP2 | |
| | MMP7 | |
| | MMP9 | |
| | Matriptase | |
| | uPA | |
| C185 | — | NT |
| | MMP2 | |
| | MMP7 | |
| | MMP9 | |
| | Matriptase | |
| | uPA | |
| C186 | — | NT |
| | MMP2 | |
| | MMP7 | |
| | MMP9 | |
| | Matriptase | |
| | uPA | |
| C187 | — | NT |
| | MMP2 | |
| | MMP7 | |
| | MMP9 | |
| | Matriptase | |
| | uPA | |
| C172 | | NT |
| | MMP2 | |
| | MMP7 | |
| | MMP9 | |
| | Matriptase | |
| | uPA | |
| C173 | — | NT |
| | MMP2 | |
| | MMP7 | |
| | MMP9 | |
| | Matriptase | |
| | uPA | |
| C189 | — | NT |
| | MMP2 | |
| | MMP7 | |
| | MMP9 | |
| | Matriptase | |
| | uPA | |
| C191 | — | NT |
| | MMP2 | |
| | MMP7 | |
| | MMP9 | |
| | Matriptase | |
| | uPA | |
| C136 | — | NT |
| | MMP2 | |
| | MMP7 | |
| | MMP9 | |
| | Matriptase | |
| | uPA | |
| C137 | — | NT |
| | MMP2 | |
| | MMP7 | |
| | MMP9 | |
| | Matriptase | |
| | uPA | |
| C314 | — | NT |
| | MMP2 | |
| | MMP7 | |
| | MMP9 | |
| | Matriptase | |
| | uPA | |
| C315 | — | NT |
| | MMP2 | |
| | MMP7 | |
| | MMP9 | |
| | Matriptase | |
| | uPA | |
| C316 | — | NT |
| | MMP2 | |
| | MMP7 | |
| | MMP9 | |
| | Matriptase | |
| | uPA | |
| C317 | — | NT |
| | MMP2 | |
| | MMP7 | |
| | MMP9 | |
| | Matriptase | |
| | uPA | |
| C318 | — | NT |
| | MMP2 | |
| | MMP7 | |
| | MMP9 | |
| | Matriptase | |
| | uPA | |
| C319 | — | NT |
| | MMP2 | |
| | MMP7 | |
| | MMP9 | |
| | Matriptase | |
| | uPA | |
| C320 | — | NT |
| | MMP2 | |
| | MMP7 | |
| | MMP9 | |
| | Matriptase | |
| | uPA | |
| C321 | — | NT |
| | MMP2 | |
| | MMP7 | |
| | MMP9 | |
| | Matriptase | |
| | uPA | |
| C322 | — | NT |
| | MMP2 | |
| | MMP7 | |
| | MMP9 | |
| | Matriptase | |
| | uPA | |
| C323 | — | NT |
| | MMP2 | |
| | MMP7 | |
| | MMP9 | |
| | Matriptase | |
| | uPA | |
| C324 | — | NT |
| | MMP2 | |
| | MMP7 | |
| | MMP9 | |
| | Matriptase | |
| | uPA | |
| C325 | — | |
| | MMP2 | |
| | MMP7 | |
| | MMP9 | |
| | Matriptase | |
| | uPA | |
| C326 | — | |
| | MMP2 | |
| | MMP7 | |
| | MMP9 | |
| | Matriptase | |
| | uPA | |

### Example 4—Stability Assessment of IL-18s

The stability of activatable and control IL-18 polypeptides was assessed using nano differential scanning fluorimity (nanoDSF). Activatable IL-18 polypeptide constructs with either the propeptide or IL-18 receptor D3 subunit attached showed enhanced stability compared to C086. Conversely, IL-18 polypeptides with a short extension peptide attached to the N-terminus showed lower stability.

| | | | ΔTm |
|---|---|---|---|
| | | Tm | vs |
| Sample | Protein | 550 nm | C086 |
| name | Description | (° C.) | (° C.) |
| C086 | C086 | 54.4 | |
| C190 | Propeptide-C086 | 62.2 | 7.8 |
| C172 | C086-LinkerX- | 61 | 6.6 |
| | RaD3 | | |
| C140 | GGGG-C086 | 51.3 | −3.1 |
| C141 | G-C086 | 52 | −2.4 |
| C142 | C086_Y1M | 54.9 | 0.5 |

In some instances, IL-18 polypeptides were subject to treatment with MMP or in MMP buffer without MMP according to the following general protocol: 100 uL samples of the indicated IL-18 at approximately 1 mg/mL were mixed with 100 μL of MMP-2 at 2 μg/mL in an MMP assay buffer (25 mM TRIS, 10 mM CaCl₂), 0.05% Brij 25, pH 7.5). Samples were incubated 16 hours in shaking conditions at 24° C.

| | | | Tm 550 nm |
|---|---|---|---|
| Sample name | Protein | Treatment | (° C.) |
| C086 | C086 | — | 54.4 |
| C086 buffer | | +MMP2 buffer | 50.2 |
| C086 mmp | | 0 | 50.2 |
| C190 | Propeptide-C086 | — | 62.2 |
| C190 buffer | | +MMP2 buffer | 57.4 |
| C190 mmp | | 0 | 57.3 |
| C127 | Propeptide-LinkerX- | — | 63.4 |
| C127 buffer | C086 | +MMP2 buffer | 58 |
| C127 mmp | | 0 | 50.9 |
| C136 | C086-2xGGS-RaD3 | — | 61.5 |
| C136 buffer | | +MMP2 buffer | 58 |
| C136 mmp | | 0 | 57.4 |
| C137 | C086-3xG4S-RaD3 | — | 62.6 |
| C137 buffer | | +MMP2 buffer | 57.2 |
| C137 mmp | | 0 | 57.2 |
| C172 | C086-LinkerX-RaD3 | — | 61 |
| C172 buffer | | +MMP2 buffer | 56.3 |
| C172 mmp | | 0 | 70.5 |
| C173 | C086-PLGLAG-RaD3 | — | 60 |
| C173 buffer | | +MMP2 buffer | 56.9 |
| C173 mmp | | 0 | 56.7 |

### Example 5—Formulation of Act-IL-18 Polypeptides

Lyophilized Act-IL-18 polypeptides are suspended in a solution comprising 10-50 mM Histidine buffer, 5-10% trehalose, 0.02% tween. Lyophilized Act-IL-18 polypeptide can also be resuspended in other suitable or appropriate buffers, such as PBS (pH 7.4) with mannitol (e.g., 50 mg/mL) and tween (e.g., 0.02%).

### Example 6—Conjugation of IL-18 Polypeptide With Bifunctional Linking Group

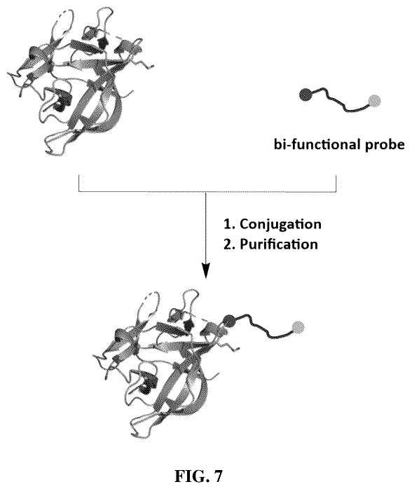

An Act-IL-18 polypeptide as provided herein can be conjugated to a bifunctional linking group prior to forming the full linker of the activatable immunocytokine composition. In some cases, the bifunctional linking group first attaches to a desired residue of the Act-IL-18 polypeptide at the point of attachment of the linker. In some instances, it is preferred that the Act-IL-18 polypeptide contains only one cysteine (e.g., the cysteine at positions C38, C76, and C127 of the IL-18 polypeptide and C10 of the IL-18 propeptide are all substituted, allowing conjugation to only residue C68 of the Act-IL-18 polypeptide). Once attached to the Act-IL-18 polypeptide, the second functionality of the bifunctional linking group is used to attach to a second portion. An exemplary schematic of conjugation of the Act-IL-18 polypeptide with bifunctional linking group is shown in FIG. 7. The process shown in FIG. 7 is not needed for all IL-18 polypeptides, as some Act-IL-18 polypeptides provided herein (e.g., an IL-18 with the desired conjugation handle for final linkage with the antibody already attached). An exemplary protocol on an IL-18 polypeptide with a cysteine residue point of attachment provided herein is described below.

Conjugation—The Act-IL-18 polypeptide is stored at a concentration of 2.4 mg/mL at −80° C. in potassium phosphate buffer (pH 7.0) containing 50 mM KCl and 1 mM DTT. The sample is thawed on ice yielding a clear solution. The protein solution is diluted in PBS, pH 7.4. A clear solution is obtained at a concentration of ˜ 0.4 mg/mL.

The protein solution is dialyzed against PBS, pH 7.4 (twice against 600 mL for 2 h and once against 800 mL for 18 h). After dialysis, a clear solution is obtained with no sign of precipitation. Protein concentration is obtained using UV absorbance at 280 nm and by BCA protein assay.

A stock solution of bi-functional linking group (e.g., bromoacetamido-PEG5-azide, CAS: 1415800-37-1) in water is prepared at a concentration of 20 mM. 500 μL of the protein solution are mixed with 25 μL of linking group solution. pH was adjusted to 7.5 and it was let to react for 3 h at 20° C.

The progress of the synthesis is monitored by reverse-phase HPLC using a gradient of 5 to 30% (2.5 min) and 30 to 75% (7.5 min) CH₃CN with 0.1% TFA (v/v) on a Acris WIDEPORE C18 200 Å column (3.6 μm, 150×4.6 mm) at a flow rate of 1 mL/min at 40° C. and by MALDI-TOF MS.

Purification—In some cases, ion-exchange chromatography is used to purify the conjugated protein. To remove the excess of probe, the reaction mixture (volume is around 500 μL) is flowed through a Hi-Trap-Q-FF-1 mL column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl, 5% glycerol) and concentrated at 0.4 mg/mL. The concentration of purified protein is determined by UV absorbance at 280 nm and by BCA protein assay. The protein solution is kept at −80° C.

Characterization—The purity and identity of the recombinant protein from commercial source and the conjugated protein is confirmed by aSEC, HPLC and MALDI-TOF MS.

### Example 7—Conjugation of IL-18 Polypeptide to Antibody

### Preparation of a Conjugatable Antibody

A modified antibody (e.g., an anti-PD-1 antibody such as Nivolumab or LZM-009) comprising a DBCO conjugation handle is prepared using a protocol modified from Examples 2-4 of US Patent Publication No. US2020019165A1 (AJICAP™ technology). An exemplary illustration of this process resulting in the attachment of one DBCO conjugation handle is shown in FIG. 20. Briefly, the anti-PD-1 antibody with a free sulfhydryl group attached to a lysine residue side chain in the Fc region is prepared by reacting the antibody with an affinity peptide configured to deliver a protected version of the sulfhydryl group (e.g., a thioester) to the lysine residue. The protecting group is then removed to reveal the free sulfhydryl. The free sulfhydryl is then reacted with a bifunctional reagent comprising a bromoacetamide group connected to the DBCO conjugation handle through a linking group

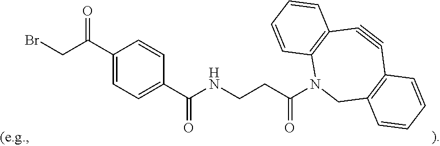 [SMILES: O=C(CBr)c1ccc(C(=O)NCCC(=O)N2Cc3ccccc3C#Cc3ccccc32)cc1]

The method can be used to produce an antibody with one DBCO group present (DAR1) and/or two DBCO groups attached to the antibody (DAR2, one DBCO group linked to each Fc of the antibody).

### Conjugation of Antibody to Act-IL-18 Polypeptide

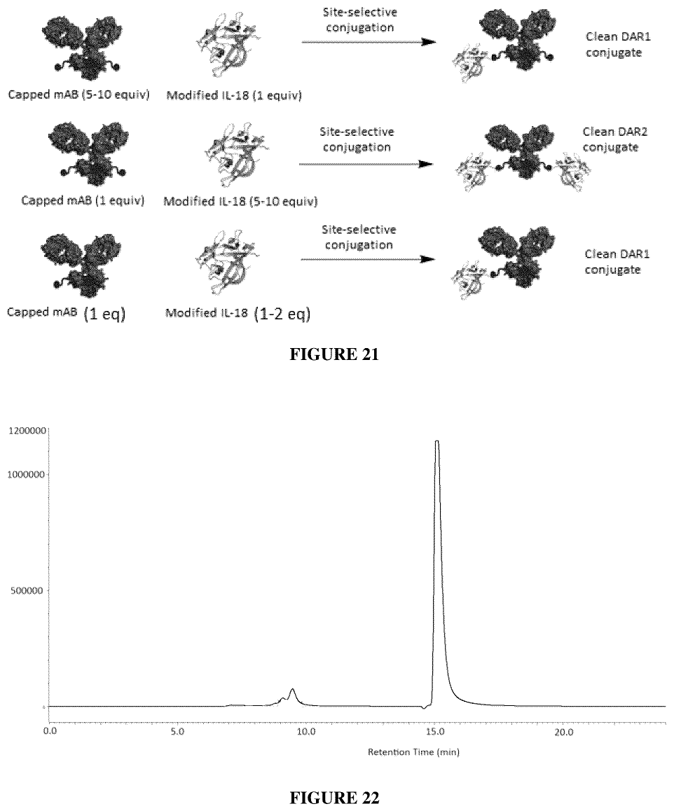

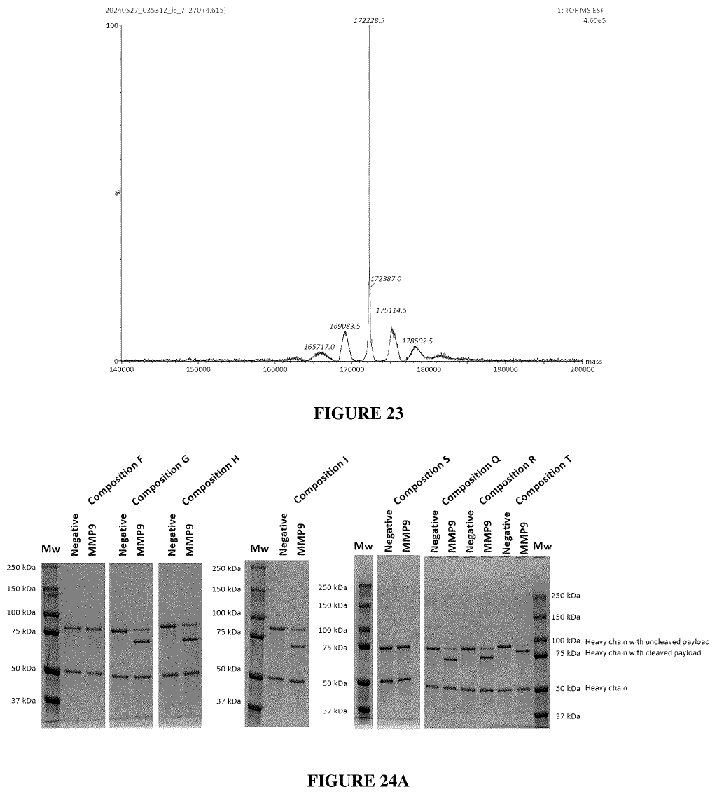

The DBCO modified antibody is then conjugated to an Act-IL-18 polypeptide comprising an azide moiety at a desired point of attachment (e.g., an Act-IL-18 polypeptide which contains an amino acid with an azide side chain or an Act-IL-18 linked to an azide using a bifunctional linking group as in Example 4). DBCO modified antibody with one (DAR1) or two (DAR2) reactive handles are reacted with 2-10 equivalents of azide containing Act-IL-18 (pH 5.2 buffer, 5% trehalose, rt, 24 h). In an alternative embodiment, antibody comprising two DBCO conjugation handles is reacted either as an excess reagent (e.g., 5-10 equivalents) with 1 equivalent of Act-IL-18 comprising an azide functionality to produce a DAR1 antibody or the antibody comprising two DBCO conjugation handles is reacted with I equivalent of antibody with excess reagent of Act-IL-18 comprising an azide (e.g., 5-10 equivalents) to produce a DAR2 antibody. An illustration of this protocol is shown in FIG. 21.

### Purification and Characterization of Antibody-Act-IL-18 Immunocytokine

Antibody-Act-IL-18 polypeptide immunocytokine is purified from unreacted Act-IL-18 and aggregates using a desalting column, AIEX (GE Healthcare Life Sciences AKTA pure, mobile phase A: 25 mM TRIS pH 7.4, mobile phase B: 25 mM TRIS pH 7.4/1M NaCl, column: GE Healthcare Life Sciences HiTrap Q HP, flow rate: 1.5 mL/min, gradient: 0% to 100% mobile phase B in 35 min) and SEC (GE Healthcare Life Sciences AKTA pure, mobile phase: Histidine 5.2/150 mM NaCl/5% Trehalose, column: GE Healthcare Life Sciences SUPERDEX™ 200 increase 3.2/300, flow rate: 0.5 mL/min).

The purity and identity of the antibody-Act-IL-18 polypeptide immunocytokine is confirmed by RP-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN:IPA:ETOH:H2O (5:1.5:2:1.5), flow rate: 0.5 mL/min, injection amount: 10 μg (10 μL Injection of 1 mg/mL), gradient: 0% to 20% mobile phase B in 50 min), by analytical SEC (column: Waters, XBridge Premier Protein SEC Column, 7.8×300 mm, 2.5 μm, 250 Å pore size, mobile phase: PBS pH 7.2, flow rate: 0.75 mL/min, samples concentration: 0.5-1 mg/ml, injection amount: 10 uL, gradient: 100% of elution buffer for 24 min) and SDS-PAGE.

Generation of non-activatable IL-18 immunocytokines according to the above methods (as well as additional characterization of such immunocytokines) is described in detail in PCT Publication No. WO2023161854A1 and corresponding U.S. Patent. Application Publication No. US2023/0355795A1, the contents of which are incorporated herein by reference. Such characterization is not described in more detail herein, though the activatable IL-18 immunocytokines described herein are predicted to behave analogously after suitable activation of the IL-18 polypeptide.

Exemplary chromatograms and analytical characterization of aDAR1 immunocytokine referred to as Composition L herein comprising an activatable IL-18 of SEQ ID NO: 594 (also referred to herein as C329) conjugated via residue C68 and an anti-PD-1 antibody (LZM-009) prepared according to the described methods are shown in FIGS. 22 (aSEC) and 23 (Q-TOF Mass Spectrometry). Similar chromatograms and analytical characterization was observed for each of Compositions F-T described herein.

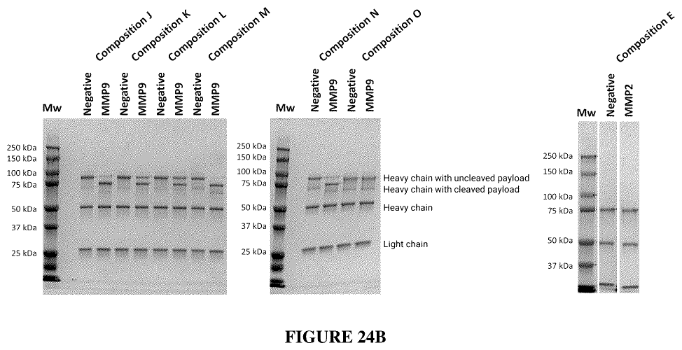

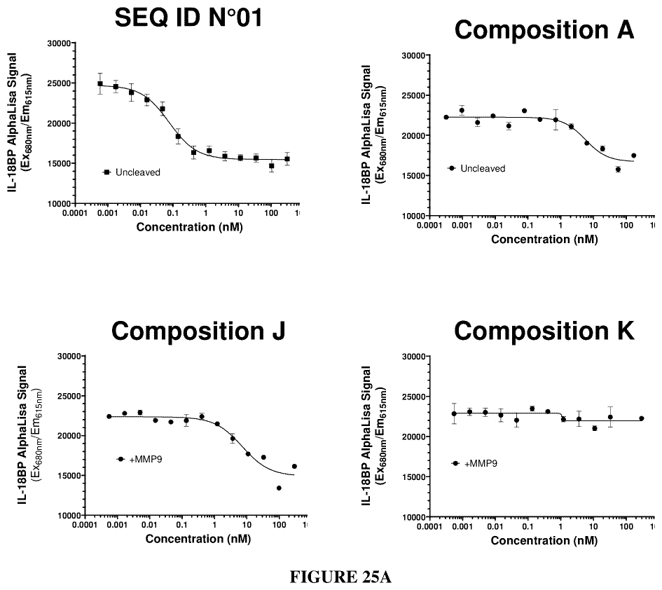

Digestion SDS-PAGE characterization of each of Compositions F—O and Q-T was performed. Results are shown in FIGS. 24A and 24B. For each Composition, samples were run untreated or digested by MMP9 (MMP2 for Composition E) for 18 hours at 37° C. shaking at 300 rpm. Samples were loaded under reducing conditions. All activatable immunocytokines showed the predicted cleavage products upon treatment (or lack of cleavage product as predicted for Composition E).

### Example 8—Characterization of Immunocytokine IL-18 Activity

The ability of the activatable immunocytokine to perform various IL-18 activities is measured as provided below, as well as relevant comparisons to non-conjugated IL-18 polypeptides. The activatable immunocytokine compositions described herein are tested both before and after protease treatment where indicated.

IL-18BP Binding alphaLISA Assay

A human IL-18BP AlphaLISA Assay Kit is used to determine the binding affinity of each IL-18 variant immunocytokine for IL-18BP, which detected the presence of the free form of IL-18BP.

Sixteen three-fold serial dilutions of activatable immunocytokines IL-18 analytes are prepared in 1× assay buffer (provided in the IFNgamma AlphaLISA kit) in the presence of 5 ng/ml of His-tagged human IL-18BP. Final IL-18 immunocytokine analytes concentration range from 2778 nM to 0.2 pM. Samples were tested using both non-cleaved and after incubation with indicated proteases according to the protocols described herein.

After 1 hr incubation at room temperature, free IL-18BP levels are measured using a Human IL-18BP AlphaLISA Assay Kit. In a 384 well OPTIplate, 5 μL of 5× Anti-IL-18BP acceptor beads are added to 7.5 μL of an IL-18/IL-18BP mix. After 30 min incubation at room temperature with shaking, 5 μL of biotinylated Anti-IL-18BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (KD) is calculated based on a variable slope, four parameter analysis using GraphPad PRISM software.

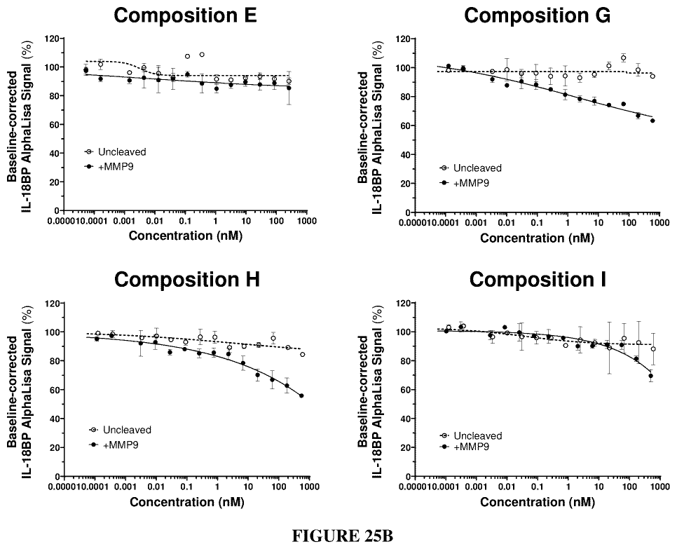

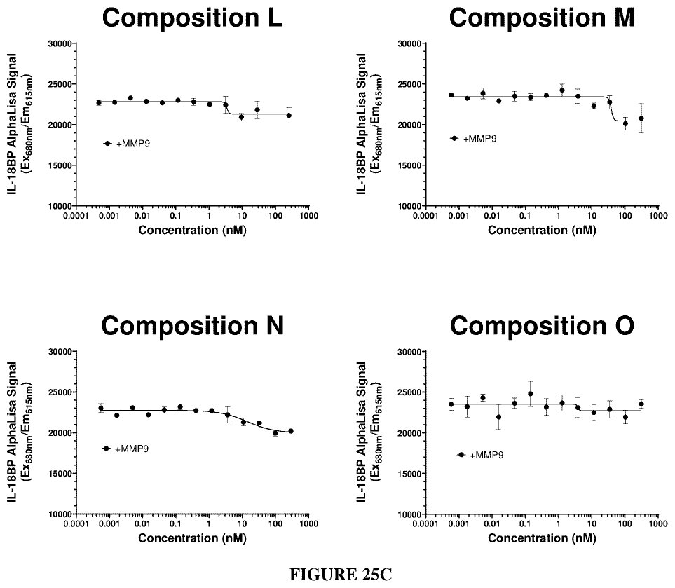

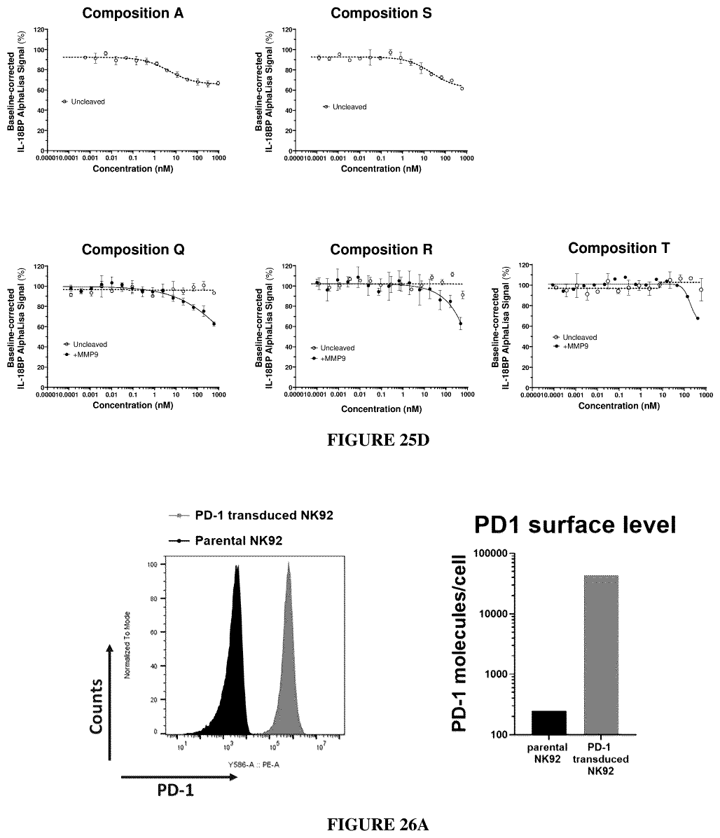

Results of this experiment are shown in FIGS. 25A-25D. None of the variants tested displayed substantial binding to IL-18BP, though some samples showed some slight binding at higher IL-18BP concentrations tested after treatment with protease.

### IFNγ Induction Cellular Assay

The ability of immunocytokines and IL-18 polypeptides provided herein were assessed for ability to induce IFNγ in a cellular assay according to the protocol below.

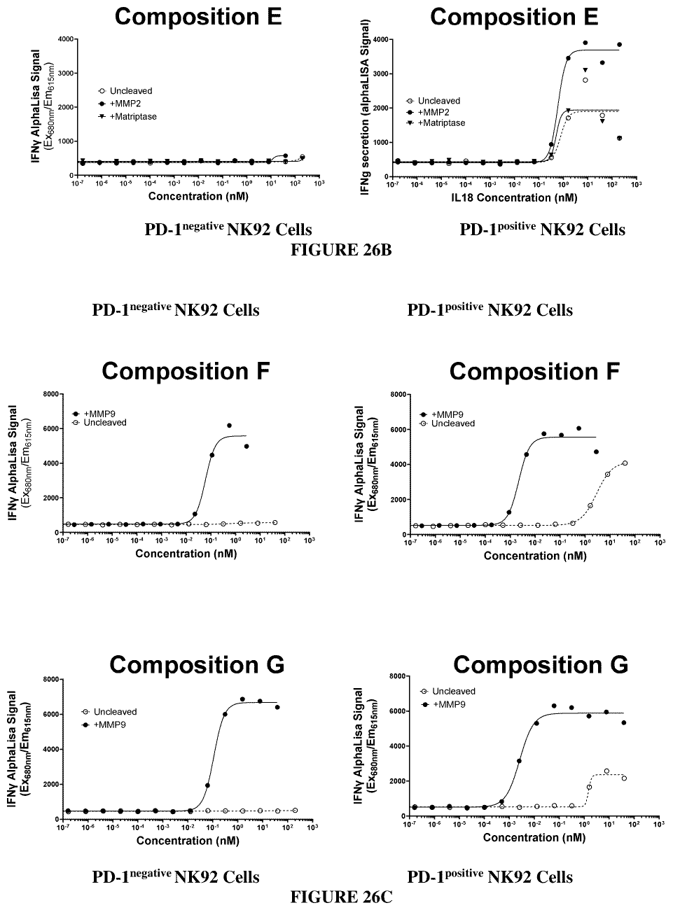

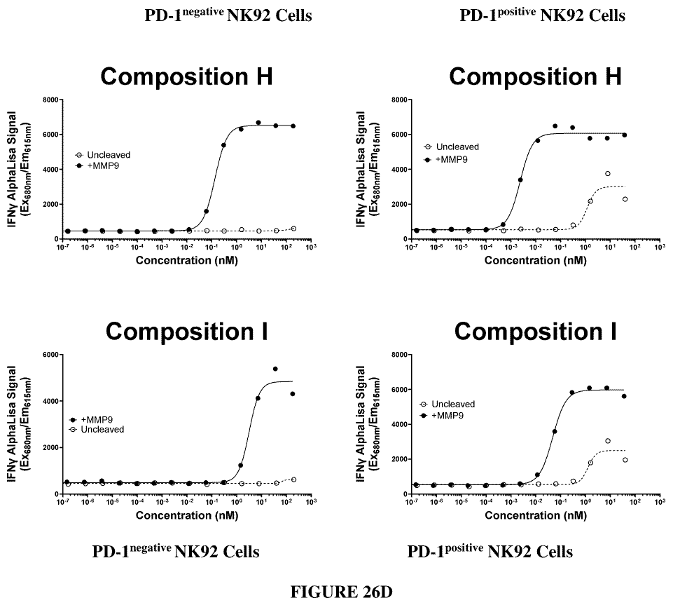

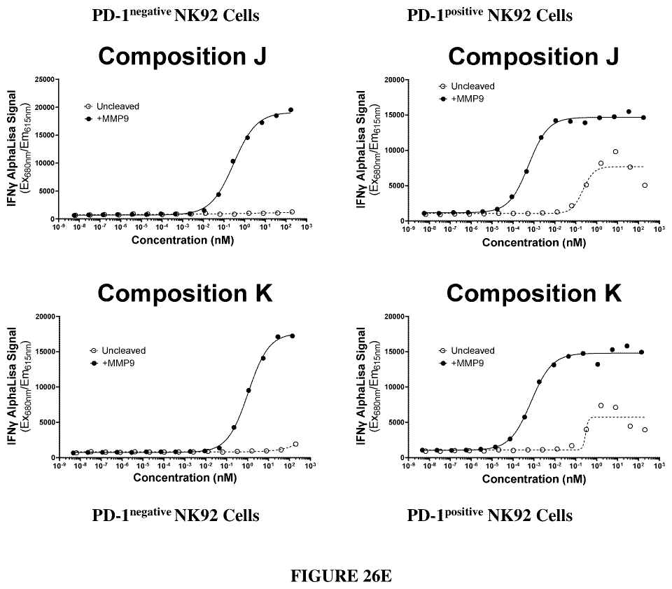

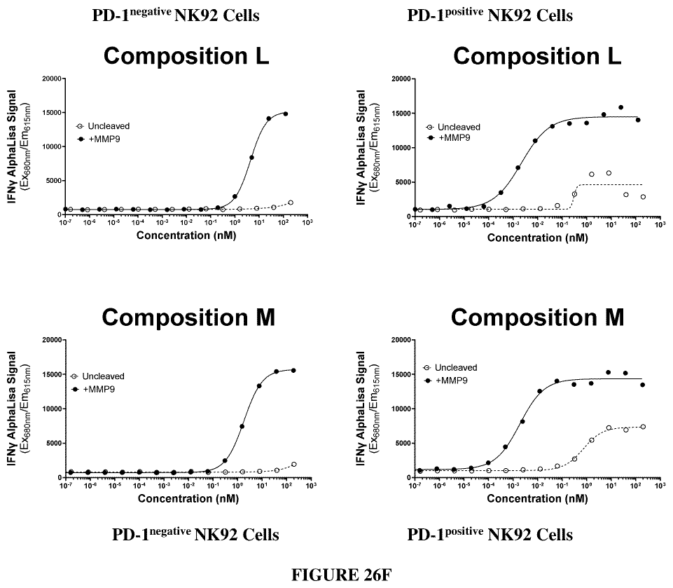

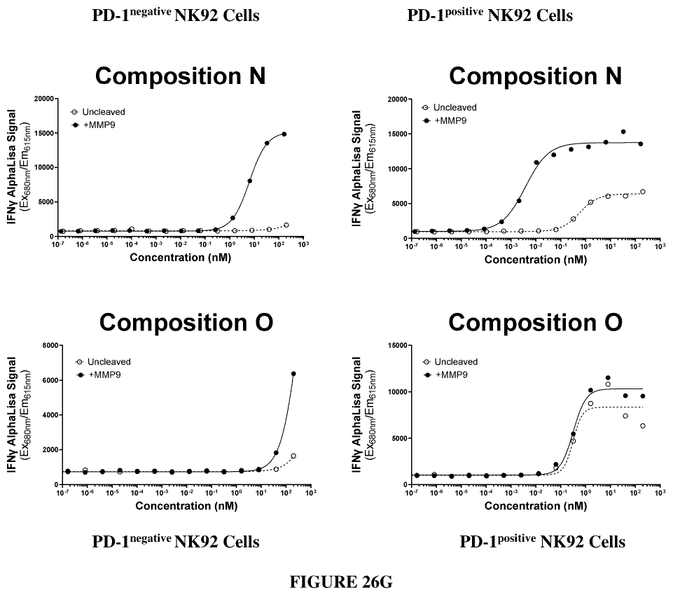

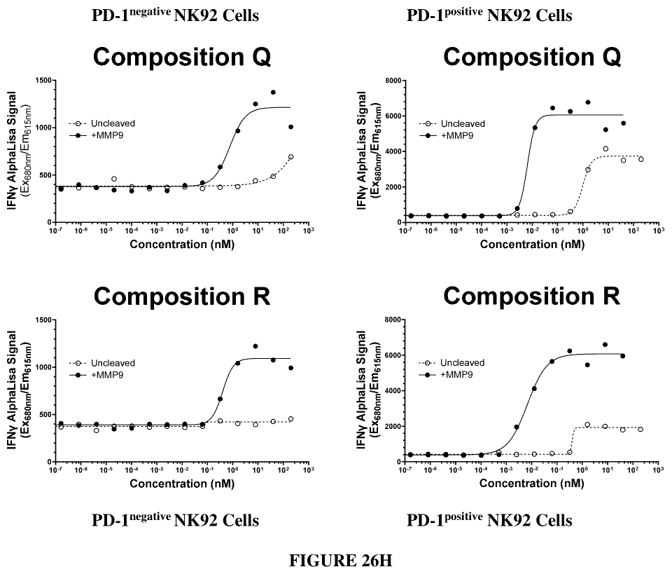

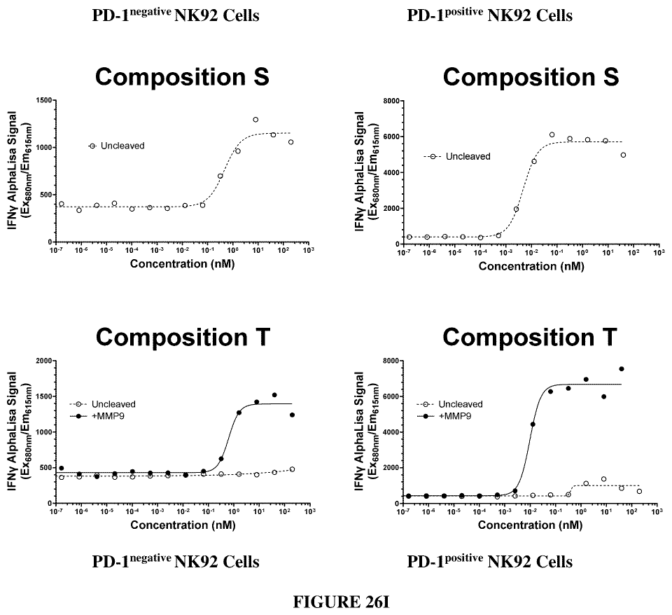

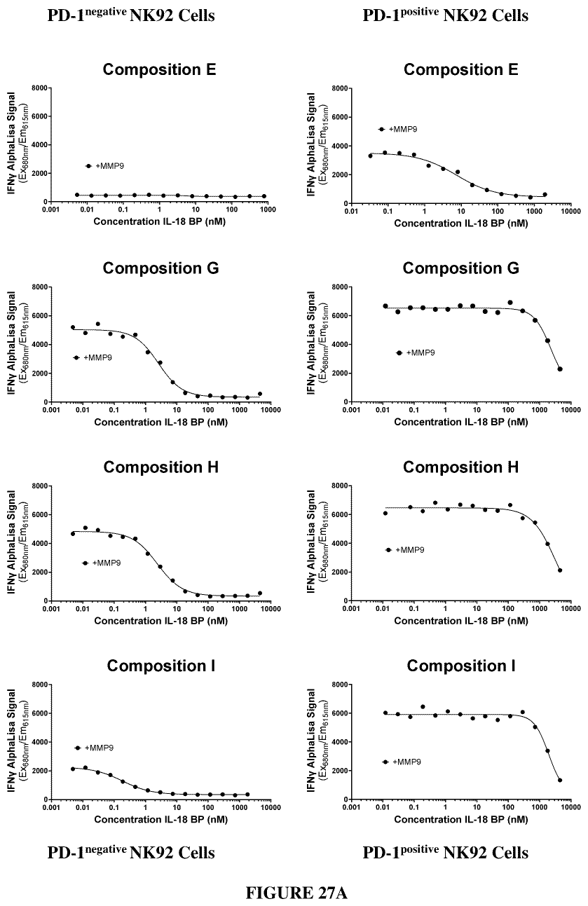

The NK cell line NK-92 derived from a patient with lymphoma (ATCC® CR^{L}-2407™) is cultured in aMEM medium supplemented with 20% FCS, Glutamax, 25 UM B-mercaptocthanol, and 100 IU/mL of recombinant human IL-2. In the below experiment, NK-92 cells were transduced to express PD-1 on their surface. FIG. 26A shows the change in PD-1 levels on the transduced cells compared to parental cells.

On the day of experiment, cells are harvested and washed with aMEM medium without IL-2 and containing 1 ng/ml of recombinant human IL-12. After counting, cells are seeded at 100,000 cells/well in a 384 well titer plate and incubated at 37° C./5% CO₂. Sixteen 4-fold serial dilutions of IL-18 analytes are prepared in aMEM medium, and 1 ng/ml of IL-12 were added to the NK-92 cells. Final IL-18 analyte concentrations range from 56 nM to 5×10-5 pM.

After incubating the cells for 16-20 hr at 37° C./5% CO₂, 5 μL of supernatant is carefully transferred to a 384 microwell OptiPlate. IFNγ levels are measured using a human IFNγ AlphaLISA Assay Kit. Briefly, 10 μL of 2.5× AlphaLISA Anti-IFNγ acceptor beads and biotinylated antibody anti-IFNγ mix are added to the 5 μL of NK-92 supernatants. The mixtures are incubated for 1 hr at room temperature with shaking. Under subdued light, 2.5 μL of 2× streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC₅₀) are calculated based on a variable slope and four parameter analysis using GraphPad PRISM software.

The indicated activatable immunocytokines were tested both before and after treatment with relevant protease. Results are shown in FIGS. 26B-I.

### IL-18 Binding Protein Inhibition Cellular Assay

The NK cell line NK-92 derived from a patient with lymphoma (ATCC® CR^{L}-2407™) is cultured in aMEM medium supplemented with 20% FCS-Glutamax, 25 UM B-mercaptocthanol, and 100 IU/mL of recombinant human IL-2. In the below experiment, some NK-92 cells were transduced to express PD-1 on their surface and compared with the parental cell line. FIG. 26A shows the change in PD-1 levels on the transduced cells compared to parental cells. This experiment gives an indication of the degree to which avidity effects of PD-1 binding influence IFNg stimulation activity and its inhibition by IL-18BP.

On the day of experiment, cells are harvested and washed with aMEM medium without IL-2 and containing 1 ng/mL of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in aMEM medium. 1 ng/mL of IL-12 containing 2 nM of each modified IL-18 polypeptide variant is added to the NK-92 cells. The final IL-18 analyte concentration is 1 nM, and the final IL-18BPa concentration ranged from 566 nM to 17 pM.

After incubating the cells for 16-20 hr at 37° C./5% CO₂, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2×SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC₅₀) are calculated based on a variable slope and four parameter analysis using GraphPad PRISM software.

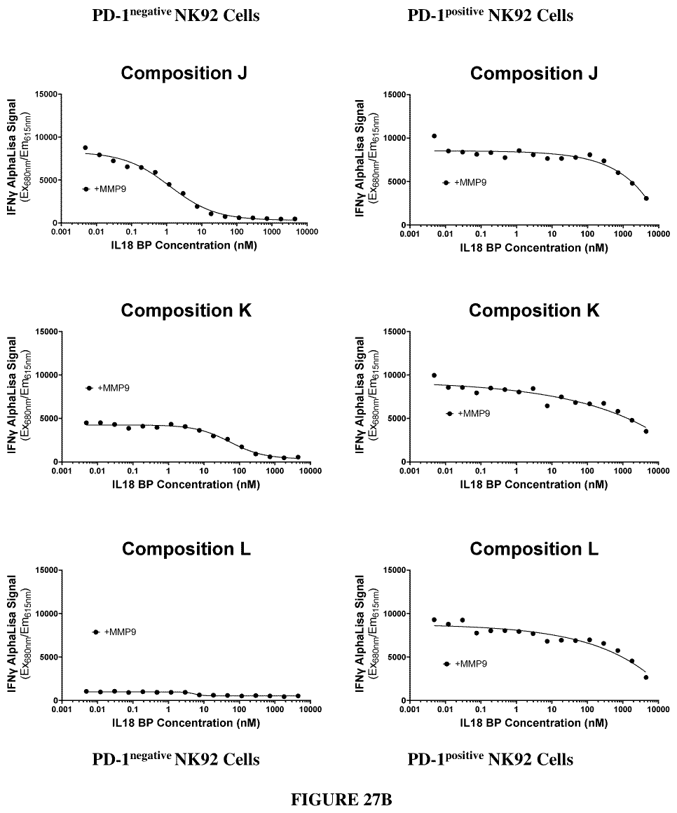

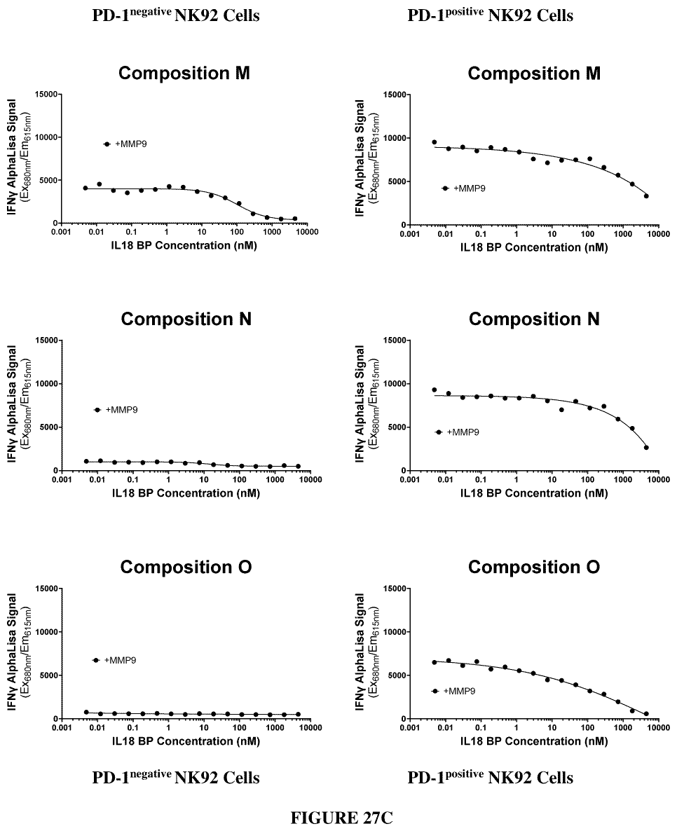

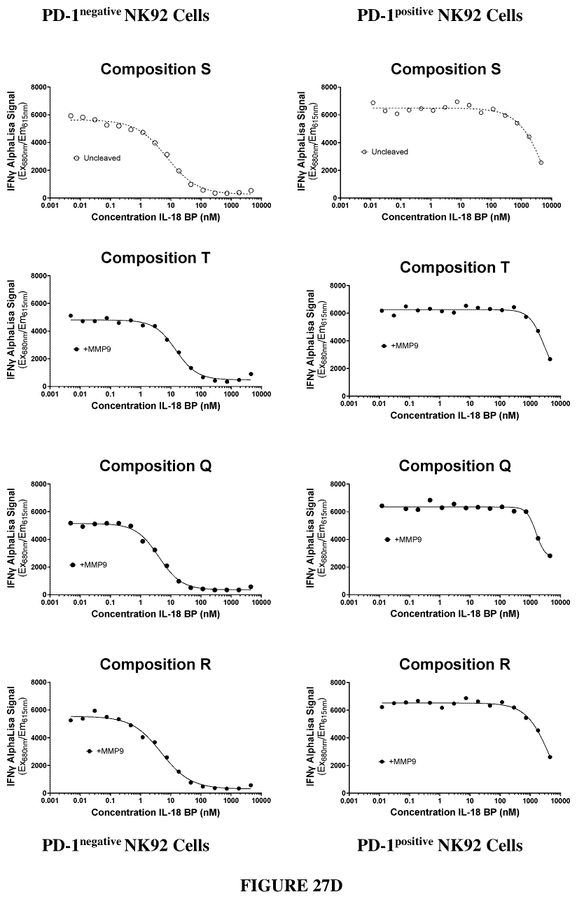

The indicated activatable immunocytokines were tested after treatment with MMP9 according to standard protocols, except for Composition S which was untreated. Results are shown in FIGS. 27A-D. In each of FIGS. 27A-D, the left panel depicts results in the parental NK-92 cell line, and the right panel shows the results in NK-92 cells transduced with PD-1. In each case, IFNg stimulating activity was less prone to inhibition in the PD-1+ cells.

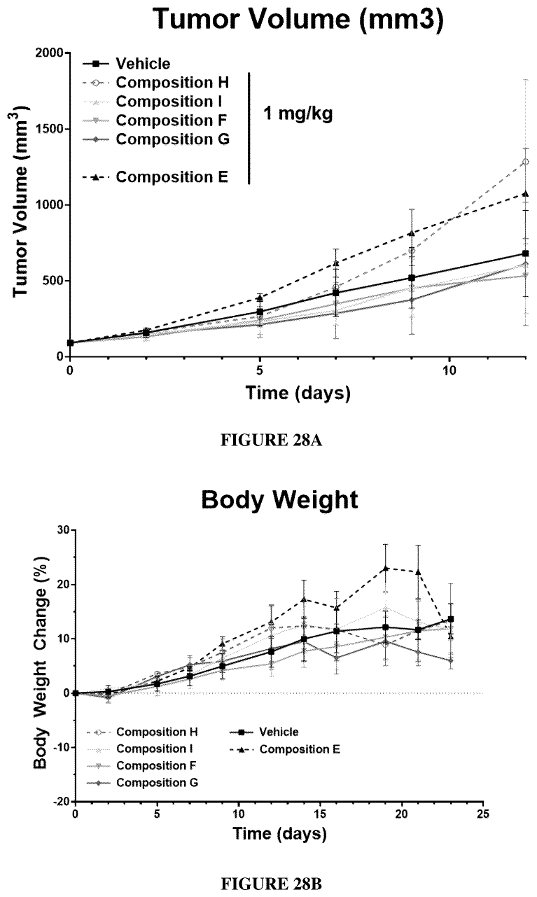

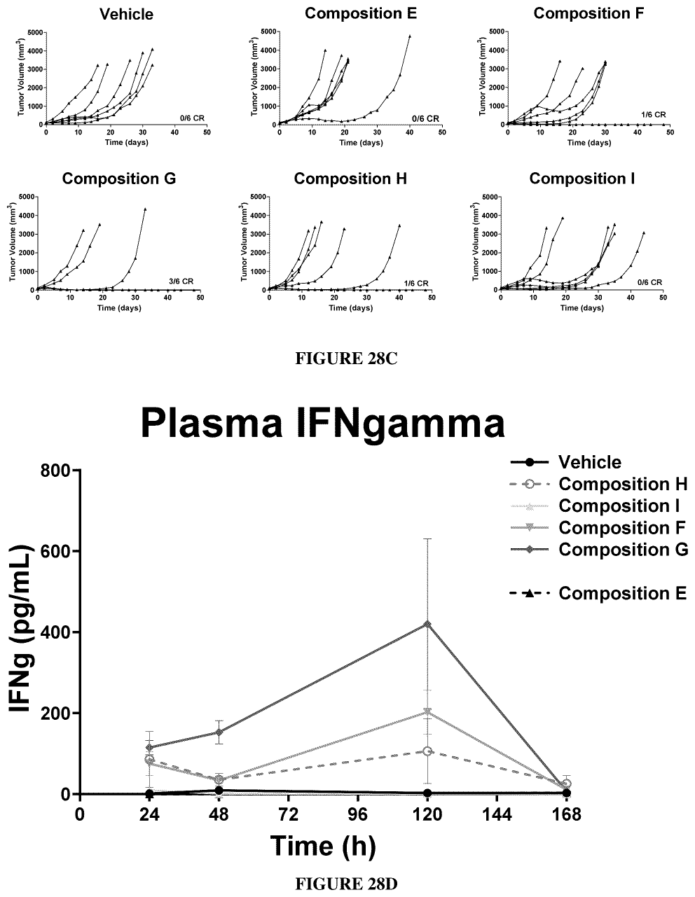

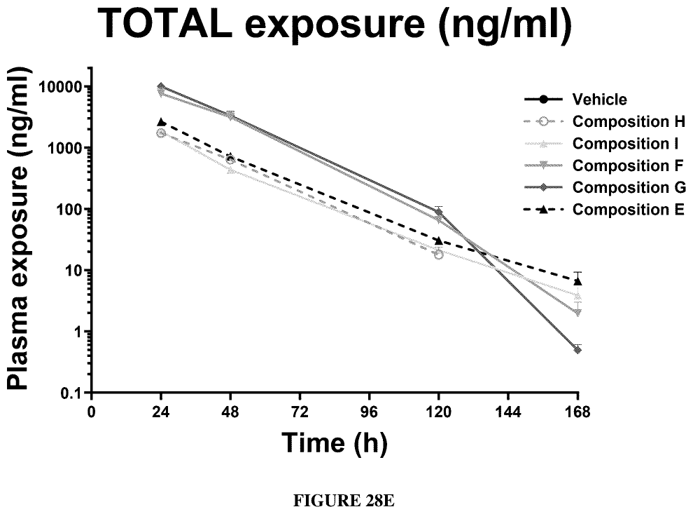

### Example 9—In Vivo Antitumor Activity in MC38 Colon Carcinoma Model (FIGS. 28A-28E)

An in vivo efficacy study was performed in mice. Naïve, 6-8 weeks old, C57BL/6-hPD1 female mice (GemPharmatech Co, Ltd, Nanjing, China) were inoculated subcutaneously at the right upper flank with MC38 tumor cells (3×10⁵) in 0.1 mL of PBS for tumor development. The animals were randomized (using an Excel-based randomization software performing stratified randomization based upon tumor volumes), and treatment started when the average tumor volume reached approximately 90 mm³. Animals treated with activatable IL-18 polypeptide conjugated antibodies received a single 1 mg/kg bolus intravenous (i.v.) injection on Day 0. After inoculation, the animals were checked daily for morbidity and mortality. 6 animals were placed into each group. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

FIG. 28A shows tumor volume (mm3) on the y-axis versus time (days) on the x-axis. Human PD1 knock-in (huPD1 ki) C57BL/6 animals bearing sub cutaneous MC38 tumors were treated intravenously with a single dose (SD) of 1 mg/kg on day 0. Tumor size at start of treatment was ˜90 mm³ (n=6; Mean±SEM).

FIG. 28B shows body weight change (%) compared to day 0 body weight on the y-axis versus time (days) on the x-axis. Human PD1 knock-in (huPD1 ki) C57BL/6 animals bearing sub-cutaneous MC38 tumors were treated intravenously with a single dose (SD) of 1 mg/kg on day 0. Tumor size at start of treatment was ˜90 mm³ (n=6; Mean±SEM).

FIG. 28C shows tumor volume (mm3) on the y-axis versus time (days) on the x-axis for individual human PD1 knock-in (huPD1 ki) C57BL/6 animals bearing sub-cutaneous MC38 tumors. Mice were treated intravenously with a single dose (SD) of 1 mg/kg on day 0. Tumor size at start of treatment was ˜90 mm³ (n=6; Mean±SEM).

FIG. 28D shows IFNgamma (pg/mL) released in plasma on the y-axis versus time (hours after dosing) on the x-axis. Human PD1 knock-in (huPD1 ki) C57BL/6 animals bearing sub-cutaneous MC38 tumors were treated intravenously with a single dose (SD) of 1 mg/kg on day 0 and plasma samples were harvested at different time points after dosing (n=3; Mean±SEM). IFNgamma values in plasma were quantified using the Legendplex Mouse Anti-virus Response Panel (Biolegend) according to manufacturer's recommendations.

FIG. 28E shows total drug levels (ng/mL) present in plasma on the y-axis versus time (hours after dosing) on the x-axis. Human PD1 knock-in (huPD1 ki) C57BL/6 animals bearing sub-cutaneous MC38 tumors were treated intravenously with a single dose (SD) of 1 mg/kg on day 0 and plasma samples were harvested at different time points after dosing (n=3; Mean±SEM). Drug in plasma was detected using a sandwich ELISA.

### Example 10: Characteristics of Additional IL-18 Polypeptides

The below examples describe analysis and characterization of additional IL-18 polypeptides as provided herein. It is expected that these additional IL-18 polypeptides would behave analogously to those of the Act-IL-18s provided herein (and corresponding immunocytokines) when incorporated into an Act-IL-18 or activatable immunocytokine.

10A—HEK-Blue Reporter Assay—An IL-18R positive HEK-Blue reporter cell line is used to determine binding of IL-18 variants to IL-18R and subsequent downstream signaling. The general protocol is outlined below.

5×10⁴ cells HEK-Blue IL18R reporter cells (InvivoGen, #hkb-hmil18) are seeded into each well of a 96 well plate and stimulated with 0-100 nM of IL-18 polypeptide variants at 37° C. and 5% CO₂. After 20 h incubation, 20 μL of cell culture supernatant is then taken from each well and mixed with 180 μL QUANTI-Blue media in a 96 well plate, incubated for 1 hour at 37° C. and 5% CO₂. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

The HEK-Blue IL-18R reporter assay described above was performed on additional IL-18 polypeptides which can be incorporated into activatable immunocytokine compositions provided herein. It is expected that the IL-18 polypeptides provided below would behave similarly to C086 (SEQ ID NO: 30) when incorporated into an activatable immunocytokine composition as those otherwise provided herein.

| SEQ ID | | |
|---|---|---|
| NO: or | | |
| Compo- | | |
| sition | | EC₅₀ |
| ID | Sequence modifications | (pM) |
| 1 | Native sequence | 3.33 |
| 34 | E6K, K53A, S55A | 272.5 |
| 39 | E6K, K53A | 0.72 |
| 42 | E6K, K53A, S55A, T63A | 0.79 |
| 50 | E6K, K53A, T63A | 1.77 |
| 54 | E6K, C38S, K53A, C68S, K70C, C76S, C127S | 9.12 |
| 56 | E6K, K53A, C38S, C76S, C127S | 3.73 |
| 57 | E6K, C38S, K53A | 0.86 |
| 30 | E6K, V11I, C38A, K53A, T63A, C76A, | 0.034 |
| | C127A | |
| 62 | E6K, C38A, K53A, C127A | 0.17 |
| 60 | E6K, C38Q, K53A | 0.203 |
| 59 | E6K, C38A, K53A | 0.268 |
| 57 | E6K, C38S, K53A | 0.53 |
| C143 | V11I, C38A, K53A, C76A, C127A | 0.98 |
| C144 | V11I, C38A, K53A, T63A, C76A, C127A | 0.17 |
| C145 | V11I, C38A, K53A, S55A, C76A, C127A | 3.63 |
| C146 | V11I, C38A, M51G, K53A, C76A, C127A | 0.8 |
| C147 | V11I, C38A, K53A, D54A, C76A, C127A | 1 |
| C148 | F2A, V11I, C38A, K53A, C76A, C127A | 7.28 |
| C149 | V11I, E31A, C38A, K53A, C76A, C127A | 6.6 |
| C150 | V11I, T34A, C38A, K53A, C76A, C127A | 0.7 |
| C151 | V11I, D35A, C38A, K53A, C76A, C127A | 13.12 |
| C152 | V11I, S36A, C38A, K53A, C76A, C127A | 0.25 |
| C153 | V11I, D37A, C38A, K53A, C76A, C127A | 14.12 |
| C154 | V11I, E31A, D37A, C38A, K53A, C76A, C127A | 11.95 |
| C155 | V11I, C38A, D40A, K53A, C76A, C127A | 0.52 |
| C156 | V11I, C38A, N41A, K53A, C76A, C127A | 11.7 |
| C157 | V11I, C38A, K53A, C76A, C127A, D132A | 1.95 |
| C158 | V11I, C38A, K53A, C76A, G108A, C127A | 15.56 |
| C159 | V11I, C38A, K53A, C76A, H109A, C127A | 19.5 |
| C160 | V11I, C38A, K53A, C76A, D110A, C127A | 2.02 |
| C161 | K8R, V11I, C38A, C76A, Q103E, C127A | 2.01 |
| C162 | K8E, V11I, C38A, C76A, Q103R, C127A | 2.3 |
| C163 | V11I, C38A, C76A, Q103K, C127A | 1.5 |
| C164 | V11I, C38A, S55H, C76A, C127A | 3.14 |
| C165 | V11I, C38A, S55R, C76A, C127A | 1.91 |
| C166 | V11I, C38A, S55T, C76A, C127A | 4.73 |
| C167 | V11I, C38A, C76A, S105I, C127A | 5.37 |
| C168 | V11I, C38A, C76A, S105K, C127A | 7.73 |
| C174 | K8L, E6K, V11I, C38A, K53A, T63A, C76A, | 0.29 |
| | C127A | |
| C176 | E6K, V11I, C38A, I49M, K53A, T63A, C76A, | 0.07 |
| | C127A | |
| C177 | E6K, V11I, C38A, I49R, K53A, T63A, C76A, | 0.04 |
| | C127A | |
| C178 | E6K, V11I, C38A, K53A, T63A, C76A, Q103R, | 0.26 |
| | C127A | |
| C179 | E6K, K8E, V11I, C38A, K53A, T63A, C76A, | 0.4 |
| | Q103R, C127A | |
| C181 | E6K, V11I, C38A, K53A, T63A, C76A, C127A, | 0.1 |
| | V153E | |
| C182 | E6K, V11I, C38A, K53A, T63A, C76A, C127A, | 0.08 |
| | V153Y | |
| C183 | E6K, V11I, C38A, M51G, K53A, T63A, C76A, | 0.1 |
| | C127A | |
| C184 | E6R, V11I, C38A, K53A, T63A, C76A, C127A | 0.04 |
| C140 | E6K, V11I, C38A, K53A, T63A, C76A, C127A | 2.5 |
| C141 | E6K, V11I, C38A, K53A, T63A, C76A, C127A | 1.68 |
| C142 | Y1M, E6K, V11I, C38A, K53A, T63A, C76A, | 0.02 |
| | C127A | |
| C192 | E6K, V11I, C38A, M51G, K53A, T63A, C76A, | 13.99 |
| | C127A | |

10B—IL-18 BP AlphaLISA assay—An IL-18 binding protein AlphaLISA experiment substantially as described above was performed on IL-18 polypeptide which can be incorporated into activatable immunocytokine compositions as provided herein to assess ability to bind to IL-18BP. Results are shown in the Table below.

| SEQ ID NO: or | | KD |
|---|---|---|
| Composition ID | Sequence modifications | (nM) |
| 1 | Native Sequence | 0.67 |
| 34 | E06K, K53A, S55A | >1500 |
| 35 | Y01G, F02A, E06K, M51G, K53A, D54A, S55A, T63A | 969.0 |
| 36 | K53A | 513.8 |
| 37 | S55A | 10.7 |
| 38 | E06K | 0.13 |
| 39 | E06K, K53A | 130.3 |
| 40 | E06K, S55A | 12.3 |
| 41 | K53A, S55A | 500.0 |
| 42 | E06K, K53A, S55A, T63A | 822.0 |
| 43 | E06K, K53A, S55A, Y01G | |
| 44 | E06K, K53A, S55A, F02A | >1000 |
| 45 | E06K, K53A, S55A, D54A | >1000 |
| 46 | E06K, K53A, S55A, M51G | >1000 |
| 47 | C38S, C68S, C76S, C127S | 0.03 |
| 48 | C38S, C68S, C76S, C127S, K70C | 0.21 |
| 49 | E06K, K53A, S55A, C38S, C68S, C76S, C127S, K70C | >1000 |
| 50 | E06K, K53A, T63A | 339.8 |
| 51 | T63A | 2.59 |
| 52 | E06K, T63A | 0.83 |
| 53 | K53A, T63A | 198 |
| 54 | E06K, K53A, C38S, C68S, C76S, C127S, K70C | 446.0 |
| 55 | K53A, T63A, C38S, C68S, C76S, C127S, K70C | 913 |
| 56 | E6K, K53A, C38S, C76S, C127S | 435.5 |
| 57 | E6K, K53A, C38S | 50.2 |
| C143 | V11I, C38A, K53A, C76A, C127A | 8.86 |
| C144 | V11I, C38A, K53A, T63A, C76A, C127A | 0.66 |
| C145 | V11I, C38A, K53A, S55A, C76A, C127A | 9.74 |
| C146 | V11I, C38A, M51G, K53A, C76A, C127A | 373.30 |
| C147 | V11I, C38A, K53A, D54A, C76A, C127A | 25.77 |
| C148 | F2A, V11I, C38A, K53A, C76A, C127A | 57.21 |
| C149 | V11I, E31A, C38A, K53A, C76A, C127A | 0.64 |
| C150 | V11I, T34A, C38A, K53A, C76A, C127A | 1.24 |
| C151 | V11I, D35A, C38A, K53A, C76A, C127A | 2.88 |
| C152 | V11I, S36A, C38A, K53A, C76A, C127A | 1.12 |
| C153 | V11I, D37A, C38A, K53A, C76A, C127A | 4.55 |
| C154 | V11I, E31A, D37A, C38A, K53A, C76A, C127A | 2.12 |
| C155 | V11I, C38A, D40A, K53A, C76A, C127A | 0.74 |
| C156 | V11I, C38A, N41A, K53A, C76A, C127A | 18.47 |
| C157 | V11I, C38A, K53A, C76A, C127A, D132A | 13.70 |
| C158 | V11I, C38A, K53A, C76A, G108A, C127A | 1.24 |
| C159 | V11I, C38A, K53A, C76A, H109A, C127A | 0.55 |
| C160 | V11I, C38A, K53A, C76A, D110A, C127A | 0.71 |
| C161 | K8R, V11I, C38A, C76A, Q103E, C127A | 0.06 |
| C162 | K8E, V11I, C38A, C76A, Q103R, C127A | 0.85 |
| C163 | V11I, C38A, C76A, Q103K, C127A | 0.05 |
| C164 | V11I, C38A, S55H, C76A, C127A | 0.08 |
| C165 | V11I, C38A, S55R, C76A, C127A | 0.15 |
| C166 | V11I, C38A, S55T, C76A, C127A | 0.02 |
| C167 | V11I, C38A, C76A, S105I, C127A | 0.04 |
| C168 | V11I, C38A, C76A, S105K, C127A | 0.05 |
| C174 | K8L, E6K, V11I, C38A, K53A, T63A, C76A, C127A | 0.14 |
| C176 | E6K, V11I, C38A, I49M, K53A, T63A, C76A, C127A | 25.84 |
| C177 | E6K, V11I, C38A, I49R, K53A, T63A, C76A, C127A | >2800 |
| C178 | E6K, V11I, C38A, K53A, T63A, C76A, Q103R, C127A | >2800 |
| C179 | E6K, K8E, V11I, C38A, K53A, T63A, C76A, Q103R, | >2800 |
| | C127A | |
| C180 | E6K, V11I, C38A, K53A, T63A, C76A, C127A, V153R | |
| C181 | E6K, V11I, C38A, K53A, T63A, C76A, C127A, V153E | >2800 |
| C182 | E6K, V11I, C38A, K53A, T63A, C76A, C127A, V153Y | >2800 |
| C183 | E6K, V11I, C38A, M51G, K53A, T63A, C76A, C127A | >2800 |
| C184 | E6R, V11I, C38A, K53A, T63A, C76A, C127A | 5.46 |
| C140 | E6K, V11I, C38A, K53A, T63A, C76A, C127A | >2800 |
| C141 | E6K, V11I, C38A, K53A, T63A, C76A, C127A | >2800 |
| C142 | Y1M, E6K, V11I, C38A, K53A, T63A, C76A, C127A | 2.25 |
| C192 | E6K, V11I, C38A, M51G, K53A, T63A, C76A, C127A | >2800 |
| 62 | E6K, C38A, K53A, C127A | 69.62 |
| 60 | E6K, C38Q, K53A | 24.8 |
| 59 | E6K, C38A, K53A | 35.95 |

10C—IFNγ Stimulation and IL-18BP Inhibition Assay—The experiments described in supra for determination of IFNγ stimulation in NK92 cells (and inhibition by IL-18 BP) were performed substantially as described on modified IL-18 polypeptides in order to assess their activities and their suitability for incorporation into activatable immunocytokine compositions. Results are shown in the table below.

| SEQ ID NO: or | | IC₅₀ | EC₅₀ |
|---|---|---|---|
| Composition ID | Sequence modifications | (nM) | (nM) |
| 1 | Native sequence | 1.47 | 0.276 |
| 34 | E06K, K53A, S55A | 229 | 0.824 |
| 35 | Y01G, F02A, E06K, M51G, K53A, D54A, S55A, T63A | >55.0 | >55.0 |
| 36 | K53A | 27.3 | 0.444 |
| 37 | S55A | 4.46 | 0.108 |
| 38 | E06K | 7.79 | 0.0567 |
| 39 | E06K, K53A | >703 | 0.0192 |
| 40 | E06K, S55A | 15 | 0.067 |
| 41 | K53A, S55A | 37.3 | 1.58 |
| 42 | E06K, K53A, S55A, T63A | 1060 | 0.144 |
| 43 | E06K, K53A, S55A, Y01G | 27.8 | 6.12 |
| 44 | E06K, K53A, S55A, F02A | NT | >1000 |
| 45 | E06K, K53A, S55A, D54A | NT | 30 |
| 46 | E06K, K53A, S55A, M51G | 0.189 | 7.4 |
| 47 | C38S, C68S, C76S, C127S | 0.444 | 0.115 |
| 48 | C38S, C68S, C76S, C127S, K70C | 0.114 | 0.488 |
| 49 | E06K, K53A, S55A, C38S, C68S, C76S, C127S, K70C | NT | 58.5 |
| 50 | E06K, K53A, T63A | >1000 | 0.0268 |
| 51 | T63A | 0.239 | 0.449 |
| 52 | E06K, T63A | 47.1 | 0.011 |
| 53 | K53A, T63A | 18.2 | 0.155 |
| 54 | E06K, K53A, C38S, C68S, C76S, C127S, K70C | 23.5 | 0.962 |
| 55 | K53A, T63A, C38S, C68S, C76S, C127S, K70C | >1000 | 17.2 |
| 6 | E6K, V11I, C38A, K53A, T63A, C68A, C76A, | 5.847 | 1.366 |
| | C127A, D98C | | |
| 5 | E6K, V11I, C38A, K53A, T63A, C68A, C76A, | 62.37 | 0.075 |
| | C127A, M86C | | |
| 9 | E6K, C38A, K53A, C68A, D98C | 960.8 | 0.069 |
| 4 | E6K, C38A, K53A, C68A, M86C | 396.3 | 0.022 |
| 62 | E6K, C38A, K53A, C127A | 283.6 | 0.026 |
| 60 | E6K, C38Q, K53A | 780.5 | 0.006 |
| 59 | E6K, C38A, K53A | 653.5 | 0.015 |
| 57 | E6K, C38S, K53A | 146.2 | 0.045 |
| C143 | V11I, C38A, K53A, C76A, C127A | 1.625 | 0.138 |
| C144 | V11I, C38A, K53A, T63A, C76A, C127A | 7.522 | 0.012 |
| C145 | V11I, C38A, K53A, S55A, C76A, C127A | 10.24 | 0.087 |
| C146 | V11I, C38A, M51G, K53A, C76A, C127A | 732.9 | 0.037 |
| C147 | V11I, C38A, K53A, D54A, C76A, C127A | 47.63 | 0.079 |
| C148 | F2A, V11I, C38A, K53A, C76A, C127A | 5.055 | 0.256 |
| C149 | V11I, E31A, C38A, K53A, C76A, C127A | 1.167 | 0.187 |
| C150 | V11I, T34A, C38A, K53A, C76A, C127A | 21.27 | 0.015 |
| C151 | V11I, D35A, C38A, K53A, C76A, C127A | 3.622 | 0.061 |
| C152 | V11I, S36A, C38A, K53A, C76A, C127A | 7.85 | 0.033 |
| C153 | V11I, D37A, C38A, K53A, C76A, C127A | 2.222 | 0.175 |
| C154 | V11I, E31A, D37A, C38A, K53A, C76A, C127A | 3.709 | 0.062 |
| C155 | V11I, C38A, D40A, K53A, C76A, C127A | 3.233 | 0.067 |
| C156 | V11I, C38A, N41A, K53A, C76A, C127A | 0.681 | 0.558 |
| C157 | V11I, C38A, K53A, C76A, C127A, D132A | 6.082 | 0.056 |
| C158 | V11I, C38A, K53A, C76A, G108A, C127A | 3.981 | 0.073 |
| C159 | V11I, C38A, K53A, C76A, H109A, C127A | 1.807 | 0.123 |
| C160 | V11I, C38A, K53A, C76A, D110A, C127A | 3.181 | 0.028 |
| C161 | K8R, V11I, C38A, C76A, Q103E, C127A | 1.073 | 0.057 |
| C162 | K8E, V11I, C38A, C76A, Q103R, C127A | 7.292 | 0.061 |
| C163 | V11I, C38A, C76A, Q103K, C127A | 0.823 | 0.093 |
| C164 | V11I, C38A, S55H, C76A, C127A | 0.456 | 0.414 |
| C165 | V11I, C38A, S55R, C76A, C127A | 0.885 | 0.176 |
| C166 | V11I, C38A, S55T, C76A, C127A | 0.44 | 0.098 |
| C167 | V11I, C38A, C76A, S105I, C127A | 0.809 | 0.103 |
| C168 | V11I, C38A, C76A, S105K, C127A | 0.176 | 0.098 |

Although the present disclosure and its advantages have been described in detail, it should be understood that various changes, substitutions and alterations can be made herein without departing from the spirit and scope of the disclosure as defined in the appended claims.

1. An activatable immunocytokine, comprising:
an antibody or antigen binding fragment thereof;
an activatable IL-18 (Act-IL-18) polypeptide, comprising:
an artificial polypeptide attached to an IL-18 polypeptide, wherein the artificial polypeptide comprises a protease cleavage site, wherein cleavage at the protease cleavage site enhances an IL-18 related activity of the activatable immunocytokine; and
a linker comprising:
a first point of attachment to the antibody or antigen binding fragment thereof; and
a second point of attachment to the Act-IL-18 polypeptide.

2. (canceled)

3. (canceled)

4. The activatable immunocytokine of claim 1, wherein the protease is selected from: kallikrein, thrombin, chymase, carboxypeptidase A, an elastase, proteinase 3 (PR-3), granzyme M, urokinase plasminogen activator (uPA), a calpain, a matrix metalloproteinase (MMP), a disintegrin and metalloproteinase (ADAM), a fibroblast activation protein alpha (FAP), a matriptase, a plasminogen activator, a cathepsin, a caspase, a tryptase, and a tumor cell surface protease.

5. (canceled)

6. The activatable immunocytokine of claim 1, further comprising a protease recognition sequence comprising the protease cleavage site, wherein the protease recognition sequence comprises the amino acid sequence set forth in any one of SEQ ID NOs: 646, 647, 648, 649, 650, 651, 652, 653, 657, 658, or 659.

7. (canceled)

8. The activatable immunocytokine of claim 1, wherein the artificial polypeptide comprises multiple protease cleavage sites.

9-14. (canceled)

15. The activatable immunocytokine of claim 1, further comprising a blocking moiety, wherein the blocking moiety comprises an IL-18 propeptide or a portion thereof.

16. The activatable immunocytokine of claim 15, wherein the IL-18 propeptide comprises an amino acid sequence having at least 80%, 85%, 90%, 95%, 96%, 97%, 98%, 99%, or 100% sequence identity to the sequence set forth in SEQ ID NO: 89, SEQ ID NO: 91, SEQ ID NO: 96, or SEQ ID NO: 97.

17-28. (canceled)

29. The activatable immunocytokine of claim 1, wherein the IL-18 polypeptide comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1.

30-35. (canceled)

36. The activatable immunocytokine of claim 1, wherein the IL-18 polypeptide comprises an amino acid sequence set forth in any one of SEQ ID Nos: 2-67 or 79-86.

37-43. (canceled)

44. The activatable immunocytokine of claim 1, wherein the linker comprises a polymer.

45. (canceled)

46. (canceled)

47. The immunocytokine composition of claim 44, wherein the polymer comprises poly(ethylene glycol).

48. (canceled)

49. (canceled)

50. (canceled)

51. The activatable immunocytokine of claim 1, wherein the second point of attachment is at residue 68 of the IL-18 polypeptide, wherein residue position numbering is based on SEQ ID NO: 1 as a reference sequence.

52. The activatable immunocytokine of claim 1, wherein the first point of attachment is at an amino acid residue in an Fc region of the antibody or antigen binding fragment, wherein the first point of attachment is at a position of a K246 amino acid residue, a K248 amino acid residue, a K288 amino acid residue, a K290 amino acid residue, or a K317 amino acid residue of the Fc region (Eu numbering).

53. (canceled)

54. (canceled)

55. The activatable immunocytokine of claim 1, wherein the antibody or antigen binding fragment thereof is a monoclonal antibody.

56. (canceled)

57. (canceled)

58. The activatable immunocytokine of claim 1, wherein the antibody or antigen binding fragment thereof comprises an IgG1 or an IgG4.

59. (canceled)

60. (canceled)

61. The activatable immunocytokine of claim 1, wherein the antibody or antigen binding fragment thereof binds to PD-1.

62. (canceled)

63. The activatable immunocytokine of claim 61, wherein the antibody or antigen binding fragment thereof comprises LZM-009.

64-68. (canceled)

69. An activatable IL-18 (Act-IL-18) polypeptide having a structure
BM-CS-IL-18;
BM-LP₁-CS-IL-18;
BM-LP₁-CS-LP₂-IL-18; or
BM-CS-LP₁-IL-18;
wherein IL-18 is an IL-18 polypeptide, CS is a moiety comprising a specific cleavage site, LP₁ is a first linking peptide, LP₂ is a second linking peptide, and BM is a blocking moiety, and wherein the orientations are shown in an N-terminal to C-terminal order, preferably wherein the Act-IL-18 polypeptide as a structure BM-CS-IL-18.

70. (canceled)

71. The Act-IL-18 polypeptide of claim 69, wherein the blocking moiety comprises an IL-18 propeptide comprising an amino acid sequence having at least 80%, 85%, 90%, 95%, 96%, 97%, 98%, 99%, or 100% sequence identity to the sequence set forth in SEQ ID NO: 89, SEQ ID NO: 91, SEQ ID NO: 96, or SEQ ID NO: 97.

72. (canceled)

73. (canceled)

74. The Act-IL-18 polypeptide of claim 69, wherein the IL-18 polypeptide comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1-67 or 79-86.

75-78. (canceled)

79. A method of treating cancer in a subject in need thereof, comprising:
administering to the subject a pharmaceutically effective amount of an activatable immunocytokine, wherein the activatable immunocytokine comprises:
an antibody or antigen binding fragment thereof specific for an immune associated antigen;
an activatable IL-18 (Act-IL-18) polypeptide, comprising:
an artificial polypeptide attached to an IL-18 polypeptide, wherein the artificial polypeptide comprises a protease cleavage site, wherein cleavage at the protease cleavage site enhances an IL-18 related activity of the activatable immunocytokine; and
a linker comprising:
a first point of attachment to the antibody or antigen binding fragment thereof; and
a second point of attachment to the Act-IL-18 polypeptide.

80-86. (canceled)
